(19) United States
(12) Patent Application Publication
(10) Pub. No.: US 2026/0335402 A1
(43) Pub. Date: Oct. 29, 2026

(54) COMPOSITIONS AND METHODS FOR TREATMENT AND PREVENTION OF NEURODEGENERATIVE DISEASES AND DISORDERS

(71) Applicant: The Trustees of the University of Pennsylvania, Philadelphia, PA (US)

(72) Inventors: Xiaolu Yang, Wynnewood, PA (US); Ruifang Wang, Philadelphia, PA (US)

(21) Appl. No.: 19/486,240

(22) PCT Filed: May 23, 2024
(86) PCT No.: PCT/US2024/030830

§ 371 Date: Nov. 20, 2025

Related U.S. Application Data
(60) Provisional application No. 63/503,835, filed on May 23, 2023.

(51) Int. Cl. A61K 38/17; A61K 48/00; A61P 25/28

(52) U.S. Cl. CPC A61K 38/1709; A61K 48/0033; A61K 48/005; A61P 25/28

(57) ABSTRACT

The present invention relates to compositions and methods for promoting the removal of misfolded proteins and protein aggregates. The compositions and methods may be used to treat or prevent a neurodegenerative disease or disorder associated with misfolded proteins or protein aggregates. In various embodiments, the compositions and methods relate to activators of one or more TRIM proteins.

### CROSS-REFERENCE TO RELATED APPLICATIONS

This application claims priority to U.S. Provisional Application No. 63/503,835, filed on May 23, 2023, incorporated herein by reference in its entirety.

### STATEMENT REGARDING FEDERALLY SPONSORED RESEARCH OR DEVELOPMENT

This invention was made with government support under TR001878 and CA243520 awarded by the National Institutes of Health. The government has certain rights in the invention.

### BACKGROUND OF THE INVENTION

Neurodegenerative diseases are pathologically and genetically linked to protein misfolding and aggregation and the ensuing loss of neurons (F. Chiti, C. M. Dobson, Annu Rev Biochem 75, 333-366, 2006). These diseases-including Alzheimer's disease (AD), Parkinson's disease (PD), amyotrophic lateral sclerosis (ALS), Huntington's diseases (HD), spinocerebellar ataxias (SCAs)—have become a major threat to human health and welfare in the modern world as the population ages. Yet, there are no effective treatments for any of them.

The conversion of the microtubule-associated protein tau from a soluble, monomeric state to a hyperphosphorylated and filamentous state is associated with many neurodegenerative diseases, collectively referred to as tauopathies (V. M. Lee, M. Goedert, J. Q. Trojanowski, Annu Rev Neurosci 24, 1121-1159, 2001; M. G. Spillantini, M. Goedert, Lancet Neurol 12, 609-622, 2013; J. Gotz, G. Halliday, R. M. Nisbet, Annu Rev Pathol 14, 239-261, 2019) These diseases include AD, Pick's disease (PiD), frontal temporal dementia (FTD), chronic traumatic encephalopathy (CTE), corticobasal degeneration (CBD), frontotemporal dementia and parkinsonism linked to chromosome 17 (FTDP-17), progressive supranuclear palsy (PSP), primary age-related tauopathy (PART), argyrophilic grain disease (AGD), and others. Among them, Alzheimer's disease (AD) is the most common form of dementia (C. L. Masters et al., Nat Rev Dis Primers 1, 15056, 2015) (D. S. Knopman et al., Nat Rev Dis Primers 7, 33, 2021). The number of AD patients in the US is currently estimated to be 6 million, and is projected to reach nearly 13 million by 2050 (Alzheimers Dement, 2020). There is no treatment that can effectively prevent or slow the progression of AD and other tauopathies, and the development of a meaningful therapy is hindered by the limited knowledge of PQC systems, especially those in animal cells.

Thus, there is a need in the art for improved compositions and methods for treating neurodegenerative diseases with. This invention satisfies this unmet need.

### SUMMARY OF THE INVENTION

In some embodiments, the present invention provides a composition for treating or preventing a disease or disorder associated with aggregation of one or more selected from the group consisting of tau, α-Synuclein (α-Syn), superoxide dismutase 1 (SOD1), TAR DNA binding protein 43 (TDP-43), FUsed in Sarcoma/Translocated in LipoSarcoma (FUS/TLS), ataxin 1, huntingtin (Htt), Aβ42, and heterogeneous ribonucleoprotein A1 (hnRNPA1), the composition comprising an activator of the level or activity of one or more tripartite motif (TRIM) proteins, wherein the one or more TRIM proteins are one or more selected from the group consisting of human TRIM10, TRIM2, TRIM3, TRIM4, TRIM5, TRIM9, TRIM11, TRIM17, TRIM18, TRIM19, TRIM21, TRIM24, TRIM26, TRIM29, TRIM30, TRIM31, TRIM34, TRIM36, TRIM37, TRIM39, TRIM40, TRIM41, TRIM42, TRIM43, TRIM46, TRIM47, TRIM48, TRIM49, TRIM52, TRIM54, TRIM55, TRIM56, TRIM58, TRIM63, TRIM64, TRIM65, TRIM68, TRIM69, TRIM70, TRIM71, TRIM73, and TRIM77. In some embodiments, the activator is one or more selected from the group consisting of a chemical compound, a protein, a peptide, a peptidomimetic, an antibody, a ribozyme, a small molecule chemical compound, a nucleic acid, a vector, an antisense nucleic acid, siRNA, shRNA, and a guide RNA.

In some embodiments, the disease or disorder is associated with aggregation of tau; and the one or more TRIM proteins are selected from the group consisting of TRIM10, TRIM2, TRIM3, TRIM4, TRIM5, TRIM9, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70.

In some embodiments, the disease or disorder is associated with aggregation of α-Syn; and the one or more TRIM proteins are selected from the group consisting of TRIM10, TRIM2, TRIM3, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73.

In some embodiments, the disease or disorder is associated with aggregation of SOD1; and the one or more TRIM proteins are selected from the group consisting of TRIM10, TRIM11, TRIM24, TRIM36, and TRIM58.

In some embodiments, the disease or disorder is associated with aggregation of TDP-43; and the one or more TRIM proteins are selected from the group consisting of TRIM10, TRIM11, TRIM17, TRIM36, TRIM 37, TRIM 40, TRIM49, and TRIM55.

In some embodiments, the disease or disorder is associated with aggregation of FUS/TLS, ataxin 1, Htt, AB42, and hnRNPA1; and the one or more TRIM protein is TRIM10.

In some embodiments, the activator of the TRIM protein is a peptide comprising the amino acid sequence of the TRIM protein or a functional variant thereof.

In some embodiments, the activator of the TRIM protein is a nucleic acid encoding the TRIM protein or a functional variant thereof.

In some embodiments, the activator of the TRIM protein is a vector comprising a nucleic acid encoding the TRIM protein or a functional variant thereof. In some embodiments, the vector is a virus. In some embodiments, the virus is an adeno-associated virus.

In some embodiments, the present invention provides a method of treating or preventing a neurodegenerative disease or disorder associated with aggregation of one or more proteins selected from the group consisting of tau, α-Syn, SOD1, TDP-43, FUS/TLS, ataxin 1, Htt, AB42, and hnRNPA1 comprising administering to the subject a composition according to the present invention.

In some embodiments, the neurodegenerative disease or disorder associated with tau is selected from the group consisting of Alzheimer's disease, frontotemporal lobar degeneration (FTLD-tau), progressive supranuclear palsy (PSP), corticobasal degeneration (CBD), argyrophilic grain disease (AGD), frontotemporal dementia and parkinsonism linked to chromosome 17 (FTDP-17), vacuolar tauopathy, Lytico-bodig disease, globular glial tauopathy (GGT), ageing-related tau astrogliopathy (ARTAG), Pick's disease, and amyotrophic lateral sclerosis (ALS), primary age-related tauopathy (PART), tangle only dementia (TOD), chronic traumatic encephalopathy (CTE), anti-IgLON5-related tauopathy, Guadeloupean parkinsonism, multisystem proteinopathy (MSP), Nodding Syndrome (NS), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis; and the activator of the one or more TRIM proteins is an activator of one or more selected from the group consisting of TRIM10, TRIM11, and TRIM55.

In some embodiments, administering the composition is effective in one or more selected from the group consisting of: a) reducing tau aggregates by at least about 60%; b) reducing the ratio of insoluble tau to soluble tau by at least about 50%; and c) reducing tau aggregates by about 90% six to eight days after administration of the composition.

In some embodiments, the neurodegenerative disease or disorder associated with α-Syn is selected from the group consisting of Parkinson's disease (PD), dementia with Lewy bodies (DLB), multiple system atrophy (MSA), Shy-Drager syndrome, striatonigral degeneration, olivopontocerebellar atrophy, Hallervorden-Spatz syndrome, REM sleep behavior disorder (RPD), and Alzheimer's disease with amygdala restricted Lewy bodies (AD/ALB); and the activator of one or more TRIM proteins is an activator of one or more selected from the group consisting of TRIM10, TRIM36, TRIM55, and TRIM68.

In some embodiments, the neurodegenerative disease or disorder associated with SOD1 is selected from the group consisting of amyotrophic lateral sclerosis (ALS) and Parkinson's disease (PD); and the activator of one or more TRIM proteins is an activator of one or more selected from the group consisting of TRIM10, TRIM11, TRIM24, TRIM36, and TRIM58.

In some embodiments, the neurodegenerative disease or disorder associated with TDP-43 is selected from the group consisting of frontotemporal dementia (FTD), frontotemporal lobar degeneration (FTLD-TDP), multiple system proteinopathy (MSP), Perry disease, facial onset sensory and motor neuronopathy (FOSMN), Alzheimer's disease (AD), cerebral age-related TDP-43 with sclerosis (CARTS), limbic-predominant age-related TDP-43 encephalopathy (LATE), sporadic inclusion body myositis (sIBM), chronic traumatic encephalopathy (CTE), primary lateral sclerosis (PLS), progressive muscular atrophy (PMA), Guam Parkinson-dementia complex (G-PDC), Guam amyotrophic lateral sclerosis (G-ALS); Parkinson's disease (PD), and Huntington's disease (HD); and the activator of one or more TRIM proteins is an activator of one or more selected from the group consisting of TRIM10, TRIM11, TRIM17, TRIM36, TRIM37, TRIM40, TRIM49, and TRIM55.

In some embodiments, the composition is administered to the subject in their cerebrospinal fluid (CSF). In some embodiments, the composition is administered by intracerebroventricular (ICV) injection.

In some embodiments, the composition is administered to the subject before onset of symptoms of the disease or disorder. In some embodiments, the composition is administered to the subject after onset of symptoms of the disease or disorder.

In some embodiments, the method further comprises administering one or more additional therapeutic agents.

### BRIEF DESCRIPTION OF THE DRAWINGS

The following detailed description of embodiments of the invention will be better understood when read in conjunction with the appended drawings. It should be understood that the invention is not limited to the precise arrangements and instrumentalities of the embodiments shown in the drawings.

FIG. 1, comprising FIG. 1A through FIG. 1I, depicts representative results of screening TRIM proteins. FIG. 1A depicts a representative image of Western blots for a variety of TRIM proteins in HEK293T cells transfected with GFP-tau P301L and a control vector (−) or a vector for the indicated TRIM. Cells were lysed in NP-40-containing buffer, separated into soluble supernatant (SN) and insoluble pellet (PE) fractions by sedimentation. These fractions (top two rows) and whole cell lysates (WCL, bottom two rows) were analyzed. The expected full-length TRIM bands are indicated by arrowheads. TRIM proteins that substantially reduced insoluble GFP-tau P301L species are boxed. FIG. 1B depicts representative images of Western blots for a variety of TRIM proteins in HEK293T cells as in FIG. 1A. FIG. 1C depicts relative GFP-tau P301L (PE)/HSP90 ratios in HEK293T cells for the given TRIM proteins depicted in FIG. 1A and FIG. 1B. FIG. 1D depicts a representative Western blot of the TRIM proteins that substantially reduced insoluble GFP-tau P301L in FIG. 1A and FIG. 1B expressed in SH-SY5Y cells. FIG. 1E depicts relative GFP-tau P301L (PE) ratios in SH-SY5Y cells in the presence of the indicated TRIM proteins depicted in FIG. 1D. FIG. 1F depicts a representative Western blot of TRIM expression in the indicated TRIM-knockout HEK293T cells transfected with GFP-tau P301L. FIG. 1G depicts relative GFP-tau P301L (PE)/HSP90 ratios of the indicated TRIM proteins depicted in FIG. 1F. FIG. 1H depicts a representative Western blot of TRIM expression in the indicated TRIM-knockdown N2a cells transfected with GFP-tau P301L. FIG. 1I depicts relative GFP-tau P301L (PE)/HSP90 ratios of the indicated TRIM proteins depicted in FIG. 1H. For FIG. 1C, FIG. 1E, FIG. 1G, and FIG. 1I, data are mean±SD; n=3; *, p<0.05; **, p<0.01; ***, p<0.001; unpaired Student's t test.

FIG. 2, comprising FIG. 2A through FIG. 2J, depicts representative results of downregulation of TRIM11 in sporadic Alzheimer's disease (AD) brains. FIG. 2A depicts a representative table summarizing demographic data of control and AD subjects used in this study. FIG. 2B depicts representative images of Western blots of postmortem frontal cortices gray matter from 14 control and 23 AD individuals. FIG. 2C depicts relative levels of TRIM10 and TRIM55 as shown in FIG. 2B. FIG. 2D depicts relative levels of TRIM11 as shown in FIG. 2B. The #1 control and #1 AD samples were used in each blot for comparison between blots. p-Tau species were modified at residues S202 and T205 (reactive to AT8); Ser262; T231 (reactive to AT180); and S396 and S404 (reactive to PHF-1). FIG. 2E depicts representative IHC images of TRIM11 and AT8-reactive p-tau in frontal cortices (scale bar, 50 μm). FIG. 2F depicts representative quantification of TRIM11 and AT8 signals as shown in FIG. 2E (mean±SD, n=4). FIG. 2G depicts representative images of TRIM11 and NeuN staining in frontal cortices of control and AD samples (scale bar, 10 μm). FIG. 2H depicts quantification of TRIM11 signal, as shown in FIG. 2G, normalized to numbers of neurons (mean±SD, n=4). Individual neurons are indicated by white arrows. FIG. 2I depicts representative negative correlation between TRIM11 expression and levels of different p-tau species among AD and control tissues. FIG. 2J depicts representative negative correlation between TRIM11 expression and levels of different p-tau species among AD tissues only. For FIG. 2E and FIG. 2H, data are presented as mean±SD; n=4; *, p<0.05; ns, not significant; unpaired Student's t test. For FIG. 2I and FIG. 2J, the r and p values of Pearson correlation coefficient are shown.

FIG. 3, comprising FIG. 3A through FIG. 3M, depicts representative results of TRIM11 targeting tau for proteasomal degradation. FIG. 3A depicts a representative image of a Western Blot of tau or tau P301L expressed in HEK293T together with increasing amounts of TRIM11. Cells were lysed in sarkosyl-containing buffer and analyzed by Western blot. FIG. 3B depicts representative images of Western Blots for a CHX chase assay of GFP-tau-P301L turnover in HEK293T cells in the presence or absence of TRIM11. To better compare the half-life of GFP-tau-P301L under different conditions, the blots on the left and their corresponding ones on the right were exposed for different times to achieve similar band intensity at time 0. Relative GFP-tau p301L/actin ratios are shown in FIG. 13A. FIG. 3C depicts a representative Western Blot of QBI293/tau P301L-GFP cells stably expressing mCherry or mCherry plus TRIM11 cultured in medium containing doxycycline (Dox) to induce tau P301L-GFP expression and then in medium without Dox. Turnover of preexisting tau P301L-GFP over time after Dox withdrawal were analyzed by western blot. Quantification is shown in FIG. 13B. FIG. 3D depicts representative blots for GFP-tau expressed alone or together with Flag-TRIM11 in the HEK293T cells and treated with or without okadaic acid (OA, 100 nM). To achieve comparable levels of GFP-tau, the amount of GFP-tau plasmid was increased when expressed together with TRIM11 (FIG. 13E). Quantification is shown in FIG. 13F through FIG. 13I. FIG. 3E depicts representative images of a BiFC assay for TRIM11-VN and tau-VC expressed in HEK293T cells (scale bar, 100 μm). Quantification of BiFC signals is depicted in FIG. 14D. FIG. 3F depicts representative images of Western blots of a co-IP assay of interaction between Flag-TRIM11 and GFP-tau in HEK293T cells treated with or without OA (100 nM). FIG. 3G depicts representative images of Western blots when GST or GST-TRIM11 immobilized on beads were incubated with 6×His-GFP-tau or 6×His-GFP-tau P301L. The pulldown and input samples were analyzed by western blot and/or Coomassie blue staining. FIG. 3H depicts representative images demonstrating localization of endogenous TRIM11 and tau in SH-SY5Y cells treated with or without OA (100 nM) (scale bar, 10 μm). FIG. 3I depicts quantification of colocalization analyzed by Manders' colocalization coefficient from FIG. 3H (n=6). Individual TRIM11 and tau images are shown in FIG. 15A. FIG. 3J depicts representative images of a PLA assay of endogenous TRIM11-tau interaction in SH-SY5Y cells treated with or without OA (100 nM) (scale bar, 10 μm). FIG. 3K depicts quantification of PLA signals from FIG. 3J (n=10). Individual PLA and DAPI images are shown in FIG. 15D. FIG. 3L depicts representative images of a Western blot of purified recombinant Flag-TRIM11 or Flag-TRIM11^{2EA} incubated with GST-tau P301L in the presence or absence of ATP as indicated, together with SUMO E1, E2, and 6×His-SUMO2. The reaction mixtures were denatured and analyzed for GST-tau P301L SUMOylation by IP (d-IP). FIG. 3M depicts representative images of a Western blot demonstrating levels of GFP-tau-P301L in HEK293T cells in the presence of increasing amounts of TRIM11 or TRIM112EA. For FIG. 3I and FIG. 3K, data are presented as mean±SD; ***, p<0.001; unpaired Student's t test.

FIG. 4, comprising FIG. 4A through FIG. 4P, depicts representative results of testing TRIM11 as both a molecular chaperone and a disaggregase for tau, increasing its solubility. FIG. 4A depicts representative images of a Western blot demonstrating that TRIM11 increases tau solubility in cells. HEK293T cells were transfected with different amounts of GFP-tau plasmid so that its levels were comparable in the presence or absence of TRIM11. Cells were treated with MG132 (10 μM) or NH₄Cl (20 μM) as indicated. FIG. 4B depicts representative images of HEK293T cells transfected with tau-VN, tau-VC, and TRIM11 as indicated (scale bar, 100 μm). FIG. 4C depicts the relative fluorescence signals (top) and protein expression (bottom) as shown in FIG. 4B. FIG. 4D depicts representative images of HEK293/RD (LM)-YFP cells transfected with empty vector (EV) or TRIM11 and treated with or without tau PFFs. Intracellular tau inclusions were analyzed by IF (scale bar, 100 μm). FIG. 4E depicts quantification of tau inclusions shown in FIG. 4D (n=12). FIG. 4F depicts representative images of QBI293/tau P301L-GFP cells stably expressing mCherry or mCherry plus TRIM11 treated with Dox to induce tau P301L expression, and incubated with or without tau PFFs. Tau inclusions in cells were analyzed by IF (scale bar, 50 μm). FIG. 4G depicts quantification of tau inclusions shown in FIG. 4F (n=8 to 10). FIG. 4H depicts representative images of Western blots of insoluble tau species. FIG. 4I depicts representative formation of tau amyloid fibrils when tau protein (10 μM) was incubated with heparin (30 μM) in the presence of GST (1 μM) or GST-TRIM11 (0.25, 0.5, or 1 μM) and examined by ThT binding. FIG. 4J depicts representative images of Western blots demonstrating formation of tau amyloid fibrils as in FIG. 4I detected by sedimentation. FIG. 4K depicts representative images of formation of tau amyloid fibrils as in FIG. 4I by detected by electron microscopy (scale bar, 500 nm). FIG. 4L depicts representative formation of tau amyloid fibrils when Tau P301L protein was incubated with buffer, GST (1 μM), or GST-TRIM11 (0.25, 0.5, or 1 μM) and examined by ThT binding. FIG. 4M depicts representative images of a Western blot demonstrating generation of high molecular weight species of tau, as shown in FIG. 4L. FIG. 4N depicts representative results of re-formed tau PFFs (1 μM) treated with GST or GST-TRIM11 at the indicated concentrations analyzed by ThT-binding (n=3). FIG. 4O depicts representative images of Western blot results of re-formed tau PFFs treated as in FIG. 4N detected by sedimentation. FIG. 4P depicts representative images depicting results of re-formed tau PFFs treated as in FIG. 4N detected by electron microscopy (scale bar, 200 nm). For FIG. 4C, FIG. 4E, FIG. 4G, and FIG. 4N, data are presented as mean±SD; **, p<0.01; ***, p<0.001; unpaired Student's t test.

FIG. 5, comprising FIG. 5A through FIG. 5U, depicts representative results of testing TRIM11 for maintaining neural integrity and connectivity. FIG. 5A depicts representative images demonstrating localization of endogenous TRIM11 and tau in wild-type cortical neurons (Scale bar, 10 μm). Quantification of colocalization is shown in FIG. 19A. FIG. 5B depicts representative images of PS19 cortical neurons transduced with control or TRIM11 ASO #5 with tau aggregates detected with AT8 (scale bar, 10 μm). Cells were treated with myc-K18/P301L PFFs. FIG. 5C depicts representative quantification of intracellular tau aggregates shown in FIG. 5B (n=4). FIG. 5D depicts representative images of PS19 cortical neurons transduced with AAV9-GFP or AAV9-TRIM11 with tau aggregates detected with AT8 (scale bar, 50 μm). Cells were treated with myc-K18/P301L PFFs. FIG. 5E depicts representative quantification of intracellular tau aggregates shown in FIG. 5D (n=3). FIG. 5F depicts representative images of wild-type cortical neurons treated with control or TRIM11 ASO #5 analyzed for the expression of SYP (scale bar, 10 μm). FIG. 5G depicts quantification of SYP-reactive puncta shown in FIG. 5F (n=3). FIG. 5H depicts representative images of wild-type cortical neurons treated with control or TRIM11 ASO #5 analyzed for the expression of PSD95 (scale bar, 10 μm). FIG. 5I depicts quantification of PSD95-reactive puncta shown in FIG. 5H (n=3). FIG. 5J depicts representative images of wild-type cortical neurons treated with control or TRIM11 ASO #5 analyzed for the expression of NFL and MAP2 (scale bar, 10 μm). FIG. 5K depicts quantification of relative NFL intensity shown in FIG. 5J (n=3). FIG. 5L depicts quantification of dendrite length shown in FIG. 5J (n=3). Individual images are depicted in FIG. 12C. FIG. 5M depicts representative viability of wild-type cortical neurons treated with control ASO or the indicated TRIM11 ASOs for 14 days (n=3). FIG. 5N depicts representative images of wild-type cortical neurons transduced with AAV9-GFP or AAV9-TRIM11 (the latter being tagged with HA) stained with antibodies against SYP and HA (scale bar, 10 μm). FIG. 5O depicts quantification of SYP-puncta shown in FIG. 5N (n=3). FIG. 5P depicts representative images of wild-type cortical neurons as in FIG. 5N stained with antibodies against PSD95 and HA (scale bar, 10 μm). FIG. 5Q depicts quantification of PSD95-puncta shown in FIG. 5P (n=3). FIG. 5R depicts representative images of wild-type cortical neurons transduced with AAV9-GFP or AAV9-TRIM11 probed with antibodies against NFL and MAP2 (scale bar, 10 μm). FIG. 5S depicts quantification of relative NFL intensity normalized to neuronal cell number (n=3). FIG. 5T depicts quantification of relative MAP2 intensity normalized to neuronal cell number (n=3). FIG. 5U depicts representative viability of wild-type cortical neurons transduced with AAV9-GFP or AAV9-TRIM11 treated with or without tau PFFs (n=3). For FIG. 5C, FIG. 5E, FIG. 5G, FIG. 5I, FIG. 5K through FIG. 5M, FIG. 5O, FIG. 5Q, and FIG. 5S through FIG. 5U, data presented as mean±SD; *, p<0.05; **, p<0.01; ***, p<0.005; ns, not significant; unpaired Student's t test.

FIG. 6, comprising FIG. 6A through FIG. 6O, depicts representative results of testing TRIM11 protecting against tau pathology and cognitive/behavioral impairments in PS19 mice. FIG. 6A depicts a schematic representation of the study on the effect of TRIM11 on PS19 mice. Human TRIM11 was tagged with HA in AAV9 and was detected with an anti-HA antibody to distinguish it from the endogenous mouse TRIM11. FIG. 6B depicts representative images demonstrating TRIM11 reduces tau pathology in the hippocampus of PS19 mice. Brains of PS19 mice injected with AAV9-GFP or AAV9-TRIM11 were stained with AT8. Shown are representative images of the hippocampus and the CA1 region (left; scale bar: 0.2 mm) and relative AT8 intensity (right; n=6 mice). Additional images are presented in FIG. 21A. FIG. 6C depicts a representative image of Western blot analysis of total tau and p-tau species in hippocampi of mice injected with AAV9-GFP or AAV9-TRIM11. Each line indicates a single mouse. Quantification is depicted in FIG. 21B through FIG. 21D. FIG. 6D depicts representative images demonstrating that TRIM11 reduces astrogliosis. Brains of age-matched wild-type mice, or PS19 mice injected with AAV9-GFP or AAV9-TRIM11, were stained with anti-GFAP antibody. Shown are representative images of the hippocampus (scale bar, 0.2 mm). FIG. 6E depicts quantification of GFAP immunoreactive area in FIG. 6D (n=5 WT or 6 GFP- or TRIM11-injected mice). FIG. 6F depicts representative images demonstrating that TRIM11 reduces microgliosis. Brains of age-matched wild-type mice, or PS19 mice injected with AAV9-GFP or AAV9-TRIM11, were stained with anti-Iba1 antibody (scale bar, 0.2 mm). FIG. 6G depicts quantification of Iba1 immunoreactive area in FIG. 6F (n=5 WT or 6 GFP- or TRIM11-injected mice). Additional images are depicted in FIG. 21E and FIG. 21F. FIG. 6H depicts representative images demonstrating TRIM11 rescues dendritic degradation. Brains of wild-type mice, or PS19 mice injected with AAV9-GFP or AAV9-TRIM11, were stained with antibodies detecting MAP2. Shown are images of hippocampi (scale bar, 0.2 mm) FIG. 6I depicts quantification of MAP2-immunoreactivity in FIG. 6H (n=4 WT, 6 GFP-AAV, or 7 AAV-TRIM11 PS19 mice). FIG. 6J depicts representative images demonstrating TRIM11 rescues axonal degeneration. Brains of wild-type mice, or PS19 mice injected with AAV9-GFP or AAV9-TRIM11, were stained with antibodies detecting NFL (scale bar: 0.2 mm). FIG. 6K depicts quantification of NFL-immunoreactivity from FIG. 6J (n=4 WT, 6 GFP-AAV, or 7 AAV-TRIM11 PS19 mice). Additional images are depicted in FIG. 21G and FIG. 21H. FIG. 21L depicts representative images demonstrating that TRIM11 prevents neuronal loss. Brains of wild-type mice, or PS19 mice injected with AAV9-GFP or AAV9-TRIM11, were stained with antibodies detecting NeuN. Shown are representative images of the hippocampus (scale bar: 0.2 mm). FIG. 6M depicts quantification of NeuN-immunoreactivity from FIG. 6L (n=3 mice). Additional images are depicted in FIG. 21I. FIG. 6N depicts representative results demonstrating AAV9-TRIM11-injected mice show increased preference for novel objects (n=6 WT or GFP or 7 TRIM11 mice). FIG. 6O depicts representative results demonstrating that TRIM11 improves grip strength (n=4 WT, 5 GFP group, or 6 TRIM11). For FIG. 6B (left), FIG. 6E, FIG. 6G, FIG. 6I, FIG. 6K, and FIG. 6M through FIG. 6O, data presented as mean±SD; *, p<0.05; **, p<0.01; ***, p<0.005; ns, not significant; unpaired Student's t test.

FIG. 7, comprising FIG. 7A through FIG. 7J, depicts representative results of testing TRIM11 protection against PFFs-accelerated tau pathology and cognitive and behavioral impairments in PS19 mice. FIG. 7A depicts a schematic representation of the study on the effect of TRIM11 on PFFs-injected PS19 (PS19-PFFs) mice. FIG. 7B depicts representative results demonstrating TRIM11 reduces tau pathology. Brains of PS19 mice injected with AAV9-GFP or AAV9-TRIM11, together with tau PFFs, were stained with AT8. Representative images of the hippocampus and the CA1 region (left; scale bar, 0.2 mm) and relative AT8 intensity (right; n=6 mice) are shown. Additional images are depicted in FIG. 22A. FIG. 7C depicts representative images of Western blot analysis of total tau and p-tau species in hippocampi of mice injected with AAV9-GFP or AAV9-TRIM11, together with PFFs. Each line indicates a single mouse. Quantification is shown in FIG. 22B through FIG. 22D. FIG. 7D depicts representative results demonstrating that TRIM11 reduces astrogliosis. Brains of PS19 mice injected with AAV9-GFP or AAV9-TRIM11, together with PFFs, were stained with anti-GFAP antibody. Representative images of hippocampi (left; scale bar, 0.2 mm) and quantification of GFAP immunoreactive area (right, n=5 WT or 6 GFP- or TRIM11-injected PS19 mice) are shown. FIG. 7E depicts representative results demonstrating that TRIM11 reduces microgliosis. Brains of PS19 mice were treated as in FIG. 7D and stained with anti-Iba1 antibody. Representative images of hippocampi (left; scale bar, 0.2 mm) and quantification of Iba1 immunoreactive area (right, n=5 WT or 6 GFP- or TRIM11-injected PS19 mice) are shown. Additional images are shown in FIG. 22E and FIG. 22F. FIG. 7F depicts representative results demonstrating that TRIM11 maintains the ability of mice to recognize novel subject. Data are percentages of preference for the novel subject (n=9 WT or TRIM11 or 7 GFP mice). FIG. 7G depicts representative results demonstrating an increased alternation in Y-Maze test for AAV9-TRIM11-injected mice compared to AAV9-GFP-injected mice (n=9 WT or TRIM11 or 10 GFP mice). FIG. 7H depicts representative open field track plots demonstrating increased travel distance of AAV9-TRIM11-injected mice compared to AAV9-GFP-injected mice. FIG. 7I depicts quantification of distance traveled shown in FIG. 7H (n=9 WT or GFP or 10 TRIM11 mice). FIG. 7J depicts representative results demonstrating reduced freezing time of AAV9-TRIM11-injected mice compared to AAV9-GFP-injected mice (n=9 WT or GFP or 10 TRIM11 mice). For FIG. 7B (right), FIG. 7D (right), FIG. 7E (right), FIG. 7F, FIG. 7G, FIG. 7I, and FIG. 7J, data are presented at mean±SD; *, p<0.05; **, p<0.01; ***, p<0.005; ns, not significant; unpaired Student's t test.

FIG. 8, comprising FIG. 8A through FIG. 8Q, depicts representative results of testing TRIM11 ameliorating tau pathology and cognitive deficits of 3×Tg-AD mice. FIG. 8A depicts a schematic representation of bilateral hippocampus injection of 3×Tg-AD mice. FIG. 8B depicts representative results demonstrating intraparenchymal injection of AAV9-TRIM11 according to FIG. 8A reduces tau aggregates in the hippocampus of 3×Tg-AD mice. Representative images of hippocampi (left; scale bar, 0.2 mm) and quantification of tau aggregates (right; n=6 mice) are shown. FIG. 8C depicts representative images of Western blot analysis of total tau and p-tau species in hippocampi of treated as in FIG. 8A. Each line indicates a single mouse. Quantification is presented in FIG. 23B through FIG. 23D. FIG. 8D depicts representative results demonstrating that TRIM11 reduces astrogliosis in mice treated as in FIG. 8A. Shown are representative images (left; scale bar, 0.2 mm) and quantification (right; n=5 to 6 mice) of GFAP-immunoreactive areas. Additional Images are shown in FIG. 23E.

FIG. 8E depicts representative results demonstrating TRIM11 reduces microgliosis in mice treated as in FIG. 8A. Shown are representative images (left; scale bar, 0.2 mm) and quantification (right; n=5 to 6 mice) of Iba1-immunoreactive areas. Additional images are shown in FIG. 23F. FIG. 8F depicts representative results demonstrating that mice treated as in FIG. 8A with TRIM11 have increased time spent at novel objects (n=9 GFP or 13 TRIM11 mice). FIG. 8G depicts representative results demonstrating that mice treated as in FIG. 8A with TRIM11 display increased correct alternation in Y-maze trials (n=9 GFP or 14 TRIM11 mice). FIG. 8H depicts representative results demonstrating that mice treated as in FIG. 8A with TRIM11 have increased travel distance in open field trials (n=14 GFP or 17 TRIM11 mice). FIG. 8I depicts representative results demonstrating that mice treated as in FIG. 8A with TRIM11 have decreased freeze time in open field trials (n=14 GFP or 17 TRIM11 mice). FIG. 8J depicts a schematic representation of unilateral ICV injection of 3×Tg-AD mice. FIG. 8K depicts representative results demonstrating ICV injection of AAV9-TRIM11 according to FIG. 8J reduces tau aggregates in the hippocampus of 3×Tg-AD mice. Representative images of hippocampi (left; scale bar, 0.2 mm) and quantification of tau aggregates (right; n=6 mice) are shown. FIG. 8L depicts representative images of Western blot analysis of total tau and p-tau species in hippocampi of treated as in FIG. 8J. Each line indicates a single mouse. Quantification is presented in FIG. 24A through FIG. 24C. FIG. 8M depicts representative results demonstrating that TRIM11 reduces astrogliosis in mice treated as in FIG. 8J. Quantification of GFAP-immunoreactive areas is shown (n=5 to 6 mice). Additional images are shown in FIG. 24D. FIG. 8N depicts representative results demonstrating TRIM11 reduces microgliosis in mice treated as in FIG. 8J. Quantification of Iba1-immunoreactive areas is shown (n-5 to 6 mice). Additional images are shown in FIG. 24E. FIG. 8O depicts representative results demonstrating that mice treated as in FIG. 8J with TRIM11 have increased time spent at novel objects (n=6 GFP or 8 TRIM11 mice). FIG. 8P depicts representative results demonstrating that mice treated as in FIG. 8J with TRIM11 display increased correct alternation in Y-maze trials (n=7 mice). FIG. 8Q depicts representative results demonstrating that mice treated as in FIG. 8J with TRIM11 have increased travel distance in open field trials (n=7 mice). For FIG. 8B (right), FIG. 8D (right), FIG. 8E (right), FIG. 8F through FIG. 8I, FIG. 8K (right), and FIG. 8M through FIG. 8Q, data presented as mean±SD; *, p<0.05; **, p<0.01; ***, p<0.001; ns, not significant; unpaired Student's t test.

FIG. 9 depicts a schematic representation of structures of TRIM proteins. All TRIMs are human proteins except for TRIM12 and TRIM30, which are of mouse origin. TRIM53 (a pseudogene) and TRIM57 (the same as TRIM59) are not listed. ARF, ADP ribosylation factor-like; B, B-box; BR, bromodomain; CC, coiled-coil; COS, C-terminal subgroup one signature; FN3, fibronectin type 3; FIL, filamin-type immunoglobulin; MATH, meprin and tumor-necrosis factor receptor-associated factor homology; MID, midline; N-terminal, amino-terminal; PHD, plant homeodomain; PRY, SPRY-associated domain; R, RING finger; SPRY, SPIa and the ryanodine receptor domain; TM, transmembrane. Domains that are missing are indicated in parentheses. Not drawn to scale.

FIG. 10, comprising FIG. 10A through FIG. 10H, depicts representative results of forced expression of TRIM10, TRIM11, and TRIM55. FIG. 10A depicts representative relative GFP-tau P301L (SN)/HSP90 ratios in SH-SY5Y cells in the presence of the indicated TRIM proteins. Related to FIG. 1D and FIG. 1E. FIG. 10B depicts representative images of Western blot analysis of GFP-tau P301L transfected together with control vector or the indicated TRIMs in N2a cells. Cells were lysed in buffer containing NP40, separated into soluble supernatant (SN) and insoluble pellet (PE) fractions by sedimentation. FIG. 10C depicts relative tau/HSP90 ratios in the PE and SN fractions from FIG. 10B. FIG. 10D depicts representative images of Western blot analysis of N2 cells treated as in FIG. 10B with Sarkosyl buffer used in place of NP40 buffer. FIG. 10E depicts relative tau/HSP20 ratios in the PE and SN fractions from FIG. 10D. FIG. 10F depicts representative relative GFP-tau P301L mRNA levels in HEK293T cells in the presence of the indicated TRIM proteins. FIG. 10G depicts representative relative GFP-tau P301L mRNA levels in SH-SY5Y cells in the presence of the indicated TRIM proteins. FIG. 10H depicts representative relative GFP-tauP301L mRNA levels in N2a cells in the presence of the indicated TRIM proteins. For FIG. 10A, FIG. 10C, and FIG. 10E through FIG. 10H, data are mean±SEM; n=3; *, p<0.05; **, p<0.01; ***, p<0.001; ns, not significant; unpaired Student's t test.

FIG. 11, comprising FIG. 11A through FIG. 11H, depicts representative results of knockout and knockdown of TRIM10, TRIM11, or TRIM55. FIG. 11A depicts representative relative GFP-tau P301L ratios in the supernatant (SN) of the indicated TRIM-knockout HEK293T cells. FIG. 11B depicts representative relative GFP-tau P301L mRNA ratios of the indicated TRIM-knockout HEK293T cells. FIG. 11C depicts representative images of Western blots of N2a cells transfected with GFP-tau P301L and pretreated with control or TRIM10-siRNA. Cells were lysed in NP-40-containing buffer. FIG. 11D depicts representative images of Western blots of N2a cells as in FIG. 11C with TRIM11-siRNA in place of TRIM-10 siRNA. FIG. 11E depicts representative images of Western blots of N2a cells as in FIG. 11C with TRIM36-siRNA in place of TRIM10-siRNA. FIG. 11F depicts representative images of Western blots of N2a cells as in FIG. 11C with TRIM55-siRNA in place of TRIM10-siRNA. FIG. 11G depicts the relative GFP-tau P301L/HSP90 ratios in the pellet (PE) and supernatant (SN) fractions shown in FIG. 11C through FIG. 11F. FIG. 11H depicts relative GFP-tau P301L mRNA levels shown in FIG. 11C through FIG. 11F. For FIG. 11A, FIG. 11B, FIG. 11G, and FIG. 11H, data are mean±SEM; n=3; *, p<0.05; **, p<0.01; ns, not significant; unpaired Student's t test.

FIG. 12, comprising FIG. 12A through FIG. 12G, depicts representative effects of downregulation of TRIM11 in sporadic AD brains. FIG. 12A depicts representative relative ratios of AT8, pSer262, AT180, and PHF-1 tau species to GAPDH in control and AD brain tissues. FIG. 12B depicts representative images of Western blots of high MW tau species in frontal cortex gray matter from AD and control individuals. The #1 control and #1 AD samples were used in each blot for comparison between blots. FIG. 12C depicts representative results of qRT-PCR analysis of MAPT and TRIM mRNA levels in control and AD frontal cortices. FIG. 12D depicts representative images of anti-TRIM11 staining and DAPI in frontal cortices of control and AD samples (scale bar, 10 μm). FIG. 12E depicts quantification of TRIM11 signal normalized to numbers of total neural cells shown in FIG. 12D. FIG. 12F depicts representative relative TRIM11 mRNA (+) and protein (*) levels in individual control and AD samples. FIG. 12G depicts representative correlation analysis of TRIM11 expression and tau AT8 and AT180 species among AD samples. The r and p values of Pearson correlation coefficient are shown. For FIG. 12A, FIG. 12B, and FIG. 12E, data are mean±SEM; n=3; *, p<0.05; **, p<0.01; ns, not significant; unpaired Student's t test.

FIG. 13, comprising FIG. 13A through FIG. 13M, depicts representative results of testing TRIM11 targeting tau for degradation. FIG. 13A depicts relative insoluble GFP-tau P301L/actin ratios for the experiment shown in FIG. 3B. FIG. 13B depicts relative levels of PHF-1 for the experiment shown in FIG. 3C. FIG. 13C depicts representative images of Western blots of GFP-tau P301L expressed in control and TRIM11-knockout HEK293T cells. Cells were treated with CHX for the indicated durations. FIG. 13D depicts relative GFP-tau P301L/actin ratios shown in FIG. 13C. FIG. 13E depicts representative images of GFP-tau expressed alone or together with Flag-TRIM11 in HEK293T cells treated with or without okadaic acid (OA, 100 nM; scale bar, 100 μm). To achieve comparable levels of GFP-tau, more GFP-tau plasmid was expressed together with TRIM11. FIG. 13F depicts representative quantification of total tau relative to actin in pellet (PE), supernatant (SN), or whole cell lysate (WCL) of cells treated as in FIG. 13E. FIG. 13G depicts representative quantification of p-tau species (AT8 and Ser-396) relative to actin in the pellet (PE) of cells treated as in FIG. 13E. FIG. 13H depicts representative quantification of p-tau species (AT8 and ser-396) relative to actin in the supernatant (SN) of cells treated as in FIG. 13E. FIG. 13I depicts representative quantification of p-tau species (AT8 and Ser-396) relative to total tau in the supernatant (SN) of cells treated as in FIG. 13E. FIG. 13J depicts representative images of Western blots of GFP-tau expressed alone or with TRIM11 in HEK293T cells. Cells were treated with CHX for the indicated durations and lysed. FIG. 13K depicts relative GFP-tau/actin ratios quantified from FIG. 13J. FIG. 13L depicts representative images of Western blots of GFP-tau expressed in control and TRIM11-knockout cells treated with CHX for the indicated time and lysed. FIG. 13M depicts relative GFP-tau/actin ratios quantified from FIG. 13L. For FIGS. 13C, 13J, and 13L, to better compare the half-life of GFP-tau under different conditions, the blots on the left and their corresponding ones on the right were exposed for different times to achieve similar band intensity at time 0. For FIG. 13A, FIG. 13B, FIG. 13D, FIG. 13F through FIG. 13I, FIG. 13K, and FIG. 13M, data are means #SEM; n=3; *, p<0.05; **, p<0.01, ***, p<0.001; ns, not significant; unpaired Student's t test.

FIG. 14, comprising FIG. 14A through FIG. 14I, depicts representative results demonstrating TRIM11 interaction with and SUMOylation of tau, for promoting its degradation. FIG. 14A depicts representative images of Western blots for HEK293T cells expressing GFP-tau P301L with increasing amounts of TRIM11. Cells were treated with MG132 (with c-Myc as a control) or NH₄Cl (with p62 and LC3 as controls). FIG. 14B depicts representative images of Western blots for HEK293T cells as in FIG. 14A expressing GFP-tau. FIG. 14C depicts schematic representations of a BiFC assay for TRIM11-tau interaction (left and middle) and tau-tau interaction (right). VN173 and VC155 contain amino acids 1 to 172 and 155 to 238 of Venus, respectively. FIG. 14D depicts representative quantification of a BiFC assay for TRIM11-VN and tau-VC expressed in HEK293T cells. Related to FIG. 3E. FIG. 14E depicts representative images of BiFC signals in HEK293T cells expressing Tau-VN and TRIM-VC (scale bar, 100 μm). FIG. 14F depicts quantification of BiFC signals from FIG. 14E. FIG. 14G depicts representative images of Western blots for HEK293T cells transfected with TRIM11 together with GFP-tau and GFP-tauP301L, demonstrating that TRIM11 interacts with tau and preferentially mutant or phosphorylated forms. Cell lysates were immunoprecipitated with anti-FLAG antibody. Immunoprecipitates and WCL were analyzed. FIG. 14H depicts representative images of Western blots for HEK293T cells as in FIG. 14G transfected with TRIM11 together with GFP-tau and GFP-tau AT8. FIG. 14I depicts representative images of SDS-PAGE gels for recombinant GST-TRIM11, GFP-tau-6×His, and GFP-tau P301L-6×His proteins purified from E. coli, and Flag-TRIM11 and Flag-TRIM112EA proteins purified from HEK293T cells stained with Coomassie blue. BSA was used as a protein standard. For FIG. 14D and FIG. 14F, data are presented as mean±SD; n=10.

FIG. 15, comprising FIG. 15A through FIG. 15J, depicts representative results demonstrating that endogenous TRIM11 and tau co-localize and interact in N2a and SH-SY5Y cells. FIG. 15A depicts representative immunofluorescence images of endogenous TRIM11 and tau in SH-SY5Y treated with or without OA (100 nM; scale bar, 10 μm). Related to FIG. 3H and FIG. 3I. FIG. 15B depicts representative immunofluorescence images of endogenous TRIM11 and tau in N2a treated with or without OA (100 nM; scale bar, 5 μm). FIG. 15C depicts quantification of co-localization from FIG. 15A and FIG. 15B analyzed by Manders' colocalization coefficient (n=6). FIG. 15D depicts representative images of interactions between endogenous TRIM11 and tau in SH-SY5Y cells treated with or without OA (100 nM) examined by PLA (scale bar, 10 μm). Related to FIG. 3J and FIG. 3K. FIG. 15E depicts representative images of interactions between endogenous TRIM11 and tau in N2a cells treated with or without OA (100 nM) examined by PLA (scale bar, 10 μm). FIG. 15F depicts quantification of PLA signal from FIG. 15E (n=10). FIG. 15G depicts representative results of SH-SY5Y cells treated with or without OA (100 nM). TRIM11 and p-tau levels were analyzed by Western blot and TRIM11 relative to HSP90 was quantified. FIG. 15H depicts quantification of TRIM11 mRNA from cells treated as in FIG. 15G by qRT-PCR. FIG. 15I depicts representative results of N2a cells treated with or without OA (100 nM). TRIM11 protein and p-tau levels were analyzed by western blot and TRIM11 relative to HSP90 was quantified. FIG. 15J depicts quantification of TRIM11 mRNA from cells treated as in FIG. 15I by qRT-PCR. For FIG. 15G through FIG. 15J, n=3. For FIG. 15 For FIG. 15C, FIG. 15D, and FIG. 15G through FIG. 15J, data are means±SEM; *, p<0.05; **, p<0.01; ***, p<0.001; ns, not significant; unpaired Student's t test.

FIG. 16, comprising FIG. 16A and FIG. 16B, depicts representative images demonstrating that TRIM11 promotes SUMOylation of tau in cells and in a cell-free system. FIG. 16A depicts representative images of Western blots of HEK293T cells were transfected with TRIM11, GFP-tau, and GFP-tau P301L as indicated, demonstrating TRIM11 promotes SUMOylation of GFP-tau and GFP-tau P301L in cells. Cells were treated with MG132 and lysed in SDS-containing buffer. After dilution in buffer without SDS, cell lysates were immunoprecipitated (d-IP) by anti-GFP antibody. Immunoprecipitates and inputs were analyzed for tau and total SUMOylation, as well as protein expression. FIG. 16B depicts representative images of Western blots of purified recombinant Flag-TRIM11 incubated with GST-tau or GST-tau P301L in the presence or absence of ATP as indicated, together with SUMO E1, E2, and 6×His-SUMO2. The reaction mixtures were analyzed for SUMOylation of GFP-tau and GFP-tau P301L by d-IP.

FIG. 17, comprising FIG. 17A through FIG. 17J, depicts representative results demonstrating that TRIM11 increases tau solubility in cells. FIG. 17A depicts representative images of Western blots of HEK293T cells expressing GFP-tau in the presence or absence of Flag-TRIM11. FIG. 17B depicts quantification of the ratios of insoluble and soluble GFP-tau from FIG. 17A. FIG. 17C depicts representative fluorescence images of cells in the experiment shown in FIG. 4A (scale bar, 100 μm). FIG. 17D depicts representative images of Western blots of HEK293T cells transfected with GFP or GFP-tau P301 alone or together with Flag-TRIM11 or Flag-TRIM112EA demonstrating that TRIM11 prevents tau P301L aggregation in cells. FIG. 17E depicts representative fluorescence images of HEK293T cells transfected with tau-VN or tau-VC pre-treated with control or TRIM11 siRNA (scale bar, 100 μm). FIG. 17F depicts quantification of relative fluorescence signals (top) and protein expression (bottom) in the cells of FIG. 17E. FIG. 17G depicts representative fluorescence images of HEK293T or TRIM11-knockout HEK293T cells transfected with tau-VN and tau-VC individually or together in (scale bar, 100 μm). FIG. 17H depicts quantification of relative fluorescence signals (top) and protein expression (bottom) in the cells of FIG. 17G. FIG. 17I depicts representative images of Western blots for HEK293/RD (P301L/V337M)-YFP cells transfected with empty vector (−) or TRIM11 treated with or without tau PFFs. FIG. 17J depicts relative tau (SN)/tau (WCL) and tau (SN)/tau (WCL) ratios quantified from FIG. 17I. Related to FIG. 4, D and E. For FIG. 17B, FIG. 17F, FIG. 17H, and FIG. 17J, data are presented as mean±SD; n=3; *, p<0.05; **, p<0.01; ns, not significant; unpaired Student's t test.

FIG. 18, comprising FIG. 18A through FIG. 18E, depicts representative results demonstrating that TRIM11 is both a molecular chaperone and disaggregase for tau. FIG. 18A depicts representative results of tau amyloid fibril formation by ThT binding, demonstrating that TRIM11 inhibits spontaneous tau aggregation. Purified GST-tau (20 μM) was incubated with heparin (30 μM) for 24 h in the absence or presence of GST or GST-TRIM11 at the indicated concentrations. FIG. 18B depicts representative images of a Western blot of tau amyloid fibril formation by sedimentation. FIG. 18C depicts representative electron microscopy images demonstrating inhibition of tau amyloid fibril formation by TRIM11. (scale bar, 500 nm). FIG. 18D depicts representative images of a Western blot demonstrating that GST-TRIM11 dissolves preformed tau aggregation. PFFs formed by GST-tau (1 μM) were treated with GST or GST-TRIM11 at the indicated concentrations and obtained by sedimentation. FIG. 18E depicts representative electron microscopy images demonstrating that TRIM11 dissolves preformed tau aggregates (scale bar, 500 nm).

FIG. 19, comprising FIG. 19A through FIG. 19M, depicts representative results demonstrating that TRIM11 interacts with tau and abrogates its aggregation in neurons. FIG. 19A depicts representative co-localization of endogenous TRIM11 and tau in cortical neurons derived from wild-type (WT) mice analyzed Pearson's correlation coefficient. Related to FIG. 5A. FIG. 19B depicts representative images of PLA of endogenous TRIM11-tau interaction in WT cortical neurons treated with or without OA (100 nM; scale bar 10 μm). FIG. 19C depicts quantification of PLA signal from FIG. 19B (n=10). FIG. 19D depicts representative Western blot analysis of WT cortical neurons treated with or without OA (100 nM) (top) and quantification of TRIM11 levels (bottom; n=3). FIG. 19E depicts representative images of WT cortical neurons treated with or without SCR CTRL-FAR RED for 3 days and stained for MAP (scale bar, 20 μm). FIG. 19F depicts representative images of a Western blot for WT cortical neurons treated with AUMInc-scrctrl or AUMSiI-TRIM11-1/2/3/4/5 for 3 days. Cells were lysed in RIPA buffer. FIG. 19G depicts representative images of cortical neurons derived from PS19 mice transduced with control or TRIM11 #5 ASO and treated with PFFs generated from myc-K18/P301L. Tau aggregates were detected with MC1 (scale bar, 10 μm). FIG. 19H depicts quantification of MC1 intensity from FIG. 19G (n=8). FIG. 19I depicts representative images of PS19 cortical neurons transduced with AAV9-GFP or AAV9-TRIM11 and stained for DAPI, p-tau, GFP, and TRIM11 (scale bar, 50 μm). Related to FIG. 4D. FIG. 19J depicts representative images of PS19 cortical neurons transduced with AAV9-GFP or AAV9-TRIM11 treated with myc-K18/P301L PFFs. Cells were treated with myc-K18/P301L PFFs, and tau aggregates were detected with the antibody MC1. Cells were stained for DAPI, p-tau, GFP, and TRIM11 (tagged with HA and detected with an anti-HA antibody) (scale bar, 50 μm). FIG. 19K depicts quantification of MC1 signal from FIG. 19J (n=9). FIG. 19L depicts representative images of PS19 hippocampal neurons transduced with AAV9-GFP or AAV9-TRIM11 vectors and treated with myc-K18/P301L PFFs. Tau aggregates were detected with AT8 (scale bar, 100 μm). FIG. 19M depicts representative images of PS19 hippocampal neurons as in FIG. 19M detected with MC1 (scale bar, 100 μm). For FIG. 19A, FIG. 19C, FIG. 19D, FIG. 19H, and FIG. 19K, data are presented as mean±SEM; *, p<0.05; **, p<0.01; ns, not significant; unpaired Student's t test.

FIG. 20, comprising FIG. 20A through FIG. 20C, depicts representative results demonstrating that TRIM11 maintains neural integrity and connectivity. FIG. 20A depicts representative images of WT cortical neurons treated with control or TRIM11 ASO #5 analyzed for the expression of SYP and PSD95 (scale bar, 5 μm). FIG. 20B depicts colocalization of SYP-reactive and PSD95-reactive puncta from FIG. 20A (mean±SEM, n=6). FIG. 20C depicts representative images of NFL and MAP2 staining in WT cortical neurons treated with control or TRIM11 ASO #5 (scale bar, 10 μm). Related to FIG. 5, J to L. For FIG. 20B, **, p<0.01; unpaired Student's t test.

FIG. 21, comprising FIG. 21A through FIG. 21I, depicts representative results demonstrating that TRIM11 reduces tau pathology and neuroinflammation in PS19 mice. FIG. 21A depicts representative images of the CA1 and DG regions of the brains of PS19 mice injected with AAV-GFP or AAV9-TRIM11 and stained with AT8 (scale bar, 0.2 mm). Related to FIG. 6B. FIG. 21B depicts quantification of the Western blot shown in FIG. 6C normalized to loading control, demonstrating that TRIM11 strongly reduced p-tau species in SN and PE fractions of hippocampal lysates (AT8 in PE, by ~81%; PHF1 in PE, by ~98%; AT8 in SN, by ~52%; PHF1 in SN, by ~89%). FIG. 21C depicts quantification of the Western blot shown in FIG. 6C normalized to total tau in the corresponding fractions, demonstrating that TRIM11 strongly reduced p-tau species in SN and PE fractions of hippocampal lysates (AT8 in PE, by ~59%; PHF1 in PE, by ~83%; AT8 in SN, by ~56%; PHF1 in SN, by ~92%). FIG. 21D depicts quantification of the Western blot shown in FIG. 6C normalized to loading control. FIG. 21E depicts representative images of the hippocampus and CA3 region of brains of PS19 mice injected with AAV9-GFP or AAV9-TRIM11, as well as those of age-matched wild-type littermates, stained with antibodies against GFAP. FIG. 21F depicts representative images of the hippocampus and CA3 region of brains of PS19 mice treated as in FIG. 21E stained with antibodies against Iba1. Related to FIG. 6D through FIG. 6G. FIG. 21G depicts representative images of the hippocampus of brains of PS19 mice injected with AAV9-GFP or AAV9-TRIM11, as well as those of wild-type mice, probed with antibody against MAP2 (G). FIG. 21H depicts representative images of the hippocampus of brains of PS19 mice as treated in FIG. 21G probed with antibody against NFL. Related to FIG. 6H through FIG. 6K. FIG. 21I depicts representative images of the brains of PS19 mice injected with AAV9-GFP or AAV9-TRIM11, as well as wild-type mice, stained with antibodies detecting NeuN. Related to FIG. 6, L and M. For FIG. 21A and FIG. 21E through FIG. 21I, scale bar=0.2 mm. For FIG. 21B through FIG. 21D, data are presented as mean±SD; n=3; *, p<0.05; **, p<0.01; ***, p<0.001; ns, not significant; unpaired Student's t test.

FIG. 22, comprising FIG. 22A through FIG. 22F, depicts representative results demonstrating that TRIM11 ameliorates PFF-accelerated tau pathology and neuroinflammation in PS19 mice. FIG. 22A depicts representative images of the CA1 and DG regions of brains of PS19 mice injected with tau K18 PFFs together with AAV-GFP or AAV9-TRIM11 and stained with AT8. Related to FIG. 7B. FIG. 22B depicts quantification of the Western blot shown in FIG. 7C normalized to sample loading demonstrating that TRIM11 strongly reduces p-tau species in SN and PE fractions of hippocampal lysates (AT8 in PE, by ~86%; PHF1 in PE, by ~76%; AT8 in SN, by ~100%; PHF1 in SN, by ~89%). FIG. 22C depicts quantification of the Western blot shown in FIG. 7C normalized to total tau in the corresponding fractions demonstrating that TRIM11 strongly reduces p-tau species in SN and PE fractions of hippocampal lysates (AT8 in PE, by ~74%; PHF1 in PE, by ~57%; AT8 in SN, by ~100%; PHF1 in SN, by ~93%). FIG. 22D depicts quantification of the Western blot shown in FIG. 7C normalized to sample loading, demonstrating that TRIM11 reduces total tau in the insoluble fraction (by ~45%) and increases total tau in the soluble fraction (~38%), while having minimal effect on overall tau in WCL. FIG. 22E depicts representative images of the hippocampus and CA3 region of brains of PS19 mice injected with tau K18 PFFs together with AAV9-GFP or AAV9-TRIM11 and stained with antibody for GFAP. FIG. 22F depicts representative images of the hippocampus and CA3 regions of the brains of PS19 mice treated as in FIG. 22E stained with antibodies against Iba1. Related to FIG. 7D and FIG. 7E. For FIG. 22A, FIG. 22E, and FIG. 22F, scale bar=0.2 mm. For FIG. 22A through FIG. 22D, data are presented as mean±SD; n=5; *, p<0.05; **, p<0.01; ***, p<0.001; ns, not significant; unpaired Student's t test.

FIG. 23, comprising FIG. 23A through FIG. 23F, representative results demonstrating that the delivery of AAV9-TRIM11 via intraparenchymal (IP) injection ameliorates tau pathology and neuroinflammation in 3×Tg-AD mice. FIG. 23A depicts representative images of the CA1 region of brains of un-injected 3×Tg-AD mice at 12 months of age, and AAV-GFP- or AAV9-TRIM11-injected 3×Tg-AD mice at 13 months of age stained with AT8. Related to FIG. 7B. FIG. 23B depicts quantification of the Western blot shown in FIG. 8C normalized to sample loading, demonstrating that TRIM11 strongly reduces p-tau species in SN and PE fractions of hippocampal lysates (by ~70 to 90%). FIG. 23C depicts quantification of the Western blot shown in FIG. 8C normalized to the total tau in the corresponding fractions, demonstrating that TRIM11 strongly reduces p-tau species in SN and PE fractions of hippocampal lysates. FIG. 23D depicts quantification of the Western blot in FIG. 8C normalized to sample loading, demonstrating that TRIM11 strongly reduces total tau in PE (by ~45%) and moderately reduce total tau in SN and WCL (both by ~20%). FIG. 23E depicts representative images of the CA3 and DG regions of the brains of 3×Tg-AD mice injected with tau K18 PFFs together with AAV9-GFP or AAV9-TRIM11 and stained for antibody for GFAP. FIG. 23F depicts representative images of the CA3 and DG regions of the brains of 3×Tg-AD mice as treated in FIG. 23E stained for antibody for iBl1. Related to FIG. 8D and FIG. 8E. For FIG. 23A, FIG. 23E, and FIG. 23F, scale bar=0.2 mm. For FIG. 23B through FIG. 23C, data are presented as mean+SD; n=5; *, p<0.05; ns, not significant; unpaired Student's t test.

FIG. 24, comprising FIG. 24A through FIG. 24E, depicts representative results demonstrating that delivery of AAV9-TRIM11 via ICV injection ameliorates tau pathology and neuroinflammation in 3×Tg-AD mice. FIG. 24A depicts quantification of the Western blot shown in FIG. 8L normalized to sample loading demonstrating that TRIM11 reduces p-tau species in SN and PE fractions of hippocampal lysates (by ~80 to 93%). FIG. 24B depicts quantification of the Western blot shown in FIG. 8L normalized to loading demonstrates that TRIM11 reduces total tau in the insoluble fraction (by ~80%) and WCL (by ~65%) and moderately reduced total tau in the soluble fraction (by ~43%). FIG. 24C depicts quantification of the Western blot shown in FIG. 8L normalized to total tau in the respective fractions, demonstrating that TRIM11 strongly reduces SN p-tau (by ~68-80%), but minimally or moderately reduced PE p-tau (~45% to 0%). FIG. 24D depicts representative images of the hippocampus and CA1, CA3, and DG regions of brains of 3×Tg-AD mice injected with tau K18 PFFs together with AAV9-GFP or AAV9-TRIM11 and stained for antibody for GFAP. FIG. 24E depicts representative images of the hippocampus and CA1, CA3, and DG regions of brains of 3×Tg-AD mice treated as in FIG. 24D stained for antibody for Iba1. Related to FIG. 8M and FIG. 8N. For FIG. 24D and FIG. 24E, scale bar=0.2 mm. For FIG. 24A through FIG. 24C, data are presented as mean±SD; n=6; *, p<0.05; **, p<0.01; ***, p<0.001; ns, not significant; unpaired Student's t test.

FIG. 25, comprising FIG. 25A through FIG. 25C, depicts representative results demonstrating the effect of TRIM proteins on tau protein levels. FIG. 25A depicts a schematic representation of a BiFC assay for tau self-association. VN173 and VC155 contain amino acids 1 to 172 and 155 to 238 of Venus, respectively. FIG. 25B depicts representative images of BiFC signals in HEK293T cells expressing tau-VN and tau-VC individually or together (top) and quantification of the BiFC signal (bottom). FIG. 25C depicts representative images of Western blots for HEK293T cells co-expressing Tau-VN and tau-VC were co-expressed along with the indicated TRIM proteins. The TRIM proteins that reduced levels of BiFC signal are boxed.

FIG. 26 depicts representative images of HEK293T cells co-expressing tau-VN and tau-VC along with the indicated TRIM proteins, demonstrating the effect of TRIM proteins on tau self-association. Related to FIG. 25C.

FIG. 27 depicts representative quantification of BiFC fluorescence in HEK293T cells expressing tau-VN and tau-VC along with the indicated TRIM proteins, demonstrating the effect of TRIM proteins on tau self-association. The TRIM proteins that decreased the fluorescence signal are indicated in red. Related to FIG. 1C and FIG. 2. Data are means±SD; n=3; ***, p<0.005; unpaired Student's t test.

FIG. 28 depicts representative images of Western blots of HEK293T cells expressing GFP-α-Syn-A53T and the indicated TROM proteins, demonstrating the effect of TRIM proteins on α-Syn A53T aggregation. TRIM proteins that reduced levels of α-Syn A53T are labeled in red color. The expected full-length TRIM bands are indicated by red arrowheads.

FIG. 29, comprising FIG. 29A through FIG. 29C, depicts representative effects of TRIM proteins on α-Syn self-association. FIG. 29A depicts a schematic representation of a BiFC assay based on Venus, an improved version of yellow fluorescent protein (YFP), for the self-association of α-Syn. FIG. 29B depicts representative images of HEK293T cells expressing VIS and SV2 individually or together (top) and quantification of BiFC signal (bottom). FIG. 29C depicts representative images of Western blots from HEK293T cells co-expressing VIS and SV2 and the indicated TRIM proteins. TRIM proteins that reduced BiFC signal are labeled in red color. The expected full-length TRIM bands are indicated by red arrowheads.

FIG. 30 depicts representative fluorescence images of tHEK293T cells co-expressing VIS and SV2 along with the indicated TRIM proteins, demonstrating the effect of TRIM proteins on α-Syn self-association. Related to FIG. 29C.

FIG. 31 depicts representative quantification of BiFC fluorescence signals in HEK293T cells co-expressing VIS and SV2 along with the indicated TRIM proteins, demonstrating the effect of TRIM proteins on α-Syn self-association. Data are means±SD; n=3. Related to FIG. 29C and FIG. 30.

FIG. 32 depicts representative images of Western blots of HEK293T cells transfected with SOD1 G93A-GFP and control vector (−) or the indicated TRIM proteins lysed in NP-40 containing buffer and separated into soluble supernatant (SN) and insoluble pellet (PE) fractions by sedimentation, demonstrating the effect of TRIM proteins on SOD1. The expected full-length TRIM bands are indicated by green arrowheads. TRIM proteins that substantially reduced insoluble SOD1 G93A-GFP species are labeled in red color.

FIG. 33, comprising FIG. 33A through FIG. 33F, depicts representative results of testing TRIM11 targeting pathogenic SOD1 for proteasomal degradation. FIG. 33A depicts representative images of Western blots of HEK293T cells expressing SOD1 93A-GFP and transfected with empty vector (EV), TRIM11, or TRIM112EA, demonstrating that TRIM11 reduces levels of SOD1 G93A. FIG. 33B depicts representative images of Western blots for HEK293T cells transfected with SOD1 G93A-GFP and EV or TRIM11, and treated with CHX for the indicated durations, demonstrating that TRIM11 accelerates degradation of SOD1 G93A-GFP. FIG. 33C depicts representative images of Western blots for NSC-34 cells expressing WT-hSOD-GFP or G93A-hSOD-GFP treated with 1 μg Dox for 24 h to induce SOD1 expression, and then transfected with empty vector (EV) or increasing dose of FLAG-TRIM11, plus 0.5 μg mCherry-N1 as a control for transfection efficiency. Cells were treated with proteasome inhibitor MG132 (10 μM) for 8 hours or lysosomal inhibitors leupeptin (10 mM) plus NH₄Cl (20 mM) (L+N) for 16 hours. c-Myc and p62 were used to verify proteasomal and lysosomal inhibition, respectively. Data are representative of three independent experiments. FIG. 33D depicts representative images of Western blots for Neuo-2A cells treated with 100 μM H₂O₂ for 48 h and transiently transfected with EV or increasing amount of FLAG-TRIM11, followed by fractionation analysis to probe endogenous SOD1. 0.5 μg mCherry-N1 was co-transfected with FLAG vectors as indicator for equal transfection. FIG. 33E depicts representative images of Western blots for NSC-34 cells expressing WT-hSOD-GFP or G93A-hSOD-GFP treated with 1 μg Dox for 24 h to induce SOD1 expression and transiently transfected with 2 μg EV or FLAG-TRIM11, plus 0.5 μg mCherry-N1. Cells were then cultured in medium without Dox for the indicated time, followed by Western blot analysis of the SN and PE fractions and the whole cell lysates (WCL). FIG. 33F depicts representative images of Western blots for NSC-34 cells expressing WT-hSOD-GFP and G93A-hSOD-GFP treated with Dox to induce SOD1 express and transfected with EV or FLAG-TRIM11 (2 μg each). Cells were lysed in RIPA buffer 40 h post-transfection, followed by reciprocal IP using anti-FLAG (M2) beads or anti-GFP-conjugated agarose beads. Input indicates 10% of RIPA lysis. TRIM11-expressing NSC-34 stable cells not induced with Dox were used as a control (1^{st} lane) to show binding specificity. Data are representative of three independent experiments.

FIG. 34, comprising FIG. 34A through FIG. 34J, depicts representative results of testing TRIM11 ameliorating pathology and neuroinflammation of SOD1-G93A mice. FIG. 34A depicts a schematic representation of the study. FIG. 34B depicts representative images of cortex and spinal cord of control mice and mice injected with AAV9-GFP or AAV9-TRIM11, demonstrating that TRIM11 reduced SOD1-G93A staining in the cortex and spinal cord. FIG. 34C depicts quantification of SOD1 G93A staining in the cortex of FIG. 34B (n=15 mice each group). FIG. 34D depicts quantification of SOD1 G93A staining in the spinal cord of FIG. 34B (n=7 for WT, 8 for GFP, and 10 for TRIM11). FIG. 34E depicts representative images of cortex and spinal cord of control SOD1-G93A mice and SOD1-G03A mice injected with AAV9-GFP- or AAV9-TRIM11 stained with antibody specific for GFAP, demonstrating that TRIM11 reduces astrogliosis in SOD1-G93A mice. FIG. 34F depicts quantification of GFAP immunoreactivity in the cortex of FIG. 34E (n=13 for WT, 15 for GFP and TRIM11). FIG. 34G depicts quantification of GFAP immunoreactivity in the spinal cord of FIG. 34E (n=7 WT, 8 GFP, or 10 TRIM11). FIG. 34H depicts representative images of cortex and spinal cord of mice treated as in FIG. 34E stained with antibody specific for Iba1, demonstrating that TRIM11 reduces microgliosis in SOD1 G93A mice. FIG. 34I depicts quantification of Iba1 immunoreactivity in the cortex of FIG. 34H. FIG. 34J depicts quantification of Iba1 immunoreactivity in the spinal cord of FIG. 34H. For FIG. 34I and FIG. 34J, n=7 WT, 8 GFP, or 10 TRIM11. For FIG. 34B, FIG. 34E, and FIG. 34H, scale bar=0.2 mm. For FIG. 34C, FIG. 34F, and FIG. 34I, 6-7 cortical fields were analyzed for each mouse. For FIG. 34D, FIG. 34G, and FIG. 34J, 3-4 spinal cord fields were analyzed for each mouse. For FIG. 34C, FIG. 34D, FIG. 34F, FIG. 34G, FIG. 34I, and FIG. 34J, data are presented as mean±SD; **, p<0.01; ***, p<0.005; ****, p<0.001; unpaired Student's t test.

FIG. 35, comprising FIG. 35A and FIG. 35B, depicts representative results of testing TRIM11 alleviating protein aggregation, neuroinflammation, and apoptosis in SOD1 G93A mouse. FIG. 35A depicts representative images of Western blots for the cortex from non-transgenic (Ntg) and hSOD1 G93A transgenic mice injected with AAV9-GFP or AAV9-TRIM11-HA fractioned into NP-40 soluble (SN) and 2% SDS soluble (PE) components. FIG. 35B depicts representative images of Western blots for the spinal cord from the mice of FIG. 35A. GFP and HA indicate successful intracerebroventricular (ICV) injection of AAV into central and peripheral nervous system. GFAP and Iba1 were used as astrocyte and microglia marker, respectively. Cleaved caspase 3 (C-CASP3) is a marker for apoptosis. Arrows indicate SOD1 monomer and dimer. SE: short exposure; LE: long exposure. Data are representative of two independent experiments.

FIG. 36, comprising FIG. 36A through FIG. 36E, depicts representative results of testing TRIM11 rescue of behavioral defects in SOD1 G93A mice. FIG. 36A depicts representative quantification of travel distance in AAV9-GFP-injected mice and AAV9-TRIM11-injected mice (n=27 for GFP 29 for TRIM11). FIG. 36B depicts representative quantification of movement time in AAV9-GFP-injected mice and AAV9-TRIM11-injected mice (n=22 for GFP and 21 for TRIM11). FIG. 36C depicts representative improvement in motor performance of AAV9-TRIM11-injected mice compared to AAV9-GFP-injected mice as shown by increased latency to fall on wire hang (n=21). FIG. 36D depicts representative improvement in motor performance of AAV9-TRIM11-injected mice compared to AAV9-GFP-injected mice as shown by increased latency to fall on rotarod, with accelerating speed from 4 to 40 RPM (n=22 for GFP and 21 for TRIM11). FIG. 36E depicts representative correlation of TRIM11 expression levels and improvement in motor function in rotarod test. For FIG. 36A through FIG. 36D, data are presented as mean±SD; *, p<0.05; **, p<0.01; ***, p<0.005; unpaired Student's t test.

FIG. 37, comprising FIG. 37A through FIG. 37C, depicts representative results of a systematic analysis of the effect of TRIMs on TDP43. FIG. 37A depicts representative images of Western blot of HEK293T cells transfected with TDP43-Q331K-GFP and control vector (−) or the indicated TRIM proteins. FIG. 37B depicts representative images of Western blot of HEK293T cells transfected with TDP43-Q331K-GFP and control vector (−) or the indicated TRIM proteins. Cells were lysed in NP-40-containing buffer, separated into soluble supernatant (SN), and insoluble pellet (PE) fractions by sedimentation, and analyzed by Western blot. For FIG. 37A and FIG. 37B, the expected full-length TRIM bands are indicated by blue arrowheads. TRIM proteins that substantially reduced insoluble TDP43-Q331K-GFP species are labeled in red color. FIG. 37C depicts representative images of Western blots for SH-SY5Y cells stably expressing GFP-TDP-43 5FL or GFP-TDP-43 Q331K transduced with control lentiviral vector (−) or lentiviral vector expressing TRIM11, demonstrating that TRIM11 reduces levels of insoluble, but not soluble, TDP43 mutants. Cells were lysed in NP40-containing buffer, and the soluble supernatant (SN) and insoluble fractions were separated by sedimentation. The insoluble pellets were dissolved in SDS-containing buffer. SDS-resistant pellets (SR) were analyzed by dot blot. WCL, whole cell lysates.

FIG. 38, comprising FIG. 38A through FIG. 38J, depicts representative results of testing TRIM11 ameliorating protein aggregation and neuroinflammation in the TDP-43-ALS mouse model. FIG. 38A depicts a schematic representation of the study. FIG. 38B depicts representative images of the cortex and spinal cord of control Tar4/4 mice and Tar4/4 mice injected with AAV9-GFP or AAV9-TRIM11, demonstrating TRIM11 reduced levels of TDP-43 in the cortex and the spinal cord. FIG. 38C depicts quantification of TDP-43 staining in the cortex of FIG. 28B (n=9 for WT and 13 for GFP and TRIM11). FIG. 38D depicts quantification of TDP-43 staining in the spinal cord of FIG. 38B (n=5 for WT and 13 for GFP and TRIM11). FIG. 38E depicts representative images of the cortex and spinal cord of control Tar4/4 mice and Tar4/4 mice injected with AAV9-GFP or AAV9-TRIM11 stained with antibody specific for GFAP, demonstrating that TRIM11 reduces astrogliosis in Tar4/4 mice. FIG. 38F depicts quantification of GFAP immunoreactivity in the cortex of FIG. 38E (n=5 WT, 6 GFP, or 10 TRIM11). FIG. 38G depicts quantification of GFAP immunoreactivity in the spinal cord of FIG. 38E (n=4 for WT or 6 for GFP and TRIM11). FIG. 38H depicts representative images of the cortex and spinal cord of mice treated as in FIG. 38E stained with antibody specific for Iba1, demonstrating that TRIM11 reduces microgliosis. FIG. 38I depicts quantification of Iba1 immunoreactivity in the cortex of FIG. 38H (n=5 for WT, 6 for GFP, or 8 for TRIM11). FIG. 38J depicts quantification if Iba1 immunoreactivity in the spinal cord (n=5 for WT and GFP or 7 for TRIM11). For FIG. 38B, FIG. 38E, and FIG. 38H, scale bar=0.2 mm. For FIG. 38C, FIG. 38D, FIG. 38F, FIG. 38G, FIG. 38I, and FIG. 38J, data are presented as mean±SD; *, p<0.05; **, p<0.01; ***, p<0.005; ****, p<0.001; unpaired Student's t test.

FIG. 39 depicts representative results of testing TRIM11 reducing TDP-43 aggregation and apoptosis in TDP-43-ALS mouse model. Whole brain lysates from non-transgenic (Ntg) and ALS disease mice (TAR4/4) injected with AAV9-GFP or AAV9-TRIM-HA were fractioned into NP-40 soluble (SN) and 2% SDS soluble (PE) components. GFP and HA indicate successful intracerebroventricular (ICV) injection of AAV into the brain. GFAP and Iba1 were used as astrocyte and microglia marker, respectively. Cleaved PARP and cleaved caspase 3 (C-CASP3) are both markers for apoptosis. Arrows indicate full-length, 35 kDa, or 25 kDa form of TDP-43. The 25 kDa fragment is associated with TDP-43 protein aggregation burden. GAPDH and Lamin A/C were chosen as the marker for cytoplasmic and nuclear fraction, respectively. SE: short exposure; LE: long exposure. Data are representative of two independent experiments.

FIG. 40, comprising FIG. 40A through FIG. 40P, depicts representative results of testing TRIM10 reducing levels of various misfolding-prone proteins. FIG. 40A depicts representative images of Western blots for HEK293T cells transfected with GFP-tagged tau or tau P301L along with increasing amounts of Flag-TRIM10 for 36 hours. FIG. 40B depicts representative images of Western blots for HEK293T cells transfected with GFP-tagged α-Syn-A53T along with increasing amounts of Flag-TRIM10 for 36 hours. FIG. 40C depicts representative images of Western blots for HEK293T cells transfected with GFP-tagged SOD1 along with increasing amounts of Flag-TRIM10 for 36 hours. FIG. 40D depicts representative images of Western blots for HEK293T cells transfected with GFP-tagged TDP-40, TDP-43 M337V, or TDP-43 Q331K along with increasing amounts of Flag-TRIM10 for 36 hours. FIG. 40E depicts representative images of Western blots for HEK293T cells transfected with GFP-tagged FUS along with increasing amounts of Flag-TRIM10 for 36 hours. FIG. 40F depicts representative images of Western blots for HEK293T cells transfected with GFP-tagged Atxn1 82Q along with increasing amounts of Flag-TRIM10 for 36 hours. FIG. 40G depicts representative images of Western blots for control (left) or TRIM10-knockdown (right) HEK293T cells transfected with Flag-TRIM10 and GFP-Atxn1 82Q or 30Q for 36 hours. FIG. 40H depicts representative images of Western blots for TRIM10-knockdown HEK293T cells transfected with or without GFP-Atxn1 82Q for 36 hours. FIG. 40I depicts representative images of Western blots for HEK293T cells transfected with GFP-tagged Htt-72Q along with increasing amounts of Flag-TRIM10 for 36 hours. FIG. 40J depicts quantification of tau mRNA from cells treated as in FIG. 40A determined by RT-PCR. FIG. 40K depicts quantification of α-Syn-A53T mRNA from cells treated as in FIG. 40B determined by RT-PCR. FIG. 40L depicts quantification of SOD1 mRNA from cells treated as in FIG. 40C determined by RT-PCR. FIG. 40M depicts quantification of TDP-43 mRNA from cells treated as in FIG. 40D determined by RT-PCR. FIG. 40N depicts quantification of FUS mRNA from cells treated as in FIG. 40E determined by RT-PCR. FIG. 40O depicts quantification of Htt 72Q mRNA from cells treated as in FIG. 40G determined by RT-PCR. FIG. 40P depicts quantification of ataxin mRNA from cells treated as in FIG. 40H determined by RT-PCR. determined by RT-PCR. For FIG. 40J through FIG. 40P, data are presented as mean±SD; n=3; ns, not significant; unpaired Student's t test. WCL, whole cell lysate; PE, pellet fraction; SN, soluble supernatant fraction.

FIG. 41, comprising FIG. 41A through FIG. 41K, depicts representative results of testing TRIM10 promoting proteasomal degradation of misfolding-prone proteins. FIG. 41A depicts representative images of Western blots for HEK293T cells expressing GFP-Atxn1 82Q together with increasing amounts of TRIM10. Cells were treated with 10 μM MG132 for 6 hours and/or NH₄Cl and leupeptins (LN) (with p62 and LC3 as controls). FIG. 41B depicts representative images of Western blots for HEK293T cells transfected with GFP-tagged tau or tau P301L in the presence or absence of Flag-TRIM10 for 36 hours and treated with MG132 for 6 hours. FIG. 41C depicts representative images of Western blots for HEK293T cells transfected with GFP-tagged α-Syn-A53T in the presence or absence of Flag-TRIM10 for 36 hours and treated with MG132 for 6 hours. FIG. 41D depicts representative images of Western blots for HEK293T cells transfected with GFP-tagged SOD1 or SOD1 G93A in the presence or absence of Flag-TRIM10 for 36 hours and treated with MG132 for 6 hours. FIG. 41E depicts representative images of Western blots for HEK293T cells transfected with GFP-tagged TDP-43, TDP-43 M337V, or TDP-43 Q331K in the presence or absence of Flag-TRIM10 for 36 hours and treated with MG132 for 6 hours. FIG. 41F depicts representative images of Western blots for HEK293T cells transfected with GFP-tagged FUS in the presence or absence of Flag-TRIM10 for 36 hours and treated with MG132 for 6 hours. FIG. 41G depicts representative images of Western blots for HEK293T cells transfected with GFP-tagged Htt-72Q in the presence or absence of Flag-TRIM10 for 36 hours and treated with MG132 for 6 hours. FIG. 41H depicts representative images of Western blots for cycloheximide (CHX) chase assay of GFP-Atxn1 82Q turnover in HEK293T cells in the presence or absence of Flag-TRIM10. FIG. 41I depicts quantification of relative GFP-Atxn1 82Q/actin ratios analyzed by Image J. Data are presented as mean±SD. ***P<0.001. FIG. 41J depicts representative confocal images of HEK293T cells expressing GFP-Atxn1 82Q and mCherry-TRIM10 treated with 10 μM MG132 for 6 hours, fixed, and stained with Hoechst. FIG. 41K depicts representative quantification of the percentage of cells with TRIM10 accumulation in the nucleus from FIG. 41J.

FIG. 42, comprising FIG. 42A through FIG. 42I, depicts representative results of testing TRIM10 as a molecular chaperone for various client proteins. FIG. 42A depicts representative results of ThT-binding assays of α-Syn monomer fibrilization (70 μM) in the presence or absence of GST or Flat-TRIM10 at the indicated concentrations. Quantification is depicted on the left and Western blot on the right. FIG. 42B depicts representative electron microscopy images of α-Syn monomer fibrilization (70 μM) in the presence of GST or 0.5 μM Flag-TRIM10 (scale bar, 500 nm). FIG. 42C depicts representative results of ThT-binding assays of α-Syn (70 μM) fibrillization in the presence or absence of GST, Flag-TRIM10, or Hsp70/Hsp40-Hsp104 (0.5 μM each). An ATP-regeneration system was included with heat-shock proteins, but not TRIM10 (all subsequent experiments with heat-shock proteins, but not experiments with TRIM10, also contained ATP and an ATP-regeneration system). FIG. 42D depicts representative images of Western blots for fibrillization of α-Syn (70 μM) in the presence of GST and TRIM10 (50-500 nM) and dot blot for pellet (PE) and SDS-resistant (SR) aggregates and total α-Syn and by disuccinimidyl suberate (DSS) cross-linking (for soluble oligomers). FIG. 42E depicts representative images of Western blots for HEK293T cells transfected with Flag-TRIM10 and GFP-α-Syn-A53T for 36 hours. FIG. 42F depicts representative Western blots for fibrillization of Aβ42 monomers (10 μM) in the absence or presence of TRIM10 (50-200 nM). FIG. 42G depicts representative results of ThT-binding assays of Aβ42 monomers (10 μM) in the presence or absence of TRIM10 (50-200 nM). FIG. 42H depicts representative viability of SH-SY5Y cells treated with Aβ42 pre-incubated with or without TRIM10. FIG. 42I depicts representative images of Western blots of Flag-TRIM10 (0.5 μM)-mediated suppression of Atxn1 82Q aggregation. Relative amounts of Atxn1 82Q in each fraction are shown. For FIG. 42A, FIG. 42C, and FIG. 42H, data are mean±SD; n=3; *, p<0.05; **, p<0.01; unpaired Student's t test.

FIG. 43, comprising FIG. 43A through FIG. 43H, depicts representative result of testing TRIM10 as a disaggregase for various client proteins. FIG. 43A depicts representative results of ThT-binding assays of α-Syn PFFs (0.5 μM) treated with GST, Flag-TRIM10 (at increasing concentrations), or HSPs. FIG. 43B depicts a representative image of a Western blot of α-Syn PFFs (0.5 μM) treated with GST, Flag-TRIM10 (0.1, 0.2, and 0.5 μM), or HSPs. FIG. 43C depicts representative images of dot blots of α-Syn fibrils (0.5 μM) treated with GST (0.2 μM), Glag-TRIM10 at the indicated concentrations, or HSPs, demonstrating that TRIM10 dissolves α-Syn. FIG. 43D depicts representative electron microscopy images of α-Syn PFFs (0.5 μM) fibril disaggregation when treated with GST or 0.5 μM Flag-TRIM10 (scale bar, 500 nm). FIG. 43E depicts representative images of dot blots of α-Syn fibrils (0.5 μM) treated with GST (0.2 μM), Flag-TRIM10 (0.5 μM), HSPs, or TRIM10 and HSPs, demonstrating that TRIM10 dissolves α-Syn. Soluble α-Syn relative to total α-Syn was quantified (n=3). FIG. 43F depicts representative images of Western blots and dot blots of Aβ42 fibrils incubated with GST, TRIM10 (50-500 μM), or DAXX (400 nM). FIG. 43G depicts representative results of ThT binding assays of Aβ42 fibrils incubated with GST, TRIM10 (50-500 nM), or DAXX (400 nM). FIG. 43H depicts representative images of Western blots and dot blots of 50 nM pre-formed Atxn1 82Q aggregates incubated with GST or Flag-TRIM10 (each at 0, 200 nM, and 500 nM) for 3 hours. For FIG. 43A and FIG. 43G, data are presented as mean±SD; n=4; **, p<0.01; ***, p<0.001; unpaired Student's t test.

FIG. 44, comprising FIG. 44A through FIG. 44E, depicts representative experimental results of TRIMs that can prevent TDP-43 aggregation. GFP-TDP-43^{NLRm} was transfected into HeLa cells alone (−) or together with the indicated TRIMs. Cells were treated with (+) or without (−) sodium arsenite (Ars) and stained with anti-HA antibody (for TRIMs) and Hoechst 33342 (for DNA). FIG. 44A depicts representative immunofluorescent images of cells with cytoplasmic GFP-TDP-43^{NLRm} aggregates in the presence or absence of TRIM10, TRIM11, TRIM17, and TRIM55. FIG. 44B, FIG. 44C, FIG. 44D, and FIG. 44E depict quantification of cells with cytoplasmic GFP-TDP-43^{NLRm} aggregates in the presence or absence of TRIM10, TRIM11, TRIM17, and TRIM55, respectively. Data are mean±SD, n=3. (scale bar, 10 μm) **P<0.01, ***P<0.001, unpaired Student's t test.

FIG. 45, comprising FIG. 45A through FIG. 45G, depicts representative experimental results demonstrating that downregulation of TRIM11 and TRIM10 in FTLD-TDP brains. FIG. 45A depicts a representative immunoblot of postmortem frontal cortex gray matter from 11 control and 11 FTLD-TDP individuals. FIG. 45B depicts a quantification of relative TRIM11/actin ratios. FIG. 45C depicts representative IHC images of TRIM11 and pS409/410 TDP-43 in frontal cortices. (Scale bar, 160 mm). FIG. 45D depicts a graph illustrating the negative correlation between levels of TRIM11 and pS409/410 TDP-43 among FTLD-TDP and control tissues. r and p values of the Pearson correlation coefficient are shown. FIG. 45E depicts a representative immunoblot of postmortem frontal cortex gray matter from 11 control and 11 FTLD-TDP individuals. FIG. 45F depicts a quantification of relative TRIM10/GAPDH protein ratios. FIG. 45G depicts a quantification of relative mRNA levels. The #1 control and #1 and #7 FTLP-TDP samples were used in both blots for comparison between these blots. **P<0.01, ***P<0.001; n.s, not significant; unpaired Student's t test.

FIG. 46, comprising FIG. 46A through FIG. 46G, depicts representative experimental results demonstrating the role of endogenous TRIM11 and TRIM10 in regulating TDP-43. FIG. 46A, FIG. 46B, and FIG. 46C depict representative immunofluorescent images, a quantification of pS409/410 intensity, and a representative immunoblot, respectively, of cortical neurons treated with control or TRIM11 ASO. FIG. 46D depicts representative immunofluorescent images of endogenous TDP-43 in control and TRIM10 KO HeLa cells treated with control or TRIM10 ASOs (scale bar, 10 μm). FIG. 46E depicts a quantification of cells with TDP-43 mis-localization in HeLa cells (***P<0.001, unpaired Student's t test). FIG. 46F depicts representative immunofluorescent images of endogenous TDP-43 in control and TRIM10 KO in mouse primary cortical neurons treated with control or TRIM10 ASOs (scale bar, 10 μm). FIG. 46G depicts a quantification of cells with TDP-43 mis-localization in primary neurons (***P<0.001, unpaired Student's t test).

FIG. 47, comprising FIG. 47A through FIG. 47D, depicts representative experimental results demonstrating that TRIM10 is a molecular chaperone and disaggregase for TDP-43. FIG. 47A depicts representative electron microscopy images of experiments and FIG. 47B depicts representative western blots from sedimentation assays where MBP-TDP-43 (5 μM) was treated with TEV and incubated for 24 h in the presence or absence of TRIM10 (0.25 or 0.5 μM). FIG. 47C depicts representative electron microscopy images of experiments and FIG. 47D depicts representative western blots from sedimentation assays where TDP-43 fibrils (5 μM monomer concentration) were incubated with or without TRIM10 (0.25 or 0.5 μM). SN, supernatant; PE, SDS-soluble pellet; SR, SDS-resistant pellet (detected by dot blot). Scale bar, 500 nm. Kapβ2 (2.5 and/or 5 μM), which does not act on TDP-43, was used as a negative control.

FIG. 48, comprising FIG. 48A through FIG. 48Q, depicts representative experimental results demonstrating that TRIM10 maintains nuclear localization of FUS. FIG. 48A depicts representative immunofluorescent images of HeLa cells transfected with EGFP-FUS plus empty vector (EV), TRIM10-FLAG-HA or Kapβ2-FLAG-HA treated with (+Ars) or without (−Ars) 0.5 mM sodium arsenite for 90 min. Cells were stained with anti-HA, G3BP1 and Hoechst 33342. (Scale bar, 10 μm). FIG. 48B depicts a quantification of percentage of cells with GFP-FUS in SGs. FIG. 48C depicts representative western blots from experiments where HeLa cells were transfected with EGFP-FUS with empty vector or TRIM10 for 24 hours and then treated with 0.5 mM sodium arsenite for 90 min, the cytoplasmic and nuclear fractions were isolated for western blot. Lamin A/C and GAPDH were used as the loading control for nuclear and cytoplasmic fractions, respectively. FIG. 48D depicts representative immunofluorescent images of HeLa cells transfected with mCherry-N1 or mCherry-N1-TRIM10 were treated with (+Ars) or without (−Ars) 0.5 mM sodium arsenite for 90 min. Cells were stained with anti-G3BP1, FUS and Hoechst 33342. (scale bar, 10 μm). FIG. 48E depicts a quantification of percentage of cells with FUS in SGs. FIG. 48F depicts representative immunofluorescent images of WT spinal cord neurons transfected with empty vector or TRIM10 for 24 h. Cells were treated with or without arsenite (0.5 mM) for one additional hour and stained with anti-HA, FUS and MAP2 antibodies and Hoechst 33342. (scale bar, 10 μm). FIG. 48G depicts a quantification of cells with FUS in cytoplasm. FIG. 48H depicts representative immunofluorescent images showing knockout of TRIM10 promotes SG association of GFP-FUS. GFP-FUS was transfected into control- or TRIM10-knockout HeLa cells for 24 hours. Cells were treated with or without arsenite (0.5 mM) for additional 30 min and stained with anti-G3BP1 antibody and Hoechst 33342. (scale bar, 10 μm). FIG. 48I depicts a quantification of cells with FUS in SGs. FIG. 48J depicts representative immunofluorescent images showing knockout of TRIM10 promotes SG association of endogenous FUS. Control- and TRIM10-knockdown HeLa cells were stained with anti-FUS antibody and Hoechst 33342. (scale bar, 10 μm). FIG. 48K depicts a quantification of percentage of cells with FUS in the cytoplasm. FIG. 48L depicts representative immunofluorescent images showing WT Cortical neurons transduced with control or TRIM10 #1 ASO for 3 days, and stained with anti-FUS, MAP2 antibodies and Hoechst 33342. (scale bar, 10 μm). FIG. 48M depicts a quantification of cells with FUS in cytoplasm. FIG. 48N depicts representative immunofluorescent images showing HeLa cells transfected with EGFP-FUS (G156E) together with TRIM10. Cells were treated with (+Ars) or without (−Ars) 0.5 mM sodium arsenite for 90 min, stained with anti-HA and anti-G3BP1 antibodies and Hoechst 33342, and analyzed by confocal microscopy. (scale bar, 10 μm). FIG. 48O depicts a quantification of percentage of cells with FUS G156E in SGs in the presence or absence of TRIM10. FIG. 48P depicts representative immunofluorescent images showing wild type spinal cord neurons transfected with EGFP-FUS (R521H) together with empty vector or TRIM10 for 24 h. Cells were treated with or without arsenite (0.5 mM) for one additional hour and stained with anti-HA and MAP2 antibodies and Hoechst 33342. (scale bar, 10 μm). FIG. 48Q depicts a quantification of cells with FUS inclusions in cytoplasm. Data shown are mean±SD [n=3, in each >20 cells were counted for FIG. 48B, FIG. 48E, FIG. 48G, FIG. 48I, FIG. 48M, FIG. 48O and FIG. 48Q; in each >60 cells were counted for FIG. 48K]. *P<0.05, **P<0.01, ***P<0.001, unpaired Student's t test.

FIG. 49, comprising FIG. 49A through FIG. 49D, depicts representative experimental results demonstrating that TRIM10 prevents and reverses SG association of hnRNPA1. FIG. 49A depicts representative immunofluorescent images from confocal microscopy of HeLa cells transfected with GFP-hnRNPA1 alone or together with TRIM10 for 24 h. Cells were treated with (+Ars) or without (−Ars) 0.5 mM sodium arsenite for 90 min, stained with anti-HA and anti-TIA1 antibodies and Hoechst 33342 (scale bar, 10 μm). FIG. 49B depicts a quantification of the percentage of cells with hnRNPA1 in SGs. FIG. 49C depicts representative immunofluorescent images of endogenous hnRNPA1 in WT cortical neurons transduced with control or TRIM10 ASO (scale bar, 10 μm). FIG. 49D depicts a quantification of FIG. 49C. Data shown are mean±SD, n=3 independent experiments. 25 cells or more were counted in each experiment. ***P<0.001, unpaired Student's t test.

FIG. 50, comprising FIG. 50A through FIG. 50K, depicts representative experimental results demonstrating that TRIM10 is a molecular chaperone and disaggregase for prion-like RBPs. FIG. 50A depicts a representative western blot from sedimentation assay and FIG. 50B depicts representative EM images of experiments where GST-FUS (5 μM) was treated with TEV, incubated for 24 h in the presence or absence of TRIM10 (0.5 μM). (scale bar, 500 nm). FIG. 50C depicts a representative western blot from sedimentation assay and FIG. 50D depicts representative EM images of experiments where GST-hnRNPA1 (5 μM) was treated with TEV and incubated for 24 h in the presence or absence of 0.5 or 5 μM of TRIM10 or Kapβ2 (FIG. 50C) or 0.5 μM of TRIM10 or 5 μM of Kapβ2 (FIG. 50D). FIG. 50E depicts a representative western blot from sedimentation assay and FIG. 50F depicts representative EM images of experiments where FUS-EGFP fibrils (5 μM monomer concentration) were incubated with indicated concentrations of TRIM10 or Kapβ2. (scale bar, 500 nm). Arrows indicate fibrillary FUS assemblies formed upon PSC cleavage. FIG. 50G depicts representative western blots of sedimentation assay and FIG. 50H depicts representative EM images of experiments where hnRNPA1 fibrils (5 μM monomer concentration) were incubated with buffer, 0.5 or 5 μM of TRIM10 or Kapβ2 (FIG. 50G), or 0.5 μM of TRIM10 or 5 μM of Kapβ2 (FIG. 50H). (scale bar, 500 nm). FIG. 50I depicts a representative image and FIG. 50J depicts a representative EM analysis of FUS hydrogel (480 μM monomer concentration) treated with buffer, TRIM10 (20 μM), or Kapβ2 (54 μM). FIG. 50K depicts representative images from experiments where hnRNPA1 hydrogels (3.3 mM monomer concentration) were treated with buffer, TRIM10 (20 μM), or Kapβ2 (270 μM). Dissolvement was assessed by EM. Molar ratio of TRIM10: hnRNPA1=1:83, Kapβ2: hnRNPA1=1:6. Scale Bar, 500 nm.

FIG. 51 through FIG. 53 depict representative immunofluorescent images of a systematic analysis of the effect of TRIMs on TDP-43 aggregation. GFP-TDP-43^{NLRmn} was transfected into HeLa cells alone (−) or together with the indicated TRIMs. Cells were treated with sodium arsenite (Ars) and stained with anti-HA antibody (for TRIMs) and Hoechst 33342 (for DNA). Shown are representative images. (scale bar, 10 μm).

FIG. 54 through FIG. 56 depict representative confocal images of a systematic analysis of the effect of TRIMs on FUS cytoplasmic mis-localization and SG association. HeLa cells were transfected with GFP-FUS together with the indicated TRIM proteins for 36 h. Cells were treated with 0.5 mM sodium arsenite (Ars) for 90 min, fixed, and stained with anti-HA and anti-TIA1 antibodies and Hoechst 33342. (scale bar, 10 μm).

FIG. 57, comprising FIG. 57A through FIG. 57K, depicts representative experimental results demonstrating that TRIM10 maintains nuclear localization of wild-type FUS. FIG. 57A through FIG. 57C depict representative results from experiments where HeLa cells were transfected with or without HA-TRIM10 for 24 h, cells were treated with (+Ars) or without (−Ars) 0.5 mM sodium arsenite for 90 min. Cells were fixed and stained with anti-HA and anti-G3BP1 antibodies and Hoechst 33342. FIG. 57A depicts a representative western blot showing expression of indicated proteins. FIG. 57B depicts representative cell images. (scale bar, 10 μm). FIG. 57C depicts a quantification of mean fluorescence intensity of SGs. FIG. 57D and FIG. 57E depict representative results from experiments where cortical neurons derived from wild-type mice were transfected with empty vector or TRIM10 for 24 h. Cells were treated with or without arsenite (0.5 mM) for one additional hour and stained with anti-HA, FUS, and MAP2 antibodies and Hoechst 33342. FIG. 57D depicts representative cell images. (scale bar, 10 μm). FIG. 57E depicts a quantification of cells with FUS in cytoplasm. FIG. 57F depicts a representative western blot showing protein levels in Ctr and TRIM10-knockout cell lines. FIG. 57G through FIG. 57I depict representative results from experiments where Control- or TRIM10-knockout HeLa cells were treated with or without 0.5 mM sodium arsenite for 60 min and stained with anti-G3BP1 antibody and Hoechst 33342. FIG. 57G depicts a representative western blot showing TRIM10 knockdown efficiency and G3BP1 expression. FIG. 57H depicts representative cell images. (scale bar, 10 μm). FIG. 57I depicts a quantification of mean fluorescence intensity of SGs. FIG. 57J depicts representative fluorescence images of primary cortical neurons treated with or without SCR CTRL-FAR RED for 3 days. (scale bar, 10 μm). FIG. 57K depicts a representative western blot from of primary cortical neurons incubated with AUMInc-scrctrl or AUMSiI-TRIM10-1/3 for 3 days. Cells were lysed in RIPA buffer. Data shown are mean±SD [n=3. in each ≥25 cells, ≥20 SGs were counted for FIG. 57C and FIG. 57I; in each >20 cells were counted for FIG. 57E]. *P<0.05. ns, not significant, unpaired Student's t test.

FIG. 58, comprising FIG. 58A through FIG. 58C, depicts representative experimental results demonstrating that TRIM10 maintains nuclear localization of FUS mutants. FIG. 58A depicts representative immunofluorescent images of wild type spinal cord neurons transfected with EGFP-FUS (R521H) together with empty vector or TRIM10 for 24 h. Cells were treated with or without arsenite (0.5 mM) for one additional hour and stained with anti-HA, G3BP1 and MAP2 antibodies and Hoechst 33342. FIG. 58B depicts representative immunofluorescent images of wild type cortical neurons transfected with EGFP-FUS (R521H) together with empty vector or TRIM10 for 24 h. Cells were treated with or without arsenite (0.5 mM) for one additional hour and stained with anti-HA, G3BP1 and MAP2 antibodies and Hoechst 33342. (scale bar, 10 μm). FIG. 58C depicts a quantification of cells with FUS inclusions in cytoplasm. Data shown are mean±SD [n=3. ≥20 cells each]. *P<0.05, **P<0.01, unpaired Student's t test.

FIG. 59, comprising FIG. 59A through FIG. 59K, depicts representative experimental results demonstrating that TRIM10 prevents SG association of hnRNPA1. FIG. 59A depicts representative immunofluorescent images of HeLa cells were transfected with GFP-hnRNPA1 alone or together with TRIM10 for 24 h. Cells were treated with (+Ars) or without (−Ars) 0.5 mM sodium arsenite for 90 min, stained with anti-HA and anti-TIA1 antibodies and Hoechst 33342, and analyzed by confocal microscope. (scale bar, 10 μm). FIG. 59A is related with FIG. 45L. FIG. 59B depicts a representative western blot of the cytoplasmic and nuclear fractions isolated from HeLa cells transfected with EGFP-hnRNPA1 plus empty vector or TRIM10 for 24 hours and treated with 0.5 mM sodium arsenite for additional 90 min. Lamin A/C and GAPDH were used as the loading control for nuclear and cytoplasmic fractions, respectively. FIG. 59C depicts a representative western blot of the expression of proteins from HeLa cells transfected with GFP-hnRNPA1 alone or together with TRIM10 for 24 hours. Cells were treated with (+Ars) or without (−Ars) 0.5 mM sodium arsenite for 90 min. FIG. 59D depicts representative immunofluorescent images of HeLa cells transfected with GFP-hnRNPA1 for 24 hours and then transfected with TRIM10 as indicated. 24 hours later, cells were treated with (+Ars) or without (−Ars) 0.5 mM sodium arsenite for 90 min before fixed and stained with anti-TIA1 antibody and Hoechst 33342 for confocal microscopy. (scale bar, 10 μm). FIG. 59E depicts a quantification of percentage of cells with hnRNPA1 in SGs. FIG. 59F depicts a representative western blot of protein expression analysis. FIG. 59G depicts a representative western blot of HeLa cells transfected with empty vector or TRIM10 for 24 h and treated with 0.5 mM sodium arsenite for additional 90 min. Total cell lysates and cytoplasmic and nuclear fractions were analyzed by western blot. Lamin A/C and GAPDH were used as the control for nuclear and cytoplasmic fractions, respectively. FIG. 59H depicts representative immunofluorescent images of GFP-hnRNPA1 transfected into control- or TRIM10 knockdown-HeLa cells for 36 hours before being fixed and stained with anti-G3BP1 antibody and Hoechst 33342 for confocal microscopy (scale bar, 10 μm). FIG. 59I depicts a quantification of percentage of cells with hnRNPA1 in cytoplasm. FIG. 59J depicts representative immunofluorescent images of endogenous hnRNPA1 in control- or TRIM10 knockdown-Hela cells. (scale bar, 10 μm). FIG. 59K depicts a quantification percentage of cells with hnRNPA1 in cytoplasm. Data shown are mean±SD [n=3. >20 cells were counted in each for FIG. 59F, FIG. 59I and FIG. 59K]. ***P<0.001, unpaired Student's t test.

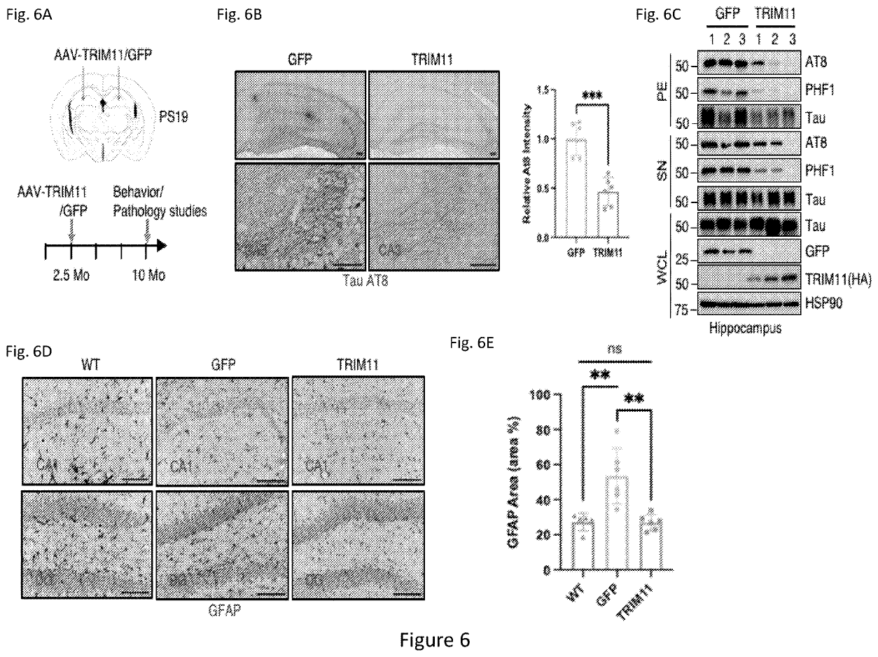

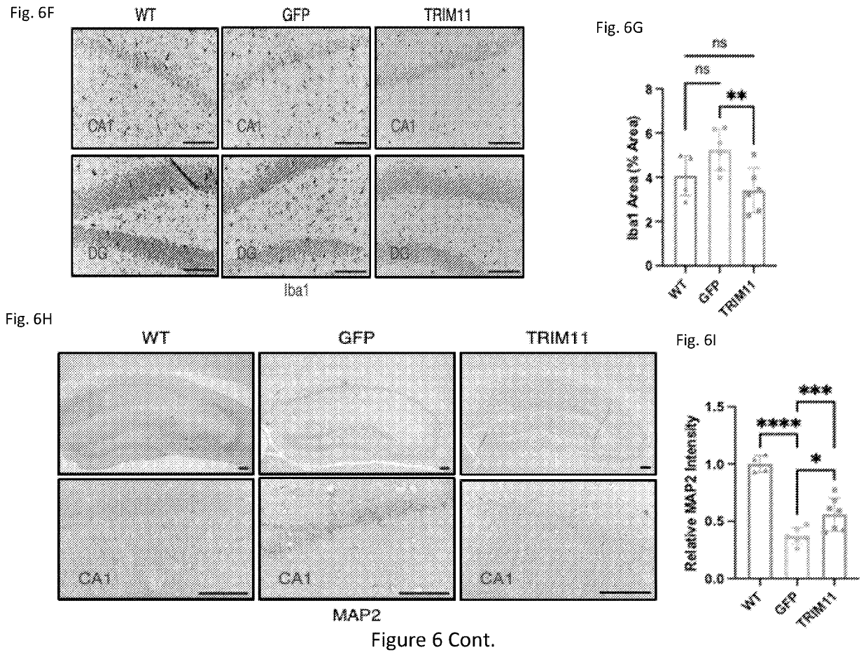

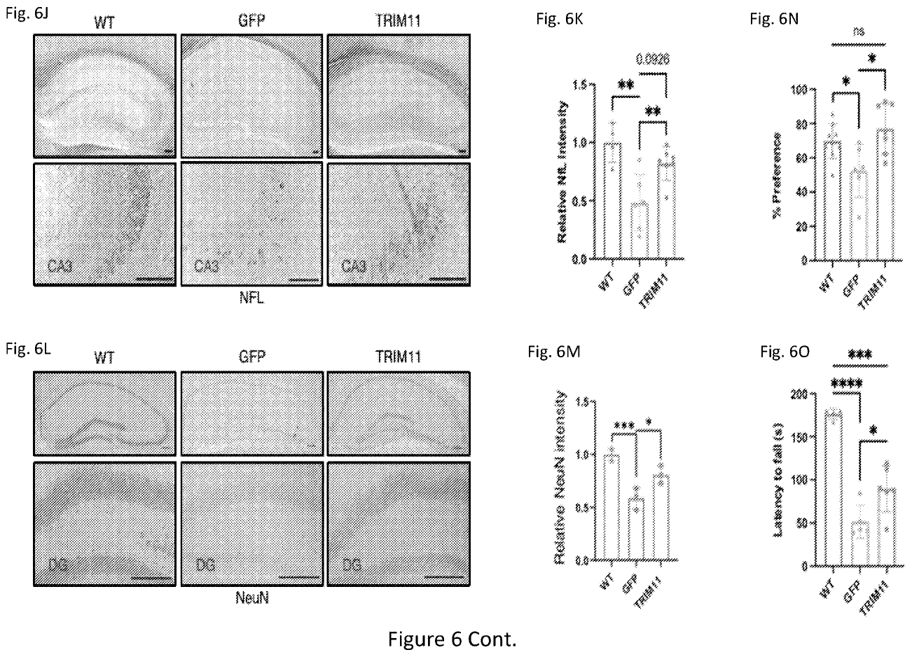

FIG. 6O, comprising FIG. 60A and FIG. 60B, depicts a scheme of expression constructs and analysis of purified proteins. FIG. 60A depicts a schematic representation of recombinant proteins used in this study. Human FUS and hnRNPA1 were N-terminally fused with Glutathione S-transferases (GST). The GST is cleaved off the fusions using TEV to initiate protein aggregation. FIG. 60B depicts representative western blot images of recombinant TRIM10 protein (human TRIM10 was tagged with both HA and Flag epitopes at the C-terminus) expressed in HEK293T cells, FUS and hnRNPA1 proteins expressed in E. coli purified and analyzed by SDS-PAGE and Coomassie blue staining. BSA was used as a protein standard.

### DETAILED DESCRIPTION

The present invention is related to the discovery of the role TRIM proteins as molecular chaperones, disaggregases, and identifiers of proteins for degradation, which play a role in the pathology of a variety of neurodegenerative disorders.

In one aspect, the present invention provides compositions and methods to treat or prevent a neurodegenerative disease or disorders. In some embodiments, the neurodegenerative disease or disorder is associated with misfolded proteins or protein aggregates. Thus, in certain aspects, the present invention can be used to eliminate intracellular or extracellular misfolded proteins, protein aggregates, or protein inclusions.

In some embodiments, the invention provides compositions and methods for the treatment of disorders associated with misfolded proteins or protein aggregates. For example, in certain embodiments, the compositions and methods are used for the treatment of diseases and disorders associated with misfolded proteins and/or protein aggregates of amyloid-beta, alpha-synuclein, tau, prions, SOD1, TDP-43, FUS, p53, p53 mutants, or proteins associated with polyglutamine repeats, such as huntingtin and ataxins.

In certain embodiments, the invention provides compositions and methods to treat or prevent a neurodegenerative disorder in a subject in need thereof. In some embodiments, the invention provides compositions and methods for the treatment or prevention of neurodegenerative disorders that are poly-glutamine (polyQ) disorders, where repeats of the CAG codon encode proteins with polyglutamine tracts that can result in misfolded protein aggregates. Exemplary polyQ disorders include, but are not limited to Spinocerebellar ataxia (SCA) Type 1 (SCA1), SCA2, SCA3, SCA6, SCA7, SCA17, Huntington's disease, and Dentatorubral-pallidoluysian atrophy (DRPLA).

Exemplary neurodegenerative diseases associated with misfolded proteins or protein aggregates include, but are not limited to, SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), dementia with Lewy bodies (DLB), multiple system atrophy (MSA), Frontotemporal lobar degeneration (FTLD), AL amyloidosis, AA amyloidosis, Familial Mediterranean fever, senile systemic amyloidosis, familial amyloidotic polyneuropathy, Icelandic hereditary cerebral amyloid angiopathy, hereditary cerebral hemorrhage with amyloidosis, pituitary prolactinoma, frontotemporal lobar degeneration (FTLD-tau), progressive supranuclear palsy (PSP), corticobasal degeneration (CBD), argyrophilic grain disease (AGD), frontotemporal dementia and parkinsonism linked to chromosome 17 (FTDP-17), vacuolar tauopathy, Lytico-bodig disease, globular glial tauopathy (GGT), ageing-related tau astrogliopathy (ARTAG), Pick's disease, primary age-related tauopathy (PART), tangle only dementia (TOD), chronic traumatic encephalopathy (CTE), anti-IgLON5-related tauopathy, Guadeloupean parkinsonism, Nodding Syndrome (NS), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, Shy-Drager syndrome, striatonigral degeneration, olivopontocerebellar atrophy, Hallervorden-Spatz syndrome, REM sleep behavior disorder (RPD), Alzheimer's disease with amygdalar restricted Lewy bodies (AD/ALB), frontotemporal lobar degeneration (FTLD-TDP), multiple system proteinopathy (MSP), Perry disease, facial onset sensory and motor neuronopathy (FOSMN), cerebral age-related TDP-43 with sclerosis (CARTS), limbic-predominant age-related TDP-43 encephalopathy (LATE), sporadic inclusion body myositis (sIBM), chronic traumatic encephalopathy (CTE), primary lateral sclerosis (PLS), progressive muscular atrophy (PMA), Guam Parkinson-dementia complex (G-PDC), and Guam amyotrophic lateral sclerosis (G-ALS).

In one aspect, the present invention provides compositions and methods to increase the expression, activity, or both of one or more TRIM proteins. In certain embodiments, the composition comprises a nucleic acid molecule, expression vector, protein, peptide, small molecule, or the like, which increases the expression, activity, or both of one or more TRIM proteins.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20%, ±10%, ±5%, ±1%, or ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

As used herein, “cell therapy” refers to the administration of viable cells to a subject as a therapeutic to treat or prevent one or more diseases or disorders. The cells can be unmanipulated or manipulated, such as cells genetically engineered to overexpress a therapeutic protein of interest. The cells for use in cell therapy can be xenogeneic, allogeneic, syngeneic, or autologous.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a sign or symptom of the disease or disorder, the frequency with which such a sign or symptom is experienced by a patient, or both, is reduced.

An “effective amount” or “therapeutically effective amount” of a compound is that amount of a compound which is sufficient to provide a beneficial effect to the subject to which the compound is administered. An “effective amount” of a delivery vehicle is that amount sufficient to effectively bind or deliver a compound.

As used herein, an “instructional material” includes a publication, a recording, a diagram, or any other medium of expression which can be used to communicate the usefulness of a compound, composition, vector, or delivery system of the invention in the kit for effecting alleviation of the various diseases or disorders recited herein. Optionally, or alternately, the instructional material can describe one or more methods of alleviating the diseases or disorders in a cell or a tissue of a mammal. The instructional material of the kit of the invention can, for example, be affixed to a container which contains the identified compound, composition, vector, or delivery system of the invention or be shipped together with a container which contains the identified compound, composition, vector, or delivery system. Alternatively, the instructional material can be shipped separately from the container with the intention that the instructional material and the compound be used cooperatively by the recipient.

The terms “patient,” “subject,” “individual,” and the like are used interchangeably herein, and refer to any animal, or cells thereof whether in vitro or in vivo, amenable to the methods described herein. In certain non-limiting embodiments, the patient, subject or individual is a human.

A “therapeutic” treatment is a treatment administered to a subject who exhibits signs or symptoms of a disease or disorder, for the purpose of diminishing or eliminating those signs or symptoms.

As used herein, “treating a disease or disorder” means reducing the severity and/or frequency with which a sign or symptom of the disease or disorder is experienced by a patient.

The phrase “biological sample” as used herein, is intended to include any sample comprising a cell, a tissue, or a bodily fluid in which expression of a nucleic acid or polypeptide is present or can be detected. Samples that are liquid in nature are referred to herein as “bodily fluids.” Biological samples may be obtained from a patient by a variety of techniques including, for example, by scraping or swabbing an area of the subject or by using a needle to obtain bodily fluids. Methods for collecting various body samples are well known in the art.

As used herein, an “immunoassay” refers to any binding assay that uses an antibody capable of binding specifically to a target molecule to detect and quantify the target molecule.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific.

In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

A “coding region” of a mRNA molecule consists of the nucleotide residues of the mRNA molecule which are matched with an anti-codon region of a transfer RNA molecule during translation of the mRNA molecule or which encode a stop codon. The coding region may thus include nucleotide residues comprising codons for amino acid residues which are not present in the mature protein encoded by the mRNA molecule (e.g., amino acid residues in a protein export signal sequence).

“Complementary” as used herein to refer to a nucleic acid, refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

“Isolated” means altered or removed from the natural state. For example, a nucleic acid or a peptide naturally present in its normal context in a living animal is not “isolated,” but the same nucleic acid or peptide partially or completely separated from the coexisting materials of its natural context is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes a recombinant DNA which is part of a hybrid gene encoding additional polypeptide sequence.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

As used herein, “conjugated” refers to covalent attachment of one molecule to a second molecule.

“Variant” as the term is used herein, is a nucleic acid sequence or a peptide sequence that differs in sequence from a reference nucleic acid sequence or peptide sequence respectively, but retains essential biological properties of the reference molecule. Changes in the sequence of a nucleic acid variant may not alter the amino acid sequence of a peptide encoded by the reference nucleic acid, or may result in amino acid substitutions, additions, deletions, fusions and truncations. Changes in the sequence of peptide variants are typically limited or conservative, so that the sequences of the reference peptide and the variant are closely similar overall and, in many regions, identical. A variant and reference peptide can differ in amino acid sequence by one or more substitutions, additions, deletions in any combination. A variant of a nucleic acid or peptide can be a naturally occurring such as an allelic variant, or can be a variant that is not known to occur naturally. Non-naturally occurring variants of nucleic acids and peptides may be made by mutagenesis techniques or by direct synthesis.

As used herein, a “activator of one or more TRIM proteins” is a compound that increases the expression, activity or biological function of the TRIM proteins as compared to the expression, activity or biological function of the TRIM proteins in the absence of the activator.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the present invention provides compositions and methods to treat or prevent a neurodegenerative disease or disorder. In some embodiments, the neurodegenerative disease or disorder is associated with misfolded proteins or protein aggregates. For example, the present invention provides compositions and methods to increase the recognition and elimination of misfolded proteins. In certain embodiments, the invention assists in the folding of proteins. In certain embodiments, the invention provides for the disaggregation and refolding of protein aggregates or inclusions. The present invention can thus be used to treat or prevent misfolded proteins, protein aggregates, or protein inclusions, both intracellularly or extracellularly.

The present invention is related to the discovery of the role of TRIM proteins as molecular chaperones, disaggregases, and identifiers of proteins for degradation, which play a role in the pathology of a variety of neurodegenerative disorders.

### Compositions

In various embodiments, the present invention includes compositions for increasing the level or activity of a TRIM protein. Therefore, in some embodiments, the composition comprises an activator of the expression or activity of one or more TRIM proteins. In some embodiments, the activator increases the expression or activity of one or more TRIM proteins. In some embodiments, the compositions of the invention increase the level of one or more TRIM proteins, the amount of mRNA encoding for one or more TRIM proteins, the activity of one or more TRIM proteins, or a combination thereof. The one or more TRIM proteins include any member of the TRIM protein family, including mammalian and non-mammalian members. In certain embodiments, composition comprises an activator of one or more selected from the group consisting of human TRIM1, TRIM2, TRIM3, TRIM4, TRIM5, TRIM6, TRIM7, TRIM8, TRIM9, TRIM10, TRIM11, TRIM13, TRIM14, TRIM15, TRIM16, TRIM17, TRIM18, TRIM19, TRIM20, TRIM21, TRIM22, TRIM23, TRIM24, TRIM25, TRIM26, TRIM27, TRIM28, TRIM29, TRIM30, TRIM31, TRIM32, TRIM33, TRIM34, TRIM35, TRIM36, TRIM37, TRIM38, TRIM39, TRIM40, TRIM41, TRIM42, TRIM43, TRIM44, TRIM45, TRIM46, TRIM47, TRIM48, TRIM49, TRIM50, TRIM51, TRIM52, TRIM54, TRIM55, TRIM56, TRIM58, TRIM59, TRIM60, TRIM61, TRIM62, TRIM63, TRIM64, TRIM65, TRIM66, TRIM67, TRIM68, TRIM69, TRIM70, TRIM71, TRIM72, TRIM73, TRIM74, TRIM76, and TRIM77; and mouse TRIM12 and TRIM30. In some embodiments, the composition comprises an activator of one or more selected from the group consisting of human TRIM2, TRIM3, TRIM4, TRIM5, TRIM9, TRIM10, TRIM11, TRIM17, TRIM18, TRIM19, TRIM21, TRIM24, TRIM26, TRIM29, TRIM31, TRIM34, TRIM36, TRIM37, TRIM46, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70; and mouse TRIM12 and TRIM30. In some embodiments, the composition comprises an activator of one or more selected from the group consisting of human TRIM10, TRIM11, TRIM24, TRIM36, TRIM37, TRIM40, TRIM49, TRIM55, TRIM58, and TRIM68. In some embodiments, the composition comprises an activator of one or more selected from the group consisting of human TRIM2, TRIM3, TRIM4, TRIM5, TRIM9, TRIM10, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70. In some embodiments, the composition comprises an activator of one or more selected from the group consisting of human TRIM10, TRIM11, and TRIM55. In some embodiments, the composition comprises an activator of one or more selected from the group consisting of human TRIM2, TRIM3, TRIM10, TRIM11, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73. In some embodiments, the composition comprises an activator of one or more selected from the group consisting of human TRIM10, TRIM11, TRIM36, TRIM55, and TRIM68. In some embodiments, the composition comprises an activator of one or more selected from the group consisting of human TRIM10, TRIM11, TRIM24, TRIM36, and TRIM58. In some embodiments, the composition comprises an activator of one or more selected from the group consisting of human TRIM10, TRIM11, TRIM17, TRIM36, TRIM37, TRIM40, TRIM49, and TRIM55. In some embodiments, the composition comprises an activator of human TRIM10. In some embodiments, the composition comprises an activator of human TRIM11.

Activation of a gene, or gene product, can be assessed using a wide variety of methods, including those disclosed herein, as well as methods known in the art or to be developed in the future. That is, the routineer would appreciate, based upon the disclosure provided herein, that increasing the level or activity of a gene, or gene product, can be readily assessed using methods that assess the level of a nucleic acid encoding a gene product (e.g., mRNA), the level of polypeptide gene product present in a biological sample, the activity of polypeptide gene product present in a biological sample, or combinations thereof.

The activator compositions and methods of the invention that increase the level or activity of a gene, or gene product, include, but should not be construed as being limited to, a chemical compound, a protein, a peptide, a peptidomimetic, an antibody, a ribozyme, a small molecule chemical compound, a nucleic acid, a vector, an antisense nucleic acid molecule (e.g., siRNA, miRNA, etc.), or combinations thereof. One of skill in the art would readily appreciate, based on the disclosure provided herein, that an activator composition encompasses a chemical compound that increases the level or activity of a gene, or gene product. Additionally, an activator composition encompasses a chemically modified compound, and derivatives, as is well known to one of skill in the chemical arts.

In some embodiments, the activator composition of the present invention is an agonist, which increases the expression, activity, or biological function of a gene or gene product. For example, in certain embodiments, the activator of the present invention is an agonist of one or more TRIM proteins.

Further, one of skill in the art would, when equipped with this disclosure and the methods exemplified herein, appreciate that activators include such activators as discovered in the future, as can be identified by well-known criteria in the art of pharmacology, such as the physiological results of modulation of the genes, and gene products, as described in detail herein and/or as known in the art. Therefore, the present invention is not limited in any way to any particular activator composition as exemplified or disclosed herein; rather, the invention encompasses those activator compositions that would be understood by the routineer to be useful as are known in the art and as are discovered in the future.

Further methods of identifying and producing activator compositions are well known to those of ordinary skill in the art. Alternatively, an activator can be synthesized chemically. Further, the routineer would appreciate, based upon the teachings provided herein, that an activator composition can be obtained from a recombinant organism. Compositions and methods for chemically synthesizing activators and for obtaining them from natural sources are well known in the art and are described in the art.

One of skill in the art will appreciate that an activator can be administered as a small molecule chemical, a polypeptide, a peptide, an antibody, a nucleic acid construct encoding a protein, an antisense nucleic acid, a nucleic acid construct encoding an antisense nucleic acid, or combinations thereof. Numerous vectors and other compositions and methods are well known for administering a protein or a nucleic acid construct encoding a protein to cells or tissues. Therefore, the invention includes a peptide or a nucleic acid encoding a peptide that is an activator of a gene, or gene product. For example, the invention includes a peptide or a nucleic acid encoding a peptide that comprises one or more TRIM proteins, one or more functional TRIM peptides or a combination thereof. (Sambrook et al., 2001, Molecular Cloning: A Laboratory Manual, Cold Spring Harbor Laboratory, New York; Ausubel et al., 1997, Current Protocols in Molecular Biology, John Wiley & Sons, New York).

In some embodiments, the activators of the invention results in an increase in expression of at least one TRIM protein, including transcription, translation, or both. In some embodiments, the activators of the invention results in an increase in at least one activity of a TRIM protein (e.g., inhibition of USP14). Thus, increasing the level or activity of at least one TRIM protein includes, but is not limited to, increasing the amount of at least one TRIM protein, increasing transcription, translation, or both, of a nucleic acid encoding at least one TRIM protein; and it also includes increasing any activity of a TRIM polypeptide as well.

One of skill in the art will realize that diminishing the amount or activity of a molecule that itself diminishes the amount or activity of a TRIM protein can serve to increase the amount or activity of the trim protein. Any inhibitor of a negative regulator of a TRIM protein is encompassed in the invention. As a non-limiting example, antisense is described as a form of inhibiting a regulator of proteasome or a proteasome subunit in order to increase the amount or activity of proteasome or a proteasome subunit. Antisense oligonucleotides are DNA or RNA molecules that are complementary to some portion of a mRNA molecule. When present in a cell, antisense oligonucleotides hybridize to an existing mRNA molecule and inhibit translation into a gene product. Inhibiting the expression of a gene using an antisense oligonucleotide is well known in the art (Marcus-Sekura, 1988, Anal. Biochem. 172:289), as are methods of expressing an antisense oligonucleotide in a cell (Inoue, U.S. Pat. No. 5,190,931). The methods of the invention include the use of antisense oligonucleotide to diminish the amount of a molecule that causes a decrease in the amount or activity of a TRIM protein, thereby increasing the amount or activity of the TRIM protein. Contemplated in the present invention are antisense oligonucleotides that are synthesized and provided to the cell by way of methods well known to those of ordinary skill in the art. As an example, an antisense oligonucleotide can be synthesized to be between about 10 and about 100, more preferably between about 15 and about 50 nucleotides long. The synthesis of nucleic acid molecules is well known in the art, as is the synthesis of modified antisense oligonucleotides to improve biological activity in comparison to unmodified antisense oligonucleotides (Tullis, 1991, U.S. Pat. No. 5,023,243).

Similarly, the expression of a gene may be inhibited by the hybridization of an antisense molecule to a promoter or other regulatory element of a gene, thereby affecting the transcription of the gene. Methods for the identification of a promoter or other regulatory element that interacts with a gene of interest are well known in the art, and include such methods as the yeast two hybrid system (Bartel and Fields, eds., In: The Yeast Two Hybrid System, Oxford University Press, Cary, N.C.).

Alternatively, inhibition of a gene expressing a protein that diminishes the level or activity of a TRIM protein can be accomplished through the use of an siRNA, shRNA, antisense oligonucleotide or ribozyme. Given the nucleotide sequence of the molecule, one of ordinary skill in the art could synthesize an antisense oligonucleotide or ribozyme without undue experimentation, provided with the disclosure and references incorporated herein.

### Peptides

In some embodiments, the activator of the present invention comprises an active TRIM polypeptide, or fragment thereof. In some embodiments, a peptide of the composition comprises an amino acid sequence of one or more TRIM proteins. In some embodiments, a peptide of the composition comprises an amino acid sequence of one or more selected from the group consisting of human TRIM1, TRIM2, TRIM3, TRIM4, TRIM5, TRIM6, TRIM7, TRIM8, TRIM9, TRIM10, TRIM11, TRIM13, TRIM14, TRIM15, TRIM16, TRIM17, TRIM18, TRIM19, TRIM20, TRIM21, TRIM22, TRIM23, TRIM24, TRIM25, TRIM26, TRIM27, TRIM28, TRIM29, TRIM30, TRIM31, TRIM32, TRIM33, TRIM34, TRIM35, TRIM36, TRIM37, TRIM38, TRIM39, TRIM40, TRIM41, TRIM42, TRIM43, TRIM44, TRIM45, TRIM46, TRIM47, TRIM48, TRIM49, TRIM50, TRIM51, TRIM52, TRIM54, TRIM55, TRIM56, TRIM58, TRIM59, TRIM60, TRIM61, TRIM62, TRIM63, TRIM64, TRIM65, TRIM66, TRIM67, TRIM68, TRIM69, TRIM70, TRIM71, TRIM72, TRIM73, TRIM74, TRIM76, and TRIM77 peptides; mouse TRIM12 and TRIM30 peptides; and functional variants thereof. In some embodiments, a peptide of the composition comprises an amino acid sequence of one or more selected from the group consisting of human TRIM2, TRIM3, TRIM4, TRIM5, TRIM9, TRIM10, TRIM11, TRIM17, TRIM18, TRIM19, TRIM21, TRIM24, TRIM26, TRIM29, TRIM30, TRIM31, TRIM34, TRIM36, TRIM37, TRIM46, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70; mouse TRIM12 and TRIM30 peptides; and functional variants thereof. In some embodiments, a peptide of the composition comprises an amino acid sequence of one or more selected from the group consisting of human TRIM10, TRIM11, TRIM24, TRIM36, TRIM37, TRIM40, TRIM49, TRIM55, TRIM58, and TRIM68 peptides and functional variants thereof. In some embodiments, a peptide of the composition comprises an amino acid sequence of one or more selected from the group consisting of human TRIM2, TRIM3, TRIM4, TRIM5, TRIM9, TRIM10, TRIM11, TRIM12, TRIM17, TRIM18, TRIM19, TRIM21, TRIM26, TRIM29, TRIM30, TRIM31, TRIM34, TRIM36 TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70 peptides and functional variants thereof. In some embodiments, a peptide of the composition comprises an amino acid sequence of one or more selected from the group consisting of human TRIM10, TRIM11, and TRIM55 peptides and functional variants thereof. In some embodiments, a peptide of the composition comprises an amino acid sequence of one or more selected from the group consisting of human TRIM2, TRIM3, TRIM10, TRIM11, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73 peptides and functional variants thereof. In some embodiments, a peptide of the composition comprises an amino acid sequence of one or more selected from the group consisting of human TRIM10, TRIM11, TRIM36, TRIM55, and TRIM68 peptides and functional variants thereof. In some embodiments, a peptide of the composition comprises an amino acid sequence of one or more selected from the group consisting of human TRIM10, TRIM11, TRIM24, TRIM36, and TRIM58 peptides and functional variants thereof. In some embodiments, a peptide of the composition comprises an amino acid sequence of one or more selected from the group consisting of human TRIM10, TRIM11, TRIM17, TRIM36, TRIM37, TRIM40, TRIM49, and TRIM55 peptides and functional variants thereof. In some embodiments, a peptide of the composition comprises an amino acid sequence of human TRIM10 peptide or functional variants thereof. In some embodiments, a peptide of the composition comprises an amino acid sequence of human TRIM11 peptide or functional variants thereof.

In some embodiments, the composition comprises one or more TRIM proteins. For example, in some embodiments, in the composition comprises an amino acid sequence of one or more TRIM proteins provided by the accession numbers of Table 1.

The invention should also be construed to include any form of a peptide having substantial homology to the peptides disclosed herein. A peptide which is “substantially homologous” is at least about 50% identical, at least about 70% identical, at least about 80% identical, at least about 85% identical, at least about 86 identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, to the amino acid sequence of the peptides disclosed herein.

In some embodiments, the composition of the invention comprises a peptide, a fragment of a peptide, a homolog, a variant, a derivative or a salt of a peptide described herein. For example, in certain embodiments, the composition comprises a peptide comprising one or more TRIM proteins, a fragment of one or more TRIM proteins, a homolog of one or more TRIM proteins, a variant of one or more TRIM proteins, a derivative of one or more TRIM proteins, or a salt of one or more TRIM proteins.

In certain embodiments, the peptide comprises a targeting domain, which targets the peptide to a desired location. For example, in certain embodiments, the targeting domain binds to a targeted cell, protein, or protein aggregate, thereby delivering the therapeutic peptide to a desired location. For example, in some embodiments, the targeting domain is directed to bind to a protein or protein aggregate associated with a disease or disorder, including but not limited to the proteins and protein aggregates of amyloid-beta, alpha-synuclein, tau, prions, SOD1, TDP-43, FUS, p53 mutants, and proteins associated with polyglutamine repeats, such as huntingtin and ataxins.

In certain embodiments, the targeting domain comprises a peptide, nucleic acid, small molecule, or the like, which has the ability to bind to the targeted cell, protein, or protein aggregate. In some embodiments, the targeting domain comprises an antibody or antibody fragment which binds to a targeted cell, protein, or protein aggregate.

The peptide of the present invention may be made using chemical methods. For example, peptides can be synthesized by solid phase techniques (Roberge J Y et al (1995) Science 269:202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

The peptides of the invention can be post-translationally modified. For example, post-translational modifications that fall within the scope of the present invention include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation. A variety of approaches are available for introducing unnatural amino acids during protein translation. By way of example, special tRNAs, such as tRNAs which have suppressor properties, suppressor tRNAs, have been used in the process of site-directed non-native amino acid replacement (SNAAR). In SNAAR, a unique codon is required on the mRNA and the suppressor tRNA, acting to target a non-native amino acid to a unique site during the protein synthesis (described in WO90/05785). However, the suppressor tRNA must not be recognizable by the aminoacyl tRNA synthetases present in the protein translation system. In certain cases, a non-native amino acid can be formed after the tRNA molecule is aminoacylated using chemical reactions which specifically modify the native amino acid and do not significantly alter the functional activity of the aminoacylated tRNA. These reactions are referred to as post-aminoacylation modifications. For example, the epsilon-amino group of the lysine linked to its cognate tRNA (TRNALYS), could be modified with an amine specific photoaffinity label.

The peptides of the invention may be conjugated with other molecules, such as proteins, to prepare fusion proteins. This may be accomplished, for example, by the synthesis of N-terminal or C-terminal fusion proteins provided that the resulting fusion protein retains the functionality of the peptide of the invention.

Cyclic derivatives of the peptides of the invention are also part of the present invention. Cyclization may allow the peptide to assume a more favorable conformation for association with other molecules. Cyclization may be achieved using techniques known in the art. For example, disulfide bonds may be formed between two appropriately spaced components having free sulfhydryl groups, or an amide bond may be formed between an amino group of one component and a carboxyl group of another component. Cyclization may also be achieved using an azobenzene-containing amino acid as described by Ulysse, L., et al., J. Am. Chem. Soc. 1995, 117, 8466-8467. The components that form the bonds may be side chains of amino acids, non-amino acid components or a combination of the two. In an embodiment of the invention, cyclic peptides may comprise a beta-turn in the right position. Beta-turns may be introduced into the peptides of the invention by adding the amino acids Pro-Gly at the right position.

It may be desirable to produce a cyclic peptide which is more flexible than the cyclic peptides containing peptide bond linkages as described above. A more flexible peptide may be prepared by introducing cysteines at the right and left position of the peptide and forming a disulfide bridge between the two cysteines. The two cysteines are arranged so as not to deform the beta-sheet and turn. The peptide is more flexible as a result of the length of the disulfide linkage and the smaller number of hydrogen bonds in the beta-sheet portion. The relative flexibility of a cyclic peptide can be determined by molecular dynamics simulations.

The peptides of the invention may be converted into pharmaceutical salts by reacting with inorganic acids such as hydrochloric acid, sulfuric acid, hydrobromic acid, phosphoric acid, etc., or organic acids such as formic acid, acetic acid, propionic acid, glycolic acid, lactic acid, pyruvic acid, oxalic acid, succinic acid, malic acid, tartaric acid, citric acid, benzoic acid, salicylic acid, benezenesulfonic acid, and toluenesulfonic acids.

Peptides of the invention may also have modifications. Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are peptides which have been modified using ordinary molecular biological techniques so as to improve their resistance to proteolytic degradation or to optimize solubility properties or to render them more suitable as a therapeutic agent. Such variants include those containing residues other than naturally-occurring L-amino acids, e.g., D-amino acids or non-naturally-occurring synthetic amino acids. The peptides of the invention may further be conjugated to non-amino acid moieties that are useful in their therapeutic application. In particular, moieties that improve the stability, biological half-life, water solubility, and/or immunologic characteristics of the peptide are useful. A non-limiting example of such a moiety is polyethylene glycol (PEG).

Covalent attachment of biologically active compounds to water-soluble polymers is one method for alteration and control of biodistribution, pharmacokinetics, and often, toxicity for these compounds (Duncan et al., 1984, Adv. Polym. Sci. 57:53-101). Many water-soluble polymers have been used to achieve these effects, such as poly(sialic acid), dextran, poly(N-(2-hydroxypropyl) methacrylamide) (PHPMA), poly(N-vinylpyrrolidone) (PVP), poly(vinyl alcohol) (PVA), poly(ethylene glycol-co-propylene glycol), poly(N-acryloyl morpholine (PAcM), and poly(ethylene glycol) (PEG) (Powell, 1980, Polyethylene glycol. In R. L. Davidson (Ed.) Handbook of Water Soluble Gums and Resins. McGraw-Hill, New York, chapter 18). PEG possess an ideal set of properties: very low toxicity (Pang, 1993, J. Am. Coll. Toxicol. 12:429-456) excellent solubility in aqueous solution (Powell, supra), low immunogenicity and antigenicity (Dreborg et al., 1990, Crit. Rev. Ther. Drug Carrier Syst. 6:315-365). PEG-conjugated or “PEGylated” protein therapeutics, containing single or multiple chains of polyethylene glycol on the protein, have been described in the scientific literature (Clark et al., 1996, J. Biol. Chem. 271:21969-21977; Hershfield, 1997, Biochemistry and immunology of poly(ethylene glycol)-modified adenosine deaminase (PEG-ADA). In J. M. Harris and S. Zalipsky (Eds) Poly(ethylene glycol): Chemistry and Biological Applications. American Chemical Society, Washington, D.C., p 145-154; Olson et al., 1997, Preparation and characterization of poly(ethylene glycol) ylated human growth hormone antagonist. In J. M. Harris and S. Zalipsky (Eds) Poly(ethylene glycol): Chemistry and Biological Applications. American Chemical Society, Washington, D.C., p 170-181).

A peptide of the invention may be synthesized by conventional techniques. For example, the peptides of the invention may be synthesized by chemical synthesis using solid phase peptide synthesis. These methods employ either solid or solution phase synthesis methods (see for example, J. M. Stewart, and J. D. Young, Solid Phase Peptide Synthesis, 2nd Ed., Pierce Chemical Co., Rockford Ill. (1984) and G. Barany and R. B. Merrifield, The Peptides: Analysis Synthesis, Biology editors E. Gross and J. Meienhofer Vol. 2 Academic Press, New York, 1980, pp. 3-254 for solid phase synthesis techniques; and M Bodansky, Principles of Peptide Synthesis, Springer-Verlag, Berlin 1984, and E. Gross and J. Meienhofer, Eds., The Peptides: Analysis, Synthesis, Biology, suprs, Vol 1, for classical solution synthesis.)

The peptides may be chemically synthesized by Merrifield-type solid phase peptide synthesis. This method may be routinely performed to yield peptides up to about 60-70 residues in length, and may, in some cases, be utilized to make peptides up to about 100 amino acids long. Larger peptides may also be generated synthetically via fragment condensation or native chemical ligation (Dawson et al., 2000, Ann. Rev. Biochem. 69:923-960). An advantage to the utilization of a synthetic peptide route is the ability to produce large amounts of peptides, even those that rarely occur naturally, with relatively high purities, i.e., purities sufficient for research, diagnostic or therapeutic purposes.

Solid phase peptide synthesis is described by Stewart et al. in Solid Phase Peptide Synthesis, 2nd Edition, 1984, Pierce Chemical Company, Rockford, Ill.; and Bodanszky and Bodanszky in The Practice of Peptide Synthesis, 1984, Springer-Verlag, New York. At the outset, a suitably protected amino acid residue is attached through its carboxyl group to a derivatized, insoluble polymeric support, such as cross-linked polystyrene or polyamide resin. “Suitably protected” refers to the presence of protecting groups on both the alpha-amino group of the amino acid, and on any side chain functional groups. Side chain protecting groups are generally stable to the solvents, reagents and reaction conditions used throughout the synthesis, and are removable under conditions which will not affect the final peptide product. Stepwise synthesis of the oligopeptide is carried out by the removal of the N-protecting group from the initial amino acid, and coupling thereto of the carboxyl end of the next amino acid in the sequence of the desired peptide. This amino acid is also suitably protected. The carboxyl of the incoming amino acid can be activated to react with the N-terminus of the support-bound amino acid by formation into a reactive group, such as formation into a carbodiimide, a symmetric acid anhydride, or an “active ester” group, such as hydroxybenzotriazole or pentafluorophenyl esters.

Examples of solid phase peptide synthesis methods include the BOC method which utilized tert-butyloxcarbonyl as the alpha-amino protecting group, and the FMOC method which utilizes 9-fluorenylmethyloxcarbonyl to protect the alpha-amino of the amino acid residues, both which methods are well-known by those of skill in the art.

Incorporation of N- and/or C-blocking groups may also be achieved using protocols conventional to solid phase peptide synthesis methods. For incorporation of C-terminal blocking groups, for example, synthesis of the desired peptide is typically performed using, as solid phase, a supporting resin that has been chemically modified so that cleavage from the resin results in a peptide having the desired C-terminal blocking group. To provide peptides in which the C-terminus bears a primary amino blocking group, for instance, synthesis is performed using a p-methylbenzhydrylamine (MBHA) resin, so that, when peptide synthesis is completed, treatment with hydrofluoric acid releases the desired C-terminally amidated peptide. Similarly, incorporation of an N-methylamine blocking group at the C-terminus is achieved using N-methylaminoethyl-derivatized DVB, resin, which upon HF treatment releases a peptide bearing an N-methylamidated C-terminus. Blockage of the C-terminus by esterification can also be achieved using conventional procedures. This entails use of resin/blocking group combination that permits release of side-chain peptide from the resin, to allow for subsequent reaction with the desired alcohol, to form the ester function. FMOC protecting group, in combination with DVB resin derivatized with methoxyalkoxybenzyl alcohol or equivalent linker, can be used for this purpose, with cleavage from the support being effected by TFA in dichloromethane. Esterification of the suitably activated carboxyl function, e.g. with DCC, can then proceed by addition of the desired alcohol, followed by de-protection and isolation of the esterified peptide product.

The peptides of the invention may be prepared by standard chemical or biological means of peptide synthesis. Biological methods include, without limitation, expression of a nucleic acid encoding a peptide in a host cell or in an in vitro translation system.

Included in the invention are nucleic acid sequences that encode the peptide of the invention. In some embodiments, the invention includes nucleic acid sequences encoding the amino acid sequence of one or more TRIM proteins.

Accordingly, subclones of a nucleic acid sequence encoding a peptide of the invention can be produced using conventional molecular genetic manipulation for subcloning gene fragments, such as described by Sambrook et al., Molecular Cloning: A Laboratory Manual, Cold Springs Laboratory, Cold Springs Harbor, New York (2012), and Ausubel et al. (ed.), Current Protocols in Molecular Biology, John Wiley & Sons (New York, NY) (1999 and preceding editions), each of which is hereby incorporated by reference in its entirety. The subclones then are expressed in vitro or in vivo in bacterial cells to yield a smaller protein or polypeptide that can be tested for a particular activity.

Combined with certain formulations, such peptides can be effective intracellular agents. However, in order to increase the efficacy of such peptides, the one or more peptides of the invention can be provided a fusion peptide along with a second peptide which promotes “transcytosis,” e.g., uptake of the peptide by cells. For example, in some embodiments, the peptide may comprise a cell-penetrating domain, for example a cell-penetrating peptide (CPP) to allow for the peptide to enter a cell. In some embodiments, the CPP is derived from HIV Tat.

To illustrate, the one or more peptides of the present invention can be provided as part of a fusion polypeptide with all or a fragment of the N-terminal domain of the HIV protein Tat, e.g., residues 1-72 of Tat or a smaller fragment thereof which can promote transcytosis. In some embodiments, the peptide comprises the protein transduction domain of HIV Tat. In other embodiments, the one or more peptides can be provided as a fusion polypeptide with all or a portion of the antennapedia III protein. Other cell-penetrating domains that mediate uptake of the peptide are known in the art, and are equally applicable for use in a fusion peptide of the present invention.

### Nucleic Acids

In some embodiments, the composition of the invention comprises one or isolated nucleic acids. For example, in some embodiments, the one or more isolated nucleic acids encodes one or more TRIM proteins. In some embodiments, the one or more isolated nucleic acids encode an amino acid sequence of one or more selected from the group consisting of human TRIM1, TRIM2, TRIM3, TRIM4, TRIM5, TRIM6, TRIM7, TRIM8, TRIM9, TRIM10, TRIM11, TRIM13, TRIM14, TRIM15, TRIM16, TRIM17, TRIM18, TRIM19, TRIM20, TRIM21, TRIM22, TRIM23, TRIM24, TRIM25, TRIM26, TRIM27, TRIM28, TRIM29, TRIM31, TRIM32, TRIM33, TRIM34, TRIM35, TRIM36, TRIM37, TRIM38, TRIM39, TRIM40, TRIM41, TRIM42, TRIM43, TRIM44, TRIM45, TRIM46, TRIM47, TRIM48, TRIM49, TRIM50, TRIM51, TRIM52, TRIM54, TRIM55, TRIM56, TRIM58, TRIM59, TRIM60, TRIM61, TRIM62, TRIM63, TRIM64, TRIM65, TRIM66, TRIM67, TRIM68, TRIM69, TRIM70, TRIM71, TRIM72, TRIM73, TRIM74, TRIM76, and TRIM77 peptides; mouse TRIM12 and TRIM30 peptides; and functional variants thereof. In some embodiments, the one or more isolated nucleic acids encode an amino acid sequence of one or more selected from the group consisting of human TRIM2, TRIM3, TRIM4, TRIM5, TRIM9, TRIM10, TRIM11, TRIM17, TRIM18, TRIM19, TRIM21, TRIM24, TRIM26, TRIM29, TRIM30, TRIM31, TRIM34, TRIM36, TRIM37, TRIM46, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70 peptides; mouse TRIM12 and TRIM30 peptides; and functional variants thereof. In some embodiments the one or more isolated nucleic acids encode an amino acid sequence of one or more selected from the group consisting of human TRIM10, TRIM11, TRIM24, TRIM36, TRIM37, TRIM40, TRIM49, TRIM55, TRIM58, and TRIM68 peptides and functional variants thereof. In some embodiments, the one or more isolated nucleic acids encode an amino acid sequence of one or more selected from the group consisting of human TRIM2, TRIM3, TRIM4, TRIM5, TRIM9, TRIM10, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70 peptides and functional variants thereof. In some embodiments, the one or more isolated nucleic acids encode an amino acid sequence of one or more selected from the group consisting of human TRIM10, TRIM11, and TRIM55 peptides and functional variants thereof. In some embodiments, the one or more isolated nucleic acids encode an amino acid sequence of one or more selected from the group consisting of human TRIM2, TRIM3, TRIM10, TRIM11, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73 peptides and functional variants thereof. In some embodiments, the one or more isolated nucleic acids encode an amino acid sequence of one or more selected from the group consisting of human TRIM10, TRIM11, TRIM36, TRIM55, and TRIM68 peptides and functional variants thereof. In some embodiments, the one or more isolated nucleic acids encode an amino acid sequence of one or more selected from the group consisting of human TRIM10, TRIM11, TRIM24, TRIM36, and TRIM58 peptides and functional variants thereof. In some embodiments, the one or more isolated nucleic acids encode an amino acid sequence of one or more selected from the group consisting of human TRIM10, TRIM11, TRIM17, TRIM36, TRIM37, TRIM40, TRIM49, and TRIM55 peptides and functional variants thereof. In some embodiments, the one or more isolated nucleic acids encode an amino acid sequence of human TRIM10 peptide or functional variants thereof. In some embodiments, a peptide of the composition comprises an amino acid sequence of human TRIM11 peptide or functional variants thereof.

In some embodiments, the isolated nucleic acid comprises a nucleotide sequence encoding an amino acid sequence of one or more TRIM proteins provided by the accession numbers of Table 1.

Further, the invention encompasses an isolated nucleic acid comprising a nucleotide sequence having substantial homology to a nucleotide sequence encoding a disclosed herein. The nucleic acid sequence which is “substantially homologous” is at least about 50% identical, at least about 70% identical, at least about 80% identical, at least about 85% identical, at least about 86 identical, at least about 87% identical, at least about 88% identical, at least about 89% identical, at least about 90% identical, at least about 91% identical, at least about 92% identical, at least about 93% identical, at least about 94% identical, at least about 95% identical, at least about 96% identical, at least about 97% identical, at least about 98% identical, at least about 99% identical, to a nucleotide sequence of an isolated nucleic acid encoding a peptide of the invention.

Thus, the invention encompasses expression vectors and methods for the introduction of exogenous DNA into cells with concomitant expression of the exogenous DNA in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

The desired nucleic acid encoding one or more TRIM proteins can be cloned into a number of types of vectors. However, the present invention should not be construed to be limited to any particular vector. Instead, the present invention should be construed to encompass a wide plethora of vectors which are readily available and/or well-known in the art. For example, a desired polynucleotide of the invention can be cloned into a vector including, but not limited to a plasmid, a phagemid, a phage derivative, an animal virus, and a cosmid. Vectors of particular interest include expression vectors, replication vectors, and transgene vectors.

In specific embodiments, the expression vector is selected from the group consisting of a viral vector, a bacterial vector and a mammalian cell vector. Numerous expression vector systems exist that comprise at least a part or all of the compositions discussed above. Prokaryote- and/or eukaryote-vector based systems can be employed for use with the present invention to produce polynucleotides, or their cognate polypeptides. Many such systems are commercially and widely available.

Further, the expression vector may be provided to a cell in the form of a viral vector. Viral vector technology is well known in the art and is described, for example, in Sambrook et al. (2012), and in Ausubel et al. (1997), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers. (See, e.g., WO 01/96584; WO 01/29058; and U.S. Pat. No. 6,326,193.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

For example, vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. In a preferred embodiment, the composition includes a vector derived from an adeno-associated virus (AAV). Adeno-associated viral (AAV) vectors have become powerful gene delivery tools for the treatment of various disorders. AAV vectors possess a number of features that render them ideally suited for gene therapy, including a lack of pathogenicity, minimal immunogenicity, and the ability to transduce postmitotic cells in a stable and efficient manner. Expression of a particular gene contained within an AAV vector can be specifically targeted to one or more types of cells by choosing the appropriate combination of AAV serotype, promoter, and delivery method

In some embodiments, the encoding sequence is contained within an AAV vector. More than 30 naturally occurring serotypes of AAV are available. Many natural variants in the AAV capsid exist, allowing identification and use of an AAV with properties specifically suited for skeletal muscle. AAV viruses may be engineered using conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

Thus, expression of one or more TRIM proteins can be achieved by delivering a recombinantly engineered AAV or artificial AAV that contains one or more encoding sequences. The use of AAVs is a common mode of exogenous delivery of DNA as it is relatively non-toxic, provides efficient gene transfer, and can be easily optimized for specific purposes. Exemplary AAV serotypes include, but is not limited to AAV1, AAV2, AAV3, AAV4, AAV5, AAV6, AAV7, AAV8 and AAV9.

Desirable AAV fragments for assembly into vectors include the cap proteins, including the vp1, vp2, vp3 and hypervariable regions, the rep proteins, including rep 78, rep 68, rep 52, and rep 40, and the sequences encoding these proteins. These fragments may be readily utilized in a variety of vector systems and host cells. Such fragments may be used alone, in combination with other AAV serotype sequences or fragments, or in combination with elements from other AAV or non-AAV viral sequences. As used herein, artificial AAV serotypes include, without limitation, AAV with a non-naturally occurring capsid protein. Such an artificial capsid may be generated by any suitable technique, using a selected AAV sequence (e.g., a fragment of a vp1 capsid protein) in combination with heterologous sequences which may be obtained from a different selected AAV serotype, non-contiguous portions of the same AAV serotype, from a non-AAV viral source, or from a non-viral source. An artificial AAV serotype may be, without limitation, a chimeric AAV capsid, a recombinant AAV capsid, or a “humanized” AAV capsid. Thus, exemplary AAVs, or artificial AAVs, suitable for expression of one or more TRIM proteins, include AAV2/8 (see U.S. Pat. No. 7,282,199), AAV2/5 (available from the National Institutes of Health), AAV2/9 (International Patent Publication No. WO2005/033321), AAV2/6 (U.S. Pat. No. 6,156,303), and AAVrh8 (International Patent Publication No. WO2003/042397), among others.

For expression of the desired polynucleotide, at least one module in each promoter functions to position the start site for RNA synthesis. The best known example of this is the TATA box, but in some promoters lacking a TATA box, such as the promoter for the mammalian terminal deoxynucleotidyl transferase gene and the promoter for the SV40 genes, a discrete element overlying the start site itself helps to fix the place of initiation.

Additional promoter elements, i.e., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either co-operatively or independently to activate transcription.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

In order to assess the expression of the desired polynucleotide, the expression vector to be introduced into a cell can also contain either a selectable marker gene or a reporter gene or both to facilitate identification and selection of expressing cells from the population of cells sought to be transfected or infected through viral vectors. In other embodiments, the selectable marker may be carried on a separate piece of DNA and used in a co-transfection procedure. Both selectable markers and reporter genes may be flanked with appropriate regulatory sequences to enable expression in the host cells. Useful selectable markers are known in the art and include, for example, antibiotic-resistance genes, such as neo and the like.

Reporter genes are used for identifying potentially transfected cells and for evaluating the functionality of regulatory sequences. Reporter genes that encode for easily assayable proteins are well known in the art. In general, a reporter gene is a gene that is not present in or expressed by the recipient organism or tissue and that encodes a protein whose expression is manifested by some easily detectable property, e.g., enzymatic activity. Expression of the reporter gene is assayed at a suitable time after the DNA has been introduced into the recipient cells.

Suitable reporter genes may include genes encoding luciferase, beta-galactosidase, chloramphenicol acetyl transferase, secreted alkaline phosphatase, or the green fluorescent protein gene (see, e.g., Ui-Tei et al., 2000 FEBS Lett. 479:79-82). Suitable expression systems are well known and may be prepared using well known techniques or obtained commercially. Internal deletion constructs may be generated using unique internal restriction sites or by partial digestion of non-unique restriction sites. Constructs may then be transfected into cells that display high levels of siRNA polynucleotide and/or polypeptide expression. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In the context of an expression vector, the vector can be readily introduced into a host cell, e.g., mammalian, bacterial, yeast or insect cell by any method in the art. For example, the expression vector can be transferred into a host cell by physical, chemical or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York).

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. A preferred colloidal system for use as a delivery vehicle in vitro and in vivo is a liposome (i.e., an artificial membrane vesicle). The preparation and use of such systems is well known in the art.

Regardless of the method used to introduce exogenous nucleic acids into a host cell, in order to confirm the presence of the recombinant DNA sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Any DNA vector or delivery vehicle can be utilized to transfer the desired polynucleotide to a cell in vitro or in vivo. In the case where a non-viral delivery system is utilized, a preferred delivery vehicle is a liposome. The above-mentioned delivery systems and protocols therefore can be found in Gene Targeting Protocols, 2ed., pp 1-35 (2002) and Gene Transfer and Expression Protocols, Vol. 7, Murray ed., pp 81-89 (1991).

“Liposome” is a generic term encompassing a variety of single and multilamellar lipid vehicles formed by the generation of enclosed lipid bilayers or aggregates. Liposomes may be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. However, the present invention also encompasses compositions that have different structures in solution than the normal vesicular structure. For example, the lipids may assume a micellar structure or merely exist as nonuniform aggregates of lipid molecules. Also contemplated are lipofectamine-nucleic acid complexes.

In some embodiments, the composition of the invention comprises in vitro transcribed (IVT) RNA encoding one or more components of the one or more TRIM proteins. In some embodiments, an IVT RNA can be introduced to a cell as a form of transient transfection. The RNA is produced by in vitro transcription using a plasmid DNA template generated synthetically. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired template for in vitro transcription is one or more TRIM proteins or TRIM protein fragment.

In some embodiments, the DNA to be used for PCR contains an open reading frame. The DNA can be from a naturally occurring DNA sequence from the genome of an organism. In some embodiments, the DNA is a full length gene of interest of a portion of a gene. The gene can include some or all of the 5′ and/or 3′ untranslated regions (UTRs). The gene can include exons and introns. In some embodiments, the DNA to be used for PCR is a human gene. In another embodiment, the DNA to be used for PCR is a human gene including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

In some embodiments, the composition of the present invention comprises a modified nucleic acid encoding one or more one or more TRIM proteins described herein. For example, in some embodiments, the composition comprises a nucleoside-modified RNA. In some embodiments, the composition comprises a nucleoside-modified mRNA. Nucleoside-modified mRNA have particular advantages over non-modified mRNA, including for example, increased stability, low immunogenicity, and enhanced translation. Nucleoside-modified mRNA useful in the present invention is further described in U.S. Pat. No. 8,278,036, which is incorporated by reference herein in its entirety.

### Modified Cell

The present invention includes a composition comprising a cell which comprises one or more TRIM proteins, a nucleic acid encoding a one or more TRIM proteins, or a combination thereof. In some embodiments, the cell is genetically modified to express a protein and/or nucleic acid of the invention. In certain embodiments, genetically modified cell is autologous to a subject being treated with the composition of the invention. Alternatively, the cells can be allogeneic, syngeneic, or xenogeneic with respect to the subject. In certain embodiment, the cell is able to secrete or release the expressed protein into extracellular space in order to deliver the peptide to one or more other cells.

The genetically modified cell may be modified in vivo or ex vivo, using techniques standard in the art. Genetic modification of the cell may be carried out using an expression vector or using a naked isolated nucleic acid construct.

In some embodiments, the cell is obtained and modified ex vivo, using an isolated nucleic acid encoding one or more proteins described herein. In some embodiments, the cell is obtained from a subject, genetically modified to express the protein and/or nucleic acid, and is re-administered to the subject. In certain embodiments, the cell is expanded ex vivo or in vitro to produce a population of cells, wherein at least a portion of the population is administered to a subject in need.

In some embodiments, the cell is genetically modified to stably express the protein. In another embodiment, the cell is genetically modified to transiently express the protein.

### Therapeutic Methods

The present invention also provides therapeutic methods for a disease or disorder associated with protein misfolding, protein aggregates, or a combination thereof.

In some embodiments, the method comprises administering a composition comprising an activator of one or more TRIM proteins to a subject. In some embodiments, the subject has a disease or disorder associated with protein misfolding or protein aggregates. In some embodiments, the subject has a disease or disorder associated with misfolded proteins and/or and protein aggregates of amyloid-beta, alpha-synuclein, tau, prions, SOD1, TDP-43, FUS, p53, p53 mutants, or proteins associated with polyglutamine repeats, such as huntingtin and ataxins.

In various embodiments, diseases and disorders treatable by the methods of the invention include, but are not limited to: SCA1, SCA2, SCA3, SCA6, SCA7, SCA17, Huntington's disease, Dentatorubral-pallidoluysian atrophy (DRPLA), Alzheimer's disease, Parkinson's disease, amyotrophic lateral sclerosis (ALS), transmissible spongiform encephalopathies (prion disease), dementia with Lewy bodies (DLB), multiple system atrophy (MSA), Frontotemporal lobar degeneration (FTLD), AL amyloidosis, AA amyloidosis, Familial Mediterranean fever, senile systemic amyloidosis, familial amyloidotic polyneuropathy, Icelandic hereditary cerebral amyloid angiopathy, hereditary cerebral hemorrhage with amyloidosis, pituitary prolactinoma, frontotemporal lobar degeneration (FTLD-tau), progressive supranuclear palsy (PSP), corticobasal degeneration (CBD), argyrophilic grain disease (AGD), frontotemporal dementia and parkinsonism linked to chromosome 17 (FTDP-17), vacuolar tauopathy, Lytico-bodig disease, globular glial tauopathy (GGT), ageing-related tau astrogliopathy (ARTAG), Pick's disease, primary age-related tauopathy (PART), tangle only dementia (TOD), chronic traumatic encephalopathy (CTE), anti-IgLON5-related tauopathy, Guadeloupean parkinsonism, Nodding Syndrome (NS), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, lipofuscinosis, Shy-Drager syndrome, striatonigral degeneration, olivopontocerebellar atrophy, Hallervorden-Spatz syndrome, REM sleep behavior disorder (RPD), Alzheimer's disease with amygdala restricted Lewy bodies (AD/ALB), frontotemporal lobar degeneration (FTLD-TDP), multiple system proteinopathy (MSP), Perry disease, facial onset sensory and motor neuronopathy (FOSMN), cerebral age-related TDP-43 with sclerosis (CARTS), limbic-predominant age-related TDP-43 encephalopathy (LATE), sporadic inclusion body myositis (sIBM), chronic traumatic encephalopathy (CTE), primary lateral sclerosis (PLS), progressive muscular atrophy (PMA), Guam Parkinson-dementia complex (G-PDC), and Guam amyotrophic lateral sclerosis (G-ALS).

It will be appreciated by one of skill in the art, when armed with the present disclosure including the methods detailed herein, that the invention is not limited to treatment of a disease associated with protein misfolding or protein aggregates that is already established. Particularly, the disease or disorder need not have manifested to the point of detriment to the subject; indeed, the disease or disorder need not be detected in a subject before treatment is administered. That is, significant signs or symptoms of the disease or disorder do not have to occur before the present invention may provide benefit. Therefore, the present invention includes a method for preventing a disease or disorder associated with protein misfolding or protein aggregates, in that a activator composition, as discussed previously elsewhere herein, can be administered to a subject prior to the onset of the disease or disorder, thereby preventing the disease or disorder.

One of skill in the art, when armed with the disclosure herein, would appreciate that the prevention of a disease associated with protein misfolding or protein aggregates, encompasses administering to a subject an activator as a preventative measure against the development of, or progression of a disease associated with protein misfolding or protein aggregates. As more fully discussed elsewhere herein, methods of increasing the level or activity of a gene, or gene product, encompass a wide plethora of techniques for increasing not only the level and activity of polypeptide gene products, but also for increasing expression of a nucleic acid, including either transcription, translation, or both.

Additionally, as disclosed elsewhere herein, one skilled in the art would understand, once armed with the teaching provided herein, that the present invention encompasses methods of treating, or preventing, a wide variety of diseases associated with protein misfolding or protein aggregates, where modulating the level or activity of a gene, or gene product treats or prevents the disease. Various methods for assessing whether a disease is associated protein misfolding or protein aggregates are known in the art. Further, the invention encompasses treatment or prevention of such diseases discovered in the future.

In one aspect, the method comprises use of one or more TRIM proteins to stabilize a misfolded protein. In certain aspects, stabilization of a functional misfolded protein via one or more TRIM protein described herein can treat or prevent a disease or disorder associated with the misfolded protein.

In another aspect, the method comprises use of one or more TRIM proteins to reduce the level of protein aggregates. In some embodiments, protein aggregates levels are reduced by degrading protein aggregates. In some embodiments, at least about 10%, about 20%, about 30%, about 40%, about 50%, about 60%, about 70%, about 80%, or about 90% of protein aggregates are degraded relative to protein aggregate levels prior to administration of the composition. In some embodiments, the method is effective to reduce protein aggregate levels by between about 10% and about 90%. In some embodiments, the method is effective to reduce protein aggregate levels by between about 30% and about 90%. In some embodiments, the method is effective to reduced protein aggregate levels by between about 50% and about 90%. In some embodiments, the method is effective to reduce protein aggregate levels by about 10%, about 15%, about 20%, about 30%, about 35%, about 40%, about 45%, about 50%, about 55%, about 60%, about 65%, about 70%, about 75%, about 80%, about 85%, about 90%, or about 95%.

In some embodiments, the method the protein aggregates levels are reduced by solubilizing protein aggregates. In some embodiments, the ratio of insoluble protein to soluble protein is decreased by at least about 10%, at least about 20%, at least about 30%, at least about 40%, at least about 50%, at least about 60%, at least about 70%, at least about 80%, or at least about 90%. In some embodiments, the method is effective to reduce the ratio of insoluble protein to soluble protein by between about 10% and about 90%. In some embodiments, the method is effective to reduce the ratio of insoluble protein to soluble protein by between about 30% and about 90%. In some embodiments, the method is effective to reduce the ratio of insoluble protein to soluble protein by between about 50% and about 90%. In some embodiments, the method is effective to reduce the ratio of insoluble protein to soluble protein by about 10%, about 15%, about 20%, about 30%, about 35%, about 40%, about 45%, about 50%, about 55%, about 60%, about 65%, about 70%, about 75%, about 80%, about 85%, about 90%, or about 95%.

In some embodiments, protein aggregate levels are reduced after at least about 1 day, about 2 days, about 3 days, about 4 days, about 5 days, about 6 days, about 7 days, about 8 days, about 9 days, about 10 days, about 11 days, about 12 days, about 13 days, or about 2 weeks. In some embodiments, the method is effective to reduce protein aggregate levels for an extended period of time. In some embodiments, the protein aggregate levels are reduced for at least about 1 day, about 2 days, about 3 days, about 4 days, about 5 days, about 6 days, about 7 day, about 8 days, about 9 days, about 10 days, about 11 days, about 12 days, about 13 days, or about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 26 weeks, about 39 weeks, about 1 year, or about 2 years.

The invention encompasses administration of an activator of a gene, or gene product. To practice the methods of the invention; the skilled artisan would understand, based on the disclosure provided herein, how to formulate and administer the appropriate activator composition to a subject. The present invention is not limited to any particular method of administration or treatment regimen.

In some embodiments, the method comprises administering to the subject in need an effective amount of a composition that increases the expression or activity of one or more TRIM protein.

For example, in some embodiments, the method comprises administering to the subject in need an effective amount of a composition that increases the expression or activity of one or more TRIM protein.

In certain embodiments, the TRIM protein is one or more selected from the group consisting of human TRIM1, TRIM2, TRIM3, TRIM4, TRIM5, TRIM6, TRIM7, TRIM8, TRIM9, TRIM10, TRIM11, TRIM13, TRIM14, TRIM15, TRIM16, TRIM17, TRIM18, TRIM19, TRIM20, TRIM21, TRIM22, TRIM23, TRIM24, TRIM25, TRIM26, TRIM27, TRIM28, TRIM29, TRIM31, TRIM32, TRIM33, TRIM34, TRIM35, TRIM36, TRIM37, TRIM38, TRIM39, TRIM40, TRIM41, TRIM42, TRIM43, TRIM44, TRIM45, TRIM46, TRIM47, TRIM48, TRIM49, TRIM50, TRIM51, TRIM52, TRIM54, TRIM55, TRIM56, TRIM58, TRIM59, TRIM60, TRIM61, TRIM62, TRIM63, TRIM64, TRIM65, TRIM66, TRIM67, TRIM68, TRIM69, TRIM70, TRIM71, TRIM72, TRIM73, TRIM74, TRIM76, and TRIM77; and mouse TRIM12 and TRIM30. In some embodiments, the TRIM protein is one or more selected from the group consisting of human TRIM2, TRIM3, TRIM4, TRIM5, TRIM9, TRIM10, TRIM11, TRIM17, TRIM18, TRIM19, TRIM21, TRIM24, TRIM26, TRIM29, TRIM30, TRIM31, TRIM34, TRIM36, TRIM37, TRIM46, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70; and mouse TRIM12 and TRIM30. In some embodiments, the TRIM protein is one or more selected from the group consisting of human TRIM10, TRIM11, TRIM24, TRIM36, TRIM37, TRIM40, TRIM49, TRIM55, TRIM58, and TRIM68. In some embodiments, the TRIM protein is one or more selected from the group consisting of human TRIM2, TRIM3, TRIM4, TRIM5, TRIM9, TRIM10, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70. In some embodiments, the TRIM protein is one or more selected from the group consisting of human TRIM10, TRIM11, and TRIM55. In some embodiments, the TRIM protein is one or more selected from the group consisting of human TRIM2, TRIM3, TRIM10, TRIM11, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73. In some embodiments, the TRIM protein is one or more selected from the group consisting of human TRIM10, TRIM11, TRIM36, TRIM55, and TRIM68. In some embodiments, the TRIM protein is one or more selected from the group consisting of human TRIM10, TRIM11, TRIM24, TRIM36, and TRIM58. In some embodiments, the TRIM protein is one or more selected from the group consisting of human TRIM10, TRIM11, TRIM17, TRIM36, TRIM37, TRIM40, TRIM49, and TRIM55. In some embodiments, TRIM protein is human TRIM10. In some embodiments, the TRIM protein is human TRIM11.

Methods of using TRIM proteins, nucleic acids encoding TRIM proteins, and combinations thereof for preventing or treating protein misfolding and aggregation are disclosed in International (PCT) Publication Number: WO 2016/196328 A1, incorporated by reference herein in its entirety.

In some embodiments, the method comprises increasing the expression or activity of the one or more TRIM protein in at least one neural cell of the subject. For example, in certain embodiments, the method comprises increasing the expression or activity of the one or more TRIM protein in a at least one neuron, glial cell, astrocyte, oligodendrocyte, Purkinje cell, pyramidal cell, or the like.

In some embodiments, the method comprises contacting the neural tissue of a subject with an effective amount of a composition that increases the expression or activity of one or more components of the one or more TRIM protein. For example, in certain embodiments, the method comprises contacting a neuron, glial cell, astrocyte, oligodendrocyte, Purkinje cell, pyramidal cell, or the like, of a subject with an effective amount of a composition that increases the expression or activity of one or more TRIM protein. In some embodiments, the neural cell is affected by protein misfolding, protein aggregates, or a combination thereof.

One of skill in the art will appreciate that the activators of the invention can be administered singly or in any combination. Further, the activators of the invention can be administered singly or in any combination in a temporal sense, in that they may be administered concurrently, or before, and/or after each other. One of ordinary skill in the art will appreciate, based on the disclosure provided herein, that the activator compositions of the invention can be used to prevent or to treat a disease or disorder associated with a misfolded protein or protein aggregate, and that an activator composition can be used alone or in any combination with another activator to effect a prophylactic or therapeutic result.

In various embodiments, any of the activators of the invention described herein can be administered alone or in combination with other activators of other molecules associated with a disease associated with protein misfolding or protein aggregates. In various embodiments, any of the activators of the invention described herein can be administered alone or in combination with other therapeutic or preventative agents which may be used to treat or prevent a disease associated with protein misfolding or protein aggregates. Exemplary therapeutic agents which may be used in combination with the activators of the present invention include, but is not limited to, anti-amyloid-β antibodies and anti-tau antibodies.

### Gene Therapy

Contacting cells in a subject with a nucleic acid composition that encodes a protein that increases the expression or activity of one or more TRIM proteins can inhibit or delay the onset of one or more symptoms of a disease or disorder associated with protein misfolding or protein aggregates.

In some embodiments, the nucleic acid composition of the present invention encodes one or more peptides. For example, in some embodiments, a nucleic acid composition can encode a peptide that comprises an amino acid sequence of one or more TRIM proteins. In some embodiments, the nucleic acid composition encodes one or more TRIM proteins selected from the group consisting of: human TRIM1, TRIM2, TRIM3, TRIM4, TRIM5, TRIM6, TRIM7, TRIM8, TRIM9, TRIM10, TRIM11, TRIM13, TRIM14, TRIM15, TRIM16, TRIM17, TRIM18, TRIM19, TRIM20, TRIM21, TRIM22, TRIM23, TRIM24, TRIM25, TRIM26, TRIM27, TRIM28, TRIM29, TRIM30, TRIM31, TRIM32, TRIM33, TRIM34, TRIM35, TRIM36, TRIM37, TRIM38, TRIM39, TRIM40, TRIM41, TRIM42, TRIM43, TRIM44, TRIM45, TRIM46, TRIM47, TRIM48, TRIM49, TRIM50, TRIM51, TRIM52, TRIM54, TRIM55, TRIM56, TRIM58, TRIM59, TRIM60, TRIM61, TRIM62, TRIM63, TRIM64, TRIM65, TRIM66, TRIM67, TRIM68, TRIM69, TRIM70, TRIM71, TRIM72, TRIM73, TRIM74, TRIM76, and TRIM77; and mouse TRIM12 and TRIM30. In some embodiments, the nucleic acid composition encodes one or more TRIM proteins selected from the group consisting of: human TRIM2, TRIM3, TRIM4, TRIM5, TRIM9, TRIM10, TRIM11, TRIM17, TRIM18, TRIM19, TRIM21, TRIM24, TRIM26, TRIM29, TRIM31, TRIM34, TRIM36, TRIM37, TRIM46, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70; and mouse TRIM12 and TRIM30. In some embodiments, the nucleic acid composition encodes one or more TRIM proteins selected from the group consisting of: human TRIM10, TRIM11, TRIM24, TRIM36, TRIM37, TRIM40, TRIM49, TRIM55, TRIM58, and TRIM68. In some embodiments, the nucleic acid composition encodes one or more TRIM proteins selected from the group consisting of: human TRIM2, TRIM3, TRIM4, TRIM5, TRIM9, TRIM10, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70. In some embodiments, the nucleic acid composition encodes one or more TRIM proteins selected from the group consisting of: human TRIM10, TRIM11, and TRIM55. In some embodiments, the nucleic acid composition encodes one or more TRIM proteins selected from the group consisting of: human TRIM2, TRIM3, TRIM10, TRIM11, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73. In some embodiments, the nucleic acid composition encodes one or more TRIM proteins selected from the group consisting of: human TRIM10, TRIM11, TRIM36, TRIM55, and TRIM68. In some embodiments, the nucleic acid composition encodes one or more TRIM proteins selected from the group consisting of: human TRIM10, TRIM11, TRIM24, TRIM36, and TRIM58. In some embodiments, the nucleic acid composition encodes one or more TRIM proteins selected from the group consisting of: human TRIM10, TRIM11, TRIM17, TRIM36, TRIM37, TRIM40, TRIM49, and TRIM55. In some embodiments, the nucleic acid composition encodes human TRIM10. In some embodiments, the nucleic acid composition encodes human TRIM11.

In some embodiments, the nucleic acid composition encoding one or more TRIM proteins comprises one or more sequences of one or more Accession numbers as listed in Table 1.

According to the present invention, a method is also provided of supplying protein to a cell which carries a normal, or a mutant gene, associated with diminished or insufficient activity of one or more TRIM proteins. Supplying protein to a cell with a mutant gene should allow normal functioning of the recipient cells. The nucleic acid encoding a peptide may be introduced into the cell in a vector such that the nucleic acid remains extrachromosomal. In such a situation, the nucleic acid will be expressed by the cell from the extrachromosomal location. More preferred is the situation where the nucleic acid or a part thereof is introduced into the cell in such a way that it integrates into the cell's genome or recombines with the endogenous mutant gene present in the cell. Vectors for introduction of genes both for recombination, for integration, and for extrachromosomal maintenance are known in the art, and any suitable vector may be used. Methods for introducing DNA into cells such as electroporation, calcium phosphate co-precipitation and viral transduction are known in the art, and the choice of method is within the competence of the practitioner.

As generally discussed above, a nucleic acid, where applicable, may be employed in gene therapy methods in order to increase the level or activity of the peptides of the invention even in those persons in which the wild type gene is expressed at a “normal” level, but the gene product is insufficiently functional.

“Gene therapy” includes both conventional gene therapy where a lasting effect is achieved by a single treatment, and the administration of gene therapeutic agents, which involves the one time or repeated administration of a therapeutically effective DNA or mRNA. Oligonucleotides can be modified to enhance their uptake, e.g., by substituting their negatively charged phosphodiester groups by uncharged groups. One or more TRIM proteins of the present invention can be delivered using gene therapy methods, for example locally in neural cell or tissue or systemically (e.g., via vectors that selectively target specific tissue types, for example, tissue-specific adeno-associated viral vectors). In some embodiments, primary cells harvested from the individual can be transfected ex vivo with a nucleic acid encoding any of the peptides of the present invention, and then returned the transfected cells to the individual's body.

Gene therapy methods are well known in the art. See, e.g., WO96/07321 which discloses the use of gene therapy methods to generate intracellular antibodies. Gene therapy methods have also been successfully demonstrated in human patients. See, e.g., Baumgartner et al., Circulation 97:12, 1114-1123 (1998), Fatham, C.G. ‘A gene therapy approach to treatment of autoimmune diseases’, Immun. Res. 18:15-26 (2007); and U.S. Pat. No. 7,378,089, both incorporated herein by reference. See also Bainbridge JWB et al. “Effect of gene therapy on visual function in Leber's congenital Amaurosis”. N Engl J Med 358:2231-2239, 2008; and Maguire A M et al. “Safety and efficacy of gene transfer for Leber's Congenital Amaurosis”. N Engl J Med 358:2240-8, 2008.

There are two major approaches for introducing a nucleic acid encoding a peptide or protein (optionally contained in a vector) into a patients cells; in vivo and ex vivo. For in vivo delivery, in certain instances, the nucleic acid is injected directly into the patient, sometimes at the site where the protein is most required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. Commonly used vectors for ex vivo delivery of the gene are retroviral and lentiviral vectors.

Gene therapy would be carried out according to generally accepted methods, for example, as described by Friedman et al., 1991, Cell 66:799-806 or Culver, 1996, Bone Marrow Transplant 3: S6-9; Culver, 1996, Mol. Med. Today 2:234-236. In some embodiments, cells from a patient would be first analyzed by the diagnostic methods known in the art, to ascertain the expression or activity of one or more TRIM proteins. A virus or plasmid vector, containing a copy of the gene or a functional equivalent thereof linked to expression control elements and capable of replicating inside the cells, is prepared. The vector may be capable of replicating inside the cells. Alternatively, the vector may be replication deficient and is replicated in helper cells for use in gene therapy. Suitable vectors are known, such as disclosed in U.S. Pat. No. 5,252,479 and PCT published application WO 93/07282 and U.S. Pat. Nos. 5,691,198; 5,747,469; 5,436,146 and 5,753,500. The vector is then injected into the patient. If the transfected gene is not permanently incorporated into the genome of each of the targeted cells, the treatment may have to be repeated periodically.

Gene transfer systems known in the art may be useful in the practice of the gene therapy methods of the present invention. These include viral and nonviral transfer methods. A number of viruses have been used as gene transfer vectors or as the basis for repairing gene transfer vectors, including papovaviruses (e.g., SV40, Madzak et al., 1992, J. Gen. Virol. 73:1533-1536), adenovirus (Berkner, 1992; Curr. Topics Microbiol. Immunol. 158:39-66), vaccinia virus (Moss, 1992, Current Opin. Biotechnol. 3:518-522; Moss, 1996, PNAS 93:11341-11348), adeno-associated virus (Russell and Hirata, 1998, Mol. Genetics 18:325-330), herpesviruses including HSV and EBV (Fink et al., 1996, Ann. Rev. Neurosci. 19:265-287), lentiviruses (Naldini et al., 1996, PNAS 93:11382-11388), Sindbis and Semliki Forest virus (Berglund et al., 1993, Biotechnol. 11:916-920), and retroviruses of avian (Petropoulos et al., 1992, J. Virol. 66:3391-3397), murine (Miller, 1992, Hum. Gene Ther. 3:619-624), and human origin (Shimada et al., 1991; Helseth et al., 1990; Page et al., 1990; Buchschacher and Panganiban, 1992, J. Virol. 66:2731-2739). Most human gene therapy protocols have been based on disabled murine retroviruses, although adenovirus and adeno-associated virus are also being used.

Nonviral gene transfer methods known in the art include chemical techniques such as calcium phosphate coprecipitation; mechanical techniques, for example microinjection; membrane fusion-mediated transfer via liposomes; and direct DNA uptake and receptor-mediated DNA transfer (Curiel et al., 1992, Am. J. Respir. Cell. Mol. Biol 6:247-252). Viral-mediated gene transfer can be combined with direct in vitro gene transfer using liposome delivery, allowing one to direct the viral vectors to the tumor cells and not into the surrounding non-dividing cells. Injection of producer cells would then provide a continuous source of vector particles. This technique has been approved for use in humans with inoperable brain tumors.

In an approach which combines biological and physical gene transfer methods, plasmid DNA of any size is combined with a polylysine-conjugated antibody specific to the adenovirus hexon protein, and the resulting complex is bound to an adenovirus vector. The trimolecular complex is then used to infect cells. The adenovirus vector permits efficient binding, internalization, and degradation of the endosome before the coupled DNA is damaged. For other techniques for the delivery of adenovirus-based vectors see U.S. Pat. Nos. 5,691,198; 5,747,469; 5,436,146 and 5,753,500.

Liposome/DNA complexes have been shown to be capable of mediating direct in vivo gene transfer. While in standard liposome preparations the gene transfer process is nonspecific, localized in vivo uptake and expression have been reported in tumor deposits, for example, following direct in situ administration.

Expression vectors in the context of gene therapy are meant to include those constructs containing sequences sufficient to express a polynucleotide that has been cloned therein. In viral expression vectors, the construct contains viral sequences sufficient to support packaging of the construct. If the polynucleotide encodes a protein, expression will produce the protein. If the polynucleotide encodes an antisense polynucleotide or a ribozyme, expression will produce the antisense polynucleotide or ribozyme. Thus, in this context, expression does not require that a protein product be synthesized. In addition to the polynucleotide cloned into the expression vector, the vector also contains a promoter functional in eukaryotic cells. The cloned polynucleotide sequence is under control of this promoter. Suitable eukaryotic promoters include those described above. The expression vector may also include sequences, such as selectable markers and other sequences described herein.

In certain embodiments, the method comprises the use of gene transfer techniques which target an isolated nucleic acid directly to neural tissue. Receptor-mediated gene transfer, for example, is accomplished by the conjugation of a nucleic acid molecule (usually in the form of covalently closed supercoiled plasmid) to a protein ligand via polylysine. Ligands are chosen on the basis of the presence of the corresponding ligand receptors on the cell surface of the target cell/tissue type. These ligand-DNA conjugates can be injected directly into the blood if desired and are directed to the target tissue where receptor binding and internalization of the DNA-protein complex occurs. To overcome the problem of intracellular destruction of DNA, co-infection with adenovirus can be included to disrupt endosome function.

The therapeutic and prophylactic methods of the invention thus encompass the use of pharmaceutical compositions comprising an activator described herein, or a combination thereof to practice the methods of the invention. The pharmaceutical compositions useful for practicing the invention may be administered to deliver a dose of from ng/kg/day and 100 mg/kg/day. In some embodiments, the invention envisions administration of a dose which results in a concentration of the compound of the present invention from 1 μM and 10 μM in a mammal.

Typically, dosages which may be administered in a method of the invention to a mammal, preferably a human, range in amount from 0.5 μg to about 50 mg per kilogram of body weight of the mammal, while the precise dosage administered will vary depending upon any number of factors, including but not limited to, the type of mammal and type of disease state being treated, the age of the mammal and the route of administration. Preferably, the dosage of the compound will vary from about 1 μg to about 10 mg per kilogram of body weight of the mammal. More preferably, the dosage will vary from about 3 μg to about 1 mg per kilogram of body weight of the mammal.

The compound may be administered to a mammal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the mammal, etc.

In some embodiments, the invention includes a method comprising administering a combination of activators described herein. In certain embodiments, the method has an additive effect, wherein the overall effect of the administering a combination of activators is approximately equal to the sum of the effects of administering each individual activator. In other embodiments, the method has a synergistic effect, wherein the overall effect of administering a combination of activators is greater than the sum of the effects of administering each individual activator.

The method comprises administering a combination of activators in any suitable ratio. For example, in some embodiments, the method comprises administering two individual activators at a 1:1 ratio. In another embodiment, the method comprises administering three individual activators at a 1:1:1 ratio. However, the method is not limited to any particular ratio. Rather any ratio that is shown to be effective is encompassed.

A nucleic acid of the disclosure may be administered in combination with a carrier or lipid to increase cellular uptake. For example, the oligonucleotide may be administered in combination with a cationic lipid. Examples of cationic lipids include, but are not limited to, lipofectin, DOTMA, DOPE, and DOTAP. The publication of WO0071096, which is specifically incorporated by reference, describes different formulations, such as a DOTAP: cholesterol or cholesterol derivative formulation that can effectively be used for gene therapy. Other disclosures also discuss different lipid or liposomal formulations including nanoparticles and methods of administration; these include, but are not limited to, U.S. Patent Publication 20030203865, 20020150626, 20030032615, and 20040048787, which are specifically incorporated by reference to the extent they disclose formulations and other related aspects of administration and delivery of nucleic acids. Methods used for forming particles are also disclosed in U.S. Pat. Nos. 5,844,107, 5,877,302, 6,008,336, 6,077,835, 5,972,901, 6,200,801, and 5,972,900, which are incorporated by reference for those aspects.

The nucleic acids may also be administered in combination with a cationic amine such as poly(L-lysine). Nucleic acids may also be conjugated to a chemical moiety, such as transferrin and cholesteryls. In addition, oligonucleotides may be targeted to certain organelles by linking specific chemical groups to the oligonucleotide.

Expression vectors can be delivered to cells of a subject for the treatment or prevention of disease or disorder. The nucleic acid molecules are delivered to the cells of a subject in a form in which they can be taken up and are advantageously expressed so that therapeutically effective levels can be achieved.

Methods for delivery of the nucleic acid molecules to the cell according to the disclosure include using a delivery system, such as liposomes, polymers, microspheres, gene therapy vectors, and naked DNA vectors.

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., BACs and YACs). One of skill in the art would be well equipped to construct a vector through standard recombinant techniques, which are described in Sambrook et al., 2012 and Ausubel et al., 2003, both incorporated herein by reference. Transducing viral (e.g., retroviral, adenoviral, lentiviral and adeno-associated viral) vectors can be used for somatic cell gene therapy, especially because of their high efficiency of infection and stable integration and expression (see, e.g., Cayouette et al., Human Gene Therapy 8:423-430, 1997; Kido et al, Current Eye Research 15:833-844, 1996; Bloomer et al, Journal of Virology 71:6641-6649, 1997; Naldini et al, Science 272:263-267, 1996; and Miyoshi et al, Proc. Natl. Acad. Sci. U.S.A. 94:10319, 1997). For example, a nucleotide sequence can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al, BioTechniques 6:608-614, 1988; Tolstoshev et al, Current Opinion in Biotechnology 1:55-61, 1990; Sharp, The Lancet 337:1277-1278, 1991; Cornetta et al, Nucleic Acid Research and Molecular Biology 36:31 1-322, 1987; Anderson, Science 226:401-409, 1984; Moen, Blood Cells 17:407-416, 1991; Miller et al, Biotechnology 7:980-990, 1989; Le Gal La Salle et al, Science 259:988-990, 1993; and Johnson, Chest 107: 77S-83S, 1995). Retroviral vectors are particularly well developed and have been used in clinical settings (Rosenberg et al, N. Engl. J. Med 323:370, 1990; Anderson et al, U.S. Pat. No. 5,399,346).

Other suitable methods for nucleic acid delivery to effect expression of compositions of the present disclosure are believed to include virtually any method by which a nucleic acid (e.g., DNA, including viral and nonviral vectors) can be introduced into an organelle, a cell, a tissue or an organism, as described herein or as would be known to one of ordinary skill in the art.

The administration of a nucleic acid or peptide inhibitor of the invention to the subject may be accomplished using gene therapy. Gene therapy, which is based on inserting a therapeutic gene into a cell by means of an ex vivo or an in vivo technique. Suitable vectors and methods have been described for genetic therapy in vitro or in vivo, and are known as expert on the matter; see, for example, Giordano, Nature Medicine 2 (1996), 534-539; Schaper, Circ. Res 79 (1996), 911-919; Anderson, Science 256 (1992), 808-813; Isner, Lancet 348 (1996), 370-374; Muhlhauser, Circ. Res 77 (1995), 1077-1086; Wang, Nature Medicine 2 (1996), 714-716; WO94/29469; WO97/00957 or Schaper, Current Opinion in Biotechnology 7 (1996), 635-640 and the references quoted therein. The polynucleotide codifying the polypeptide of the invention can be designed for direct insertion or by insertion through liposomes or viral vectors (for example, adenoviral or retroviral vectors) in the cell. Preferably the cell is a cell of the germinal line, an embryonic cell or egg cell or derived from the same, more preferably the cell is a core cell. Suitable gene distribution systems that can be used according to the invention may include liposomes, distribution systems mediated by receptor, naked DNA and viral vectors such as the herpes virus, the retrovirus, the adenovirus and adeno-associated viruses, among others. The distribution of nucleic acids to a specific site in the body for genetic therapy can also be achieved by using a biolistic distribution system, such as that described by Williams (Proc. Natl. Acad. Sci. USA, 88 (1991), 2726-2729). The standard methods for transfecting cells with recombining DNA are well known by an expert on the subject of molecular biology, see, for example, WO94/29469; see also supra. Genetic therapy can be carried out by directly administering the recombining DNA molecule or the vector of the invention to a patient or transfecting the cells with the polynucleotide or the vector of the invention ex vivo and administering the transfected cells to the patient.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Expression for use in polynucleotide therapy methods can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. For any particular subject, the specific dosage regimes should be adjusted over time according to the individual need and the professional judgment of the person administering or supervising the administration of the compositions.

### Pharmaceutical Compositions and Formulations

The invention also encompasses the use of pharmaceutical compositions of the invention or salts thereof to practice the methods of the invention. Such a pharmaceutical composition may consist of at least one activator composition of the invention or a salt thereof in a form suitable for administration to a subject, or the pharmaceutical composition may comprise at least one activator composition of the invention or a salt thereof, and one or more pharmaceutically acceptable carriers, one or more additional ingredients, or some combination of these. The compound or conjugate of the invention may be present in the pharmaceutical composition in the form of a physiologically acceptable salt, such as in combination with a physiologically acceptable cation or anion, as is well known in the art.

In some embodiments, the pharmaceutical compositions useful for practicing the methods of the invention may be administered to deliver a dose of between 1 ng/kg/day and 100 mg/kg/day. In another embodiment, the pharmaceutical compositions useful for practicing the invention may be administered to deliver a dose of between 1 ng/kg/day and 500 mg/kg/day.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

Pharmaceutical compositions that are useful in the methods of the invention may be suitably developed for oral, rectal, vaginal, parenteral, topical, pulmonary, intranasal, buccal, ophthalmic, or another route of administration. A composition useful within the methods of the invention may be directly administered to the skin, vagina or any other tissue of a mammal. Other contemplated formulations include liposomal preparations, resealed erythrocytes containing the active ingredient, and immunologically-based formulations. The route(s) of administration will be readily apparent to the skilled artisan and will depend upon any number of factors including the type and severity of the disease being treated, the type and age of the veterinary or human subject being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

As used herein, a “unit dose” is a discrete amount of the pharmaceutical composition comprising a predetermined amount of the active ingredient. The amount of the active ingredient is generally equal to the dosage of the active ingredient that would be administered to a subject or a convenient fraction of such a dosage such as, for example, one-half or one-third of such a dosage. The unit dosage form may be for a single daily dose or one of multiple daily doses (e.g., about 1 to 4 or more times per day). When multiple daily doses are used, the unit dosage form may be the same or different for each dose.

Although the descriptions of pharmaceutical compositions provided herein are principally directed to pharmaceutical compositions that are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist may design and perform such modification with merely ordinary, if any, experimentation. Subjects to which administration of the pharmaceutical compositions of the invention is contemplated include, but are not limited to, humans and other primates, mammals including commercially relevant mammals such as cattle, pigs, horses, sheep, cats, and dogs.

In some embodiments, the compositions of the invention are formulated using one or more pharmaceutically acceptable excipients or carriers. In some embodiments, the pharmaceutical compositions of the invention comprise a therapeutically effective amount of a compound or conjugate of the invention and a pharmaceutically acceptable carrier. Pharmaceutically acceptable carriers that are useful, include, but are not limited to, glycerol, water, saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions may be brought about by including in the composition an agent that delays absorption, for example, aluminum monostearate or gelatin. In some embodiments, the pharmaceutically acceptable carrier is not DMSO alone.

Formulations may be employed in admixtures with conventional excipients, i.e., pharmaceutically acceptable organic or inorganic carrier substances suitable for oral, vaginal, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” that may be included in the pharmaceutical compositions of the invention are known in the art and described, for example in Genaro, ed. (1985, Remington's Pharmaceutical Sciences, Mack Publishing Co., Easton, PA), which is incorporated herein by reference.

The composition of the invention may comprise a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea and combinations thereof. A particularly preferred preservative is a combination of about 0.5% to 2.0% benzyl alcohol and 0.05% to 0.5% sorbic acid.

The composition preferably includes an anti-oxidant and a chelating agent that inhibits the degradation of the compound. Preferred antioxidants for some compounds are BHT, BHA, alpha-tocopherol and ascorbic acid in the preferred range of about 0.01% to 0.3% and more preferably BHT in the range of 0.03% to 0.1% by weight by total weight of the composition. Preferably, the chelating agent is present in an amount of from 0.01% to 0.5% by weight by total weight of the composition. Particularly preferred chelating agents include edetate salts (e.g. disodium edetate) and citric acid in the weight range of about 0.01% to 0.20% and more preferably in the range of 0.02% to 0.10% by weight by total weight of the composition. The chelating agent is useful for chelating metal ions in the composition that may be detrimental to the shelf life of the formulation. While BHT and disodium edetate are the particularly preferred antioxidant and chelating agent respectively for some compounds, other suitable and equivalent antioxidants and chelating agents may be substituted therefore as would be known to those skilled in the art.

Liquid suspensions may be prepared using conventional methods to achieve suspension of the active ingredient in an aqueous or oily vehicle. Aqueous vehicles include, for example, water, and isotonic saline. Oily vehicles include, for example, almond oil, oily esters, ethyl alcohol, vegetable oils such as arachis, olive, sesame, or coconut oil, fractionated vegetable oils, and mineral oils such as liquid paraffin. Liquid suspensions may further comprise one or more additional ingredients including, but not limited to, suspending agents, dispersing or wetting agents, emulsifying agents, demulcents, preservatives, buffers, salts, flavorings, coloring agents, and sweetening agents. Oily suspensions may further comprise a thickening agent. Known suspending agents include, but are not limited to, sorbitol syrup, hydrogenated edible fats, sodium alginate, polyvinylpyrrolidone, gum tragacanth, gum acacia, and cellulose derivatives such as sodium carboxymethylcellulose, methylcellulose, hydroxypropylmethylcellulose. Known dispersing or wetting agents include, but are not limited to, naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g., polyoxyethylene stearate, heptadecaethyleneoxycetanol, polyoxyethylene sorbitol monooleate, and polyoxyethylene sorbitan monooleate, respectively). Known emulsifying agents include, but are not limited to, lecithin, and acacia. Known preservatives include, but are not limited to, methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

Liquid solutions of the active ingredient in aqueous or oily solvents may be prepared in substantially the same manner as liquid suspensions, the primary difference being that the active ingredient is dissolved, rather than suspended in the solvent. As used herein, an “oily” liquid is one which comprises a carbon-containing liquid molecule and which exhibits a less polar character than water. Liquid solutions of the pharmaceutical composition of the invention may comprise each of the components described with regard to liquid suspensions, it being understood that suspending agents will not necessarily aid dissolution of the active ingredient in the solvent. Aqueous solvents include, for example, water, and isotonic saline. Oily solvents include, for example, almond oil, oily esters, ethyl alcohol, vegetable oils such as arachis, olive, sesame, or coconut oil, fractionated vegetable oils, and mineral oils such as liquid paraffin.

Powdered and granular formulations of a pharmaceutical preparation of the invention may be prepared using known methods. Such formulations may be administered directly to a subject, used, for example, to form tablets, to fill capsules, or to prepare an aqueous or oily suspension or solution by addition of an aqueous or oily vehicle thereto. Each of these formulations may further comprise one or more of dispersing or wetting agent, a suspending agent, and a preservative. Additional excipients, such as fillers and sweetening, flavoring, or coloring agents, may also be included in these formulations.

A pharmaceutical composition of the invention may also be prepared, packaged, or sold in the form of oil-in-water emulsion or a water-in-oil emulsion. The oily phase may be a vegetable oil such as olive or arachis oil, a mineral oil such as liquid paraffin, or a combination of these. Such compositions may further comprise one or more emulsifying agents such as naturally occurring gums such as gum acacia or gum tragacanth, naturally-occurring phosphatides such as soybean or lecithin phosphatide, esters or partial esters derived from combinations of fatty acids and hexitol anhydrides such as sorbitan monooleate, and condensation products of such partial esters with ethylene oxide such as polyoxyethylene sorbitan monooleate. These emulsions may also contain additional ingredients including, for example, sweetening or flavoring agents.

Methods for impregnating or coating a material with a chemical composition are known in the art, and include, but are not limited to methods of depositing or binding a chemical composition onto a surface, methods of incorporating a chemical composition into the structure of a material during the synthesis of the material (i.e., such as with a physiologically degradable material), and methods of absorbing an aqueous or oily solution or suspension into an absorbent material, with or without subsequent drying.

The regimen of administration may affect what constitutes an effective amount. The therapeutic formulations may be administered to the subject either prior to or after a diagnosis of disease. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present invention to a subject, preferably a mammal, more preferably a human, may be carried out using known procedures, at dosages and for periods of time effective to prevent or treat disease. An effective amount of the therapeutic compound necessary to achieve a therapeutic effect may vary according to factors such as the activity of the particular compound employed; the time of administration; the rate of excretion of the compound; the duration of the treatment; other drugs, compounds or materials used in combination with the compound; the state of the disease or disorder, age, sex, weight, condition, general health and prior medical history of the subject being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the invention is from about 1 and 5,000 mg/kg of body weight/per day. One of ordinary skill in the art would be able to study the relevant factors and make the determination regarding the effective amount of the therapeutic compound without undue experimentation.

The compound may be administered to a subject as frequently as several times daily, or it may be administered less frequently, such as once a day, once a week, once every two weeks, once a month, or even less frequently, such as once every several months or even once a year or less. It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose will be readily apparent to the skilled artisan and will depend upon any number of factors, such as, but not limited to, the type and severity of the disease being treated, the type and age of the animal, etc.

Actual dosage levels of the active ingredients in the pharmaceutical compositions of this invention may be varied so as to obtain an amount of the active ingredient that is effective to achieve the desired therapeutic response for a particular subject, composition, and mode of administration, without being toxic to the subject.

A medical doctor, e.g., physician or veterinarian, having ordinary skill in the art may readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the subjects to be treated; each unit containing a predetermined quantity of therapeutic compound calculated to produce the desired therapeutic effect in association with the required pharmaceutical vehicle. The dosage unit forms of the invention are dictated by and directly dependent on (a) the unique characteristics of the therapeutic compound and the particular therapeutic effect to be achieved, and (b) the limitations inherent in the art of compounding/formulating such a therapeutic compound for the treatment of a disease in a subject.

In some embodiments, the compositions of the invention are administered to the subject in dosages that range from one to five times per day or more. In another embodiment, the compositions of the invention are administered to the subject in range of dosages that include, but are not limited to, once every day, every two, days, every three days to once a week, and once every two weeks. It will be readily apparent to one skilled in the art that the frequency of administration of the various combination compositions of the invention will vary from subject to subject depending on many factors including, but not limited to, age, disease or disorder to be treated, gender, overall health, and other factors. Thus, the invention should not be construed to be limited to any particular dosage regime and the precise dosage and composition to be administered to any subject will be determined by the attending physical taking all other factors about the subject into account.

Compounds of the invention for administration may be in the range of from about 1 mg to about 10,000 mg, about 20 mg to about 9,500 mg, about 40 mg to about 9,000 mg, about 75 mg to about 8,500 mg, about 150 mg to about 7,500 mg, about 200 mg to about 7,000 mg, about 3050 mg to about 6,000 mg, about 500 mg to about 5,000 mg, about 750 mg to about 4,000 mg, about 1 mg to about 3,000 mg, about 10 mg to about 2,500 mg, about 20 mg to about 2,000 mg, about 25 mg to about 1,500 mg, about 50 mg to about 1,000 mg, about 75 mg to about 900 mg, about 100 mg to about 800 mg, about 250 mg to about 750 mg, about 300 mg to about 600 mg, about 400 mg to about 500 mg, and any and all whole or partial increments therebetween.

In some embodiments, the dose of a compound of the invention is from about 1 mg and about 2,500 mg. In some embodiments, a dose of a compound of the invention used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound (i.e., a drug used for treating the same or another disease as that treated by the compositions of the invention) as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In some embodiments, the present invention is directed to a packaged pharmaceutical composition comprising a container holding a therapeutically effective amount of a compound or conjugate of the invention, alone or in combination with a second pharmaceutical agent; and instructions for using the compound or conjugate to treat, prevent, or reduce one or more symptoms of a disease in a subject.

The term “container” includes any receptacle for holding the pharmaceutical composition. For example, in some embodiments, the container is the packaging that contains the pharmaceutical composition. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating or preventing a disease in a subject, or delivering an imaging or diagnostic agent to a subject.

Routes of administration of any of the compositions of the invention include oral, nasal, rectal, parenteral, sublingual, transdermal, transmucosal (e.g., sublingual, lingual, (trans) buccal, (trans) urethral, vaginal (e.g., trans- and perivaginally), (intra) nasal, and (trans) rectal), intravesical, intrapulmonary, intracerebral, epidural, intracerebroventricular, intraduodenal, intragastrical, intrathecal, subcutaneous, intramuscular, intradermal, intra-arterial, intravenous, intrabronchial, inhalation, and topical administration. In some embodiments, the composition can be administered to the cerebrospinal fluid of a subject.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

### Diagnostic Methods

The present invention provides a method to diagnose a subject having or at risk for developing a disease or disorder associated with protein misfolding or protein aggregates. For example, in some embodiments, the method comprises using the level of expression or activity of one or more TRIM proteins as diagnostic markers. In some embodiments, the method comprises detecting the presence of a genetic mutation in a nucleic acid encoding one or more TRIM proteins.

In some embodiments, the method is used to diagnose a subject as having a disease or disorder associated with protein misfolding or protein aggregates. In some embodiments, the method is used to diagnose a subject as being at risk for developing a disease or disorder associated with protein misfolding or protein aggregates.

In some embodiments, the method is used to evaluate the effectiveness of a therapy for a neurodegenerative disease or disorder associated with protein misfolding or protein aggregates.

In some embodiments, the method comprises collecting a biological sample from a subject. Exemplary samples include, but are not limited to blood, urine, feces, sweat, bile, serum, plasma, tissue biopsy, and the like. For example, in some embodiments, the sample comprises at least one cell of neural tissue. In some embodiments, the sample comprises a neuron, astrocyte, oligodendrocyte, Purkinje cell, pyramidal cell, or the like.

Methods for detecting a reduced expression or activity of one or more TRIM proteins comprise any method that interrogates a gene or its products at either the nucleic acid or protein level. Such methods are well known in the art and include, but are not limited to, nucleic acid hybridization techniques, nucleic acid reverse transcription methods, and nucleic acid amplification methods, western blots, northern blots, southern blots, ELISA, immunoprecipitation, immunofluorescence, flow cytometry, immunocytochemistry. In particular embodiments, disrupted gene transcription is detected on a protein level using, for example, antibodies that are directed against specific proteins. These antibodies can be used in various methods such as Western blot, ELISA, immunoprecipitation, flow cytometry, or immunocytochemistry techniques.

### Methods of Manufacturing Recombinant Protein

In certain embodiments, the present invention provides a method of using one or more TRIM proteins in the production of a recombinant protein of interest. It is recognized in the art the recombinant proteins can spontaneously misfold and aggregate, thus reducing their functionality and utility. Thus, the one or more TRIM proteins can be used to disaggregate protein aggregates of the recombinant protein of interest, thereby allowing for the production and collection of the functional recombinant protein of interest. In some embodiments, the one or more TRIM proteins are one or more selected from the group consisting of human TRIM1, TRIM2, TRIM3, TRIM4, TRIM5, TRIM6, TRIM7, TRIM8, TRIM9, TRIM10, TRIM11, TRIM13, TRIM14, TRIM15, TRIM16, TRIM17, TRIM18, TRIM19, TRIM20, TRIM21, TRIM22, TRIM23, TRIM24, TRIM25, TRIM26, TRIM27, TRIM28, TRIM29, TRIM30, TRIM31, TRIM32, TRIM33, TRIM34, TRIM35, TRIM36, TRIM37, TRIM38, TRIM39, TRIM40, TRIM41, TRIM42, TRIM43, TRIM44, TRIM45, TRIM46, TRIM47, TRIM48, TRIM49, TRIM50, TRIM51, TRIM52, TRIM54, TRIM55, TRIM56, TRIM58, TRIM59, TRIM60, TRIM61, TRIM62, TRIM63, TRIM64, TRIM65, TRIM66, TRIM67, TRIM68, TRIM69, TRIM70, TRIM71, TRIM72, TRIM73, TRIM74, TRIM76, and TRIM77; and mouse TRIM12 and TRIM30. In some embodiments, the one or more TRIM proteins are one or more selected from the group consisting of human TRIM2, TRIM3, TRIM4, TRIM5, TRIM9, TRIM10, TRIM11, TRIM17, TRIM18, TRIM19, TRIM21, TRIM24, TRIM26, TRIM29, TRIM31, TRIM34, TRIM36, TRIM37, TRIM46, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69, and TRIM70; and mouse TRIM12 and TRIM30. In some embodiments, the one or more TRIM proteins are one or more selected from the group consisting of human TRIM10, TRIM11, TRIM24, TRIM36, TRIM37, TRIM40, TRIM49, TRIM55, TRIM58, and TRIM68. In some embodiments, the one or more TRIM proteins are one or more selected from the group consisting of human TRIM2, TRIM3, TRIM4, TRIM5, TRIM9, TRIM10, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70. In some embodiments, the one or more TRIM proteins are one or more selected from the group consisting of human TRIM10, TRIM11, and TRIM55. In some embodiments, the one or more TRIM proteins are one or more selected from the group consisting of human TRIM2, TRIM3, TRIM10, TRIM11, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73. In some embodiments, the one or more TRIM proteins are one or more selected from the group consisting of human TRIM10, TRIM11, TRIM36, TRIM55, and TRIM68. In some embodiments, the one or more TRIM proteins are one or more selected from the group consisting of human TRIM10, TRIM11, TRIM24, TRIM36, and TRIM58. In some embodiments, the one or more TRIM proteins are one or more selected from the group consisting of TRIM10, TRIM11, TRIM17, TRIM36, TRIM37, TRIM40, TRIM49, and TRIM55. In some embodiments, TRIM protein is human TRIM10. In some embodiments, the TRIM protein is human TRIM11

In certain embodiments, the present invention provides a method of increasing production of a recombinant protein of interest using one or more TRIM proteins disclosed herein. It is recognized in the art that proteins, when overexpressed in cell-based expression systems, can misfold and aggregate at high concentrations resulting in premature cell death. Thus, proteins that can prevent or address protein misfolding, such as TRIM proteins of the present disclosure, can be used to prevent misfolding and cell death while allowing increased production of functional recombinant protein.

In certain embodiments, the method comprises administering to a cell one or more TRIM proteins, a nucleic acid molecule encoding one or more TRIM proteins, or a combination thereof. In certain embodiments, the cell is modified to express the recombinant protein of interest. The cell may be of any expression system, including, but not limited to a yeast expression system, bacterial expression system, insect expression system, or mammalian expression system.

### Methods of Cell Maintenance

In some embodiments, the present invention comprises a method of cell maintenance for use in cell therapy. It is recognized that cells for use in cell therapy, such as cells engineered to overexpress a therapeutic protein, may be subject to protein misfolding and aggregation leading to premature cell death. Thus, the TRIM proteins of the present disclosure can be used to prevent or address protein misfolding and aggregation to keep cells healthy and available for use. In some embodiments, the method comprises administering to said cell one or more TRIM proteins, a nucleic acid molecule encoding one or more TRIM proteins, or a combination thereof.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the present invention and practice the claimed methods. The following working examples therefore are not to be construed as limiting in any way the remainder of the disclosure.

### Example 1: TRIM11 Protects Against Tauopathies and is Downregulated in Alzheimer's Disease

Intracellular neurofibrillary tangles (NFTs) composed of hyperphosphorylated tau is the pathological hallmark shared by more than twenty heterogeneous dementias and movement disorders, collectively referred to as tauopathies (V. M. Lee, M. Goedert, J. Q. Trojanowski, Annu Rev Neurosci 24, 1121-1159 (2001); M. G. Spillantini, M. Goedert, Lancet Neurol 12, 609-622 (2013); J. Gotz, G. Halliday, R. M. Nisbet, Annu Rev Pathol 14, 239-261 (2019). Among them, frontotemporal lobar degeneration with tau pathology (FTLD-tau)-which includes various diseases such as progressive supranuclear palsy (PSP), corticobasal degeneration, and Pick's disease (PiD)—are primary tauopathies that display no other major pathological abnormalities. Alzheimer's disease (AD), the most common cause of dementia, is a secondary tauopathy additionally characterized by the presence of extracellular amyloid β (Aβ) plaques (C. L. Masters et al., Nat Rev Dis Primers 1, 15056 (2015); D. S. Knopman et al., Nat Rev Dis Primers 7, 33 (2021)). In a familial subset of primary tauopathies, the tau-encoding gene MAPTis mutated (M. Hutton et al., Nature 393, 702-705 (1998); M. G. Spillantini et al., Proc Natl Acad Sci USA 95, 7737-7741 (1998); C. Dumanchin et al., Hum Mol Genet 7, 1825-1829 (1998)). In both primary tauopathies and AD, NFT burden correlates with cognitive decline and neurodegeneration (P. V. Arriagada, J. H. Growdon, E. T. Hedley-Whyte, B. T. Hyman, Neurology 42, 631-639 (1992); H. Ling et al., Neuropathol Appl Neurobiol 40, 149-163 (2014); N. Kouri et al., Brain 134, 3264-3275 (2011)). Moreover, tau is required for Aß-induced neurotoxicity (E. D. Roberson et al., Science 316, 750-754 (2007)). Therefore, tau misfolding and aggregation likely represent the main disease-causing event for AD and other tauopathies.

To maintain proteins in their functional soluble form, organisms in all kingdoms of life have evolved protein quality control (PQC) systems (W. E. Balch, R. I. Morimoto, A. Dillin, J. W. Kelly, Science 319, 916-919 (2008); S. Wolff, J. S. Weissman, A. Dillin, Cell 157, 52-64 (2014; D. Balchin, M. Hayer-Hartl, F. U. Hartl, Science 353, aac4354 (2016)). These systems include degradative pathways that recycle defective proteins and superfluous normal proteins, molecular chaperones that prevent protein misfolding and aggregation, and disaggregases that dissolve pre-existing protein deposits. The conversion of tau from soluble monomers to fibrillar aggregates in tauopathies in an age-dependent manner suggests a diminishing capacity of a PQC system that can normally protect against tau aggregation. Nevertheless, the identity and nature of such a PQC system remain undefined. This lack of knowledge hinders the development of effective therapies.

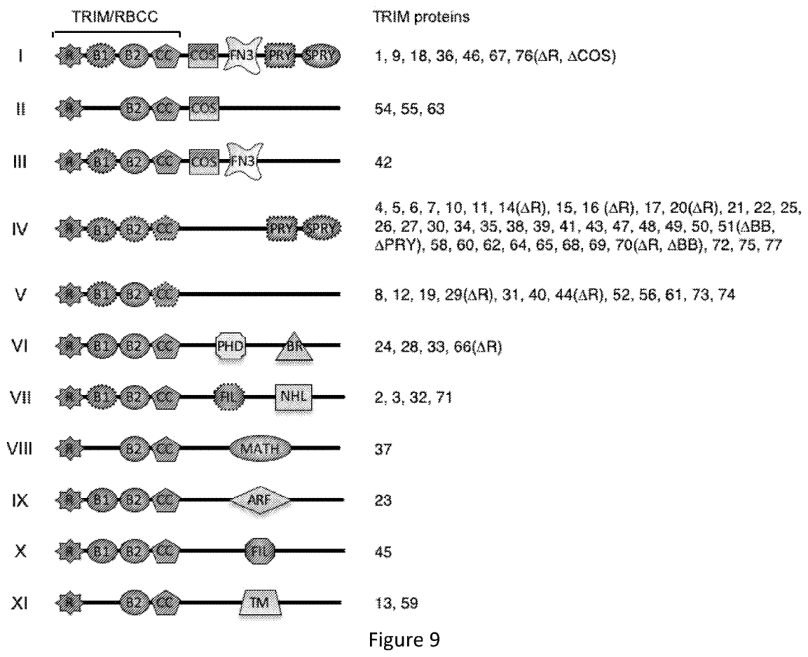

Tripartite motif (TRIM) proteins are defined by a TRIM/RBCC region consisting of a RING domain, one or two B-boxes, and a coiled-coil motif (FIG. 9). These proteins are present only in metazoans and their number has expanded rapidly during evolution, including over 70 members in humans (S. Hatakeyama, Nat Rev Cancer 11, 792-804 (2011); K. Ozato, D. M. Shin, T. H. Chang, H. C. Morse, 3rd, Nat Rev Immunol 8, 849-860 (2008)). Emerging evidence suggests that some TRIM proteins may participate in multiple aspects of PQC (L. Guo et al., Mol Cell 55, 15-30 (2014); L. Chen et al., Cell Rep 18, 3143-3154 (2017); L. Chen, G. Zhu, E. M. Johns, X. Yang, Nat Commun 9, 1223 (2018); G. Zhu et al., Cell Rep 33, 108418 (2020)) although other TRIMs can aggravate protein aggregation (M. W. Rousseaux et al., Elife 5, (2016)). Moreover, TRIM21, a cytoplasmic antibody receptor, mediates degradation of antibody-coated viruses that happen to enter the cytoplasm or endogenous proteins for which specific antibodies are delivered intracellularly D. L. Mallery et al., Antibodies mediate intracellular immunity through tripartite motif-containing 21 (TRIM21). Proc Natl Acad Sci USA 107, 19985-19990 (2010; A. Kondo et al., Nature 523, 431-436 (2015); D.

Clift et al., Cell 171, 1692-1706 e1618 (2017); W. A. McEwan et al., Proc Natl Acad Sci USA 114, 574-579 (2017)). Here, the role of the TRIM system was examined in the pathogenesis of tauopathies, its mechanism of action, and its utility for disease treatment.

The materials and methods used in the experiments are now described.

### Antibodies

Antibodies against the following proteins or epitopes were purchased from the indicated sources: TRIM10 (pAb, Abcam, Cat #ab151306), TRIM11 (pAb, Millipore, Cat #ABC926; pAb, Abcam, Cat #ab111694), TRIM55 (MURF2) (mAb, Abnova, Cat #H00084675-M02), GFP (mAb, Santa Cruz, Cat #sc-9996; pAb, GeneTex, Cat #GTX113617), HA (mAb, C29F4, Cell Signaling, Cat #3724), GAPDH (Santa Cruz, Cat #sc-32233), β-actin (Sigma-Aldrich, Cat #A5441), FLAG (mAb, Cell signaling, Cat #14793; mAb, M2, Sigma-Aldrich, Cat #F1804), Hsp90 (Cell Signaling, Cat #4874), phospho-tau (Ser202/Thr205) (mAb AT8, Thermo Fisher Scientific, Cat #MN1020), phospho-tau (Thr231) (mAb AT180, Thermo Fisher Scientific, Cat #MN1040), phospho-tau (Ser262) (pAb, Invitrogen, Cat #OPA1-03142), phospho-tau (Ser396) (pAb, Thermo Fisher Scientific, Cat #44-752G), tau (mAb tau5, Thermo Fisher Scientific, Cat #AHB0042), tau (pAb T22, Millipore, Cat #ABN454), p62 (SQSTM1) (pAb, MBL, Cat #PM045), 6×His (pAb, Cell Signaling, Cat #2365), mCherry (mAb, Santa Cruz, Cat #sc-390909), c-Myc (pAb, Santa Cruz, Cat #sc-40), LC3B (D11, XP, mAb, Cell Signaling, Cat #3868), SUMO2/3 (pAb, Abcetpa, Cat #AP1224a), and FLAG M2 agarose beads (Sigma-Aldrich, Cat #A1205). PSD95 (mAb, Cell Signaling, Cat #36233S). PSD95 (mAb, Cell Signaling, Cat #3409S). Synaptophysin (mAb, Cell Signaling, Cat #36406S). NeuN (mAb, Millipore, Cat #MAB377). MAP2 (pAb, Origene Technologies, Cat #TA309162). Anti-phospho-tau mAbs PHF-1 (Ser396/Ser404) and MC1 were kindly provided by Dr. Peter Davies.

### Reagents

Mg²⁺-ATP (Cat #A9187), FLAG Peptide (Cat #F3290), ammonium chloride (Cat #09718), Benzonase (Cat #E1014), Isopropyl-1-thio-D-galactopyranoside (IPTG) (Cat #16758), Complete protease inhibitor Cocktail (Cat #11697498001), L-glutathione reduced (Cat #G4251), cycloheximide (Cat #66819), thioflavin T (Cat #T3516), imidazole (Cat #15513), heparin (Cat #H3393), human Aβ42 peptide, Duolink® In Situ Red starter Kit (Cat #DUO92101) and Polybrene (Cat #TR-1003) were purchased from Sigma-Aldrich. Glutathione Superflow Agarose (Cat #25236), High-Capacity cDNA Reverse Transcription Kit (Cat #4368814), puromycin dihydrochloride (Cat #A1113803), Lipofectamine2000 (Cat #6031), Lipofectamine RNAiMAX (Cat #13778150), SYBR Green Master Mix (Cat #A25742) and SuperSignal™ Western Blot Substrate Bundle (Cat #A45916) were purchased from Thermo Fisher Scientific. SUMO E1 (Cat #E-315), SUMO E2 (UbcH9) (Cat #E2-465), and 6×His-SUMO2 (Cat #UL-75) were purchased from Boston Biochem. Ni-NTA Agarose (Cat #30230) was purchased from QIAGEN, TRIzol Reagent (Cat #15596) and Hochst33342 (Cat #H3570) from Invitrogen, 4′,6-diamidino-2-phenylindole (DAPI) (Cat #H-1200) from Vector Laboratories, MG132 (Cat #S2619) from Selleck Chemicals, poly-ethylenimine (PEI) (Linear, MW 25000, Cat #23966-1) from Polysciences, Phosphatase inhibitor PhosSTOP™ (Cat #04906845001) from Roche, DPX mounting medium (Cat #13510) from Electron Microscopy Science, Bradford protein assay kit (Cat #5000205) from Bio-Rad Labs, and ABC (Avidin-Biotin Complex) Kits (Cat #PK6100) from Vector Laboratories.

### Plasmids

For expression in mammalian cells, cDNAs for 75 TRIMs, including 73 human TRIMs and mouse TRIM12 and TRIM30 (Table 1), were synthesized and cloned into pCDH-EF1-FHC (Addgene, #64874), in which each TRIM was fused with an FLAG tag and an HA tag at the C-terminus (Gene Universal, Newark, DE). Flag-tau and Flag-tau P301L were cloned in pcDNA3.1. Tau-VN173 and TRIM11-VN173 were cloned in pBiFC-VN173 (Addgene plasmid #22010), and tau-VC155 and TRIM11-VC155 were cloned in pBiFC-VC155 (Addgene plasmid #22011) (gifts of Dr. Chang-Deng Hu). Tau-GFP, tau P301L-GFP, and tau AT8 (S199E, S202E, T205E)-GFP were made in pEGFP-N1, in which EGFP is fused to the C-terminus of tau proteins. pRK5-EGFP-tau and pRK5-EGFP-tau P301L, in which EGFP is fused to the N-terminus of tau proteins, were gifts of Dr. Karen Ashe (Addgene plasmids #46904 and #46908, respectively). GFP-TRIM11 (in pEGFP-C1), Flag-TRIM11, Flag-TRIM112EA (in pcDNA3.1) (G. Zhu et al., Cell Rep 33, 108418 (2020)), mCherry, and mCherry-TRIM11 (in pTRPE) (L. Chen et al., Cell Rep 18, 3143-3154 (2017)) were previously described.

For expression in bacteria, GST-tau, GST-tau P301L, and GST-tau AT8 were made in pGEX-1ZT, a derivative of pGEX-12T with additional cloning sites. GFP-tau and GFP-tau P301L were cloned in pET-28 (+). GST-TRIM11 was cloned in pGEX-1ZT (Zhu, G., et al., 2020, Cell Reports, 33:108418).

siRNAs, sgRNAs, and Antisense Oligos

siRNA targeting mouse TRIM10 (Cat #sc-76733), mouse TRIM11 (Cat #sc-76735), mouse TRIM36 (Cat #sc-154647), mouse TRIM55 (Cat #sc-149718), and human TRIM11 (Cat #sc-76734) were purchased from Santa Cruz. Negative control siRNA: 5′-GGUUAAUCGCGUAUAAUACGCGUAU-3′ (SEQ ID NO:1) was made by IDT.

sgRNAs targeting human TRIM10, TRIM11, TRIM26, TRIM36 and TRIM55 were synthesized by Integrated DNA Technologies (Coralville, IA, USA). Targeting sequences are: TRIM10, 5′-GGCAGTTGACTTCATCTGCC-3′ (SEQ ID NO: 2); TRIM11, 5′-GAGCCAGCGGCAGAACGTGC-3′ (SEQ ID NO:3); TRIM26, 5′-GCCGCTCAATGTTCTCCACC-3′ (SEQ ID NO:4); TRIM36, 5′-TACCATTAAGAATATCGAAA-3′ (SEQ ID NO:5); TRIM55: 5′-AACCCGTATTTGCCCACAAG (SEQ ID NO:6).

Antisense oligos (ASOs) used in this study were designed and synthesized by AUM LifeTech (Philadelphia, PA, USA). Sequences are: TRIM11-1, 5′-ATAAACAGCAGCGACCCATCC-3′ (SEQ ID NO:7); TRIM11-2, 5′-ACTTAGTGCTTTGGTGAGAGC-3′ (SEQ ID NO:8); TRIM11-3, 5′-ACTGTAGAATGAGAGATGGCC-3′ (SEQ ID NO:9); TRIM11-4, 5′-TAGAATGAGAGATGGCCAGCT-3′ (SEQ ID NO:10); TRIM11-5, 5′-ATTTGTTTCCGTAGGTGCTCC-3′ (SEQ ID NO:11); scrambled control (SCR CTRL), 5′-CCTTCCCTGAAGGTTCCTCC-3′ (SEQ ID NO:12). SCR CTRL-Far Red was tagged with a far-red fluorescent dye with Excitation Max at 646 nm and Emission Max at 669 nm (+/−5).

### Cell Culture

HEK293T and N2A cells were purchased from ATCC, and SH-SY5Y cells were purchased from Sigma. HEK293 cells expressing RD (LM)-YFP cells, which express the tau repeat domain (RD; aa 244 to 372 of the full-length tau 4R2N isoform) harboring the pro-aggregation mutations P301L and V337M, were generated according to published protocol (D. W. Sanders et al., Neuron 82, 1271-1288 (2014)). QBI293/tau P301L-GFP cells were previously described (J. L. Guo et al., J Biol Chem 291, 13175-13193 (2016)). HEK293T, HEK293/RD (LM)-YFP, and QBI293/tau P301L-GFP cells were cultured in DMEM medium, SH-SY5Y cells in DMEM/F12 (1:1), and N2A cells in EMEM medium. All media contained penicillin/streptomycin and 10% FBS. Cells were maintained at 37 °C in a humidified incubator with 5% CO₂.

Primary neurons were prepared from hippocampus or cerebral cortices of wild-type or PS19 mouse pups (P1). Tissues were removed and put into ice-cold Hank's Balanced Salt Solution containing 10 mM HEPES, chopped into small strips, and digested by papain (1 mg/ml) for 30 min at 37 °C. DMEM with 10% heat-inactivated fetal bovine serum was added to terminate the digestion reaction. After trituration, cells were harvested by centrifugation at 1,000×g and resuspended in neurobasal medium containing 2% B27, 1% penicillin/streptomycin, and 2 mM GlutaMAX.

### TRIM Knockout (KO) Cells

To knock out TRIM10, TRIM11, TRM26, TRIM36, or TRIM55 in HEK293T cells, control plentiCRISPRv2 vector or plentiCRISPRv2 encoding TRIM10, TRIM11, TRIM26, TRIM36, or TRIM55 sgRNA was co-transfected with the packaging plasmids pMD2.G (Addgene #12259) and psPAX2 (Addgene #12260) in a ratio of 4:1:3 with PEI in HEK293T cells. The medium was replaced 12 h later. Viral particles were collected at 60 h post-transfection and centrifuged at 1,200 rpm for 5 min, filtered with 0.45 μm sterile filter (Millipore), and concentrated by Lenti-X™ concentrator (Takara Bio, Cat #631312) at a ratio of 3:1 overnight at 4 °C, followed by centrifugation at 4 °C at 4500×g for 1 h. HEK293T cells were then infected with the concentrated viral particles in medium containing 8 μg/ml polybrene. Lentiviral-transduced cells were selected with 2 μg/ml puromycin for 7 days.

### Mice

Tau P301S (PS19) or B6;C3-Tg (Prnp-MAPT*P301S) PS19Vle/J) heterozygous female breeders (Strain #: 008169), and 3×Tg-AD or B6;129-Tg(APPSwe,tauP301L)1Lfa Psen1^{tm1Mpm}/Mmjax) (MMRRC Strain #034830-JAX) homozygous male breeders, were purchased from Jackson Laboratories. The PS19 colony was maintained through heterozygous breeding pairs with C57Bl/6J wild-type male mice, and heterozygous PS19 transgenic mice were used in this study. The 3×Tg-AD colonies were maintained through heterozygous mating, and homozygous mice were included in this study. Genotype was confirmed using PCR. The mice were housed in groups of 3-5 with food and water available ad libitum, at a constant temperature of 23 °C in a 12-hour light/dark cycle.

cDNA/siRNA transfection and lentiviral transduction cDNA plasmids were transfected into cultured cells using Lipofectamine 2000 (Invitrogen) or polyethylenimine, and siRNAs were transfected using Lipofectamine RNAiMAX. When both siRNA and cDNA were used, cells were first transfected with siRNA for 24 h and then with cDNA plasmid for another 24 h. QBI293/tau P301L-GFP cells stably expressing mCherry or mCherry-TRIM11 were generated by lentiviral transduction, using the 3rd generation lentiviral packaging system. HEK293T cells were transfected with mCherry or mCherry-TRIM11 plasmid, together with the helper plasmids Gag, Rev, and VSVG. Lentiviral vectors were obtained by centrifuging culture medium at 10,000 rpm for 18-20 h and used to transduce QBI293/tau P301L-GFP cells (J. L. Guo et al., J Biol Chem 291, 13175-13193 (2016)). After three days of viral transductions, mCherry-positive cells were selected using fluorescence-activated cell sorting (FACS) and grown in DMEM media. These cells were further selected by two rounds of FACS and used in this study.

### Screening of TRIM Proteins on Tau

HEK293T cells cultured in 6-well plates for 24 h and at ~80-90% confluence were transfected with 0.5 μg of pRK5-GFP-tau P301L and 2 μg of the indicated pCDH-EF1-FHC-TRIM-FLAG-HA plasmids using Polyethylenimine. Medium was changed 12 h later. Cells were harvested 48 h after transfection and lysed with 150 μl ice-cold lysis buffer (50 mM Tris, pH 8.8, 100 mM NaCl, 5 mM MgCl₂, 0.5% NP-40, 1 mM DTT, 250 IU/ml benzonase, 1 mM PMSF, and 1× complete protease inhibitor cocktail) for 30 minutes (min) on ice. Lysates were centrifuged for 15 min at 13,000 rpm at 4 °C. The NP-40-soluble supernatants were designated as SN fraction. The NP-40-insoluble pellets were washed once with 500 μl precooled PBS and re-suspended in 50 μl ice-cold pellet buffer (20 mM Tris, pH 8.0, 15 mM MgCl₂, 1 mM DTT, 250 IU/ml benzonase, 1 mM PMSF, and 1× complete protease inhibitor cocktail) for 30 min on ice, and then added 25 μl 3× boiling buffer (6% SDS, 20 mM Tris, pH 8.0, 150 mM DTT). The samples were heated at 95 °C for 5-10 min. Aggregated proteins in pellets that could be solubilized by SDS were designated as the SDS-soluble pellet fraction (PE).

Both the SN and PE fractions were boiled in SDS sample buffer (final concentration: 62.5 mM Tris pH 6.8, 2% SDS, 10% glycerol, 100 mM DTT, 0.01% bromophenol blue), analyzed by SDS-PAGE, and then were transferred to a nitrocellulose membrane. After blocking with 5% non-fat milk in tris-buffered saline with Tween (TBST), the membrane was incubated with anti-GFP, anti-HA, and anti-HSP90 antibodies, followed by HRP-conjugated secondary antibodies. Signals were detected by using a Western Blot Substrates and Substrate Kits in an ECL detection system (Bio-Rad Chemidoc Touch Imaging System Chemiluminescence/Fluorescence Detection). The sample loading and exposure time of the immunoblots were controlled so that all bands of western blots were detected within the linear range.

### Real-Time Quantitative PCR (RT-qPCR)

Total RNA was extracted using TRIzol according to the manufacturer's instructions. 1 μg of RNA was reversely transcribed using High Capacity cDNA Reverse Transcription Kit. Gene expression was determined by SYBR Green-based RT-PCR using an ABI ViiA 7 PCR system (Applied Biosystems, Foster City, CA, USA). Each gene expression was normalized to GAPDH. The following primers were used (5′ to 3′):

tau-F:
(SEQ ID NO: 13)
GAGGCGGGAAGGTGCAGATAATTAATAA,
tau-R:
(SEQ ID NO: 14)
CTGGTTTATGATGGATGTTGCC;
TRIM10-F:
(SEQ ID NO: 15)
CTGCCCCATCTGTCAGGGTA,
TRIM10-R,
(SEQ ID NO: 16)
GGTATCTCACAGTAGCGGGTAA;
TRIM11-F:
(SEQ ID NO: 17)
TACTGGGAGGTGGAGGTTGGG,
TRIM11-R:
(SEQ ID NO: 18)
GGATCTCGGGAAAGATGAATAGCA;
TRIM26-F:
(SEQ ID NO: 19)
TGCACTACTACTGTGAGGACG,
TRIM26-R:
(SEQ ID NO: 20)
TCCTTAGGGTACTCAGGTGGT;
TRIM36-F:
(SEQ ID NO: 21)
GAGCTGTTTACCCACCCATTG,
TRIM36-R:
(SEQ ID NO: 22)
CTGATCCCACATCGTTGAATGA;
TRIM55-F:
(SEQ ID NO: 23)
TTGTCAGCACAACCTGTGTAG,
TRIM55-R:
(SEQ ID NO: 24)
CCCATGTCTATCCAAAACCACTT;
GAPDH-F:
(SEQ ID NO: 25)
GCTAAGGCTGTGGGCAAGG,
GAPDH-R:
(SEQ ID NO: 26)
GGAGGAGTGGGTGTCGCTG.

Protein Extraction from Human Brain Samples

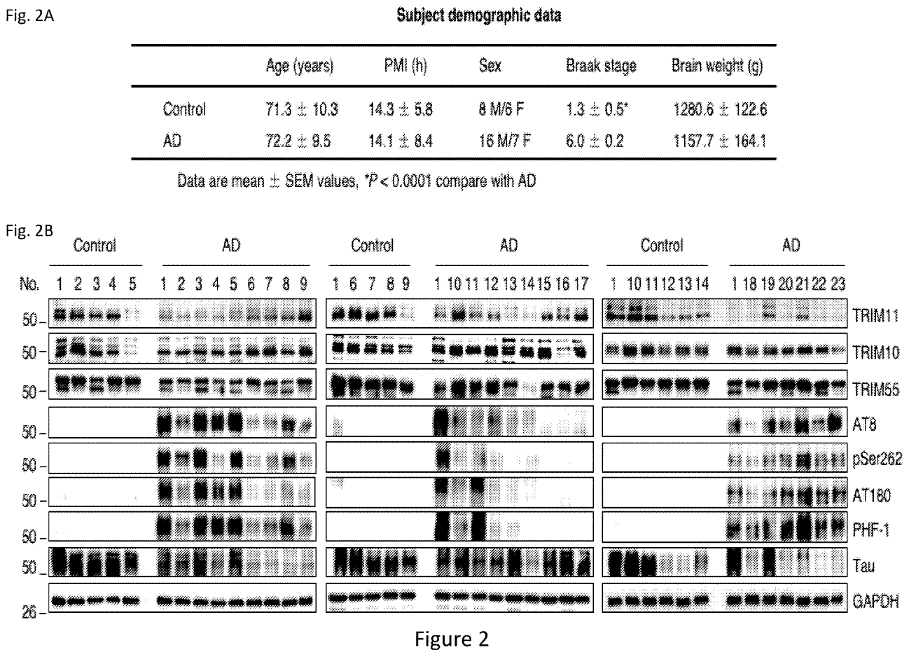

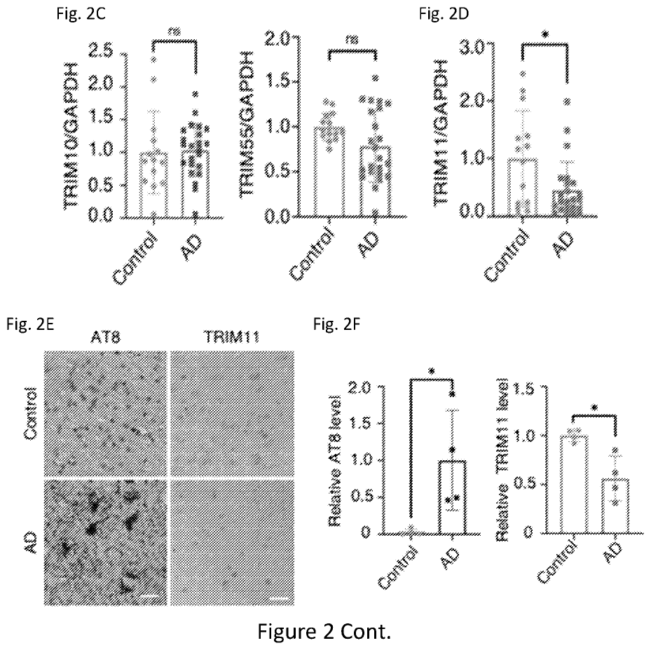

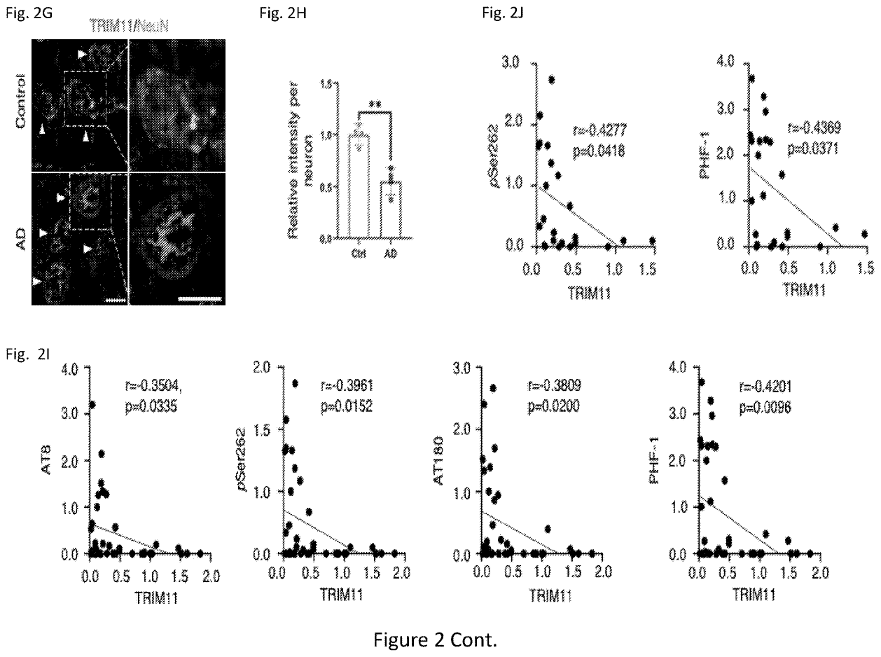

Postmortem human brain tissues from twenty-three neuropathologically confirmed AD cases and fourteen control individuals without a history of dementia or other neurologic disorders (FIG. 2A and Table 2) were obtained. Proteins were extracted from these human brain tissues as previously described (G. S. Gibbons et al., Mol Neurodegener 15, 64 (2020)). Briefly, gray matters from frontal cortices were homogenized in a high salt, sarkosyl-containing buffer (10 mM Tris-HCl pH 7.4, 800 mM NaCl, 1 mM EDTA, 2 mM DTT, protease inhibitor cocktail, 1 mM PMSF, PhosSTOP™, 0.1% sarkosyl and 10% sucrose) with homogenizer in nine volumes of buffer per gram tissue. After 30 min on ice, lysates were centrifuged at 10,000 g for 10 min at 4 °C. The supernatants were collected and protein concentrations were measured using Bradford assay (Bio-Rad Labs), boiled in SDS sample buffer and then analyzed by SDS-PAGE.

### Immunohistochemistry (IHC) and Immunofluorescence (IF) Analyses of Human Brain Tissues

Human brain tissues were fixed in paraformaldehyde, paraffin-embedded, and cut into 6 μm thick sections, deparaffinized in xylene and rehydrated in ethanol (100-50%). For IHC staining, rabbit anti-TRIM11 antibodies were diluted 1:500 in 3% goat serum in PBS and applied overnight to rehydrated tissue sections after antigen retrieval at 4 °C. After five washes with PBS, sections were incubated with biotin-conjugated secondary antibody, linked to avidin by incubating with ABC Kit for 1 hour and color developed with DAB solution. For IF staining, rabbit anti-TRIM11 antibodies, mouse anti-tau AT8 antibodies or mouse anti-NeuN antibodies were diluted 1:500 in 3% goat serum in PBS and applied overnight at 4 °C. After 5 washes with PBS, the labeled proteins were visualized by incubation with Alexa Fluor 488-conjugated and Alexa Fluor 555-conjugated secondary antibodies. Sections were then dehydrated, cleared in xylene and mounted on slides using DPX mounting medium (Electron Microscopy Science). Samples were visualized by using an inverted fluorescence microscope (Revolve, Echo Laboratories).

### Protein Purification

GST, GST-TRIM11, GST-tau, 6×His-GFP-tau, and 6×His-GFP-tau P301L were purified from bacteria. BL21 DE3 cells (Thermo Fisher Scientific, Cat #C600003) containing the corresponding plasmids were grown at 37 °C to A600 nm=0.6-0.8, and induced for protein expression with 0.5 mM IPTG at 19-20 °C for 20 h. Cells were collected and resuspended in a buffer containing 50 mM Tris-HCl, pH 7.4, 500 mM NaCl, 200 mM KCl, 10% glycerol (for GST proteins) or a buffer containing 50 mM NaH₂PO₄, 300 mM NaCl (for 6×His-tau), each supplemented with complete protease inhibitor cocktail, 1 mM phenylmethylsulphonyl fluoride, 1 mM DTT, and 1 mg/ml lysozyme. Cells were lysed by sonication. Cell lysates were centrifuged at 13,000 rpm and 4 °C for 30 min.

For purification of GST proteins, the supernatants were applied to a column (QIAGEN, cat #34694) packed with glutathione beads and incubated at 4 °C for 2 h. The column was washed extensively with washing buffer (50 mM Tris-Cl, pH 7.5 and 150 mM NaCl). The bound proteins were eluted with elution buffer (50 mM Tris-Cl, pH 7.5, 150 mM NaCl and 20 mM glutathione). Fractions were collected at 0.5 mL each, and fractions containing GST or GST fusions were concentrated and desalted with centrifugal filters (Millipore, cat #: UFC800308). For purification of 6×His-GFP-tau and 6×His-GFP-tau P301L, supernatants were incubated with Ni-NTA Agarose (QIAGEN) at 4 °C for 2 h. Beads were washed extensively with wash buffer (50 mM NaH₂PO₄, 300 mM NaCl, 20 mM Imidazole, pH 8.0). Fusion proteins were eluted by elution buffer (50 mM NaH₂PO₄, 300 mM NaCl, 400 mM imidazole, pH 8.0), concentrated and desalted with centrifugal filters.

Flag-TRIM11 and Flag-TRIM1 12EA were purified from HEK293T cells as previously described (76, 77). Briefly, HEK293T cells transfected with the Flag-TRIM11 plasmid were lysed in IP-lysis buffer (20 mM Tris-HCl at pH 7.4, 150 mM NaCl, 0.5% Triton X-100, 0.5% NP-40, and 10% glycerol) with sonication. Supernatants were incubated with anti-Flag M2 Affinity Gel at 4 °C for 4 h to overnight. The gel was washed sequentially with lysis buffers containing additional 0, 0.25, 0.5, 1, 0.5, 0.25 and 0 M KCl, and then with Tris buffer or sodium phosphate buffer. Recombinant proteins were eluted with 3×FLAG peptide at 4 °C for 1 h, concentrated, and desalted with centrifugal filters.

Tau-441 was purified as previously described (W. Li, V. M. Lee, Biochemistry 45, 15692-15701 (2006)). Tau-441 P301L (cat. #T-1014-1) was purchased from rPeptide (Watkinsville, GA).

### Cycloheximide Chase Assay

HEK293T cells were cultured in 12-well plates for 24 h and then transfected with indicated plasmids. To analyze half-life of GFP-tau or GFP-tau P301L, cells were treated with cycloheximide (CHX) (50 μg ml-1) for different durations at 24 h after transfection. Cells were harvested after treatment and SN and PE fractions were extracted as described above.

### BiFC Assay

HEK 293T cells were seeded into 6 well plates for 24 h with DMEM medium supplemented with 10% fetal bovine serum (FBS), so that cells grow to a confluence of 80-90% at the time of transfection. Cells were co-transfected with indicated plasmids. 12 h later, cells were changed with fresh medium and cultured for another 12 h. Fluorescence was observed on Revolve Microscope Demo (Echo Laboratories).

### Co-Immunoprecipitation

Co-immunoprecipitation assayed was performed as previously described (G. Zhu et al., Cell Rep 33, 108418 (2020)). Briefly, HEK293 cells transfected with the indicated expression plasmids were lysed in lysis buffer (50 mM Tris, pH 7.4, 200 mM NaCl, 0.2% Triton, 1 mM DTT, 1 mM PMSF, and 1× complete protease inhibitor cocktail) for 30 min on ice. Cell lysates were centrifuged and the supernatants were collected and incubated at 4 °C with the indicated primary antibody overnight. The protein A/G-Agarose beads were incubated with the immune complexes for 4 h. After extensive washes, the immunoprecipitates were resuspended in SDS sample buffer and boiled for 5 min. Immunoprecipitates and whole cell lysates were resolved by SDS-PAGE and western blot.

### Co-Localization Assay

To assay the co-localization of endogenous TRIM11 and tau in SH-SY5Y, N2A, and cultured neurons, cells were fixed for 10 min in PBS containing 4% paraformaldehyde and 4% sucrose at room temperature. Cells were then permeabilized with 0.5% Triton X-100 for 5 min and blocked for 30 min with 10% NGS in PBS, followed by incubation with rabbit anti-TRIM11 antibody, mouse anti-tau antibody (for all cells), and chicken anti-MAP2 antibody (for neurons only) overnight at 4 °C. After 3 washes with PBS, the labeled proteins were visualized by incubation with Alexa Fluor 488-conjugated goat anti-rabbit, Alexa Fluor 555-conjugated goat anti-mouse, and Alexa Fluor 405-conjugated goat anti-chicken (for MAP2 staining) secondary antibodies for 1 hour at room temperature. After three washes with PBS, coverslips were mounted on glass slides, and fluorescence images were captured by a confocal microscopy. To quantify the co-localization of endogenous TRIM11 and tau, SH-SY5Y, N2A cells, or same length of neurons dendrites were randomly selected, and co-localization was quantified with Image J plugin JACoP.

### Proximity Ligation Assay (PLA)

To assay PLA Fluorescence of endogenous TRIM11 and tau in SH-SY5Y, N2A, and primary cultured neurons, Duolink® In Situ Red Starter Kit from Sigma-Aldrich was used. Cells were fixed for 10 min in PBS containing 4% paraformaldehyde at room temperature. After fixing, the cells were washed with PBS, permeabilized with 0.5% Triton X-100 for 5 min and blocked for 60 min with Duolink® blocking solution at 37 °C. Cells were then incubated with rabbit anti-TRIM11 antibody and mouse anti-tau antibody overnight at 4 °C. Secondary antibodies conjugated with oligonucleotides were added to the reaction and incubated for 1 h at 37 °C. Ligation and amplification were performed by incubating with the ligation solution for 30 min at 37 °C and then incubating with amplification buffer containing polymerase for 100 min at 37 °C. The slides were mounted with mounting medium with DAPI then the fluorescence images were captured by a confocal microscopy. As a negative controls, the proximity ligation assay was performed in absence of one of the primary antibodies.

### SUMOylation Assay

For SUMOylation assays in cells, HEK 293T cells were transfected with the indicated plasmids for 48 h and treated with the proteasome inhibitor MG132 (10 mM) for 6 h. Cells were lysed in lysis buffer (50 mM Tris-HCl, pH 7.4, 150 mM NaCl, 0.5% Triton, 1 mM DTT, 1 mM PMSF, and 1× complete protease inhibitor cocktail) supplemented with 2% SDS and 50 mM DTT. Cell lysates were boiled at 95 °C for 10 min and diluted 10-fold in lysis buffer containing no SDS. A fraction of cell lysates was saved. The rest of the cell lysates were incubated with anti-GFP antibody at 4 °C overnight and with protein A/G agarose beads for an additional 2 h (denaturing immunoprecipitation, d-IP). d-IP samples were washed extensively and analyzed along with cell lysates by immunoblotting with the indicated antibodies.

In vitro SUMOylation assays were performed at 37 °C for 1.5 h in 20 μl reaction buffer (50 mM Tris pH 7.5 and 2.5 mM DTT) containing purified GST-tau or GST-tau P301L (600 ng/400 nM each), SUMO E1 (125 nM), SUMO E2 (1 μM), Flag-TRIM11 (300 nM), His-SUMO2 (25 μM), and 10 mM Mg²⁺-ATP. The reaction mixtures were stopped by adding 20 μl IP-lysis buffer containing 2% SDS and 50 mM DTT and heating at 95 °C for 10 min. A fraction of the heat-denatured reaction mixtures was saved. The rest were diluted into 1.5 ml IP-lysis buffer without SDS. GST-tau or GST-tau P301L was immunoprecipitated by anti-GST beads (anti-GST d-IP). After extensive washing, d-IP samples and reaction mixtures were analyzed by Western blot using indicated antibodies.

### Prevention and Disaggregation of Tau Fibrils

For prevention, purified tau-441 (10 μM) was induced to form tau aggregation by Heparin (30 μM) or by preformed tau fibrils (PFFs, 0.2 μM) in a reaction buffer (20 mM Tris-HCl, pH 7.4, 100 mM NaCl, 1 mM EDTA and 1 mM DTT) in the presence of the indicated concentrations GST or GST-TRIM11 at 37 °C for 24 h. Tau fibrillization was analyzed by ThT-binding as described previously (D. S. Harischandra et al., Sci Signal 12, (2019)). Tau aggregation was also examined by sedimentation assay. After centrifugation at 13,000 rpm at 4 °C for 30 min, the pellet fraction was analyzed by western blot to detect SDS-soluble (PE) amorphous aggregates, and by dot blot to detect relatively large SDS-resistant (SR) fibrillar aggregates (L. Guo et al., Mol Cell 55, 15-30 (2014); G. Zhu et al., Cell Rep 33, 108418 (2020); L. Huang et al., DAXX represents a new type of protein-folding enabler. Nature 597, 132-137 (2021)). For disaggregation, preformed tau filaments (1 μM) were incubated with or without the indicated concentrations of Flag-TRIM11 or GST-TRIM11 in a reaction solution (50 mM HEPES, pH 7.5, 50 mM KCl, 5 mM MgCl₂, and 1 mM DTT) at 37 °C for 24-48 h. The reaction mixtures were analyzed by ThT-binding and sedimentation assay as described above.

### Negative-Stain Electron Microscopy

A 5 μl volume of sample was applied to a thin carbon grid that was glow discharged for 2 min using a Pelco Easyglow instrument. A 5 μl of freshly made 2% Uranyl acetate stain solution was applied and incubated with the sample for 2 min on the grid. Excess sample and stain were blotted away with Whatman filter paper. The staining process is repeated and the grid was dried until imaged. TEM micrographs were collected using Tecnai T12 TEM microscope at 100 KeV. The images were recorded on Gatan Oneview 4K×4K camera. Each image was collected by exposing the sample for 4 sec and a total of 100 dose fractionated images were collected and into a single micrograph. The data was collected at −1.5 to 2 microns under focus at 30K-40K magnification.

### Prevention of PFFs Induced Aggregation of Endogenous Tau

Fibril transduction on HEK293T cells was performed as described (D. W. Sanders et al., Neuron 82, 1271-1288 (2014)). Briefly, tau PFFs were incubated with Lipofectamine-2000 in OptiMEM for 20 mins and then added to cells at the final concentration of 400 nM. 18 hours later, cells were washed and medium was exchanged with fresh medium. For fibril transduction of primary neurons as described before (J. L. Guo, V. M. Lee, FEBS Lett 587, 717-723 (2013)), cells were grown for four days and tested with AAVs or ASOs for three days. Tau PFFs were diluted in PBS and sonicated with 60 pulses, and added to neurons at 1.5 μg per well. Transduced neurons were harvested for immunocytochemistry at 2 weeks post-PFF transduction. For long-term ASO-mediated TRIM11 knockdown, ASO transduction was performed on neurons again at day 14.

rAAV9-TRIM11 and rAAV9-GFP Vector Production

Human TRIM11 (with a C-terminal HA tag) and GFP were cloned into the plasmid pENN.AAV9.CB7.CI.WPRE.rBG, in which their expression is driven by CB7, a chicken β-actin promoter with cytomegalovirus enhancer elements. TRIM11 and GFP plasmid DNAs were prepared by using endotoxin-free mega-prep kit (Qiagen) and characterized by structure and sequence analysis.

### AAV/ASO Transduction and Analysis of Cultured Primary Neurons

Primary neurons were allowed to grow for 4 days prior to AAV vector treatments, rAAV9-GFP was diluted to 5×10¹⁰ GC/ml and rAAV9-TRIM11 was diluted to 1.9×10¹¹ GC/ml in Neurobasal Medium for neuronal transduction. To knock down TRIM11 in neurons, ASOs were added into medium at a final concentration of 10 μM, and knockdown effects were analyzed by western blot after 3 days. For long-term TRIM11 knockdown in neurons, ASOs were added at 2 time points, and fluorescence-labeled oligos were used to monitor cellular uptake.

Hippocampal neurons and cortical neurons were washed with PBS and fixed for 10 min in PBS containing 4% paraformaldehyde and 4% sucrose at room temperature. After fixing, the cells were washed with PBS, permeabilized with 0.5% Triton X-100 for 5 min and blocked for 30 min with 10% NGS in PBS. Cells then were incubated with primary antibodies against phospho-tau (AT8 and MC1; 1:2000), GFP (1:2000), and HA (1:500), synaptophysin (1:500), PSD95 (1:300), NFL (1:500), and/or MAP2 (1:2000), overnight at 4 °C. After primary antibodies incubation, the cells were washed and incubated in the dark for 90 min with Alexa Fluor 488-, Alexa Fluor 555- or Alexa Fluor 405-conjugated secondary antibodies (Invitrogen, 1:1000). Hoechst 44432 (Invitrogen, 1:10000) was used as a nuclear stain and the coverslips were then mounted on glass slides and slides and then the fluorescence images were captured by a confocal laser scanning microscopy. AT8, MC1, and NFL signals were normalized based on cell numbers. PSD95 and synaptophysin signals were normalized based on dendrites length. MAP2 staining was used for dendrites length quantification. For this, single neurons were randomly selected, and total dendrites length of each neuron was traced and measured with Image J plugin Neuron J. For measuring cell viability, neurons treated with control or TRIM11 ASOs, or transduced with AAV9-GFP or AAV9-TRIM11 and treated with myc-K18/P301L PFFs, for 2 weeks were analyzed with Cell counting KIT-8 (Dojindo Laboratories, CK04) according to the manufacturer's instruction.

### Stereotaxic Injection

PS19 or 3×Tg-AD-AD mice of either sex were anesthetizing with a ketamine/xylazine mixture (100 mg/kg ketamine, 10 mg/kg xylazine, i.p.) and immobilized in a stereotaxic frame (Angle II, Leica Biosystems). A 10 μl Hamilton syringe was used for injection at predetermined coordinates. Mice were injected into hippocampus (bregma, AP-2.5 mm; ML, +2.0 mm; and DV, −1.8 mm) or lateral ventricle (bregma, AP-2.5 mm; ML, +2.0 mm; and DV, −1.8 mm) with the followings: (1) rAAV9-TRIM11 or rAAV9-GFP (5×10¹³ GC/ml, 5 μl), or (2) rAAV9-TRIM11 or rAAV9-GFP plus K18 tau PFFs (2 μg/μl, 2.5 μl). Specifically, PS19 mice at 10 weeks of age were injected with rAAV9-GFP or rAAV9-TRIM11 in the hippocampus, and PS19 mice at 4 weeks of age were injected with tau PFFs and rAAV9-GFP or rAAV9-TRIM11 in the hippocampus. 3×Tg-AD-AD mice at 12 months of age were injected with rAAV9-GFP or rAAV9-TRIM11 in the hippocampus, and 3×Tg-AD-AD mice at 9 months of age were injected with rAAV9-GFP or rAAV9-TRIM11 in the lateral ventricle. All animals were given analgesics (bupivacaine at 10 mg/kg) during surgery and monitored during and for 3 days after surgery.

### Western Blot Analysis of Mouse Brains

PS19 mice injected with tau PFFs and rAAV9-GFP or rAAV9-TRIM11 were euthanized at 12 weeks of age using carbon dioxide asphyxiation. Hippocampi of injected PS19 mice were carefully dissected and stored at −80 °C until use. The remaining mouse models followed the same procedures at the following time points: PS19 mice injected with rAAV9, 10 months of age; 3×Tg-AD-AD mice injected with rAAV9 in the hippocampus, 13 months of age; and 3×Tg-AD mice injected with rAAV9 in the lateral ventricle, 13.5 months of age.

Hippocampus were lysed in the lysis buffer (50 mM Tris, pH 8.8, 100 mM NaCl, 5 mM MgCl₂, 0.5% NP-40, 1 mM DTT, 250 IU/ml benzonase, 1 mM PMSF and 1× complete protease inhibitor cocktail) for 30 min on ice. Lysates were centrifuged for 15 min at 13,000 rpm and 4 °C. The NP-40-soluble supernatants were collected as SN fraction. The NP-40-insoluble pellets were resuspended in the pellet buffer (20 mM Tris, pH 8.0, 15 mM MgCl₂, 1 mM DTT, 250 IU/ml benzonase, 1 mM PMSF, and 1× complete protease inhibitor cocktail) for 30 min on ice after washed with PBS. Aggregated proteins in pellets were boiled in buffer containing 2% SDS. Clarified samples containing equal amounts of protein were separated on 10% SDS-polyacrylamide gels and analyzed by western blot.

### Immunohistochemistry Analysis of Mouse Brains

Mice were transcardially perfused with PBS and 4% paraformaldehyde (PFA) after which the brains were removed, postfixed in PFA for 48 h, thoroughly rinsed in PBS, and then embedded in paraffin blocks from which 7-μm-thick sections were cut for histological analysis. Slides were baked at 60 °C for 30 min followed by deparaffinization with xylene (2 washes×5 min) and rehydration through ethanol gradient (1 min 100% EtOH, 1 min 95% EtOH, 1 min 70% EtOH and 1 min 50% EtOH). Antigen retrieval was done as described previously (H. Kai et al., J Histochem Cytochem 60, 761-769 (2012)). Specifically, brain tissue sections were incubated in 10 mM EDTA (pH 3.0, pH 6.0, and pH 10.0) and 0.1 M sodium citrate (pH 3.0, pH 7.2, and pH 10.0), each for 5 min at 90 °C. The proteolytic digestion of tissue sections was performed at 37 °C for 30 min with 1.0 μg/ml of proteinase K and 100.0 μg/ml trypsin dissolved in 1.0 mM CaCl₂)/50 mM Tris buffer (pH 7.6), Sections were incubated in FA for 5 min at room temperature. After antigen retrieval, sections were washed with tap water for at least 5 min and then with PBS, and permeabilized with blocking buffer (10% goat serum, 2% BSA, 0.1% Triton X-100 and 0.05% Tween 20 in PBS) for 1 h at room temperature. Antibodies directed against AT8 (1:200, Thermo Fisher Scientific, MN1020) were incubated with the sections overnight at 4 °C and processed using DAB (3,3′-diaminobenzidine) staining as described previously (R. Gordon et al., Nat Commun 7, 12932 (2016)). Sections were counterstained using hematoxylin then dehydrated, cleared in xylene and mounted on slides using DPX mounting medium (Electron Microscopy Science). Samples were visualized and photomicrographs of sections were captured using an inverted fluorescence microscope (Revolve, Echo Laboratories).

AT8, GFAP, and Iba1 staining was quantified using ImageJ. The hippocampus area was outlined and isolated, and the colors were separated using “Color Deconvolution—H&E DAB”. The threshold was set automatically, and the intensity of staining was measured. For AT8 staining, the mean of the staining intensity for the GFP- or TRIM11-injected group (six mice each) was calculated, and is presented as relative intensity. For GFAP and Iba1 staining, the stained percent area of the hippocampus was calculated.

### Object Recognition Test

Mice were first habituated to the empty open field maze (referenced above) for 5 min. 24 hours later, mice were placed in the center of the chamber with 2 identical objects placed 5 cm away from the walls and allowed to explore these objects for 10 min. 12 hours later, mice were placed in the center of the chamber with the same 2 identical objects and allowed to explore for 10 min. 6 hours later, mice were placed in the center of the chamber with 1 familiar object and 1 novel object and allowed to explore for 10 min. The mice were recorded and tracked using a video-camera coupled to an automated tracking software (ANY-maze, SD Instruments). The discriminatory index was calculated by subtracting the time spent exploring the familiar object from the time spent exploring the novel object and dividing that difference by the sum of the time spent exploring the familiar object and the time spent exploring the novel object. The preference index was calculated by dividing the time spent exploring the novel object by the sum of the time exploring the novel object and time exploring the familiar object and multiplying this quotient by 100 to get a percentage (L. M. Lueptow, J Vis Exp, (2017)).

### Y-Maze Test

To measure hippocampus-dependent memory, mice were tested for spontaneous alternation behavior in a Y-Maze (ANY-maze, SD instruments). Mice were placed in the center of a 3-armed Y-maze and tracked for a period of 5 min. Spontaneous alternation behavior was scored as a proportion of alternations (entering an arm different than the previous two choices) to the total number of alternation opportunities based off the following formula: Spontaneous alternation %=No. of spontaneous alternations/(total numbers of arm entries-2)×100.

### Wire Hang Test

To measure grip strength, mice were placed on a mesh wire and allowed to acclimatize for 30 seconds before the mesh was flipped. The wire was suspended 15 cm above an empty clean cage and the latency to fall was measured. The maximum exploration time was 3 min. The average latency to fall in 3 tests performed over 1 day was analyzed.

### Open Field Test

The multiple unit open field maze (SD Instruments) consisting of four activity chambers with each chamber measuring 50 cm (length)×50 cm (width)×38 cm (height) made from white high density and non-porous plastic was used to analyze exploratory locomotor behavior. Mice were placed in the center of the chamber at the start of the test. After a 2 min accustomization period, mice were monitored for total distance traveled, total movement time, total freezing time, and time spent in each of the designated quadrants of the chamber for 10 min using a video-camera coupled to automated tracking software (ANY-maze, SD Instruments).

### Software

GraphPad Prism 7, ImageJ, ZEN lite: Carl Zeiss Microscopy.

### Statistical Analysis

Data are presented as mean±standard deviation (SD) or mean±standard error of mean (SEM). Unless otherwise stated, a two-tailed Student's t-test was used to evaluate the statistical significance in the mean value between two populations (*P<0.05; **P<0.01; ***P<0.001). Prism GraphPad 7 was used to create the figures and to complete the statistical analysis.

Each experiment was conducted at least three times or with at least three biological replicates.

The results of the experiments are now described.

### Effect of TRIM Proteins on Tau Aggregation

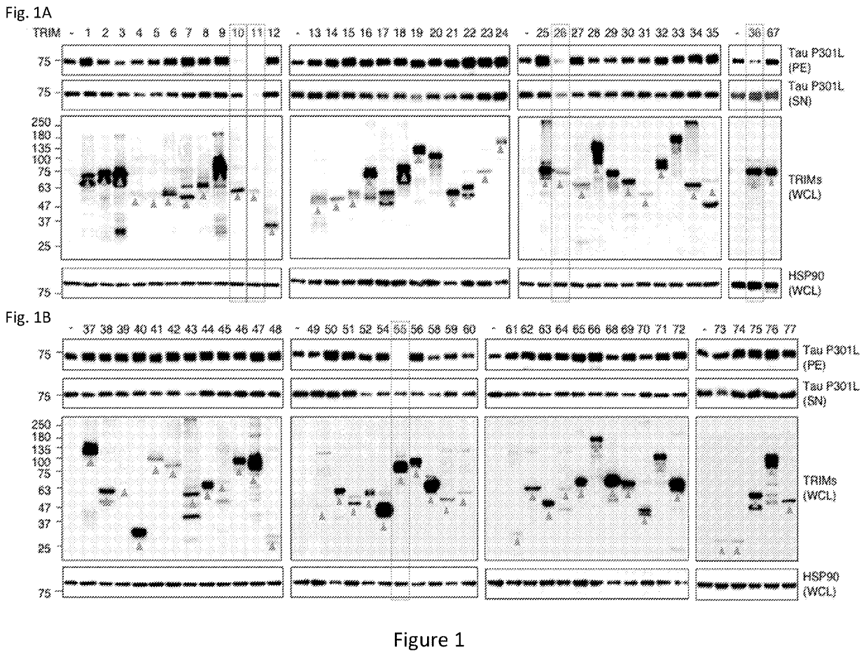

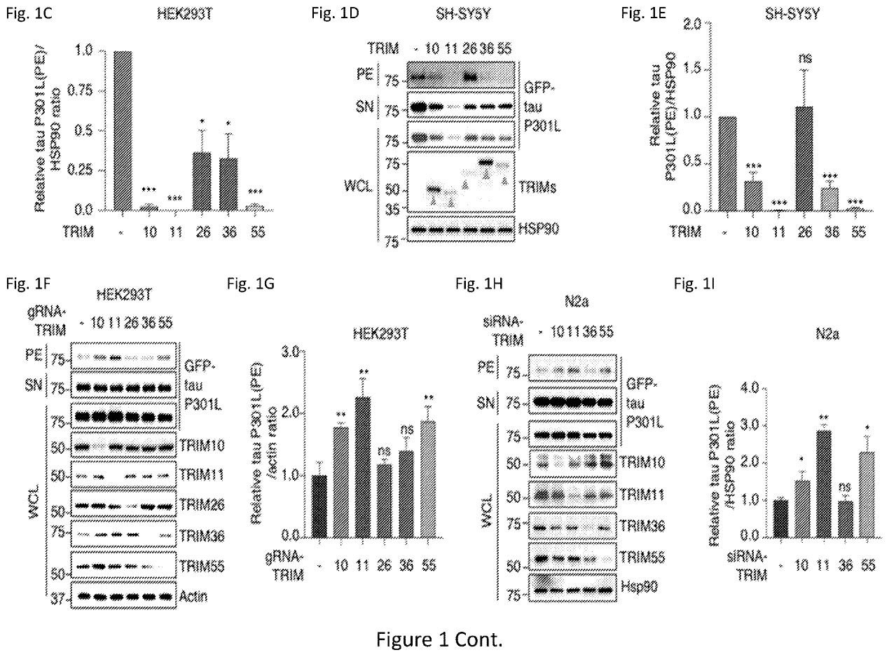

To determine the effect of TRIM proteins on tau aggregation, two approaches were combined: (1) systematically analyzing all known human TRIMs for their capability to remove tau aggregates in cultured cells, and (2) for TRIMs that exhibit a potent effect, comparing their expression in human postmortem tissues from AD and control individuals. For the systematic analysis, seventy-five TRIMs were cloned individually into a mammalian expression vector (Table 1). Each TRIM was introduced in HEK293T cells together with GFP-tau P301L, an enhanced green fluorescence protein fusion of the longest isoform of human tau (with 2 N-terminal inserts and 4 microtubule binding repeats, 2N4R) carrying P301L, a mutation associated with familial FTLD (M. Hutton et al., Nature 393, 702-705 (1998); C. Dumanchin et al., Hum Mol Genet 7, 1825-1829 (1998)). GFP-tau P301L alone generated aggregated species that were insoluble in non-ionic detergent (FIG. 1A to FIG. 1C). When co-expressed with GFP-tau P301L, the majority of TRIMs were unable to clear GFP-tau P301L aggregates. However, four TRIMs (TRIM10, −11, −36, and −55) displayed a robust effect, reducing GFP-tau P301L aggregates nearly completely, while another TRIM (TRIM26) displayed a moderate effect (FIG. 1A to FIG. 1C).

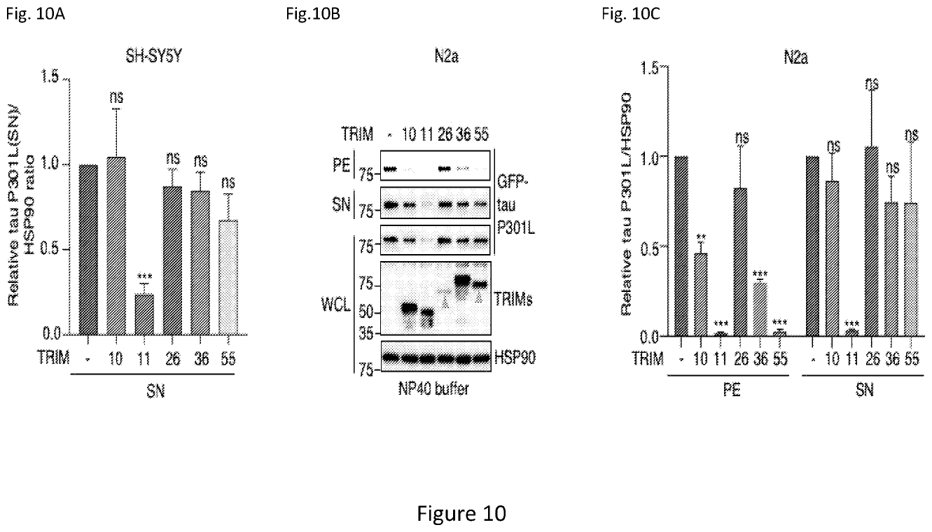

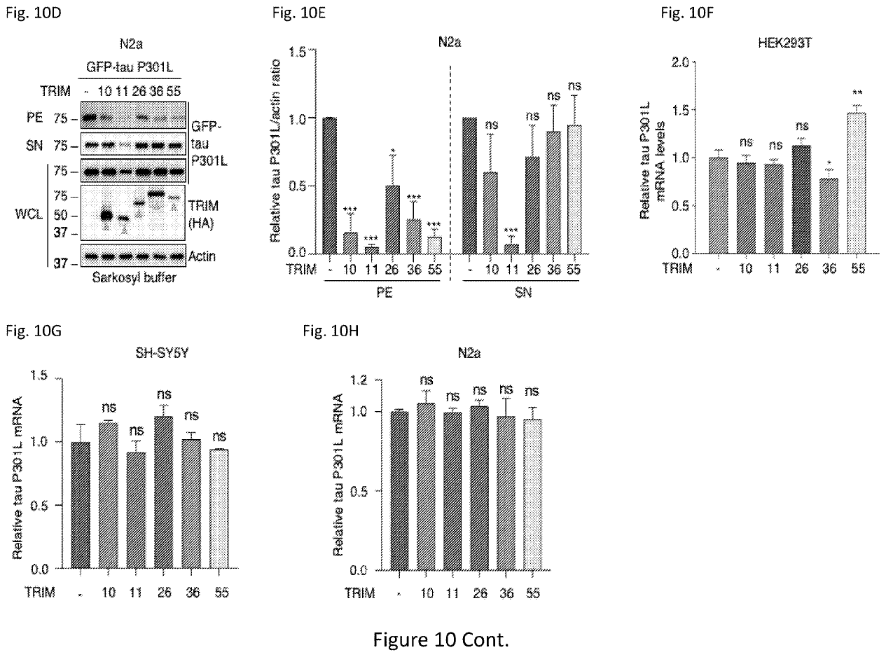

To further examine the effect of these five TRIMs on tau, two neural cell lines were used. TRIM10, −11, −36, and −55 strongly reduced GFP-tau P301L aggregates in SH-SY5Y (FIG. 1D, FIG. 1E, and FIG. 10A) and Neuro-2a (N2a) (FIG. 10B and FIG. 10C) cells. They also reduced GFP-tau P301L aggregates in N2a cells that were insoluble in the zwitterionic detergent sarkosyl (FIG. 10D and FIG. 10E), which likely contained filamentous tau (S. G. Greenberg, P. Davies, Proc Natl Acad Sci USA 87, 5827-5831 (1990)). In contrast, TRIM26 showed minimal or moderate activity (FIG. 1D, FIG. 1E, and FIG. 10A to FIG. 10E). With the exception of TRIM36 in HEK293T cells, none of these TRIMs reduced the levels of GFP-tau P301L mRNA in HEK293T, SH-SY5Y, or N2a cells (FIG. 10F to FIG. 10H).

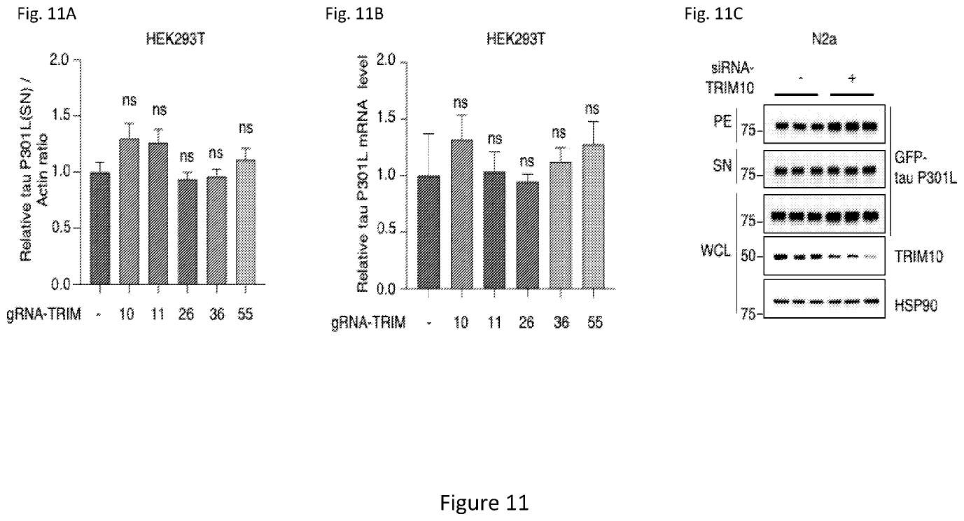

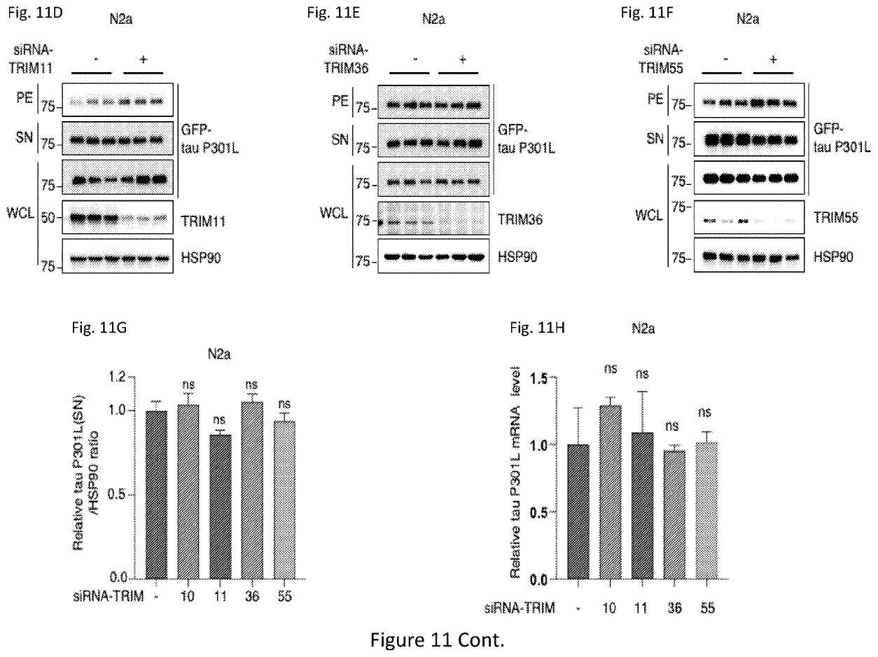

Conversely, each of these five TRIMs were knocked out in HEK293T cells by means of CRISPR-mediated gene editing. Knockout of TRIM10, −11, or −55 increased GFP-tau P301L aggregates by ~80-130% without altering GFP-tau P301L mRNA levels, whereas knockout of TRIM36, as well as TRIM26, had no effect on GFP-tau P301L aggregates (FIG. 1F, FIG. 1G, FIG. 11A, and FIG. 11B). These TRIMs were further knocked down in N2a cells by small interfering RNA (siRNA). Knockdown of TRIM10, −11, or −55 increased GFP-tau P301L aggregates by ~50-180% without affecting GFP-tau P301L mRNA, whereas knockdown of TRIM36 had no effect (FIG. 1H, FIG. 1I, and FIG. 11C to FIG. 11H). For overexpression, knockout, or knockdown, TRIM11 consistently displayed the strongest effect. Collectively, these results indicate that TRIM10, TRIM55, and especially TRIM11 possess potent activity to clear mutant tau protein.

### Downregulation of TRIM11 in AD Patients

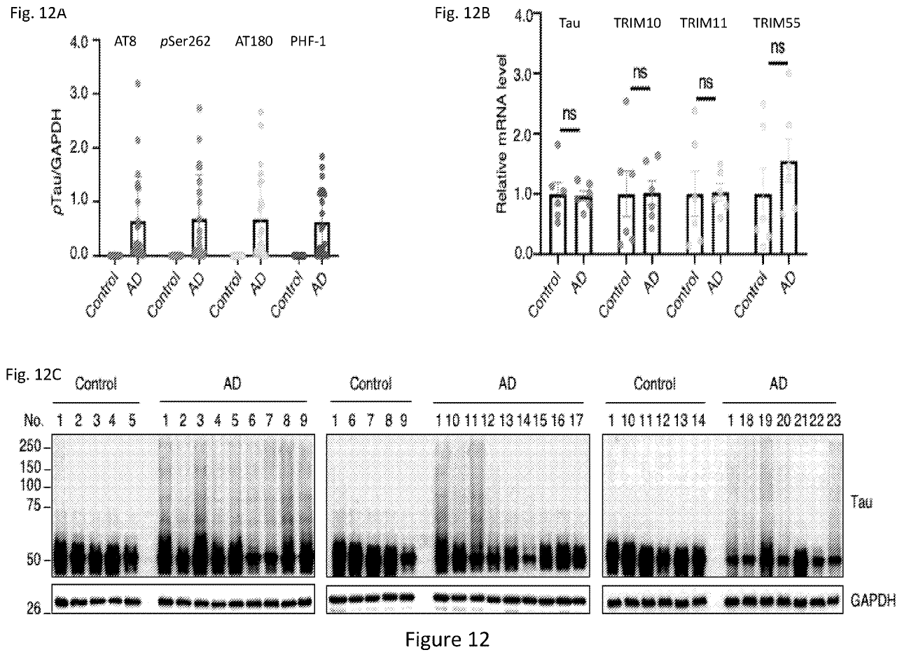

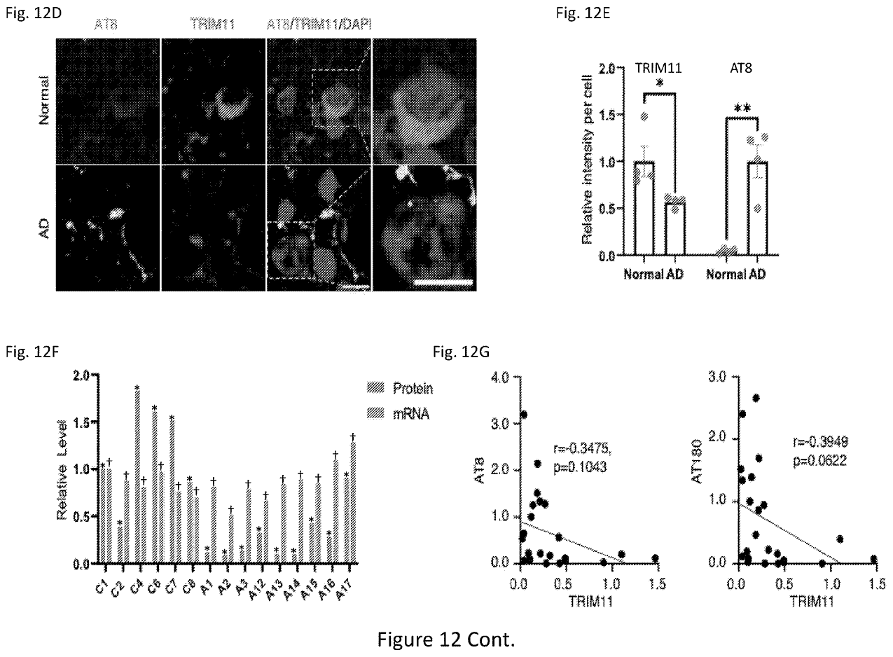

To evaluate whether TRIM10, −11, or −55 might be downregulated in AD patients, their RNA and protein levels were compared in postmortem brain tissues from twenty-three sporadic AD and fourteen age- and sex-matched control individuals with no known neurodegenerative diseases (FIG. 2A and Table 2). Tau pathology in the AD samples was verified by reactivity to a panel of antibodies that recognize abnormally phosphorylated tau (p-tau) species (FIG. 2B and FIG. 12A), as well as formation of high molecular weight tau species that were resistant to SDS (FIG. 12B). Each of the TRIM10, TRIM11, and TRIM55 transcripts was present at similar levels in AD and control tissues (FIG. 12C). Levels of TRIM10 or TRIM55 protein were also comparable in these tissues (FIG. 2B and FIG. 2C).

TABLE 2
Information on individual subjects

| | Age | | | | Brain |
|---|---|---|---|---|---|
| | (years) | PMI (h) | Sex | Braak stage | weight (g) |
| Ctr#1 | 67 | 24 | Female | I | 1245 |
| Ctr#2 | 83 | 3 | Female | I | 1026 |
| Ctr#3 | 68 | 17 | Male | II | 1371 |
| Ctr#4 | 81 | 19 | Male | II | 1343 |
| Ctr#5 | 83 | 6 | Male | II | 1183 |
| Ctr#6 | 66 | 11 | Male | I | 1426 |
| Ctr#7 | 62 | 16 | Male | I | 1321 |
| Ctr#8 | 73 | 17 | Male | II | 1330 |
| Ctr#9 | 56 | 12 | Female | I | 1416 |
| Ctr#10 | 89 | 19 | Female | I | 1138 |
| Ctr#11 | 61 | 20 | Female | I | 1341 |
| Ctr#12 | 68 | 15 | Female | I | 1151 |
| Ctr#13 | 62 | 8.5 | Male | I | 1420 |
| Ctr#14 | 59 | 17 | Male | I | 1218 |
| AD#1 | 66 | 19 | Female | VI | 931 |
| AD#2 | 79 | 17 | Male | VI | 1345 |
| AD#3 | 82 | 3 | Male | VI | 1035 |
| AD#4 | 70 | 5 | Male | VI | 1256 |
| AD#5 | 66 | 4 | Male | VI | 1124 |
| AD#6 | 89 | 18 | Female | VI | 1208 |
| AD#7 | 74 | 18 | Male | VI | 1211 |
| AD#8 | 61 | 18 | Male | VI | 1203 |
| AD#9 | 83 | 7 | Female | VI | 783 |
| AD#10 | 74 | 6 | Female | VI | 1149 |
| AD#11 | 65 | 21 | Female | VI | 1085 |
| AD#12 | 69 | 22 | Male | VI | 1182 |
| AD#13 | 89 | 18 | Male | VI | 1168 |
| AD#14 | 72 | 15 | Male | VI | 1442 |
| AD#15 | 82 | 39 | Male | VI | 1360 |
| AD#16 | 64 | 4 | Male | VI | 1353 |
| AD#17 | 77 | 17 | Male | V | 1265 |
| AD#18 | 62 | 12 | Male | VI | 1169 |
| AD#19 | 59 | 14 | Female | VI | 1048 |
| AD#20 | 75 | 17 | Male | VI | 862 |
| AD#21 | 66 | 4 | Male | VI | 1005 |
| AD#22 | 82 | 7 | Female | VI | 1113 |
| AD#23 | 55 | 20 | Male | VI | 1331 |

Interestingly, however, TRIM11 protein was present at a substantially lower level in AD compared to control tissues, with a ~55% reduction on average (FIG. 2B and FIG. 2D). This reduction was corroborated by immunohistochemistry (IHC) (FIG. 2E and FIG. 2F), and immunofluorescence (IF) controlled for numbers of neurons (FIG. 2G and FIG. 2H) or total neural cells (FIG. 12D and FIG. 12E). These observations, along with the comparable levels of the TRIM11 transcript in AD and control samples (FIG. 12C) and the unmatched TRIM11 mRNA and protein levels in individual AD samples (FIG. 12F), suggested that TRIM11 reduction in AD brains preceded neuronal loss.

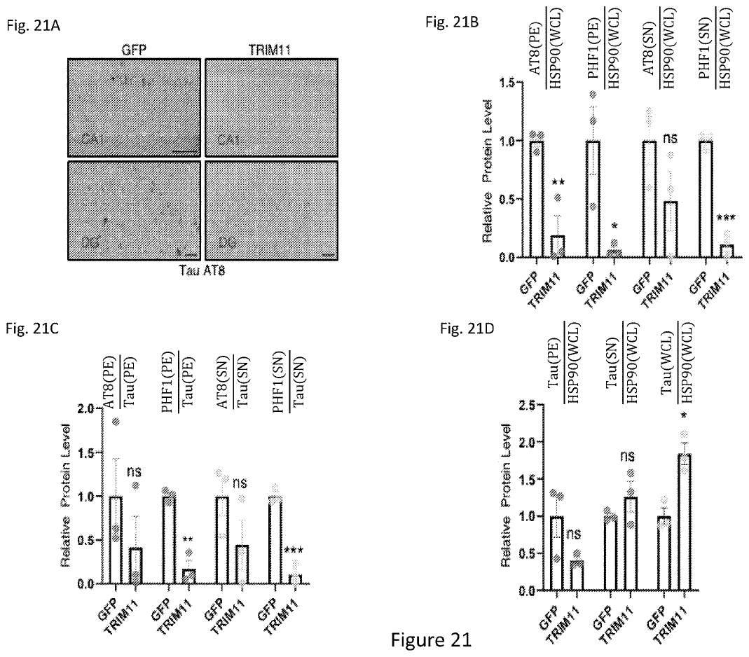

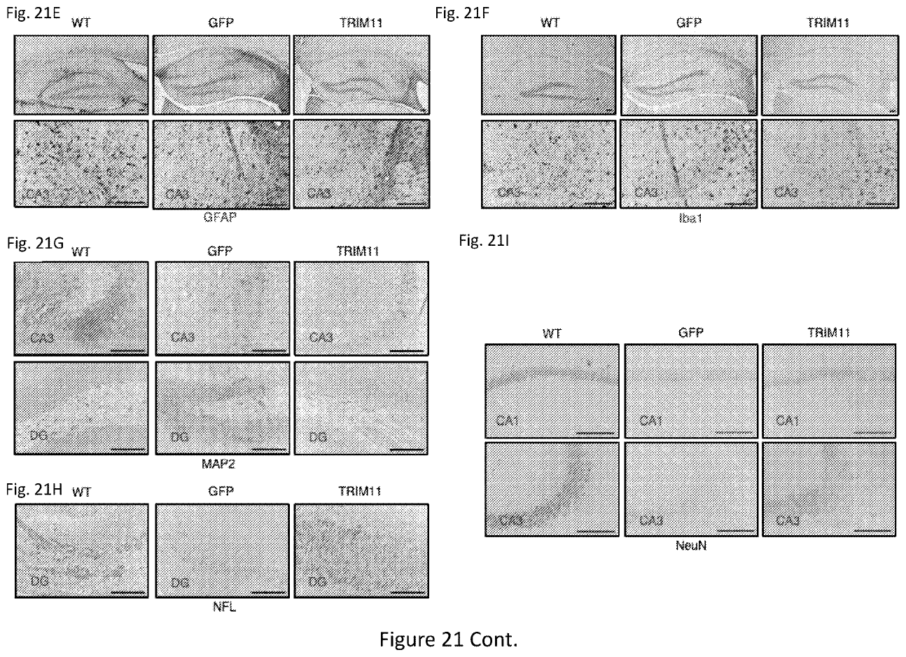

Across control and AD brain tissues, there was a strong inverse correlation between TRIM11 expression and levels of each of the p-tau species (FIG. 21). Among the AD tissues, there was also an inverse correlation between TRIM11 expression and levels of some p-tau species (FIG. 2J), albeit not the others (FIG. 12G). Together, these results indicate that TRIM11 is downregulated during sporadic AD pathogenesis with a strong association to tau pathology, and that the change in TRIM11 expression is probably due to a post-transcriptional mechanism. Of note, single nucleotide polymorphisms (SNPs) in the TRIM11 gene might be linked to rare cases of PSP, a prevalent form of sporadic tauopathy (E. Jabbari et al., Ann Neurol 84, 485-496 (2018)). Therefore, TRIM11 is the focus of the studies described below.

### TRIM11 Promotes Degradation of Tau

The strong ability of TRIM11 to clear tau aggregates, as well as its downregulation in sporadic AD brains, prompted investigation of its mechanism of action. First, it was evaluated whether TRIM11 targets tau for degradation, a possibility suggested by the screening of TRIMs (FIG. 1, FIG. 10, and FIG. 11). Tau is a substrate of the ubiquitin-proteasome system (A. S. Chesser, S. M. Pritchard, G. V. Johnson, Front Neurol 4, 122 (2013); M. J. Lee, J. H. Lee, D. C. Rubinsztein, Prog Neurobiol 105, 49-59 (2013)), whereas accumulation of insoluble tau impairs proteasome activity, further exacerbating tau pathology (N. Myeku et al., Nat Med 22, 46-53 (2016)). The commitment to proteasomal degradation occurs at the level of substrate recognition; yet, how tau is specifically recognized and marked for proteasomal degradation remains unknown.

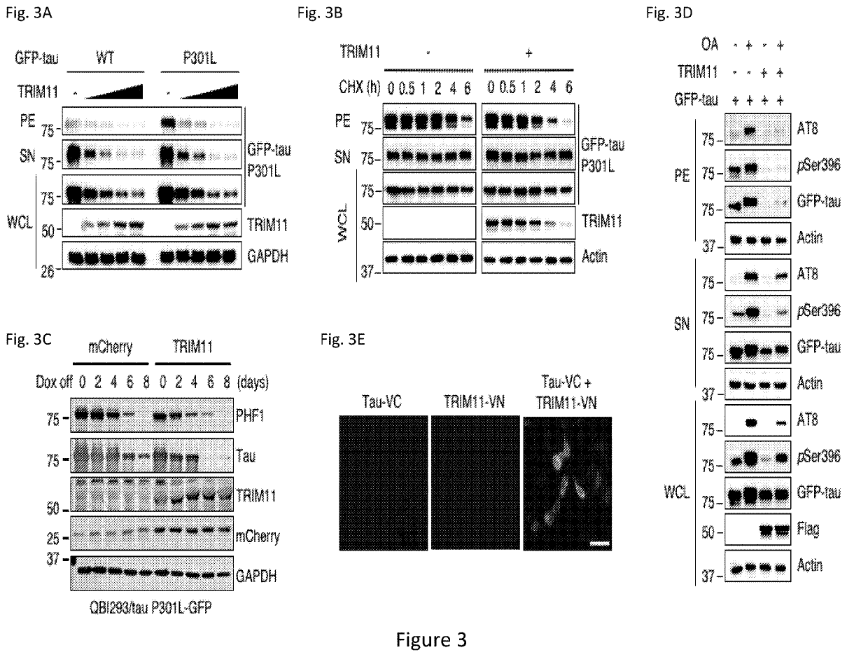

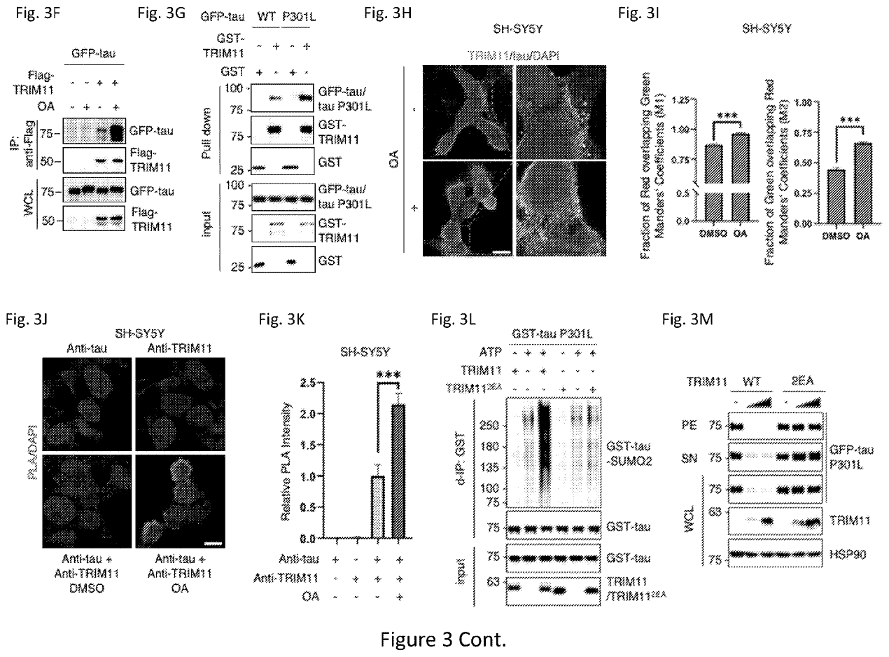

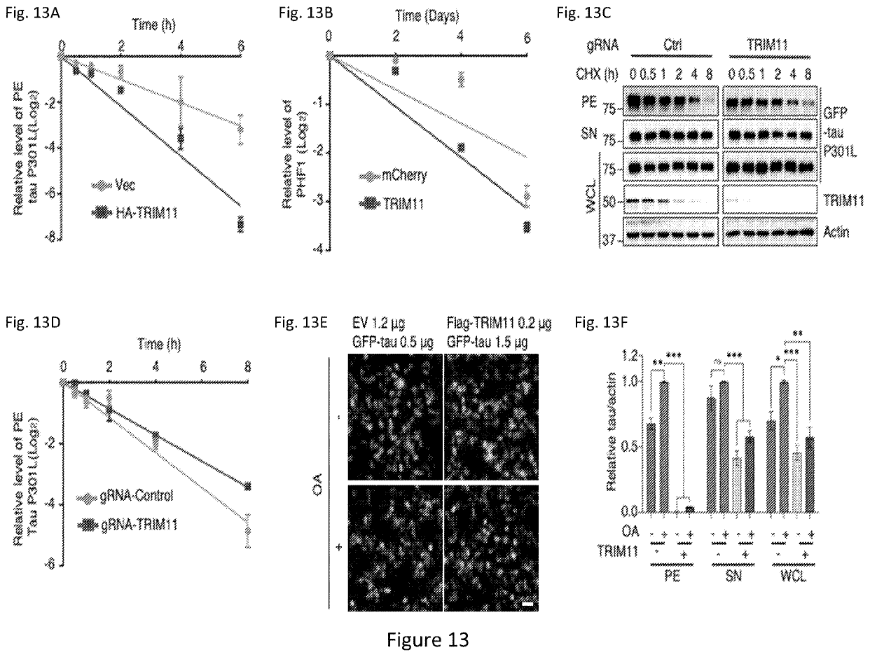

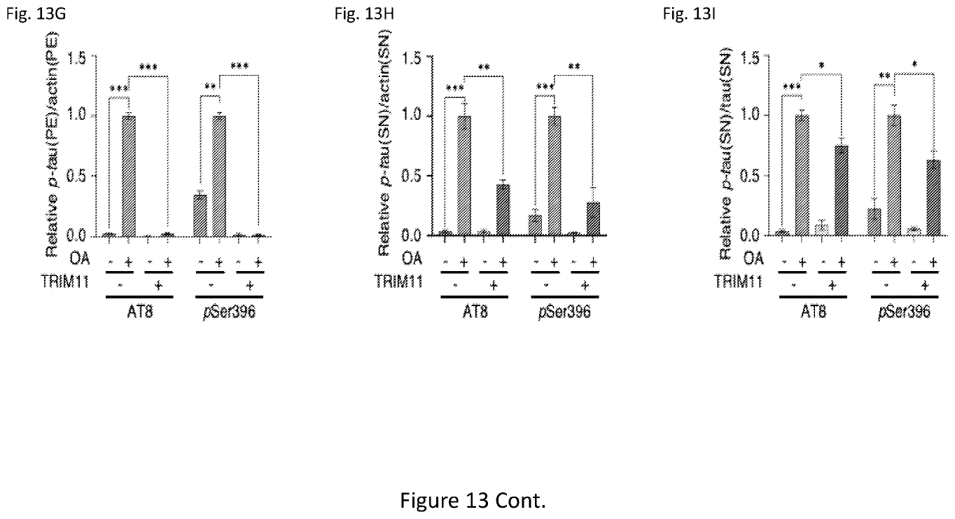

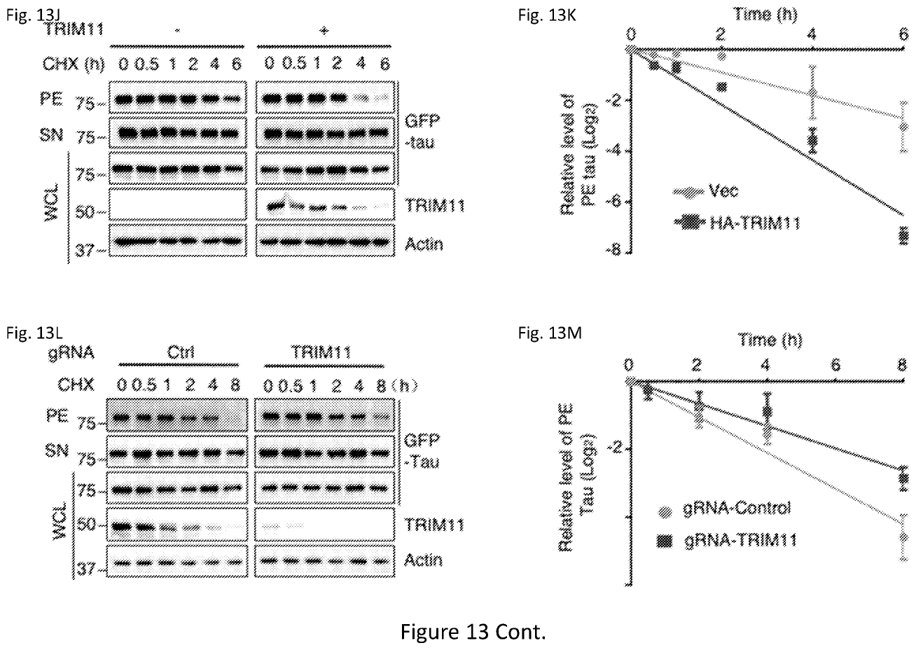

Upon co-expression in HEK293T cells, TRIM11 reduced levels of GFP-tau P301L in a dose-dependent manner (FIG. 3A). TRIM11 accelerated the turnover of aggregated GFP-tau P301L and prolonged its half-life, as shown by a cycloheximide (CHX) chase assay (FIG. 3B and FIG. 13A). To corroborate this observation, mCherry or mCherry plus TRIM11 was introduced into QBI293 cells that express tau P301L-GFP in a doxycycline (Dox)-inducible manner (QBI293/tau P301L-GFP) (J. L. Guo et al., J Biol Chem 291, 13175-13193 (2016)). Upon induction and then termination of tau P301L-GFP synthesis, the turnover of preexisting tau P301L-GFP and p-tau P301L-GFP was accelerated in TRIM11/mCherry-expressing cells compared to mCherry-expressing cells (FIG. 3C and FIG. 13B). Conversely, endogenous TRIM11 was knocked out and a slowdown in GFP-tau P301L degradation was observed (FIG. 13C and FIG. 13D).

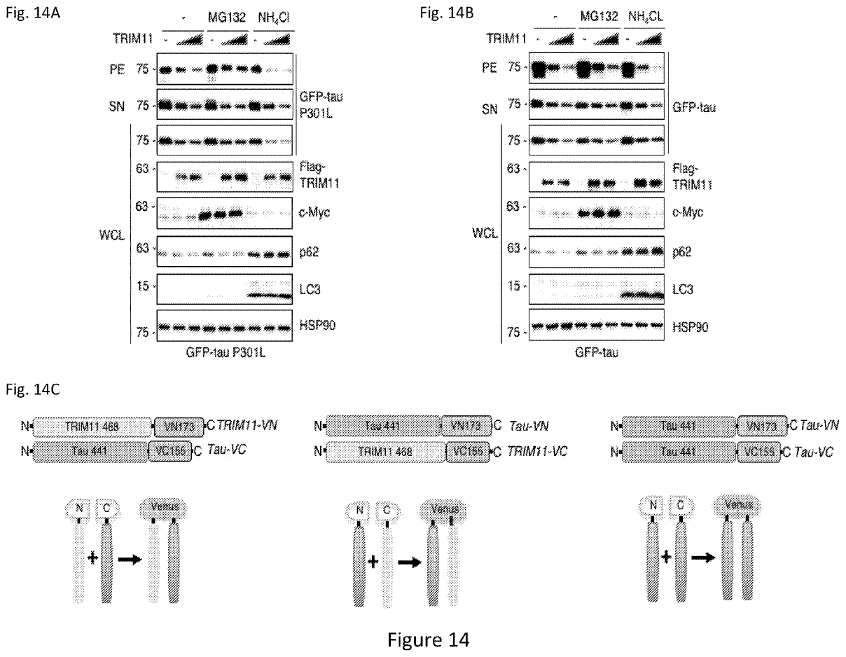

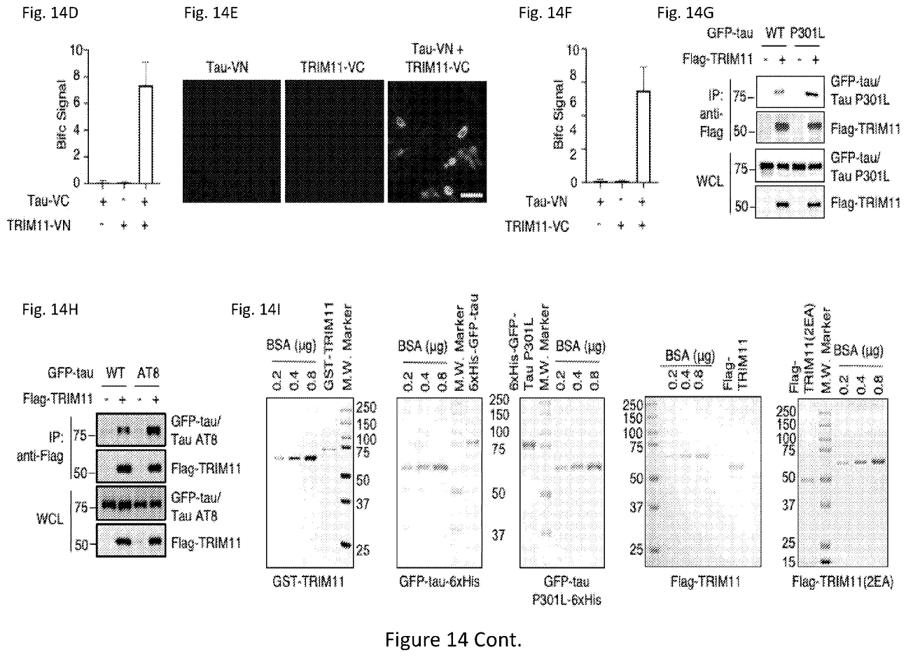

Treatment with the protein phosphatase inhibitor okadaic acid (OA) increased levels of p-tau species (C. X. Gong et al., J Biol Chem 275, 5535-5544 (2000)). (FIG. 3D and FIG. 13E). TRIM11 reduced levels of OA-induced p-tau species and nearly completely cleared them in the insoluble fraction (FIG. 3D and FIG. 13F through FIG. 13I). TRIM11 also decreased levels of overexpressed wild-type tau (FIG. 3A) and accelerated its degradation (FIG. 13J and FIG. 13K), whereas knocking out TRIM11 by CRISPR-mediated gene editing slowed tau degradation (FIG. 13L and FIG. 13M). TRIM11-mediated degradation of mutant and wild-type tau was prevented by the proteasome inhibitor MG132, but not the lysosome inhibitors leupeptin and ammonium chloride (NH₄Cl) in combination (LN) (FIG. 14A and FIG. 14B). Together, these results indicate that TRIM11 targets mutant and hyperphosphorylated tau, as well as superfluous normal tau, for proteasomal degradation.

### TRIM11 Binds to and SUMOylates Tau

To evaluate whether TRIM11 interacts with tau, a bimolecular fluorescence complementation (BiFC) assay that is based on the fluorescent protein Venus was performed (Shyu, Y. J., et al., 2006, Biotechniques, 40:61-66). TRIM11 and tau were fused to the N- and C-terminal fragments of Venus, respectively, generating TRIM11-VN and tau-VC (FIG. 14C, left panel). When TRIM11-VN and tau-VC were expressed together, but not individually, fluorescence was detected (FIG. 3E and FIG. 14D). TRIM11-VC and tau-VN (FIG. 14C, middle panel) were generated and again fluorescence was only observed when these fusions were expressed together (FIG. 14E and FIG. 14F). Thus, TRIM11 and tau interacted in cells, bringing the VN and VC moieties into close proximity to reconstitute Venus.

The interaction between exogenous TRIM11 and tau in cells was also detected by a co-immunoprecipitation assay (FIG. 3F and FIG. 14G). This interaction was stronger upon treatment with OA (FIG. 3F), or when wild-type tau was replaced with tau P301L (FIG. 14G) or tau AT8, a tau mutant (S199E/S202E/T205E) that mimics the AT8-reactive p-tau (FIG. 14H). In a cell-free assay with purified recombinant proteins (FIG. 14I), tau and especially tau P301L was pulled down by GST-TRIM11, but not GST (FIG. 3G), indicating that TRIM11 can directly bind to tau.

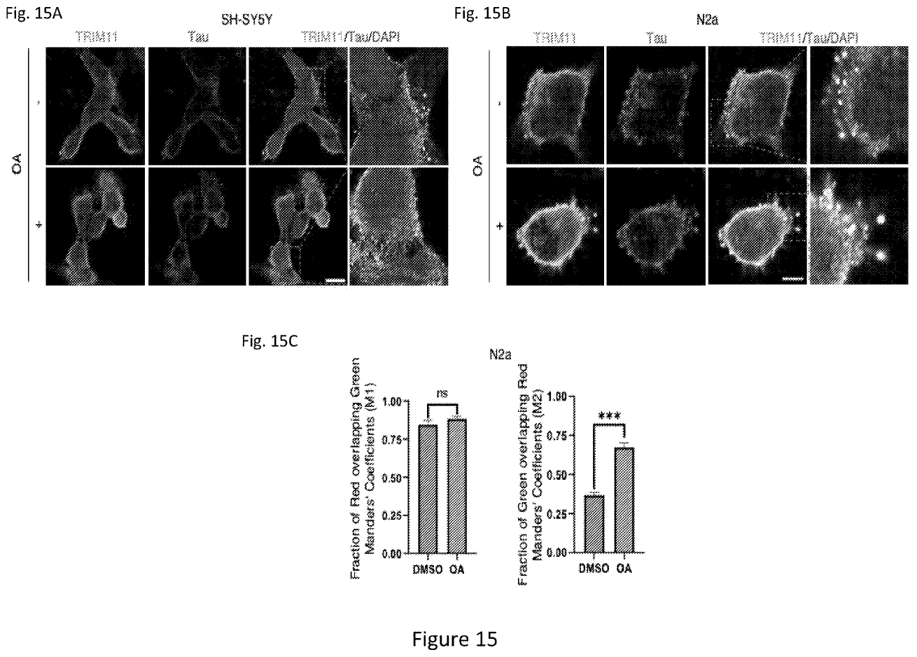

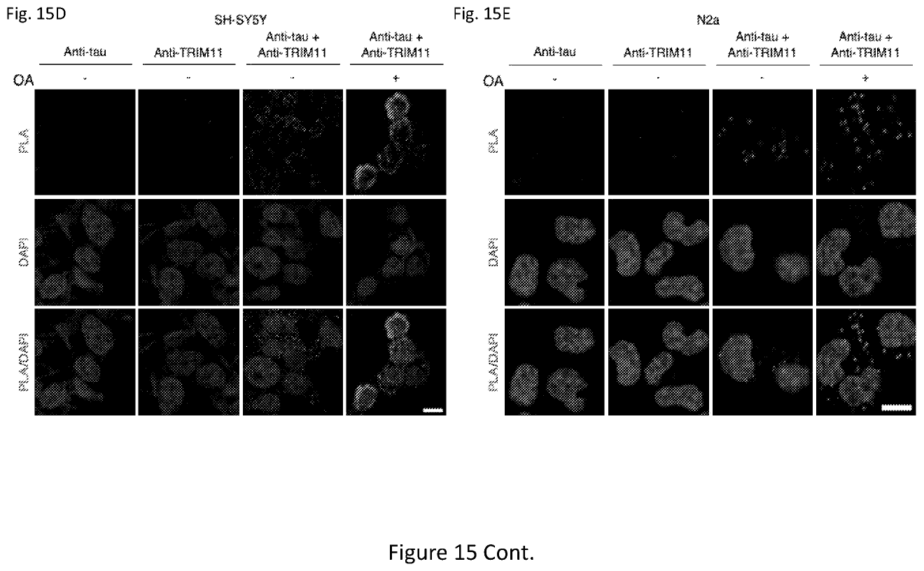

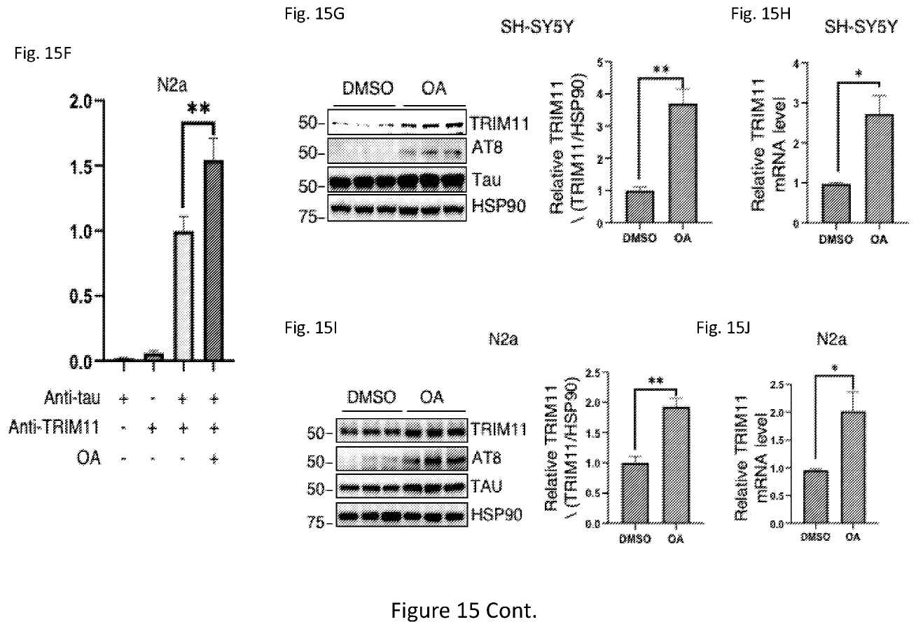

Endogenous TRIM11 and tau co-localized with each other in SH-SY5Y and N2a cells as shown by IF, and this co-localization was increased following OA treatment (FIG. 3H, FIG. 3I, and FIG. 15A through FIG. 15 C). Endogenous TRIM11 and tau also interacted with each other in SH-SY5Y and N2a cells as shown by a proximity ligation assay (PLA); this interaction was again increased upon OA treatment (FIG. 3J, FIG. 3KK, and FIG. 15D through FIG. 15F). Treatment with OA noticeably increased TRIM11 protein and mRNA levels in these cells (FIG. 15G through FIG. 15J), suggesting that TRIM11 expression is highly responsive to tau phosphorylation and aggregation. Collectively, these results indicate that TRIM11 interacts with tau, preferentially the mutant or hyper-phosphorylated tau species.

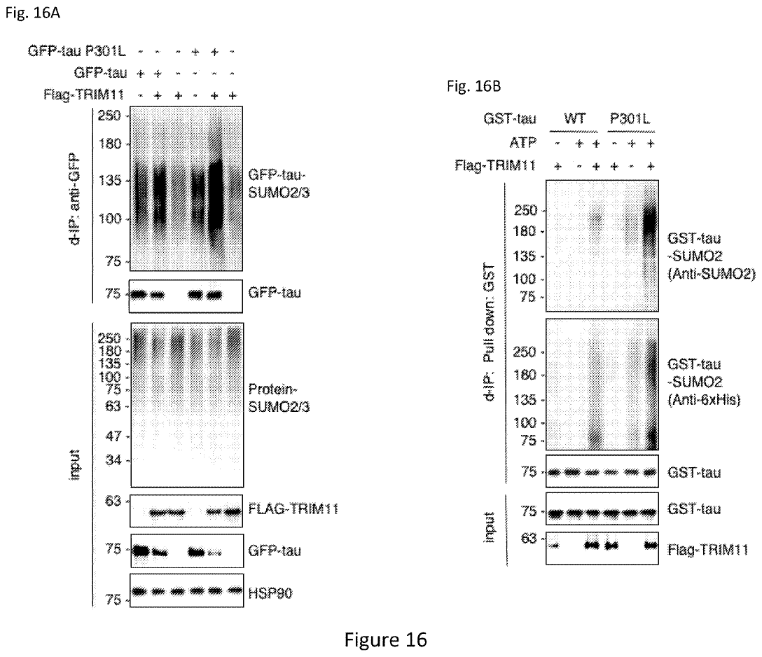

TRIM proteins possess SUMO E3 ligase activity (L. Guo et al., Mol Cell 55, 15-30 (2014); G. Zhu et al., Cell Rep 33, 108418 (2020); Y. Chu, X. Yang, Oncogene 30, 1108-1116 (2011)). For the nucleus-localized TRIM19 (a.k.a. PML), this activity promotes conjugation of nuclear misfolded proteins to poly-SUMO2/3 chains, permitting ubiquitination of these proteins by SUMO-targeted ubiquitin ligases (STUbLs) and their subsequent degradation in the proteasome (L. Guo et al., Mol Cell 55, 15-30 (2014)). TRIM11, like tau, is primarily a cytoplasmic protein (L. Chen, G. Zhu, E. M. Johns, X. Yang, Nat Commun 9, 1223 (2018); G. Zhu et al., Cell Rep 33, 108418 (2020)). TRIM11 increased SUMOylation of tau in cells, and this activity was more pronounced for tau P301L than for tau (FIG. 16A). In a cell-free assay with purified recombinant proteins (FIG. 14I), TRIM11 directly SUMOylated tau and especially the mutant form (FIG. 3L and FIG. 16B). By contrast, TRIM112FA, in which the two conserved Glu residues proximal to the RING domain were replaced with Ala (G. Zhu et al., Cell Rep 33, 108418 (2020)), exhibited no such activity (FIG. 3L). TRIM112EA also failed to promote tau degradation (FIG. 3M). Collectively, these results indicate that TRIM11 SUMOylates tau, promoting its degradation in the proteasome.

### TRIM11 Enhances Tau Solubility

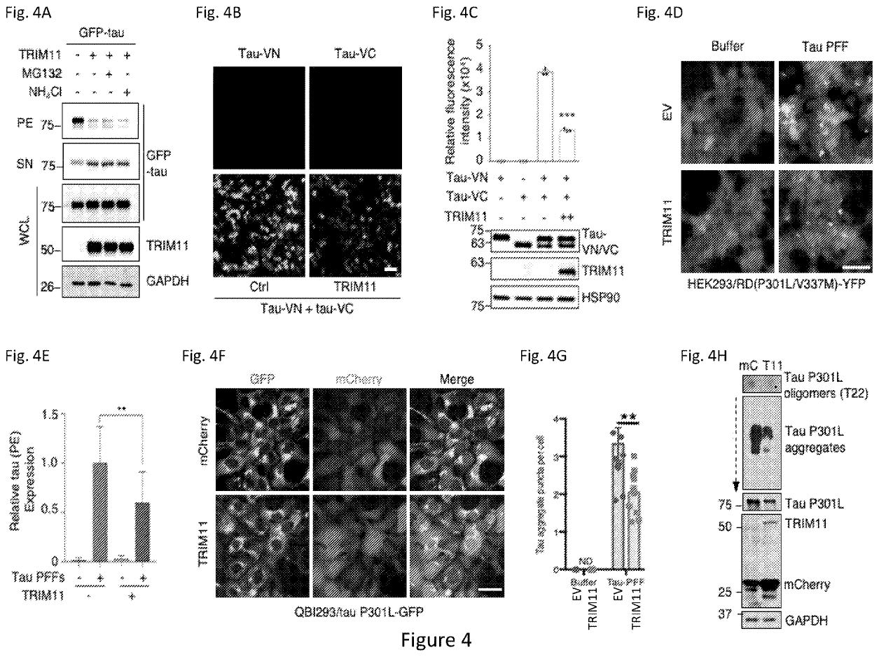

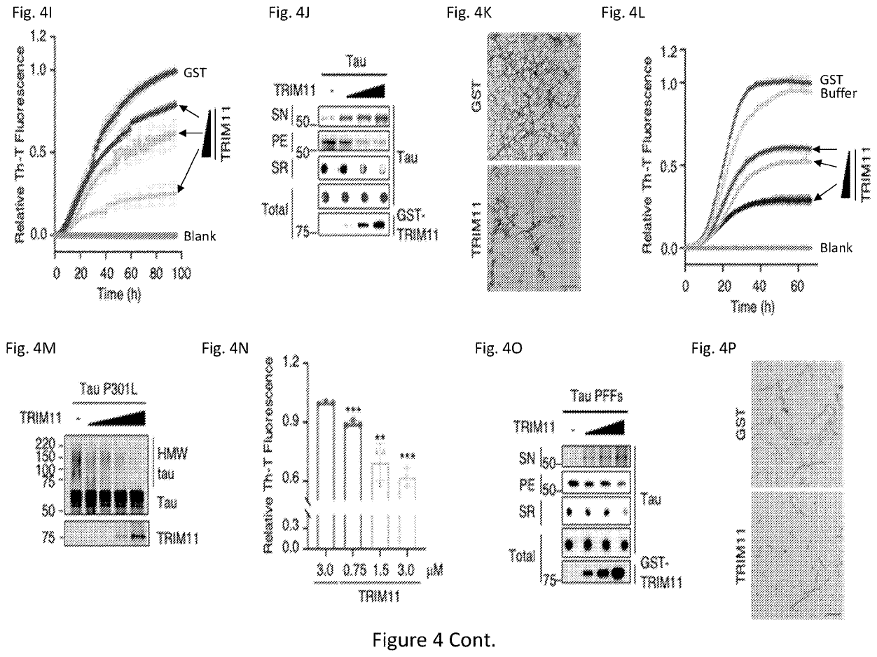

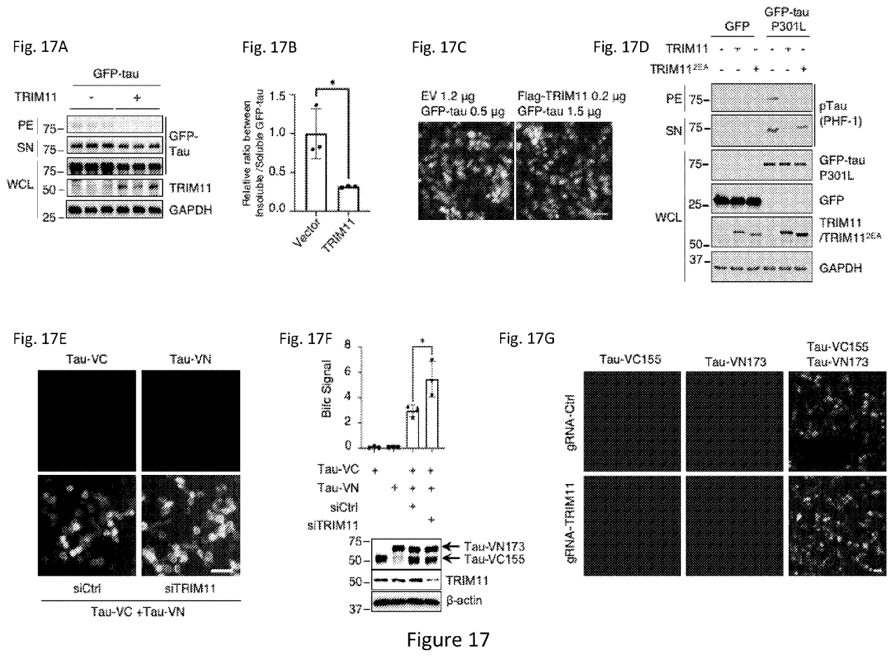

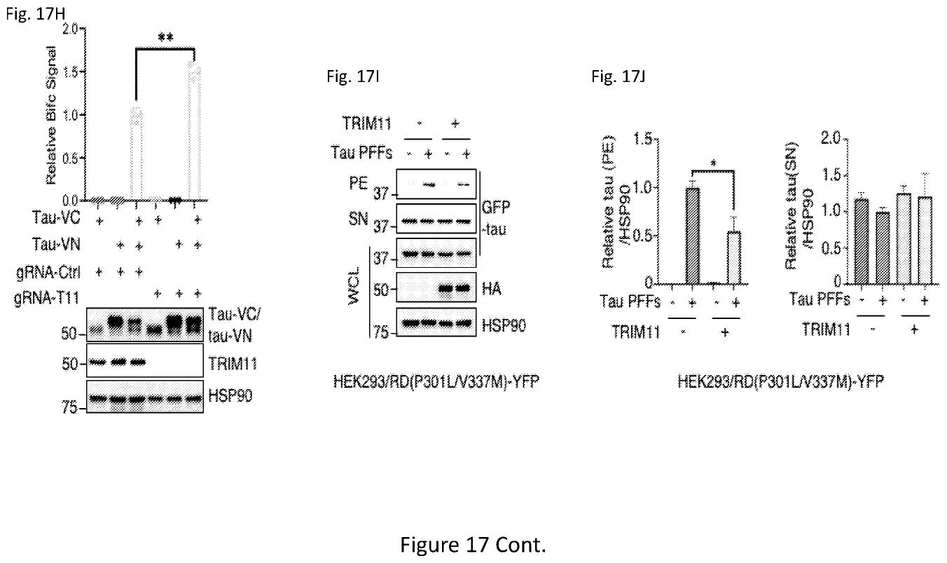

While defective proteins and superfluous normal proteins are removed by degradative systems, the vast majority of tauopathy cases are sporadic in which fibrillization occurs on normal tau protein expressed at physiological levels (C. L. Masters et al., Nat Rev Dis Primers 1, 15056 (2015)). To maintain protein solubility, cells employ molecular chaperones and disaggregases, which prevent and reverse protein aggregation, respectively (J. Tyedmers, A. Mogk, B. Bukau, Nat Rev Mol Cell Biol 11, 777-788 (2010); F. U. Hartl, A. Bracher, M. Hayer-Hartl, Nature 475, 324-332 (2011); H. Saibil, Nat Rev Mol Cell Biol 14, 630-642 (2013)). TRIM11 preferentially removed insoluble tau, hence increasing the fraction of tau molecules that was soluble (e.g., see FIG. 17A and FIG. 17B). Even under conditions when the total amounts of tau protein were comparable, TRIM11 increased tau solubility (FIG. 3D, FIG. 4A, and FIG. 17C). TRIM11-mediated tau solubilization was still evident when cells were treated with MG132 or NH₄Cl (FIG. 4A), indicating its independence of tau degradation. Moreover, under conditions where the total GFP-tau P301L levels remained comparable, TRIM11 dramatically reduced the amount of abnormally phosphorylated GFP-tau P301L species in both soluble and insoluble fractions (FIG. 17D).

Aggregation of tau occurs through a rate-limiting nucleation step that involves its self-association (P. Friedhoff, M. von Bergen, E. M. Mandelkow, P. Davies, E. Mandelkow, Proc Natl Acad Sci USA 95, 15712-15717 (1998)). To assess the effect of TRIM11 on tau self-association in cells, a BiFC assay was performed, in which tau was fused to VN or VC (H. Tak et al., PLOS One 8, e81682 (2013)) (FIG. 14C, right panel). When tau-VN and tau-VC were expressed together, but not individually, fluorescence signal was produced, indicating tau self-association (FIG. 4B and FIG. 4C). When present at low levels that did not alter tau abundance, TRIM11 noticeably reduced the fluorescence signal (FIG. 4B and FIG. 4C). Conversely, knocking down TRIM11 increased fluorescence signal in cells expressing tau-VN and tau-VC (FIG. 17E and FIG. 17F). Knockout of TRIM11 by CRISPR-mediated genome editing also led to a higher fluorescence signal in cells expressing tau-VN and tau-VC (FIG. 17G and FIG. 17H). These results indicate that TRIM11 enhances tau solubility and prevents its self-association.

### TRIM11 Inhibits Seeding of Tau Aggregates

Tauopathies are characterized by focal formation of tau aggregates and their spread along interconnected neuronal regions (M. Jucker, L. C. Walker, Nature 501, 45-51 (2013); J. Brettschneider, K. Del Tredici, V. M. Lee, J. Q. Trojanowski, Nat Rev Neurosci 16, 109-120 (2015); B. Frost, M. I. Diamond, Nat Rev Neurosci 11, 155-159 (2010)). Reflecting this prion-like, self-templating propagation, preformed fibrils (PFFs) of tau can seed the aggregation of endogenous soluble tau in cells (B. Frost, R. L. Jacks, M. I. Diamond., J Biol Chem 284, 12845-12852 (2009); J. L. Guo, V. M. Lee, J Biol Chem 286, 15317-15331 (2011); M. Iba et al., J Neurosci 33, 1024-1037 (2013); D. W. Sanders et al., Neuron 82, 1271-1288 (2014)). To examine whether TRIM11 protects against PFFs-seeded tau fibrillization, HEK293 that stably express RD (LM)-YFP were used, in which the tau repeat domain (RD) harboring the pro-aggregation mutations P301L and V337M is fused to yellow fluorescence protein (YFP) (D. W. Sanders et al., Neuron 82, 1271-1288 (2014). Treatment of the HEK293/RD (LM)-YFP cells with PFFs generated from recombinant tau protein induced aggregation of intracellular RD (LM)-YFP, as shown by the appearance of inclusions in cells (FIG. 4D and FIG. 4E) and an increase in insoluble species in cell lysates (FIG. 17I and FIG. 17J). Forced expression of TRIM11 reduced PFFs-induced RD (LM)-YFP inclusions and aggregates, while having a minimal effect on levels of soluble RD (LM)-YFP (FIG. 4E, FIG. 4F, FIG. 17I, and FIG. 17J).

In an alternative approach, QBI293/tau P301L-GFP cells were treated with PFFs, which seeded aggregation of intracellular tau P301L-GFP (FIG. 4F through FIG. 4H). When introduced into these cells, TRIM11 strongly decreased both tau inclusions in cells (FIG. 4F and FIG. 4G) and tau aggregates in cell lysates (FIG. 4H). Preceding the formation of fibrillar aggregates, tau, similar to other misfolding-prone proteins linked to neurodegeneration (R. Kayed et al., Science 300, 486-489 (2003)), assembles into soluble oligomeric species, which could be neurotoxic (C. A. Lasagna-Reeves et al., Mol Neurodegener 6, 39 (2011)). Forced expression of TRIM11 also reduced formation of tau oligomers, as assayed with the tau oligomer-specific antibody T22 (C. A. Lasagna-Reeves et al., FASEB J 26, 1946-1959 (2012)) (FIG. 4H). Therefore, TRIM11 protects against PFFs-seeded aggregation of intracellular tau into soluble oligomers and insoluble fibrils.

### TRIM11 is a Molecular Chaperone for Tau

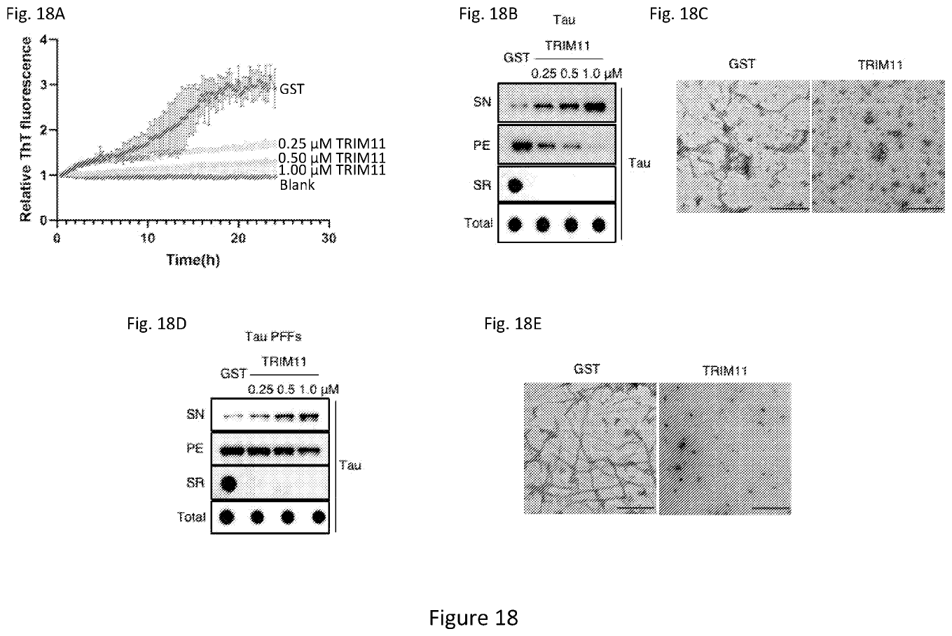

The ability of TRIM11 to maintain tau solubility even in the face of PFFs prompted us to investigate whether TRIM11 can function as a molecular chaperone, a disaggregase, or both for tau. In the presence of heparin, recombinant tau protein spontaneously produced amyloid fibrils (M. Goedert et al., Nature 383, 550-553 (1996)). This was indicated by a thioflavin T (ThT)-binding assay (FIG. 4I); western and dot blots that detected pelletable SDS-soluble (PE) and SDS-resistant (SR) aggregates, respectively (FIG. 4J); and electron microscopy (EM) that visualized mature tau fibrils (FIG. 4K). GST-TRIM11, but not GST, purified from bacterium (FIG. 14I) effectively prevented tau fibrillization, reducing it by ~30% at a 1:80 molar ratio to tau and by ~60% at a 1:20 molar ratio (FIG. 4I and FIG. 4J). EM analysis confirmed that TRIM11 blocked the formation of mature tau fibrils (FIG. 4K). Moreover, TRIM11 prevented aggregation of tau P301L into amyloid fibrils and other high molecular weight species (FIG. 4L and FIG. 4M). TRIM11 also effectively blocked fibrillization GST-tau (FIG. 18A through FIG. 18C). At a molar ratio of 1:20, TRIM11 almost completely blocked the formation of GST-tau fibrils (FIG. 18A). These observations indicate that TRIM11 is a potent molecular chaperone for tau, preventing its misfolding and aggregation. Distinctive from canonical chaperones such as the HSP70 and HSP90 systems, which are multicomponent machineries driven by energy derived from ATP hydrolysis (D. Balchin, M. Hayer-Hartl, F. U. Hartl Science 353, aac4354 (2016)), TRIM11 obviates tau aggregation at low sub-stoichiometric ratios by itself, in the absence of any other protein components or ATP (FIG. 4I through FIG. 4M and FIG. 18A through FIG. 18C).

### TRIM11 is a Disaggregase for Tau

Amyloid fibrils, which comprise of β strands that stack perpendicularly to the fibril axis (cross-β structure), are highly organized, energetically stable structures (D. Eisenberg, M. Jucker, Cell 148, 1188-1203 (2012); T. P. Knowles, M. Vendruscolo, C. M. Dobson, Nat Rev Mol Cell Biol 15, 384-396 (2014)). Their dissolution usually requires coordinated action of multiple components in an ATP-dependent manner (H. Saibil, Nat Rev Mol Cell Biol 14, 630-642 (2013); S. M. Doyle, O. Genest, S. Wickner, Nat Rev Mol Cell Biol 14, 617-629 (2013)). Bacterial, fungus, and plant cells have a disaggregation system comprised of HSP70, its co-chaperone HSP40, and the AAA⁺-ATPase HSP104 (J. R. Glover, S. Lindquist, Cell 94, 73-82 (1998)), but this system is absent in metazoans. Recent studies showed that mammalian HSP70/HSP40 and HSP110 (an atypical HSP70 family) can work together as a disaggregase (J. Shorter, PLOS One 6, e26319 (2011); X. Gao et al., Mol Cell 59, 781-793 (2015); N. B. Nillegoda et al., Nature 524, 247-251 (2015)). Yet, this disaggregation system fragments amyloid fibrils, generating small seeds that promote, rather than suppress, aggregation of tau and other misfolding-prone proteins, hence exacerbating their neurotoxicity (J. Tittelmeier et al., EMBO J 39, e103954 (2020); E. Nachman et al., J Biol Chem 295, 9676-9690 (2020)).

When incubated with pre-existing tau fibrils, TRIM11 was capable of dissolving these aggregates, reducing their bindings to ThT (FIG. 4N) and converting the majority of them to a soluble state (FIG. 4O). This activity was verified by EM analysis (FIG. 4P). Similarly, TRIM11 strongly dissolved GST-tau fibrils (FIG. 18D and FIG. 18E). Thus, TRIM11 is also a potent disaggregase for tau. As for its molecular chaperone activity, TRIM11 dissolved pre-existing tau fibrils in the absence of any other protein components or ATP. Importantly, unlike the mammalian HSP70/HSP40-HSP110 system, TRIM11 effectively abrogates the formation and seeding of tau aggregates in cells (FIG. 4A through FIG. 4H and FIG. 17).

### TRIM11 Protects Primary Neuron Against Tau Aggregation

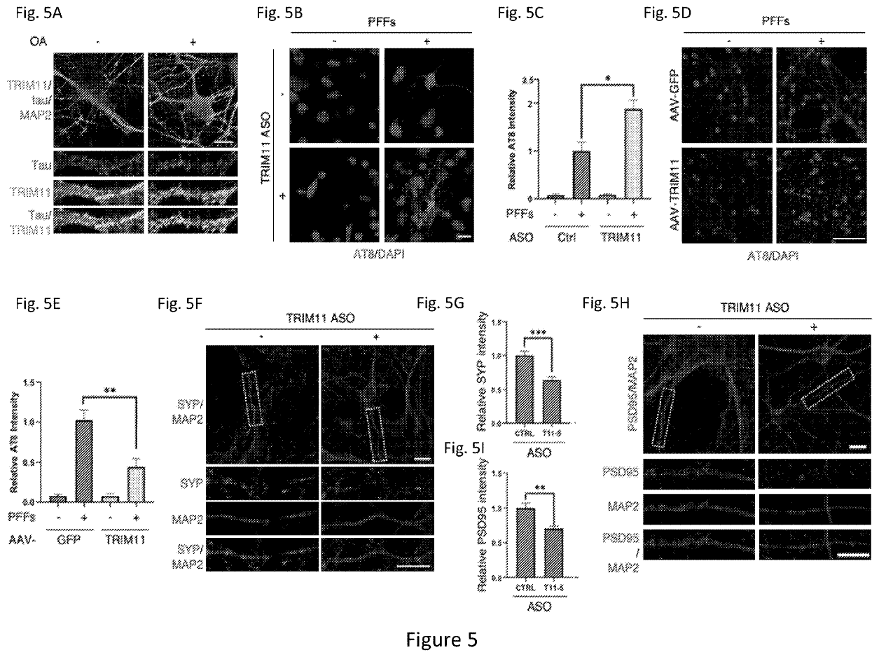

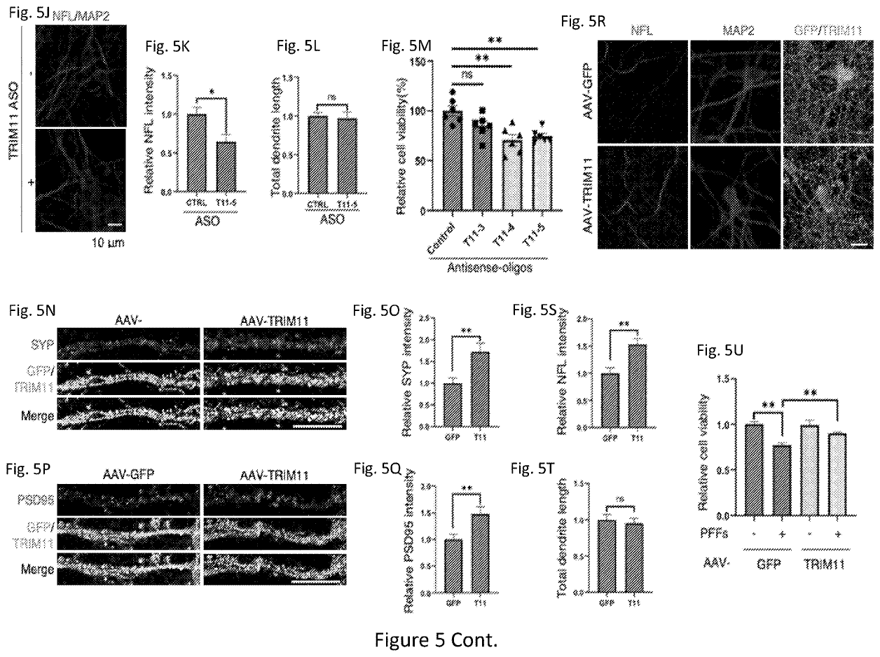

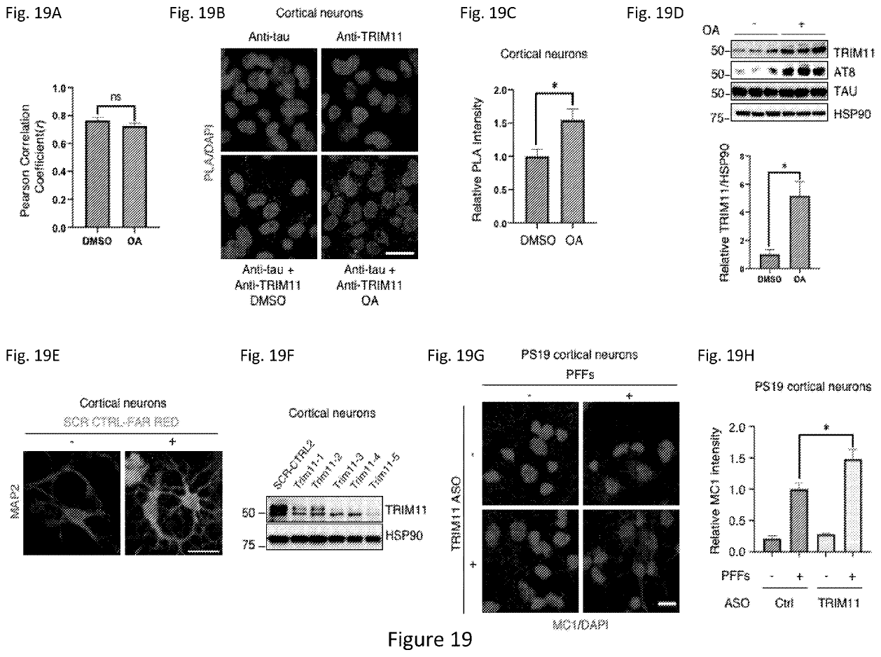

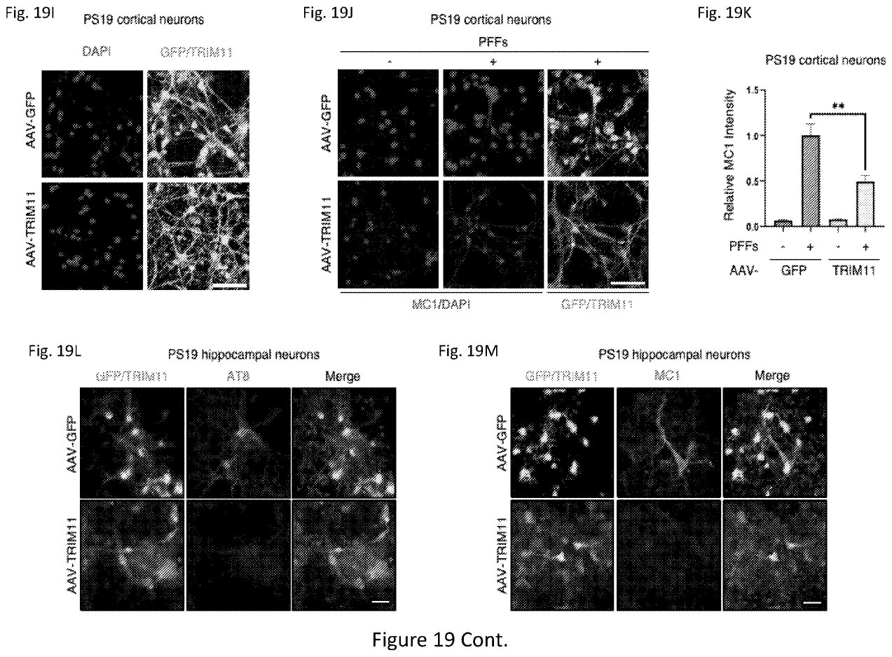

In primary neurons derived from wild-type mice, as in neural cell lines, endogenous TRIM11 and tau co-localized with each other (FIG. 5A and FIG. 19A). Endogenous TRIM11 and tau also interacted in primary neurons, as shown by a PLA assay (FIG. 19B and FIG. 19C). Treatment with OA, which elevated TRIM11 levels (FIG. 19D), augmented the TRIM11-tau interaction (FIG. 5A, FIG. 19B and FIG. 19C).

To assess the effect of TRIM11 downregulation on tau aggregation, several antisense oligonucleotides (ASOs) were generated, comprised of 2′-deoxy-2′fluoro-D-arabinonucleic acid (FANA; A. Kalota et al., Nucleic Acids Res 34, 451-461 (2006)) against Trim11. These FANA-ASOs effectively entered cultured neurons (FIG. 19E) and silenced TRIM11 expression to different extents (FIG. 19F). Cortical neurons derived from PS19 transgenic mice that express human tau P301S (Y. Yoshiyama et al., Neuron 53, 337-351 (2007)), were treated with PFFs formed by myc-K18/P301L, a truncated tau protein consisting of the microtubule-binding domain with the P301L mutation (M. Iba et al., J Neurosci 33, 1024-1037 (2013)). This led to the formation of neuritic thread-like inclusions reactive to AT8 (FIG. 5B and FIG. 5C), as well as to MC1, which recognizes a disease-specific conformation of tau (FIG. 19G and FIG. 19H). Silencing Trim11 by FANA-ASO exacerbated tau aggregation, increasing AT8- and MC1-reactive tau inclusions by ~50-90% (FIG. 5B, FIG. 5C, FIG. 19G, and FIG. 19H).

To evaluate the effect of TRIM11 upregulation, TRIM11 and, as a control, GFP were cloned into the adeno-associated virus AAV9 vector, a serotype that effectively targets cells in the central nervous system (K. D. Foust et al., Nat Biotechnol 27, 59-65 (2009)) (FIG. 19I). When treated with myc-K18/P301L PFFs, AAV9-TRIM11-transduced PS19 cortical neurons exhibited ~60% reduction in AT8- and MC1-reactive tau compared to AAV9-GFP-transduced neurons (FIG. 5D, FIG. 5E, FIG. 11J, and FIG. 11K). Likewise, when hippocampal neurons derived from PS19 mice were treated with myc-K18/P301L PFFs, substantially less AT8- and MC1-reactive tau appeared in AAV9-TRIM11-tranduced neurons compared to AAV9-GFP-transduced neurons (FIG. 19L and FIG. 19M). These results indicate that TRIM11 affords neurons protection against tau aggregation.

### TRIM11 Maintains Neuronal Integrity and Connectivity

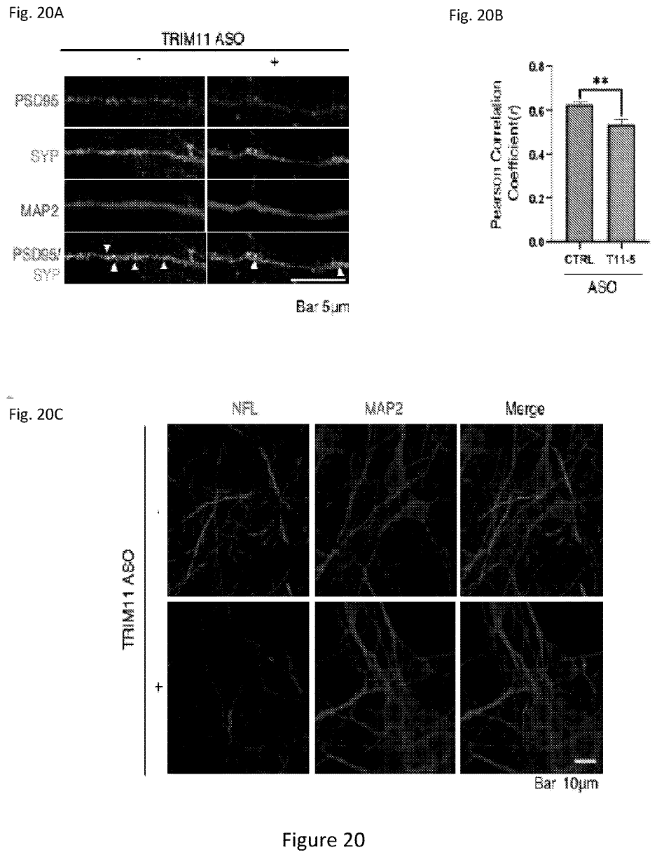

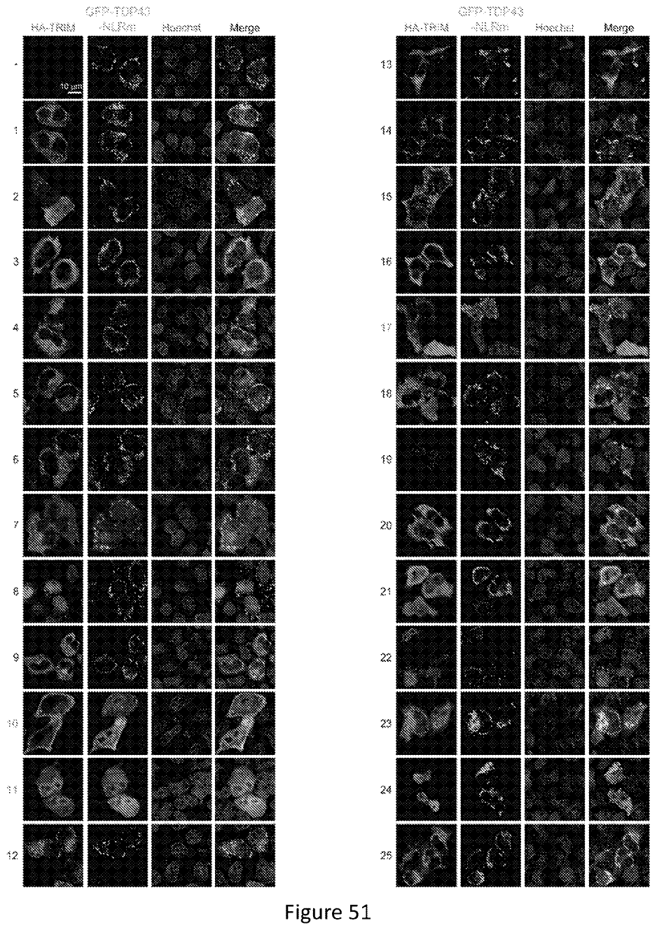

Synaptic degeneration is a prominent feature of AD patients and mouse models, proceeding neuronal loss and correlates strongly with cognitive decline (T. L. Spires-Jones, B. T. Hyman, Neuron 82, 756-771 (2014)). To evaluate the role of TRIM11 in synapse formation, neurons were probed with antibodies to the presynaptic marker synaptophysin (SYP) and postsynaptic density protein 95 (PSD95). Silencing TRIM11 in cortical neurons by ASOs led to reductions in SYP-positive puncta (~40%; FIG. 5F and FIG. 5G) and PSD95-positive puncta (~30%; FIG. 5H and FIG. 51), as well as their juxtaposition (FIG. 20A and FIG. 20B). TRIM11-depleted neurons also contained lower levels of neurofilament light chain (NFL), a component of the axonal scaffold (~40% reduction), but not microtubule-associated protein 2 (MAP2), which stabilizes microtubules in the dendrites (FIG. 5J through FIG. 5L and FIG. 20C). Moreover, silencing TRIM11 with various ASOs reduced the viability of cortical neurons in a manner that was correlated with the effect of these ASOs on TRIM11 expression (FIG. 5M and FIG. 19F). Therefore, reduction in endogenous TRIM11 impairs neuronal integrity and connectivity.

Conversely, forced TRIM11 expression via AAV9-mediated transduction increased SYP- and PSD95-positive puncta by ~70% and ~50%, respectively (FIG. 5N through FIG. 5Q). TRIM11 also elevated expression of NFL (by ~50%), but not MAP2 (FIG. 5R through FIG. 5T). Moreover, TRIM11 affords neurons resistance to tau PFF-induced cytotoxicity (FIG. 5U). Therefore, TRIM11 upregulation enhances neuronal integrity and connectivity.

### TRIM11 Ameliorates Tau Pathology and Cognitive Impairments in PS19 Mice

Next, the protective effect of TRIM11 on tau-associated pathology and cognitive/behavioral defects was examined in mouse models of tauopathy. PS19 transgenic mice, a widely-utilized tauopathy model that progressively accumulates tau inclusions, resembling human AD and other tauopathies, were first utilized (Y. Yoshiyama et al., Neuron 53, 337-351 (2007)). AAV9-TRIM11 or AAV9-GFP vectors were delivered to hippocampi of these mice via bilateral stereotaxic injection at 2.5 months of age, and brain pathology and animal behaviors were analyzed at ~10 months of age (FIG. 6A). As expected, hippocampi of AAV9-GFP-injected mice displayed strong NFT-like tau inclusions in various regions (FIG. 6B and FIG. 21A). In comparison, hippocampi of AAV9-TRIM11-injected mice contained substantially less tau pathology (~55% reduction) (FIG. 6B and FIG. 21A). This reduction was corroborated by immunoblot analysis, which showed that TRIM11 strongly reduced insoluble and soluble p-tau species (FIG. 6C and FIG. 21B through FIG. 21D). The inhibitory effect of TRIM11 was dose-dependent, as hippocampi with higher TRIM11 expression contained less p-tau (FIG. 6C).

Astrogliosis parallels the distribution and density of NFTs in tauopathies (F. Muramori, K. Kobayashi, I. Nakamura, Psychiatry Clin Neurosci 52, 593-599 (1998); T. Togo, D. W. Dickson, Acta Neuropathol 104, 398-402 (2002)) and is an early pathological manifestation of PS19 mice (Y. Yoshiyama et al., Neuron 53, 337-351 (2007)). AAV9-GFP-injected brains displayed strong staining of glial fibrillary acidic protein (GFAP) (FIG. 6D, FIG. 6E, and FIG. 21E), indicative of astrocyte activation. In contrast, GFAP immunoreactivity was decreased by ~50% in AAV9-TRIM11-injected brains, to a level observed in brains of age-matched wild-type littermates (FIG. 6D, FIG. 6E, and FIG. 21E). Activation of microglia was also detected in AAV9-GFP-injected brains, as shown in increased immunoreactivity to ionized calcium-binding adaptor molecule 1 (Iba1) (FIG. 6F, FIG. 6G, and FIG. 21F). In comparison, AAV9-TRIM11-injected brains showed a ~40% reduction of microgliosis, again to a level seen in wild-type mouse brains (FIG. 6F, FIG. 6G, and FIG. 21F).

Similar to human tauopathies, tau dysfunction in PS19 mice is associated with neuron loss, especially in the hippocampus (Y. Yoshiyama et al., Neuron 53, 337-351 (2007)). Compared to hippocampi of the GFP group of animals, hippocampi of the TRIM11 group of animals exhibited a ~50% increase in immunoreactivity for MAP2 (FIG. 6H, FIG. 6I, and FIG. 21G), as well as a ~70% increase in immunoreactivity for NFL, to a level comparable to that of the wild-type group (FIG. 6J, FIG. 6K, and FIG. 21H). Moreover, hippocampi of AAV9-TRIM11-treated animals showed an ~40% increase in the expression of NeuN (FIG. 6L, FIG. 6M, and FIG. 211). These results indicate that TRIM11 rescues axonal and dendritic degeneration and neuronal loss in PS19 mice.

AD and many other tauopathies are characterized by a progressive decline in cognitive function (C. L. Masters et al., Nat Rev Dis Primers 1, 15056 (2015); D. S. Knopman et al., Nat Rev Dis Primers 7, 33 (2021)). To evaluate learning and memory ability, an object recognition test (ORT) was performed, in which the propensity of mice to examine a novel object was observed (L. M. Lueptow., J Vis Exp, (2017)). The GFP group of mice showed no difference in the times interacting with familiar and novel objects (i.e., ~50% preference to each). In contrast, the TRIM11 group of mice exhibited a preference to the novel subject (~77%) that was similar to the wild-type mice (FIG. 6N), suggesting that TRIM11 rescues the deterioration of the long-term memory. Decline in motor strength is another tau-dependent defect in PS19 mice (H. Patel et al., Neurobiol Aging 109, 52-63 (2022)). In a wire hang test, the TRIM11 group of mice exhibited a substantially longer latency to fall (~75 sec) compared to the GFP group of mice (~50 sec), albeit they were still weaker than wild-type mice (FIG. 6O). Together, these results indicate that intracranial delivery of AAV9-TRIM11 prevents tau pathology, neurodegeneration, and gliosis, and improves cognitive and motor functions of PS19 mice.

### TRIM11 Prevents PFFs-Accelerated Tau Pathology in PS19 Mice

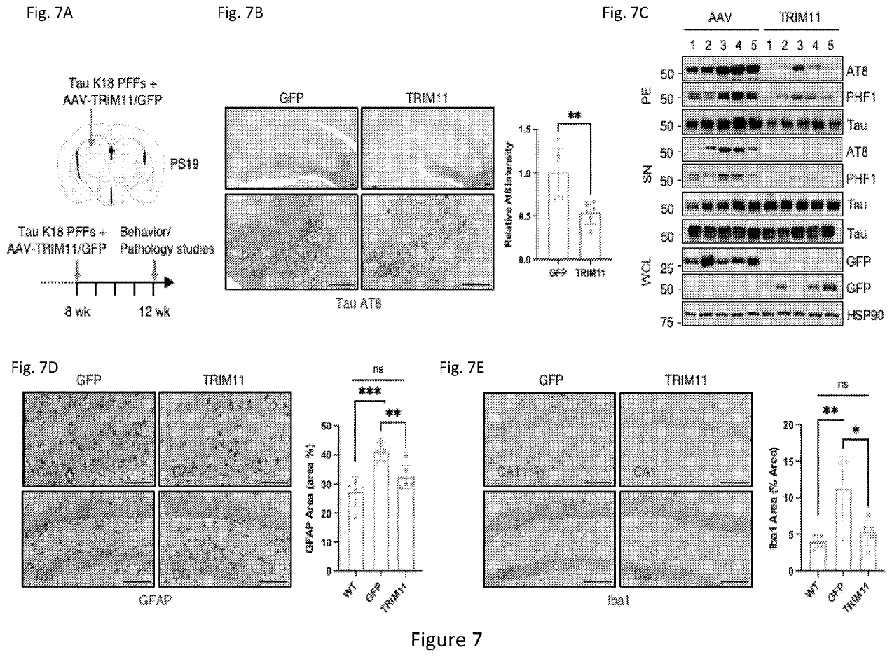

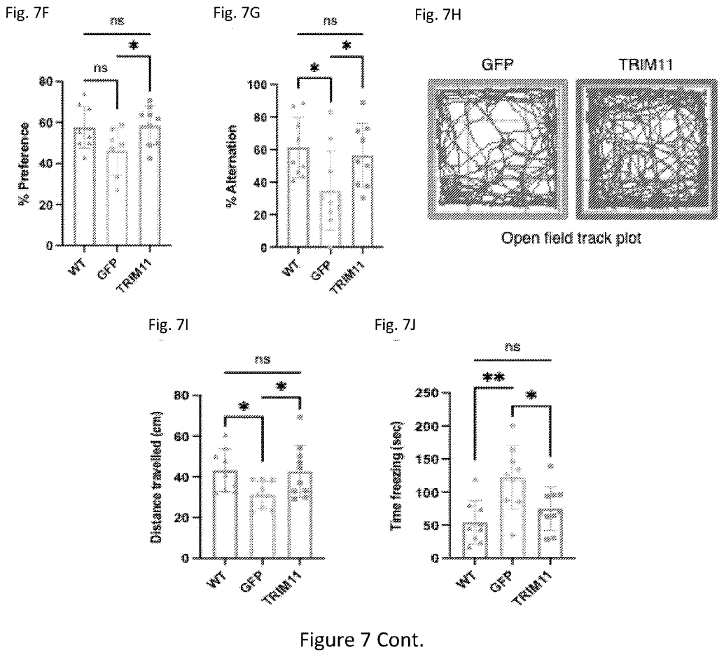

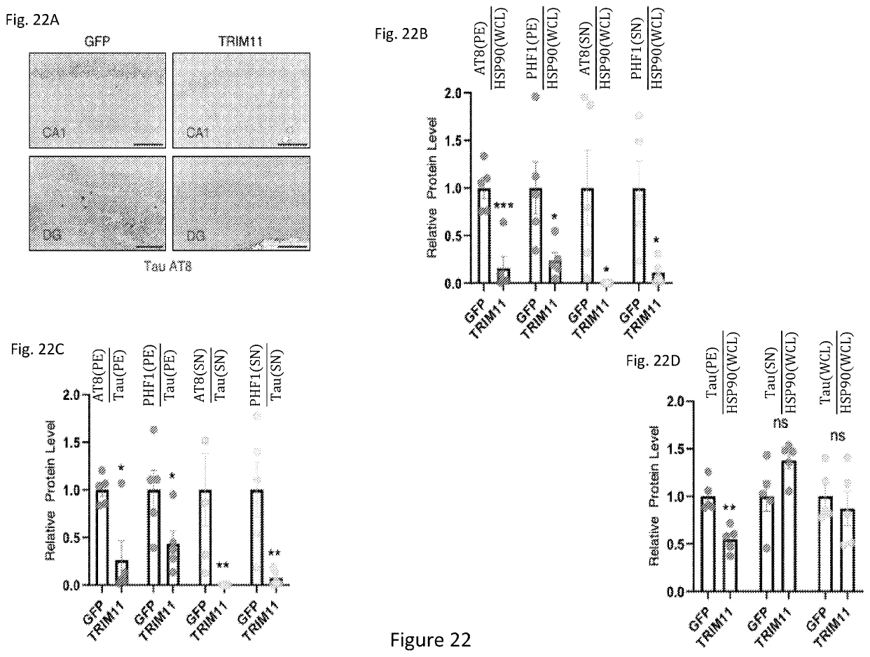

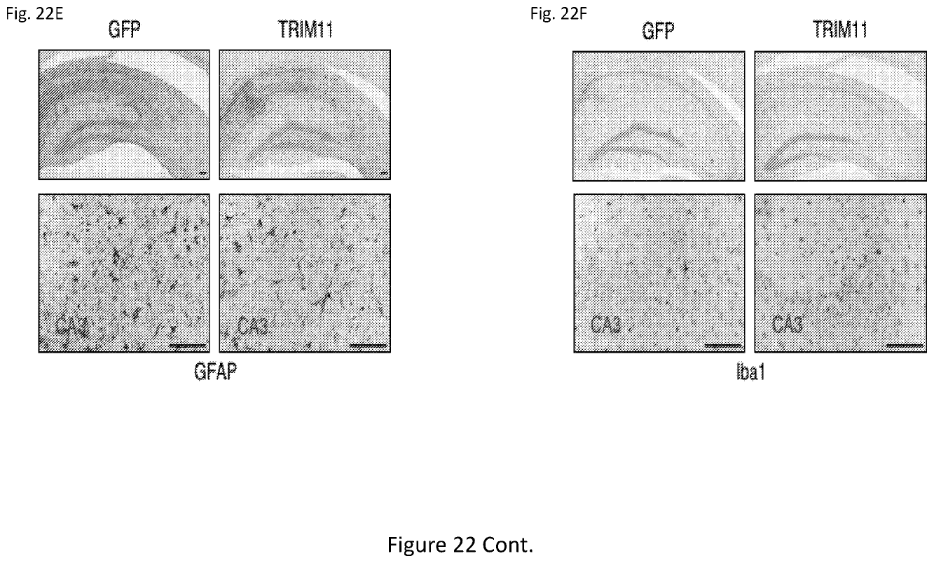

Inoculation of tau PFFs into the brain of PS19 mice before the known onset of tauopathy induces focal formation and widespread transmission of tau aggregates, accelerating neuroinflammation and cognitive/motor deterioration (M. Iba et al., J Neurosci 33, 1024-1037 (2013)). To evaluate whether TRIM11 protects against PFFs-accelerated disease phenotypes, PFFs generated from recombinant myc-K18/P301L (M. Iba et al., J Neurosci 33, 1024-1037 (2013)) were inoculated, together with either AAV9-TRIM11 or AAV9-GFP, unilaterally into the hippocampus of PS19 mice at 8 weeks of age and disease phenotypes were analyzed at 12 weeks of age (FIG. 7A). When co-inoculated with AAV9-GFP, K18 PFFs induced a substantially amount of tau aggregates in the ipsilateral hippocampus (FIG. 7B and FIG. 22A). In comparison, when co-inoculated with AAV9-TRIM11, K18 PFFs induced ~45% less tau aggregates (FIG. 7B and FIG. 22A). Levels of abnormally phosphorylated tau were also noticeably decreased in both soluble and insoluble fractions in AAV9-TRIM11-coinjected brains (FIG. 7C and FIG. 22B through FIG. 22D). In the presence of AAV9-GFP, K18 PFFs elicited astrogliosis and microgliosis. However, in the presence of AAV9-TRIM11, numbers of reactive astrocytes and microglia were not increased above those seen in wild-type mouse brains (FIG. 7D, FIG. 7E, FIG. 22E, and FIG. 22F).

In an ORT, TRIM11 group of animals showed a preference to the novel subject comparable to the wild-type animals, whereas the GFP group of animals showed no such preference (FIG. 7F). Therefore, TRIM11 obviates the decline in long-term memory. To assess the effect of TRIM11 on short-term memory, spontaneous alternation behavior (SAB) was measured in the Y-maze (A. K. Kraeuter, P. C. Guest, Z. Sarnyai, Methods Mol Biol 1916, 105-111 (2019)). The TRIM11 group exhibited significantly more spontaneous alternations (~56%) than the GFP group (~35%), reaching a level shown by wild-type mice (FIG. 7G). Thus, TRIM11 also prevents the decline in the hippocampus-dependent working memory.

To evaluate motor function and anxiety-like phenotypes, an open field test was performed. Compared to the GFP group of mice, the TRIM11 group of mice travelled a longer distance (~36% increase) (FIG. 7H and FIG. 7I), and stopped for a shorter period of time (~40% reduction) (FIG. 7J). These behaviors of TRIM11 mice were again similar to those of wild-type mice (FIG. 7H through FIG. 7J). Together, these results indicate that TRIM11 inhibits the seeding and cell-to-cell transmission of tau aggregates in PS19 mice, preventing the activation of glial cells and the decline in cognitive and motor abilities.

### TRIM11 Ameliorates Tau Pathology and Cognitive Deficits in 3× Tg-AD Mice

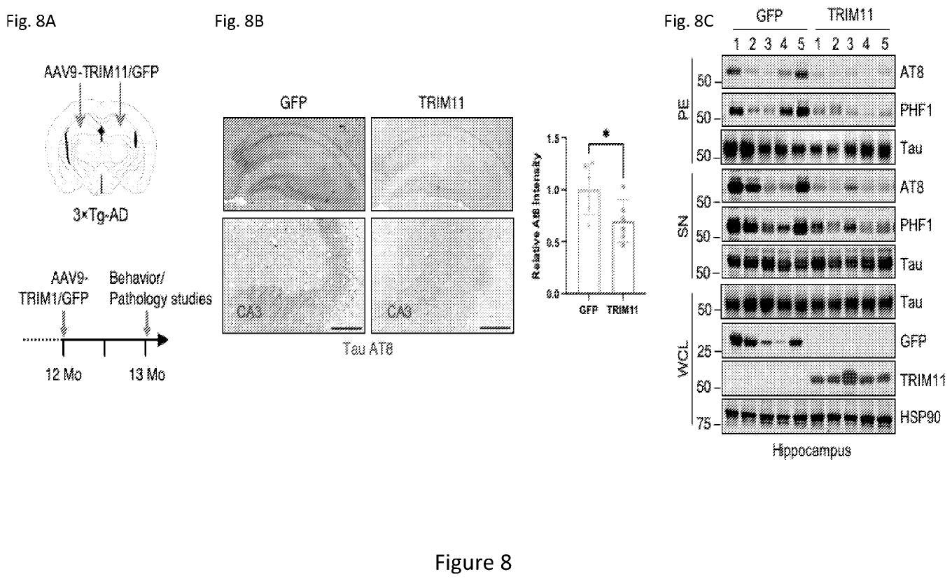

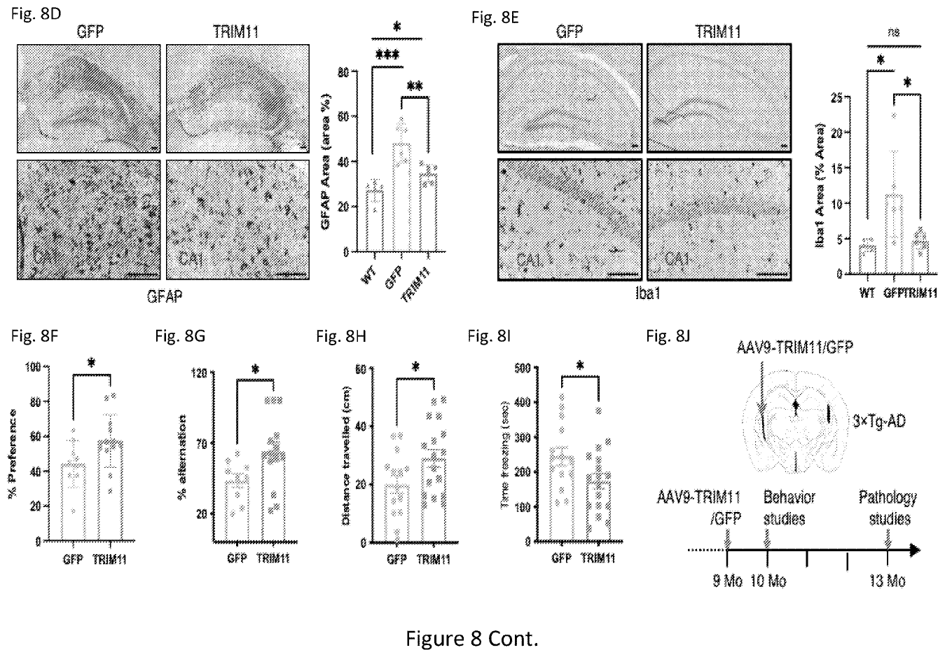

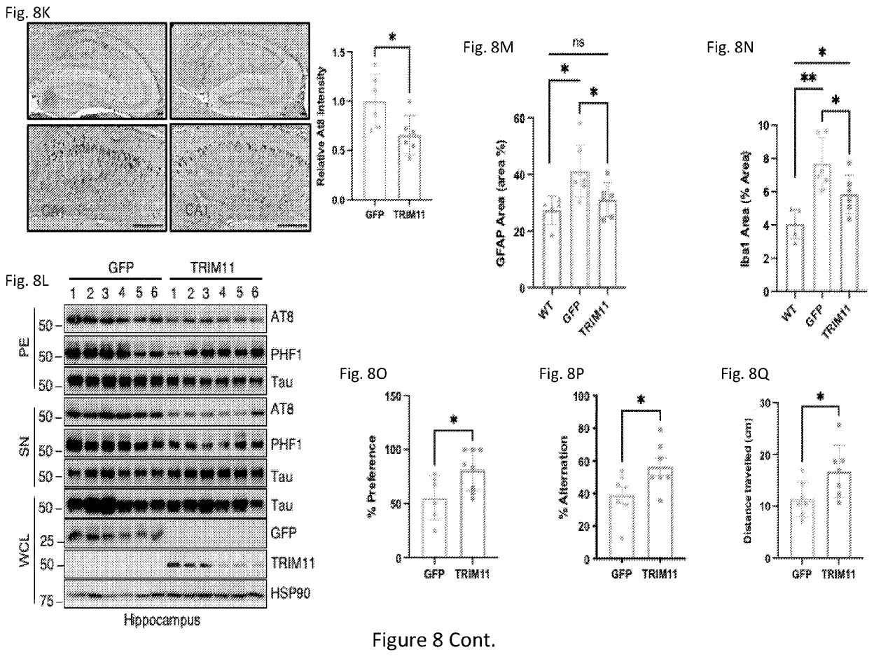

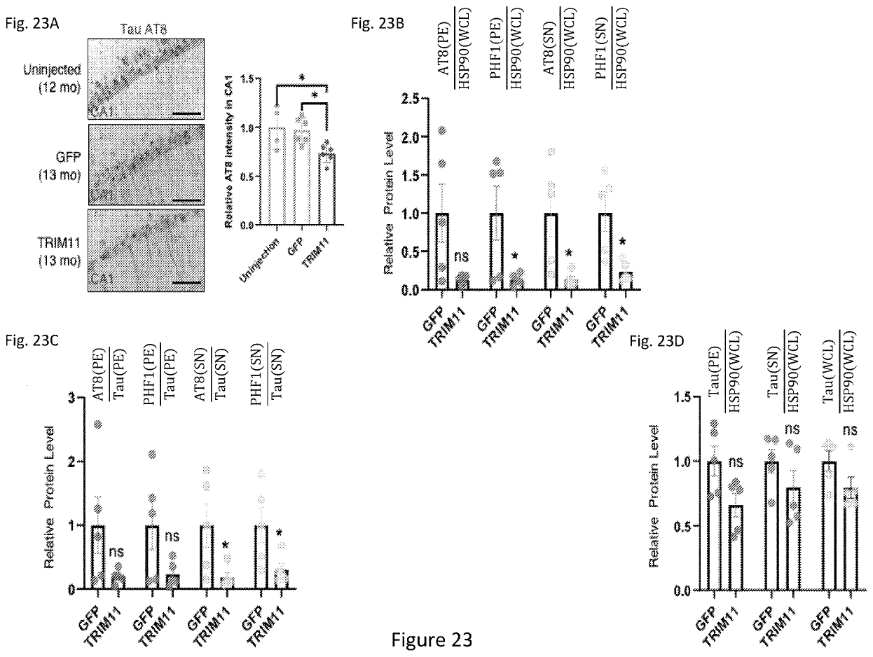

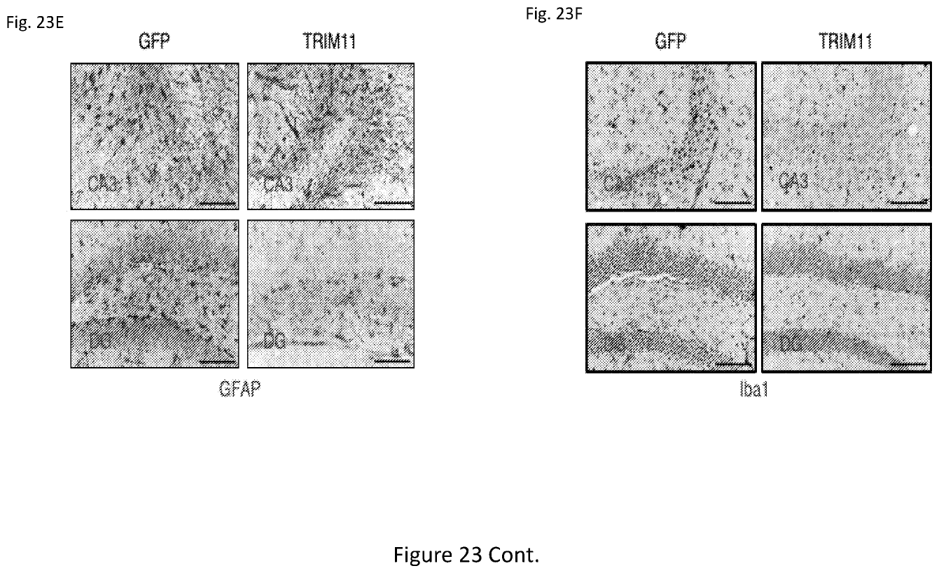

Besides tau-containing intraneuronal NFTs, AD is characterized by extraneuronal plaques containing Aβ peptide (C. L. Masters et al., Nat Rev Dis Primers 1, 15056 (2015); D. S. Knopman et al., Nat Rev Dis Primers 7, 33 (2021)). To evaluate the protective effect of TRIM11 on the combined pathological effects of tau and Aβ, a triple transgenic (3×Tg-AD) model was used that expresses tau P301L as well as familial mutations in two AD-related proteins, the amyloid precursor protein (APP) K595N/M596L (the Swedish mutation) and the presenilin 1 (PS1) M146V (S. Oddo et al., Neuron 39, 409-421 (2003)). 3×Tg-AD mice demonstrate both Aβ plaque and tau tangles, resembling human AD. AAV9-TRIM11 or AAV9-GFP was delivered to the hippocampus of 3×Tg-AD mice at 12 months of age, when they already showed a substantial amount of tau pathology in the hippocampus, and performed pathology and behavior analysis one month later (FIG. 8A). Delivery of AAV9-TRIM11 led to a substantial reduction in tau pathology compared to that of AAV9-GFP, as shown by IHC analysis (~30% reduction; FIG. 8B and FIG. 23A). This was corroborated by western blot analysis, which showed a reduction of ~80-90% in p-tau species reactive to AT8 and PHF-1 (FIG. 8C and FIG. 23B through FIG. 23D). Levels of AT8 staining in AAV9-TRIM11-injected brains were lower than those in un-injected brains at 12 months of age (FIG. 23A), suggesting that TRIM11 cleared pre-existing tau aggregates. GFAP and Iba1 immunoreactivity was also markedly lower in TRIM11 group compared to GFP group, reduced by ~30% and 60%, respectively (FIG. 8D, FIG. 8E, FIG. 23E and FIG. 23F).

In 3×Tg-AD mice, cognitive impairment initially manifests at 4 months of age and progresses as tau tangles and Aβ plaques accumulate (S. Oddo et al., Neuron 39, 409-421 (2003)). Compared to AAV9-GFP-injected animals, AAV9-TRIM11-injected animals spent significantly more time exploring the novel object and less time on the familiar object in the ORT (FIG. 8F). They also spent more time exploring different arms in the Y-maze test (FIG. 8G). Moreover, the AAV9-TRIM11 group exhibited higher locomotive activity in an open filed test, traveling longer distance and stopping for less time (FIG. 8H and FIG. 8I). Therefore, TRIM11 suppresses tau pathology and neuroinflammation and improves cognitive and motor ability of 3×Tg-AD mice.

### Effect of CSF Delivery of AAV9-TRIM11

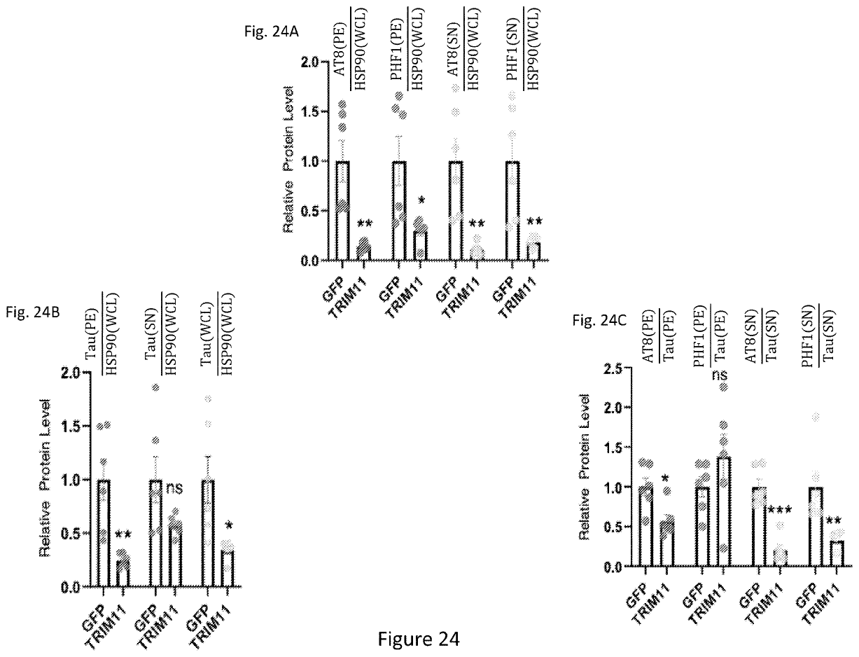

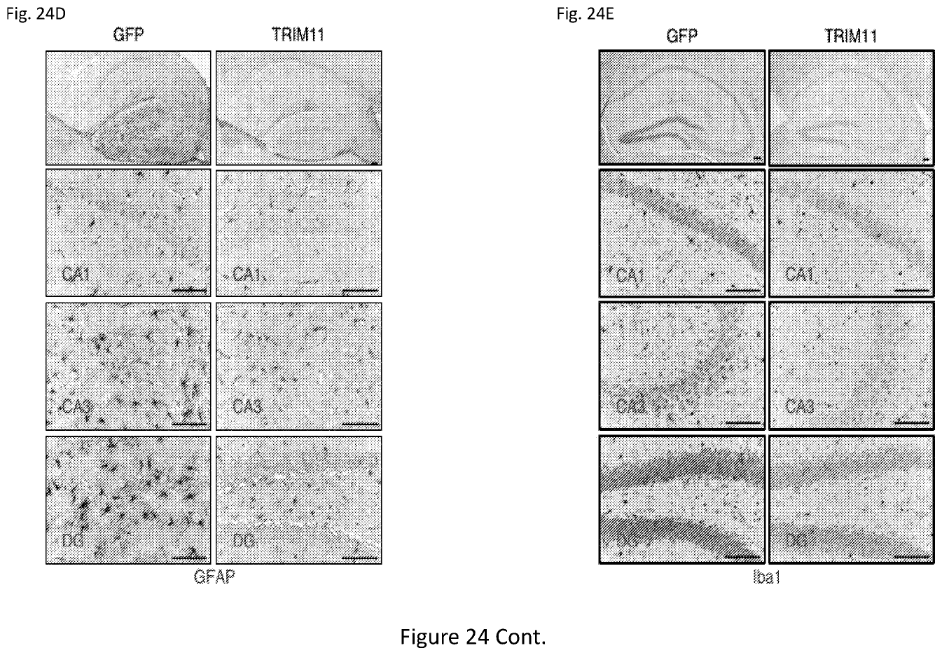

The experiments described above on animal models of tauopathies not only demonstrate the role of TRIM11 in protecting against tau-dependent neurodegeneration in mammalian brains, but also suggest potential utility of the TRIM11 gene in disease treatment. However, the intraparenchymal infusion used in these experiments is spatially restricted, whereas AD and many other tauopathies affect multiple brain regions. Thus, the effect of administering AAV vectors globally in the brain was examined via the cerebrospinal fluid (CSF). AAV9-TRIM11 and AAV9-GFP were delivered by intracerebroventricular (ICV) injection to 3×Tg-AD mice at 9 months of age and analyzed mouse behaviors and pathology 2 to 4 months later (FIG. 8J). Compared to the AAV9-GFP group of mice, the AAV9-TRIM11 group of mice showed a ~35% reduction in tau-containing, NFT-like inclusions in the hippocampus (FIG. 8K) and a ~50-70% reduction in soluble and insoluble p-tau species in cell lysates (FIG. 8L and FIG. 24A through FIG. 24C). AAV9-TRIM11 mice also showed a substantial reduction in GFAP- and Iba1-immunoreactivity, to levels that were close to or moderately higher than those in the wild-type mice (FIG. 8M, FIG. 8N, FIG. 24D and FIG. 24E). Moreover, compared to the GFP group of animals, the TRIM11 group of animals performed significantly better in the ORT and Y-maze tests (FIG. 8O and FIG. 8P) and showed higher locomotive ability in the open field test (FIG. 8Q). These results suggest that CSF administration of the AAV9-TRIM11 vector may be beneficial for treating tauopathies.

The current study reveals that TRIM11 plays a critical role in maintaining tau in its functional soluble state. TRIM11 achieves this important outcome through multiple mechanisms. It promotes the degradation of mutant and hyperphosphorylated tau as well as excess normal tau, thereby acting as a crucial link between tau and the proteasome. TRIM11 also serves as a molecular chaperone, preventing tau misfolding and aggregation. Moreover, TRIM11 functions as a disaggregase to dissolve preexisting tau deposits, including amyloid fibrils that are often intractable. Canonical PQC systems such as HSP60, HSP70, and HSP90-which are conserved in both prokaryotes and eukaryotes-rely on coordinated actions of multiple components in an ATP-dependent manner. By contrast, TRIM11 can prevent and reverse tau aggregation by itself and independent of ATP. These multiple and potent activities of TRIM11 in combination are highly effective in protecting against tau misfolding and aggregation in various cell and animal models of tauopathy. The survey of the large number of human TRIM proteins shows that these activities of TRIM11 are likely shared by at least some other TRIM proteins (FIG. 1, FIG. 10, and FIG. 11). TRIM proteins, which are only identified in metazoans, might have evolved later during evolution in part to maintain the quality of the complex proteomes that enable these intricate life forms.

The downregulation of TRIM11 among a large number of sporadic AD brains, combined with its potent protective effect on tau, suggest that the diminished capacity of TRIM11 might contribute to tau aggregation and the associated pathological and cognitive changes. AD and other neurodegenerative tauopathies are becoming increasingly prevalent as the population ages, yet remain incurable. Tau, whose abnormality is both more closely linked to the disease progression than that of Aβ and required for the cytotoxicity of Aβ, represents an important target for AD in addition to primary tauopathies (C. Li, J. Gotz, Nat Rev Drug Discov 16, 863-883 (2017)). In recent years, gene therapy is becoming an important approach to treat neurological diseases (B. E. Deverman, B. M. Ravina, K. S. Bankiewicz, S. M. Paul, D. W. Y. Sah, Nat Rev Drug Discov 17, 641-659 (2018)). AAVs can transduce the non-dividing neurons and permit permanent expression of the therapeutic gene after a single administration (B. E. Deverman, B. M. Ravina, K. S. Bankiewicz, S. M. Paul, D. W. Y. Sah, Nat Rev Drug Discov 17, 641-659 (2018); L. Naldini, Nature 526, 351-360 (2015)), with positive clinical outcomes (J. R. Mendell et al., N Engl J Med 377, 1713-1722 (2017)). The effect of intraparenchymal and intraventricular delivery of AAV9-TRIM11 in animal models provides a proof concept for the potential utility of the TRIM11 gene to restore protein homeostasis in AD and other tauopathies, hence addressing the root cause of these 5 diseases.

TABLE 1
Human and mouse TRIMs that were tested

| | | Accession | | | Amino |
|---|---|---|---|---|---|
| Name | Group | number | Isoform | ORF (bp) | Acid |
| hTRIM1 | I | NM_012216.4 | 1 | 2205 | 735 |
| hTRIM2 | VII | NM_015271.5 | 1 | 2313 | 771 |
| hTRIM3 | VII | NM_033278.4 | 2 | 2232 | 744 |
| hTRIM4 | IV | NM_033017.4 | α | 1500 | 500 |
| hTRIM5 | IV | NM_033034.3 | α | 1479 | 493 |
| hTRIM6 | IV | NM_001003818.3 | 1 | 1548 | 516 |
| hTRIM7 | IV | NM_203293.3 | 1 | 1533 | 511 |
| hTRIM8 | V | NM_030912.3 | 1 | 1653 | 551 |
| hTRIM9 | I | NM_015163.6 | 1 | 2130 | 710 |
| hTRIM10 | IV | NM_006778.4 | 1 | 1443 | 481 |
| hTRIM11 | IV | NM_145214.3 | 1 | 1404 | 468 |
| mTRIM12 | V | AF220126.1 | | 852 | 284 |
| hTRIM13 | XI | NM_001007278.3 | 4 | 1230 | 410 |
| hTRIM14 | IV | NM_033219.3 | 2 | 1326 | 442 |
| hTRIM15 | IV | NM_033229.3 | | 1395 | 465 |
| hTRIM16 | IV | NM_006470.4 | 3 | 1692 | 564 |
| hTRIM17 | IV | NM_001024940.3 | 2 | 1431 | 477 |
| hTRIM18 | I | NM_001193277.1 | 2 | 2001 | 667 |
| hTRIM19 | V | NM_033238.3 | 1 | 2646 | 882 |
| hTRIM20 | IV | NM_000243.3 | 1 | 2343 | 781 |
| hTRIM21 | IV | NM_003141.4 | | 1425 | 475 |
| hTRIM22 | IV | NM_006074.5 | 1 | 1494 | 498 |
| hTRIM23 | IX | NM_001656.4 | α | 1722 | 574 |
| hTRIM24 | VI | NM_015905.3 | 1 | 3150 | 1050 |
| hTRIM25 | IV | NM_005082.5 | | 1890 | 630 |
| hTRIM26 | IV | NM_003449.5 | 1 | 1617 | 539 |
| hTRIM27 | IV | NM_006510.5 | | 1539 | 513 |
| hTRIM28 | VI | NM_005762.3 | | 2505 | 835 |
| hTRIM29 | V | NM_012101.4 | 1 | 1764 | 588 |
| mTRIM30 | IV | NM_009099.2 | 1 | 1488 | 496 |
| hTRIM31 | V | NM_007028.5 | 1 | 1275 | 425 |
| hTRIM32 | VII | NM_012210.4 | 1 | 1959 | 653 |
| hTRIM33 | VI | NM_015906.4 | α | 3381 | 1127 |
| hTRIM34 | IV | NM_021616.6 | 1 | 1464 | 488 |
| hTRIM35 | IV | NM_171982.5 | 1 | 1479 | 493 |
| hTRIM36 | I | NM_018700.4 | 1 | 2184 | 728 |
| hTRIM37 | VIII | NM_001005207.5 | | 2892 | 964 |
| hTRIM38 | IV | NM_006355.5 | | 1395 | 465 |
| hTRIM39 | IV | NM_021253.4 | 1 | 1554 | 518 |
| hTRIM40 | V | BC060785.1 | 2 | 774 | 258 |
| hTRIM41 | IV | NM_033549.5 | 1 | 1890 | 630 |
| hTRIM42 | III | NM_152616.5 | | 2172 | 724 |
| hTRIM43 | IV | NM_138800.3 | | 1338 | 446 |
| hTRIM44 | V | NM_017583.6 | | 1032 | 344 |
| hTRIM45 | X | NM_025188.4 | 1 | 1740 | 580 |
| hTRIM46 | I | NM_025058.5 | 1 | 2277 | 759 |
| hTRIM47 | IV | NM_033452.3 | | 1914 | 638 |
| hTRIM48 | IV | NM_024114.5 | | 672 | 224 |
| hTRIM49 | IV | NM_020358.2 | | 1356 | 452 |
| hTRIM50 | IV | NM_178125.3 | 1 | 1461 | 487 |
| hTRIM51 | IV | NM_032681.4 | | 1356 | 452 |
| hTRIM52 | V | NM_032765.4 | 1 | 891 | 297 |
| hTRIM54 | II | NM_187841.3 | 2 | 1074 | 358 |
| hTRIM55 | II | NM_184085.2 | 1 | 1644 | 548 |
| hTRIM56 | V | NM_030961.3 | | 2265 | 755 |
| hTRIM58 | IV | NM_015431.4 | | 1458 | 486 |
| hTRIM59 | XI | NM_173084.3 | | 1209 | 403 |
| hTRIM60 | IV | NM_152620.3 | 2 | 1413 | 471 |
| hTRIM61 | V | NM_001012414.3 | | 627 | 209 |
| hTRIM62 | IV | NM_018207.3 | 1 | 1425 | 475 |
| hTRIM63 | II | NM_032588.4 | | 1059 | 353 |
| hTRIM64 | IV | NM_001164397.3 | | 1347 | 449 |
| hTRIM65 | IV | NM_173547.4 | 1 | 1551 | 517 |
| hTRIM66 | VI | NM_014818.2 | 1 | 3648 | 1216 |
| hTRIM67 | I | NM_001004342.5 | 1 | 2349 | 783 |
| hTRIM68 | IV | NM_018073.8 | 1 | 1455 | 485 |
| hTRIM69 | IV | NM_182985.5 | α | 1500 | 500 |
| hTRIM70 | IV | NM_001037330.3 | | 1044 | 348 |
| hTRIM71 | VII | NM_001039111.3 | | 2604 | 984 |
| hTRIM72 | IV | NM_001008274.4 | | 1431 | 477 |
| hTRIM73 | V | NM_198924.4 | | 750 | 250 |
| hTRIM74 | V | NM_198853.3 | 1 | 750 | 250 |
| mTRIM75 | IV | NM_001033429.2 | | 1401 | 467 |
| hTRIM76 | I | NM_153610.5 | | 2253 | 751 |
| hTRIM77 | IV | NM_001146162.1 | 1 | 1350 | 450 |

All TRIMs are of human origin except TRIM12 and TRIM30, which are of mouse origin and are absent in humans. TRIM53 is a pseudogene and is not listed. TRIM57 is the same as TRIM59. For each TRIM, the subfamily, accession number, isoform, length of the open reading frame, and number of amino acids are indicated. The isoform for each TRIM is usually the longest one and, when not indicated, the only isoform that has been identified.

### Example 2: TRIM Proteins Guard Against Aggregation of a Wide Range of Proteins Associated with Neurodegenerative Diseases

The vast majority of familial neurodegenerative diseases—including most Alzheimer's disease (AD), Parkinson's disease (PD), amyotrophic lateral sclerosis (ALS) cases, —are sporadic in which normal proteins expressed at physiological levels misfold and assemble into insoluble aggregates. The predominant form of these aggregates is amyloid fibril, a highly ordered structure that contains β-strands aligned perpendicularly to the fibril axis (cross-ß structure) (T. P. Knowles, M. Vendruscolo, C. M. Dobson, Nat Rev Mol Cell Biol 15, 384-396, 2014) (D. Eisenberg, M. Jucker, Cell 148, 1188-1203, 2012).

To contend with protein misfolding and aggregation, organisms in all kingdoms of life are dependent on various protein quality control (PQC) systems. These systems include molecular chaperones to prevent protein misfolding and aggregation, disaggregases to dissolve pre-existing protein aggregates permitting their refolding, and degradation pathways to recycle defective proteins or excess normal proteins (D. C. Rubinsztein, Nature 443, 780-786, 2006; W. E. Balch, R. I. Morimoto, A. Dillin, J. W. Kelly, Science 319, 916-919, 2008) (J. Tyedmers, A. Mogk, B. Bukau, Nat Rev Mol Cell Biol 11, 777-788, 2010; Y. E. Kim, M. S. Hipp, A. Bracher, M. Hayer-Hartl, F. U. Hartl, Annu Rev Biochem 82, 323-355, 2013) (H. Saibil, Nat Rev Mol Cell Biol 14, 630-642, 2013) (S. Wolff, J. S. Weissman, A. Dillin, Cell 157, 52-64, 2014; K. L. Mack, J. Shorter, Front Mol Biosci 3, 8, 2016; S. M. Doyle, O. Genest, S. Wickner, Nat Rev Mol Cell Biol 14, 617-629, 2013). The age-dependent conversion of neurodegeneration-associated proteins from soluble states to fibrillar aggregates in both familial and sporadic neurodegenerative diseases suggests the existence of a PQC system that can effectively counteract protein aggregation early in life. However, the identify of such a PQC system remains unclear.

Tripartite-motif (TRIM) proteins are characterized by an N-terminal RBCC motif consisting of a RING domain, one or two B-boxes, and a coiled-coil region. These proteins are found only in metazoans and their number has increased during evolution, with ~20 in C. elegans and over 70 in mice and humans K. Ozato, D. M. Shin, T. H. Chang, H. C. Morse, 3rd, Nat Rev Immunol 8, 849-860, 2008; S. Hatakeyama, Nat Rev Cancer 11, 792-804, 2011). Emerging evidence suggests that certain TRIM proteins can enable protein folding and prevent aggregation (L. Guo et al., Mol Cell 55, 15-30, 2014; L. Chen et al., Cell Rep 18, 3143-3154, 2017; L. Chen, G. Zhu, E. M. Johns, X. Yang, Nat Commun 9, 1223, 2018; G. Zhu et al., Cell Rep 33, 108418, 2020), although other TRIMs appear to aggravate protein aggregation (M. W. Rousseaux et al., Elife 5, 2016). Hence, it remains unclear how many TRIMs can enable protein folding. Systematic analysis of virtually all known human TRIMs was performed on various diseases-associated proteins. The results show that at least several TRIM proteins can mediate degradation of defective proteins or superfluous normal proteins. Moreover, many TRIM proteins can prevent and reverse aggregation of proteins associated with neurodegenerative diseases. These results indicate that many TRIM proteins possess potent PQC activities and that these proteins might be applied to treat a plethora of neurodegenerative diseases.

The materials and methods used in the experiments are now described.

### Antibodies

Following antibodies were purchased from vendors: SOD1 (Invitrogen, Cat #MA1-105); TDP-43 (C-terminal) (Proteintech, Cat #12892-1-AP); phospho (499/410)-TDP43 (Proteintech, Cat #66318-1-1g); Huntingtin (Millipore, Cat #MAB2166); GFAP (Cell Signaling; Cat #80788S), Iba-1 (Cell Signaling; Cat #17198S); MAP2 (Cell Signaling, Cat #4542S); choline acetyl transferase (ChAT) (Millipore: Cat #AB144P); GFP (Santa Cruz, Cat #sc-9996); FLAG (Sigma, Cat #F1804; Cell Signaling, Cat #14793S); HA (Cell Signaling; Cat #3724S); TRIM11 (Millipore; Cat #ABC926); PARP (Cell Signaling, Cat #9542S); C-CASP3 (Cell Signaling, Cat #9661S); Ubiquitin (Invitrogen, Cat #13-1600); K48-linked ubiquitin (Cat #8081S); HSP90 (Cell Signaling; Cat #4877S); GAPDH (Cell Signaling, Cat #2118S); β-tubulin (Santa Cruz; Cat #sc-5274); DsRed (Santa Cruz; Cat #sc-390909); Lamin A/C (Santa Cruz; Cat #sc-376248); Normal rabbit IgG (Cell Signaling; Cat #2729S).

### Reagents

Mg²⁺-ATP (Cat #A9187), FLAG Peptide (Cat #F3290), ammonium chloride (Cat #09718), Benzonase (Cat #E1014), complete protease inhibitor Cocktail (Cat #11697498001), cycloheximide (Cat #66819), thioflavin T (Cat #T3516), human AB42 peptide were purchased from Sigma-Aldrich. Lipofectamine2000 (Cat #6031), Lipofectamine RNAiMAX (Cat #13778150), SYBR Green Master Mix (Cat #A25742), and SuperSignal™ Western Blot Substrate Bundle (Cat #A45916) were purchased from Thermo Fisher Scientific. TRIzol Reagent (Cat #15596) and Hochst33342 (Cat #H3570) from Invitrogen, 4′,6-diamidino-2-phenylindole (DAPI) (Cat #H-1200) from Vector Laboratories, MG132 (Cat #S2619) from Selleck Chemicals, poly-ethylenimine (PEI) (Linear, MW 25000, Cat #23966-1) from Polysciences, Bradford protein assay kit (Cat #5000205) from Bio-Rad Labs, and ABC (Avidin-Biotin Complex) Kits (Cat #PK6100) from Vector Laboratories.

### Plasmids

For expression in mammalian cells, cDNAs for 75 TRIMs, including 73 human TRIMs and mouse TRIM12 and TRIM30, were synthesized and cloned into pCDH-EF1-FHC (Addgene, #64874), in which each TRIM was fused with an FLAG tag and an HA tag at the C-terminus (Gene Universal, Newark, DE). Tau-VN and tau-VC were cloned in pBiFC-VN173 (Addgene plasmid #22010) and pBiFC-VC155 (Addgene plasmid #22011), respectively. VIS and SV2 plasmids were generated by cloning α-synuclein into pBiFC-VN173 (Addgene, #22010) and pBiFC-VC155 (Addgene, #22011), respectively (L. Huang et al., Nature 597, 132-137, 2021).

pRK5-EGFP-tau and pRK5-EGFP-tau P301L, in which EGFP is fused to the N-terminus of tau proteins, were obtained (Addgene plasmids #46904 and #46908, respectively). pEGFP-N1-FUS/TLS-FLAGC, in which FUS/TLS is fused to both EGFP and FLAG at the N-terminus (referred to as EGFP-FUS in the current study), was obtained (Addgene, Cat #60362). EGFP-α-synuclein-A53T, in which α-Syn-A53T was fused to EGFP at the N-terminus (referred to as GFP-α-Syn-A53T in the current study), was obtained (Addgene, Cat #60362). pF145 pAcGFP1 SOD1G93A, in which SOD1G93A was fused to GFP at the N-terminus (referred to as GFP-SOD1G93A in the current study), was obtained (Addgene, Cat ##26406). EGFP-TDP43-WT, M337V, and Q331K were obtained. Atxn1 82Q-GFP was previously described (L. Guo et al., Mol Cell 55, 15-30 (2014)

### Cell Culture

Inducible NSC-34 cells stably overexpressing human SOD1-WT/G93A fused with green fluorescence protein (GFP) were obtained and grown as described by Y. Mali, N. Zisapels, Neurobiol Dis 32, 133-141, 2008. NSC-34 cells were grown in DMEM media supplemented with 10% FBS, 100 μg/ml penicillin and streptomycin and 1 mM GlutaMax. Media was also supplemented with G418 (700 μg/ml) and hygromycin B (200 μg/ml) to maintain the GFP positive cells. PC12 cells inducibly expressing human Htt with non-pathogenic (Q23) or pathogenic (Q74) polyglutamine expansion fused with GFP were obtained (A. Wyttenbach et al., Hum Mol Genet 10, 1829-1845, 2001). PC-12 cells were grown in DMEM media supplemented with 10% horse serum (heat-inactivated), 5% FBS, 100 μg/ml penicillin and streptomycin and 1 mM GlutaMax.

### Cell Transfection

For screening of TRIM proteins, HEK293T cells cultured in 6-well plates for 24 h and at ~80-90% confluence were co-transfected with the indicated plasmids. Medium was changed 12 h later. Cells were harvested 48 h after transfection and lysed with 150 μl ice-cold lysis buffer (50 mM Tris, pH 8.8, 100 mM NaCl, 5 mM MgCl2, 0.5% NP-40, 1 mM DTT, 250 IU/ml benzonase, 1 mM PMSF, and 1× complete protease inhibitor cocktail) for 30 minutes (min) on ice. Lysates were centrifuged for 15 min at 13,000 rpm at 4 °C. The NP-40-soluble supernatants were designated as SN fraction. The NP-40-insoluble pellets were washed once with 500 μl precooled PBS and re-suspended in 50 μl ice-cold pellet buffer (20 mM Tris, pH 8.0, 15 mM MgCl₂, 1 mM DTT, 250 IU/ml benzonase, 1 mM PMSF, and 1× complete protease inhibitor cocktail) for 30 min on ice, and then added 25 μl 3× boiling buffer (6% SDS, 20 mM Tris, pH 8.0, 150 mM DTT). The samples were heated at 95 °C for 5-10 min. Aggregated proteins in pellets that could be solubilized by SDS were designated as the SDS-soluble pellet fraction (PE).

Both the SN and PE fractions were boiled in SDS sample buffer (final concentration: 62.5 mM Tris pH 6.8, 2% SDS, 10% glycerol, 100 mM DTT, 0.01% bromophenol blue), analyzed by SDS-PAGE, and then were transferred to a nitrocellulose membrane. After blocking with 5% non-fat milk in tris-buffered saline with Tween (TBST), the membrane was incubated with anti-GFP, anti-HA, and anti-HSP90 antibodies, followed by HRP-conjugated secondary antibodies. Signals were detected by using a Western Blot Substrates and Substrate Kits in an ECL detection system (Bio-Rad Chemidoc Touch Imaging System Chemiluminescence/Fluorescence Detection). The sample loading and exposure time of the immunoblots were controlled so that all bands of western blots were detected within the linear range.

For analyzing the effect of TRIM11 in NSC-34/GFP-SOD1WT and NSC-34/GFP-SOD1^{G93A} cells, cells were seeded 5×10⁵/well in 6-well plates and incubated with doxycycline (1 μg/ml) for 16 h to induce the expression of the GFP-SOD1WT and GFP-SOD1G93A proteins. Once higher GFP expression was observed after 16 hours, cells were transfected with increasing concentrations (0-2 μg) of FLAG-TRIM11 or different TRIM11 deletion mutants for further 40 h. Relative GFP expression was documented by observing cells under a fluorescence microscope.

### BiFC Assay

HEK293T cells were seeded into 6 well plates for 24 h with DMEM medium supplemented with 10% fetal bovine serum (FBS), so that cells grow to a confluence of 80-90% at the time of transfection. Cells were co-transfected with indicated plasmids. 12 h later, cells were changed with fresh medium and cultured for another 12 h. Fluorescence was observed on Revolve Microscope Demo (Echo Laboratories).

### Cell Lysis, Fractionation, and Immunoblot Analyses

Media was aspirated, and cells were washed once with sterile phosphate buffered saline (PBS). Once PBS was aspirated, cells were lysed with lysis buffer [50 mM Tris, pH 8.8, 100 mM NaCl, 5 mM MgCl₂, 0.5% Nonidet P-40 (NP-40), 1 mM DTT, 250 IU/ml benzonase, 1 mM PMSF, and 1× complete protease inhibitor cocktail] for 30 min on ice. Lysates were centrifuged for 20 min at 13,000 rpm and 4 °C. This soluble fraction (SN) was measured using Bradford assay (Bio-Rad Labs). The NP-40-insoluble pellets (PE) were re-suspended in 2× Laemelli's buffer (4% SDS, 20% glycerol, and 120 mM Tris-HCl pH 6.8), incubated at room temperature for 10 min followed by incubation in a heat block set to 100 °C for another 10 min. Equal amounts of SN fraction were boiled in 1× Laemelli's buffer containing 2% SDS for 10 min as well. Whole cell lysates (WCL) were made 1:1 mixture of SN and PE fraction. All SN, PE, and WCL samples were analyzed by SDS-PAGE and immunoblotting.

### Negative-Stain Electron Microscopy

A 5 μl volume of sample was applied to a thin carbon grid that was glow discharged for 2 min using a Pelco Easyglow instrument. A 5 μl of freshly made 2% Uranyl acetate stain solution was applied and incubated with the sample for 2 min on the grid. Excess sample and stain were blotted away with Whatman filter paper. The staining process is repeated and the grid was dried until imaged. TEM micrographs were collected using Tecnai T12 TEM microscope at 100 KeV. The images were recorded on Gatan Oneview 4K×4K camera. Each image was collected by exposing the sample for 4 sec and a total of 100 dose fractionated images were collected and into a single micrograph. The data was collected at −1.5 to 2 microns under focus at 30K-40K magnification.

rAAV9-TRIM11 and rAAV9-GFP Production

To generate the plasmid pENN.AAV9.CB7.CI.hTRIM11-HA.WPRE.rBG (rAAV9-TRIM11) (in which TRIM11 expression is driven by CB7, a chicken β-actin promoter with cytomegalovirus enhancer elements), human TRIM11 tagged with a C-terminal HA was PCR amplified by using Phusion® High-Fidelity PCR Kit (NEB) and subsequently cloned into pENN.AAV9.CB7.CI. WPRE.RBG backbone at EcoRI and HindIII sites. rAAV9-GFP was constructed in the same vector. DNA plasmids were prepared by using endotoxin-free mega-prep kit (Qiagen) and characterized by structure and sequence analysis. AAV packaging was performed as described previously by M. Lock et al., Hum Gene Ther 21, 1259-1271, 2010.

### Stereotaxic Injection

8-week-old mice were anesthetized using ketamine hydrochloride (100 mg/kg, intraperitoneal [i/p]) and xylazine (10 mg/kg, i.p.). The Angle 2 stereotaxic instrument (Leica Biosystems) was used with a 10 mL Hamilton syringe for injection. pENN.AAV9.CB7.CI.hTRIM11-HA.WPRE.rBG (rAAV-TRIM11) or pENN.AAV9.CB7.CI.GFP.WPRE.rBG (rAAV-GFP) (~1.0×10¹¹ GC/mL) were injected into the left ventricle at the following stereotaxic coordinates in relation to Bregma (mm): AP-2.5 mm; ML, +2.0 mm; and DV, −1.8 mm. Animals were monitored regularly and provided supportive care for the first 10 days following stereotaxic injection until they fully recovered from surgery. Mice were sacrificed 3 months post injection by CO₂ until cessation of breathing. Mice brains were then collected and cut midsagittally. Left hemispheres were fixed in 4% PFA (24-48 h) and further prepared for histological analysis. Right hemispheres were snap frozen in liquid nitrogen and stored in-80 °C for subsequent protein analysis.

### Behavioral Measurements

Mice were analyzed for exploratory locomotor activity using the multiple unit open field maze (SD Instruments) consisting of four activity chambers with each chamber measuring 50 cm (length)×50 cm (width)×38 cm (height) made from white high density and non-porous plastic. Mice were placed in the center of the chamber at the start of the test. After a 2 min accustomization period, mice were monitored for total distance traveled, total movement time, and time spent in each of the designated quadrants of the chamber for 10 minutes using a video-camera coupled to automated tracking software (ANY-maze, SD Instruments).

To measure grip strength, mice were placed on a mesh wire and allowed to acclimatize for 30 seconds before the mesh was flipped. The wire was suspended 15 cm above an empty clean cage and the latency to fall was measured. The maximum exploration time was 3 min. The average latency to fall in 3 tests performed over 1 day was analyzed.

An accelerating Rotarod apparatus (47600, Ugo Basile, Italy) was used to measure motor coordination and balance. Only naïve animals were used. Mice was placed on the Rotarod with increasing speed, from 4-80 rpm, over 10 min. Their latency to fall off the Rotarod (in seconds) was recorded.

### Immunoblot Analysis of Mouse Brain and Spinal Cord

Dissected rodent brain tissues were lysed using modified radioimmunoprecipitation assay (RIPA) buffer (20 mM Tris-HCl, pH 8.0, 2 mM EDTA, 10 mM EGTA, 2 mM dithiothreitol (DTT), 1 mM phenylmethylsulfonyl fluoride (PMSF), 20 mM sodium orthovanadate, 1 mM sodium fluoride and protease and phosphatase inhibitor cocktail), as described previously (D. S. Harischandra et al., Sci Signal 12, 2019; S. Ghaisas et al., Neurotoxicology 75, 186-199, 2019). Tissues were lysed using a tissue homogenizer and incubated on ice for 20 minutes. Lysates were cleared by centrifugation at 13,000 rpm and 4 °C for 20 minutes. SN, PE, and WCL samples were analyzed by SDS-PAGE and immunoblotting.

### Immunohistochemistry of Mouse Brain and Spinal Cord

Dissected mouse spinal cord and brain tissues were fixed in 4% paraformaldehyde (PFA) for 24 hours. Tissues placed in cassettes and fixed for a further 24 hours. Thereafter they were washed twice first with PBS followed by 70% ethanol. Tissues were embedded and sectioned at 7 μm. Paraffin-embedded sections were deparaffinized in two washed of xylene followed by a series of decreasing alcohol concentrations. Heat-mediated antigen retrieval was performed using 10 mM citrate buffer (pH 6.0) for 30 min at 90 °C. Sections were then incubated with blocking reagent (5% normal goat serum, 2% bovine serum albumin and 0.5% Triton X-100 in PBS) for 60 min before being incubated with HA (1:500), GFP (1:1000), GFAP (1:1000), Iba1 (1:1000) or ChAT (1:500) primary antibodies for 16 h at 4 °C. Sections were then washed several times in PBS followed by 1-hour incubation with biotinylated anti-rabbit or mouse secondary antibody (Vector Laboratories, Burlingame, CA, BA-1000 or BA-9200, 1:500) followed by overnight incubation in ABC avidin-HRP conjugate (Vector Laboratories, PF-6100). HRP was detected with DAB staining kit (Vector laboratories, SK-4100). Tissues were counterstained with hematoxylin according to manufacturer's instructions (Vector laboratories, H-3404). Tissues were dehydrated and mounted in permount.

### Software

GraphPad Prism 7, ImageJ, ZEN lite: Carl Zeiss Microscopy.

### Quantification and Statistical Analysis

A two-tailed Student's t-test was used to evaluate the statistical significance in the mean value between two populations unless otherwise indicated. *P<0.05, **P<0.01, ***P<0.001.

The results of the experiments are now described.

### Effect of TRIM Proteins on Tau Aggregation

It has recently been found that a few TRIM proteins, including TRIM10, TRIM11, and TRIM55, can effectively clear tau aggregates. In the case of TRIM11, this effect is likely due to a combination of three activities: (1) molecular chaperone activity that prevents tau misfolding and aggregation, (2) disaggregase activity that dissolve pre-existing tau aggregates, and (3) degradative activity that promotes degradation of mutant tau associated with familial tauopathies and excess normal tau. The first two activities would keep tau in a soluble form, and are likely the main activities that protect against tauopathies in animal models where mutant tau is moderately overexpressed. Given the importance of the chaperone and disaggregase activities, all human TRIM proteins were systematically examined for their ability to prevent and/or reverse tau aggregation.

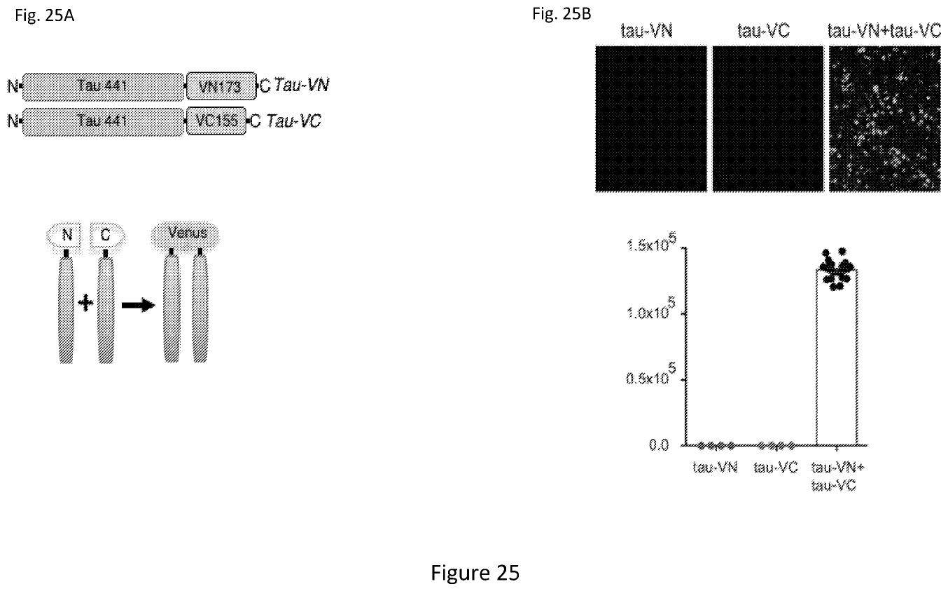

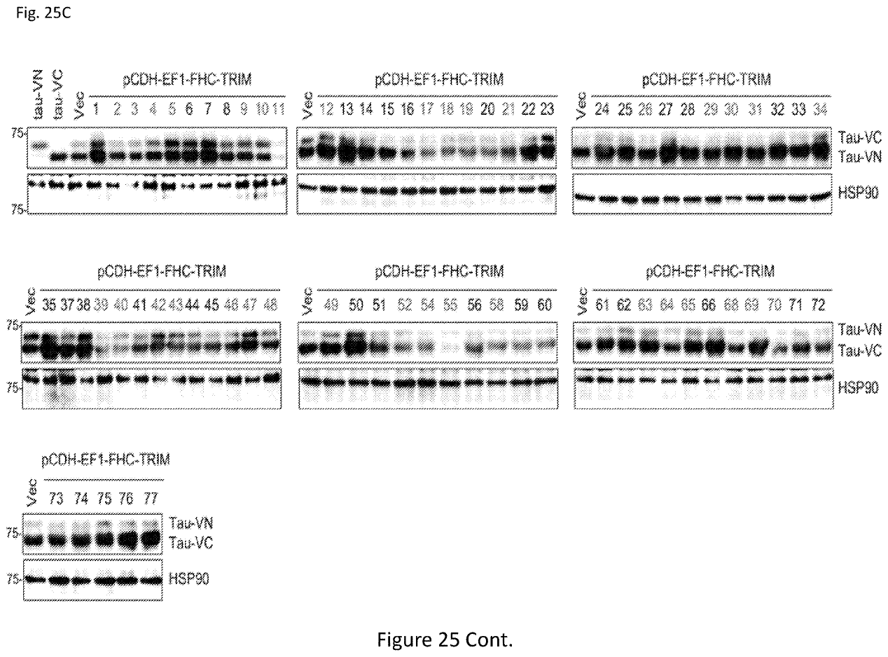

Aggregation of tau, like other disease-associated proteins, occurs through a rate-limiting nucleation step that involves its self-association (P. Friedhoff, M. von Bergen, E. M. Mandelkow, P. Davies, E. Mandelkow, Proc Natl Acad Sci USA 95, 15712-15717, 1998). To examine the effect of TRIM on tau self-association, a bimolecular fluorescence complementation (BiFC) assay, that is based on the yellow fluorescence protein variant Venus, was used (Y. J. Shyu, H. Liu, X. Deng, C. D. Hu, Biotechniques 40, 61-66, 2006). Full-length human tau (441 a.a.) was fused to the N-terminal fragment of Venus (1-172 a.a., VN) and the C-terminal fragment of Venus (155-238 a.a., VC), respectively, generating tau-VN and tau-VC (H. Tak et al., PLOS One 8, e81682 (2013) (FIG. 25). In cells that tau-VN and tau-VC were expressed together, but not individually, aggregation of tau brings the two fragments of Venus into close proximity to reconstitute the Venus fluorescence signal (FIG. 25B).

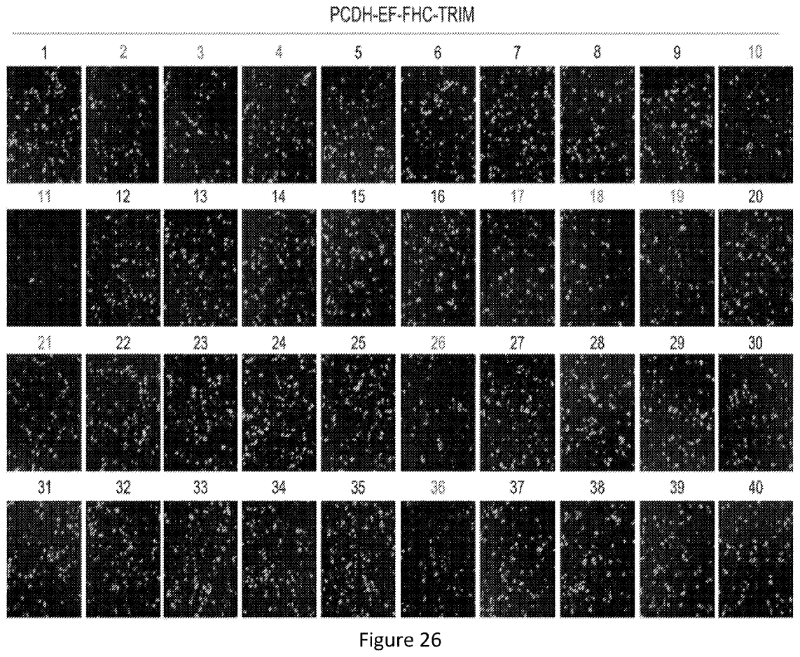

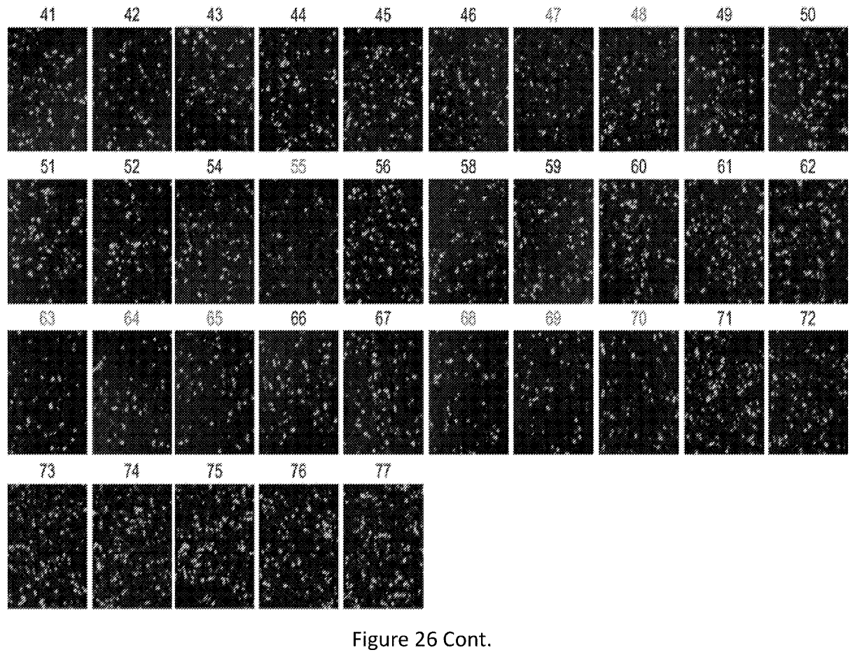

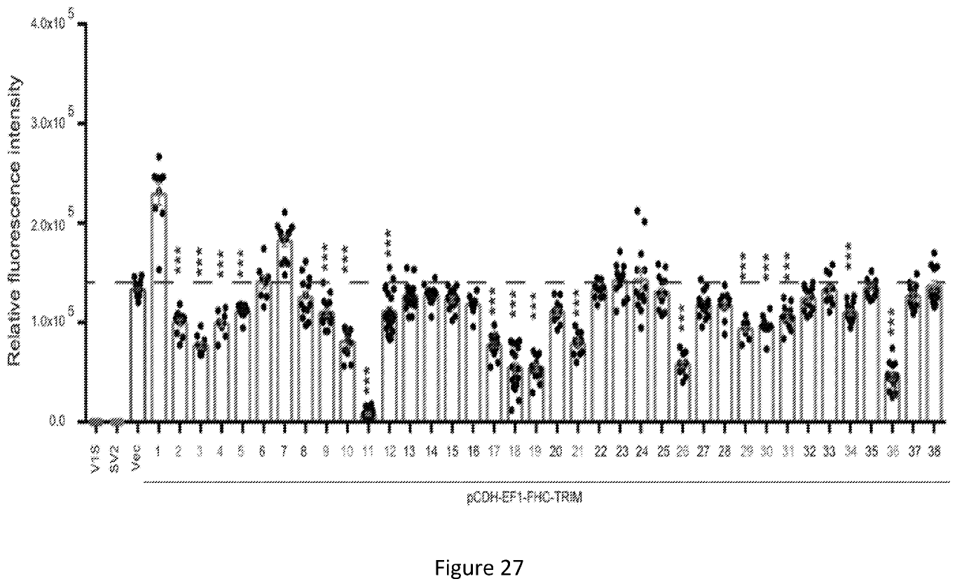

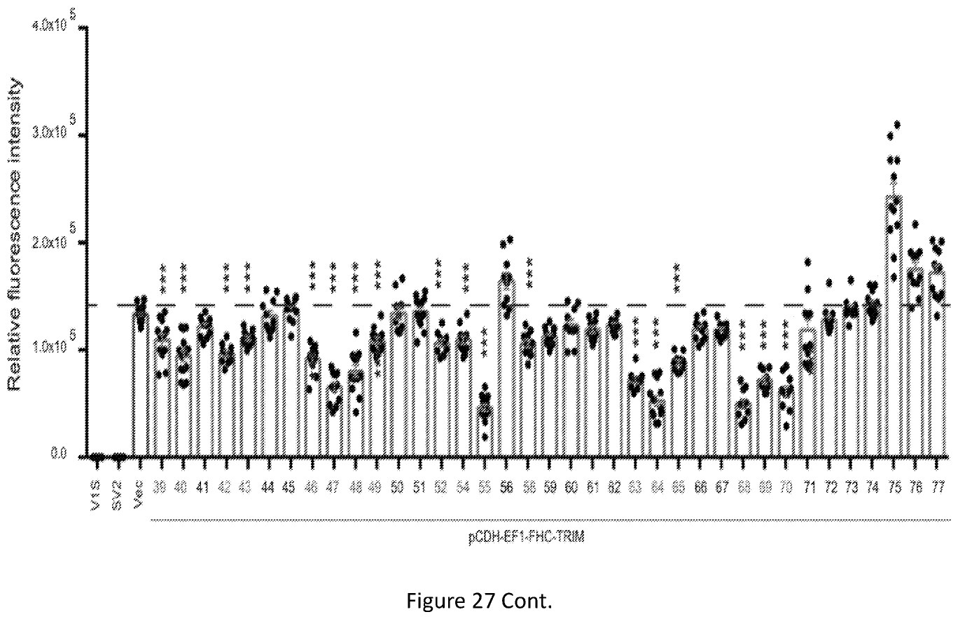

To systematically analyze TRIM proteins, 73 human TRIMs and two mouse TRIMs (TRIM12 and TRIM30) were individually cloned into a mammalian expression plasmid, pCDH-EF1-FHC, in which they were fused with an FLAG tag and an HA tag at the C-terminus. Each TRIM plasmid was co-transfected with tau-VN173 and tau-VC155 in HEK293T cells, and levels of Venus signal from three independent experiments were quantified. Interestingly, nearly one half of these TRIM proteins (~36) noticeably reduced the Venus fluorescent signal in cells expressing tau-VN and tau-VC. These TRIMs included TRIM2, −3, −4, −5, −9, −10, −11, −12, −17, −18, −19, −21, −26, −29, −30, −31, −34, −36, −39, −40, −42, −43, −46, −47, −48, −49, −52, −54, −55, −58, −63, −64, −65, −68, −69 and −70 (FIG. 26 and FIG. 27). This result suggests that the ability to prevent and/or reverse tau aggregation is prevalent among TRIM proteins.

### Effect of TRIM Proteins on α-Synuclein Aggregation

Misfolding and aggregation of α-synuclein (α-Syn) are associated with a group of movement disorders and dementias, collectively referred to as synucleinopathies (J. E. Galvin, V. M. Lee, J. Q. Trojanowski, Arch Neurol 58, 186-190, 2001; M. Goedert, M. G. Spillantini, K. Del Tredici, H. Braak, Nat Rev Neurol 9, 13-24, 2013; M. Goedert, R. Jakes, M. G. Spillantini, J Parkinsons Dis 7, S51-S69, 2017). The main types of synucleinopathies are PD, dementia with Lewy bodies (DLB), and multiple system atrophy (MSA). In addition, various neuroaxonal dystrophies and a small fraction of sporadic AD have α-synuclein pathologies. Among these diseases, PD is the most common neurodegenerative movement disorder, characterized by progressive degeneration of dopaminergic neurons that follows the appearance of α-Syn pathology (W. Poewe et al., Nat Rev Dis Primers 3, 17013, 2017; D. J. Surmeier, J. A. Obeso, G. M. Halliday, Nat Rev Neurosci 18, 101-113, 2017). α-Syn is an intrinsically unstructured protein, which is abundantly expressed in neurons and is normally soluble. In synucleinopathies, however, α-Syn forms fibrillar aggregates in the forms of Lewy bodies (LBs) and/or Lewy neurites (LNs) (W. Poewe et al., Nat Rev Dis Primers 3, 17013, 2017; D. J. Surmeier, J. A. Obeso, G. M. Halliday, Nat Rev Neurosci 18, 101-113, 2017). Moreover, missense mutations or multiplications of the α-Syn gene (SNCA), both of which increase the propensity of α-Syn to aggregate, cause a familial subset of PD (W. Poewe et al., Nat Rev Dis Primers 3, 17013, 2017).

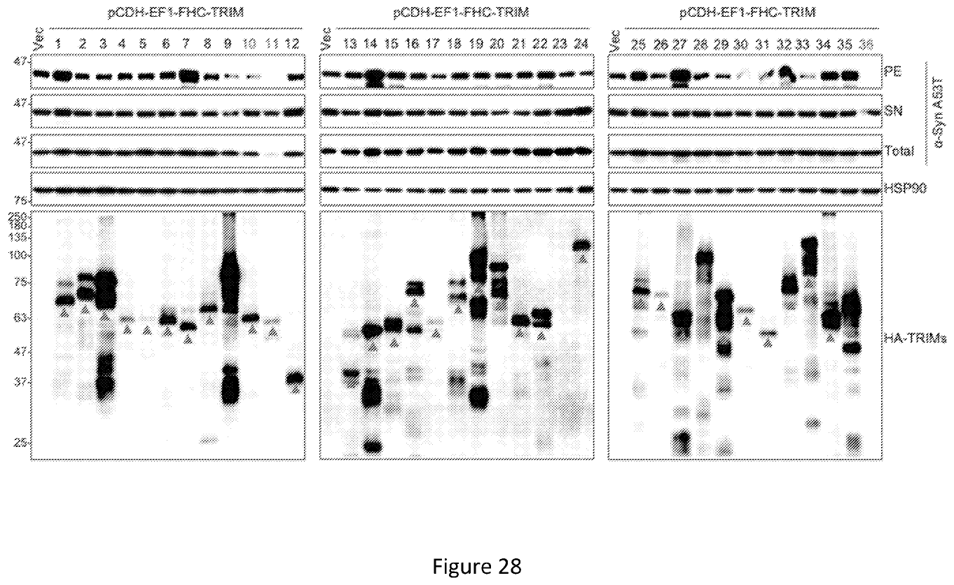

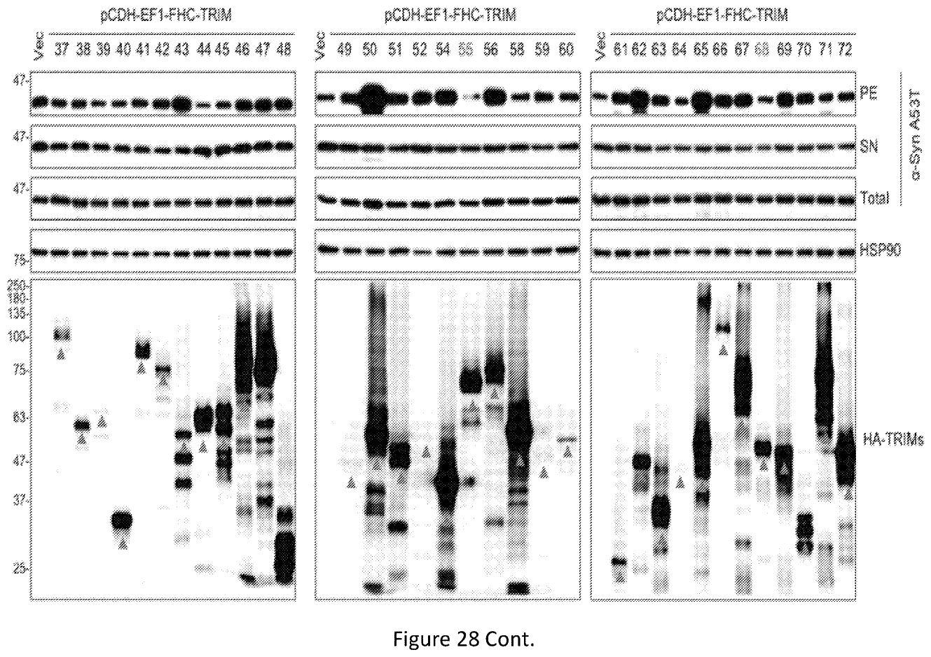

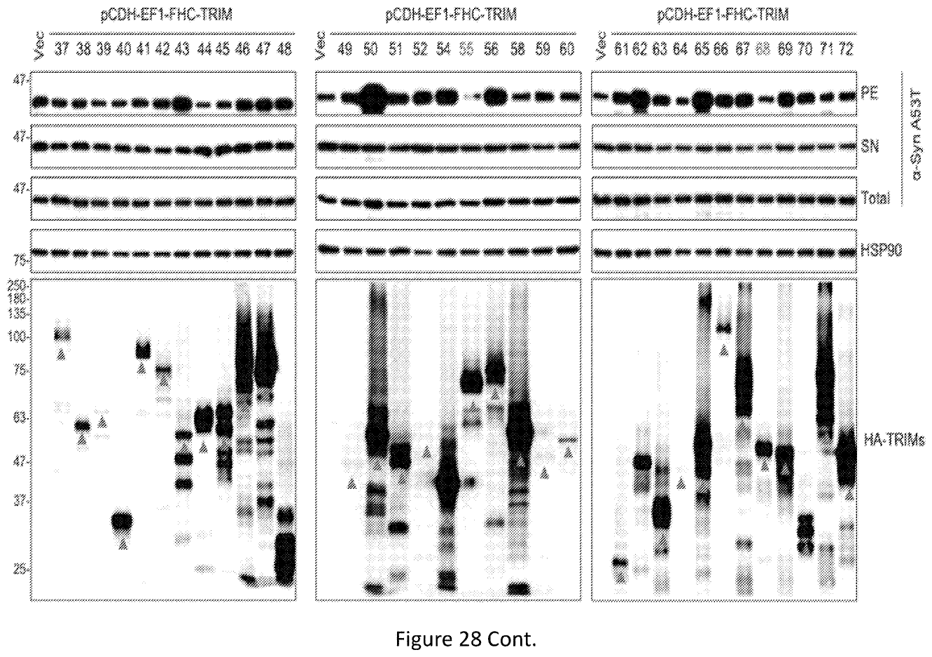

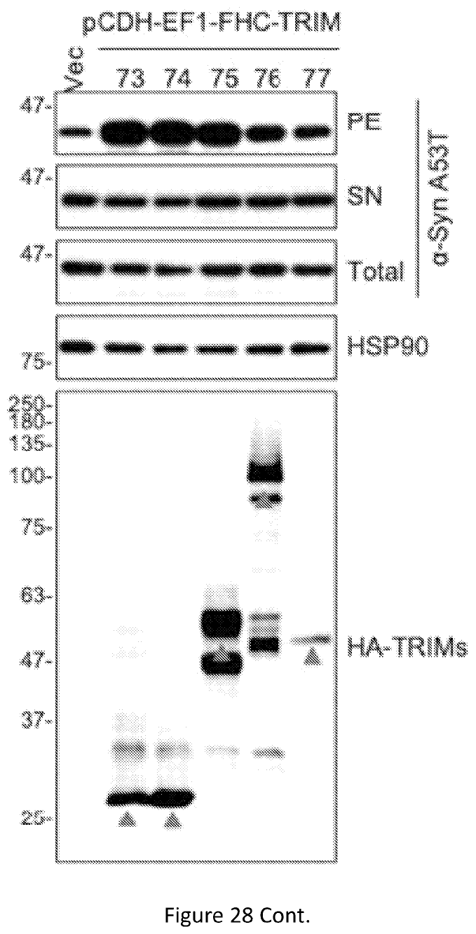

To systemically analyze the role of TRIM protein in protecting against synucleinopathies, each of the 75 mammalian TRIMs were expressed in HEK293T cells together with a green fluorescence protein (GFP) fusion of α-Syn A53T, a mutant linked to familial PD. When expressed alone, α-Syn A53T formed aggregates that were insoluble in non-ionic detergent. Upon co-expression, several TRIMs displayed a strong activity to reduce α-Syn A53T aggregates. These TRIMs included TRIM10, TRIM11, TRIM36, TRIM55, and TRIM68 (FIG. 28).

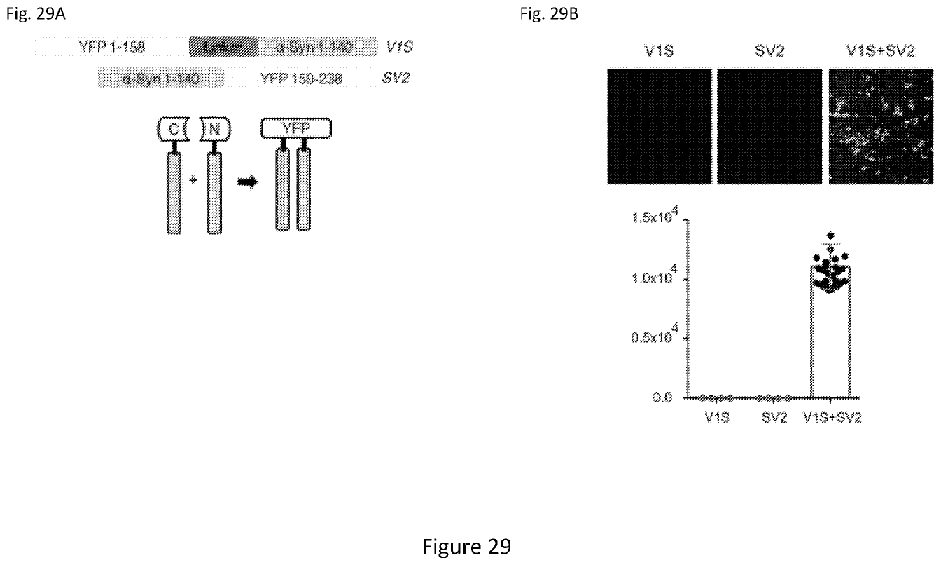

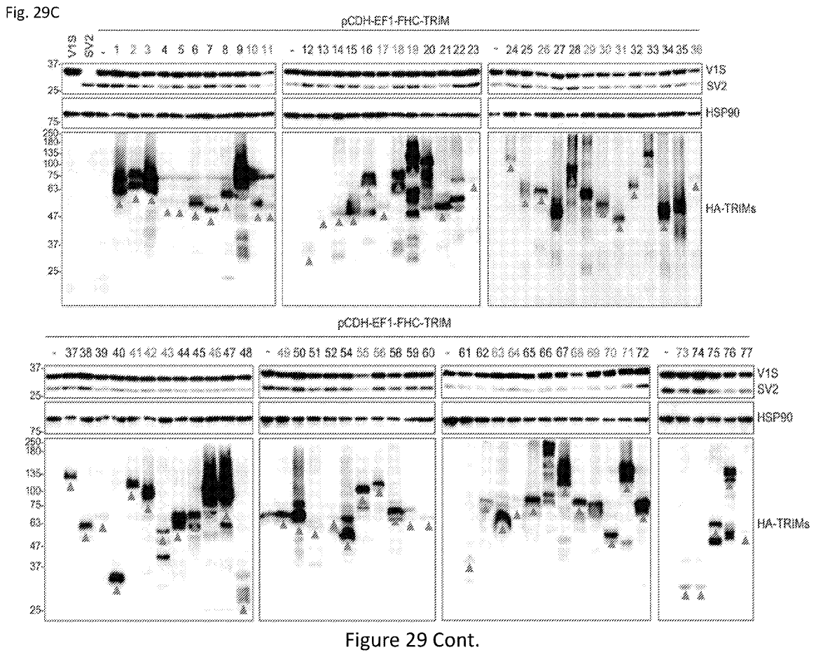

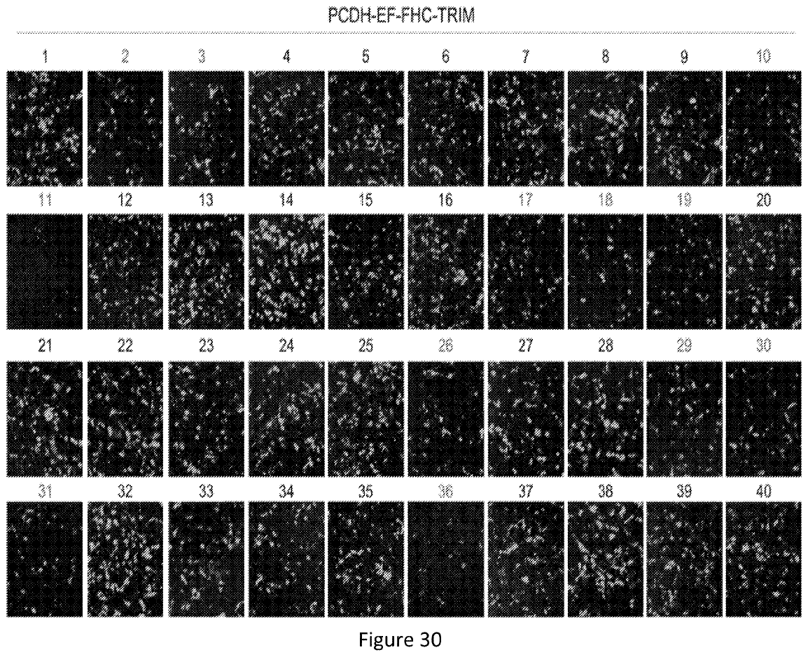

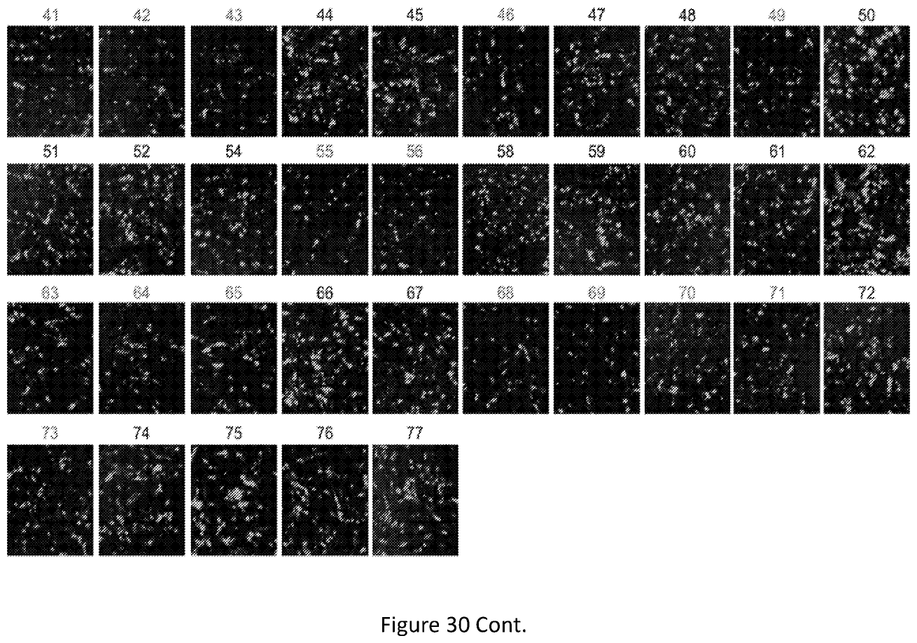

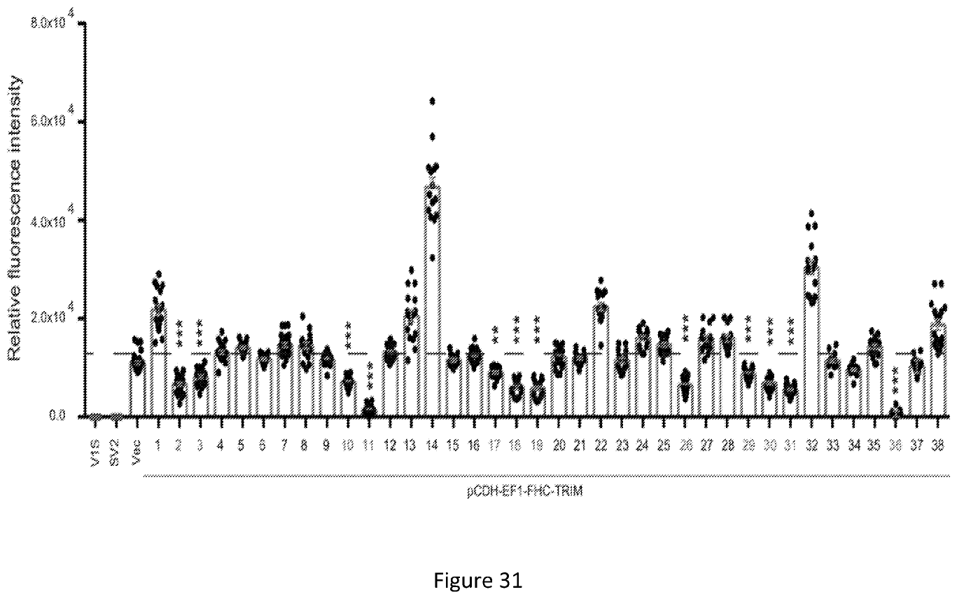

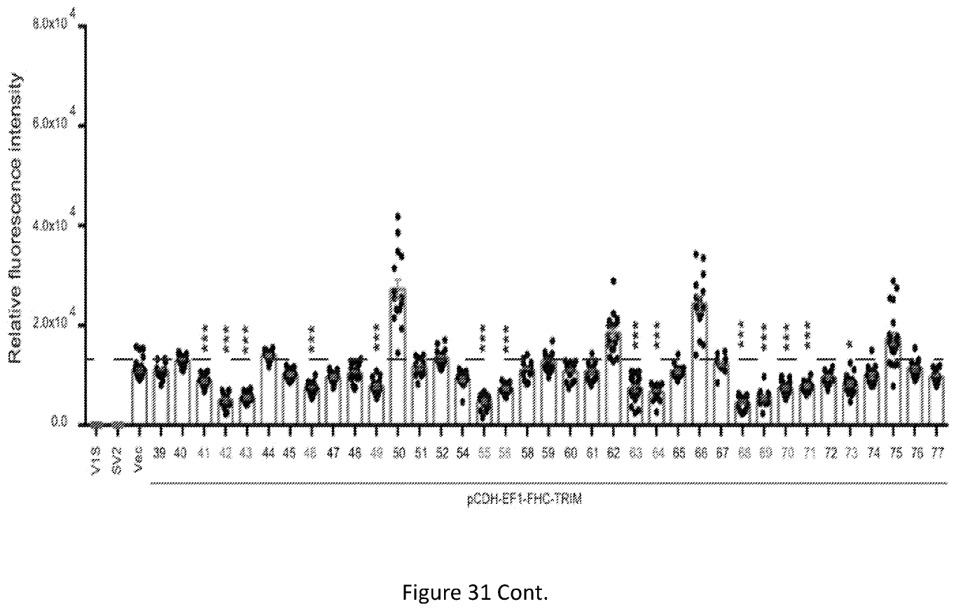

To investigate which TRIM proteins can prevent the self-association of α-Syn, a BiFC system was used, in which α-Syn was fused to the N-terminal (V1) or the C-terminal (V2) fragment of the Venus protein, generating V1-α-Syn (V1S) and α-Syn-V2 (SV2), respectively (FIG. 5A) (T. F. Outeiro et al., PLOS One 3, e1867, 2008). When VIS and SV2 were expressed together, but not individually, reconstitution of the Venus fluorescence occurred (FIG. 29B), reflecting α-Syn oligomerization that brought the split Venus moieties into proximity. When co-expressed with VIS and SV2, the five TRIMs mentioned above-TRIM10, TRIM11, TRIM36, TRIM55, and TRIM68—also reduced levels of VIS and SV2, confirming their ability to clear α-Syn (FIG. 29C). Moreover, about twenty-six TRIM proteins can reduce the Venus fluorescence signal. These TRIMs included TRIM2, −3, −10, −11, −17, −18, −19, −26, −29, −30, −31, −36, −41, −42, −43, −46, −49, −55, −56, −63, −64, −68, −69, −70, −71, and −73 (FIG. 30 and FIG. 31).

### Effect of TRIM Proteins on SOD1

ALS is a fatal neurodegenerative disease of motor neurons, characterized by weakness of limb, respiratory, and bulbar muscles (J. P. Taylor, R. H. Brown, Jr., D. W. Cleveland, Nature 539, 197-206, 2016; R. H. Brown, A. Al-Chalabi, N Engl J Med 377, 162-172, 2017). The disease onset begins focally and spreads relentlessly, and most patients die within 2-5 years. Approximately 10% of ALS are familial cases caused by germline mutations, while the remaining ~90% ALS cases are sporadic with no known family history of the disease. Nevertheless, familial ALS cases share a high similarity in pathological and clinical features among themselves, as well as with sporadic ALS cases, suggesting common underlying molecular and cellular events. Like most other neurodegenerative diseases, ALS is a disorder of protein misfolding and aggregation, with ubiquitin-containing cytoplasmic and/or nuclear inclusions as pathological hallmarks. For familial ALS, the aggregates often contain proteins encoded by mutant genes. Among them, mutated Cu,Zn superoxide dismutase 1 (SOD1) was the first ALS gene to be identified and is one of the most common familial ALS gene, associated with ~20% of all familial cases (D. R. Rosen et al., Nature 362, 59-62, 1993).

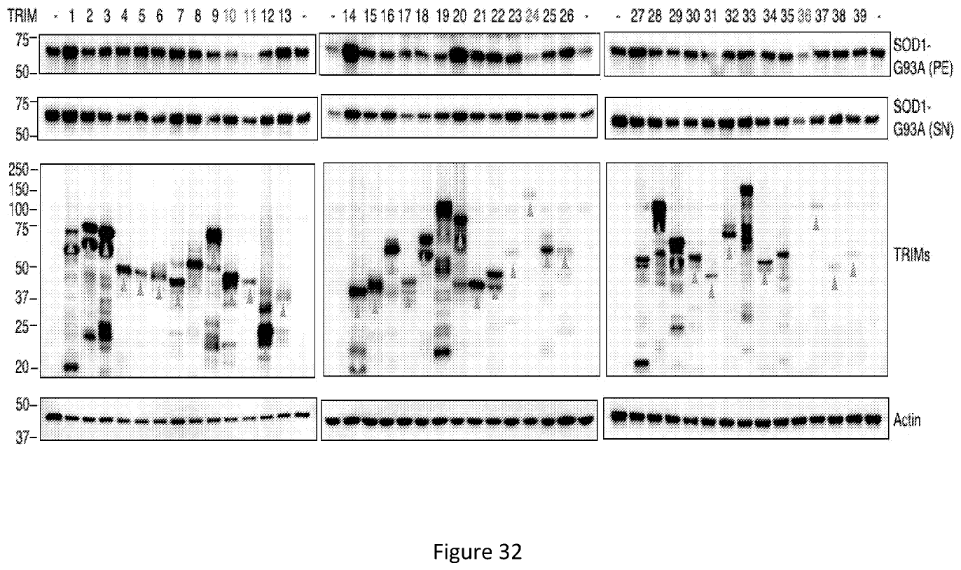

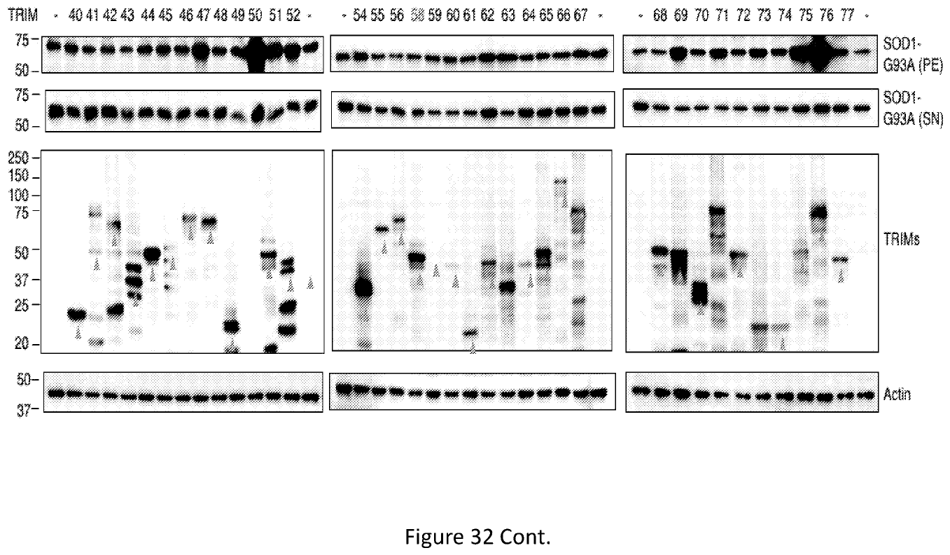

To systematically evaluate the effect of TRIMs on SOD1, each of the 75 mammalian TRIM proteins were co-expressed with a GFP fusion of SOD1 G93A, a human mutant associated with familial ALS. Five human TRIMs—TRIM10, TRIM11, TRIM24, TRIM36, and TRIM58—were capable of clearing SOD1 G93A-GFP aggregates (FIG. 32).

### TRIM11 Accelerates Degradation of SOD1

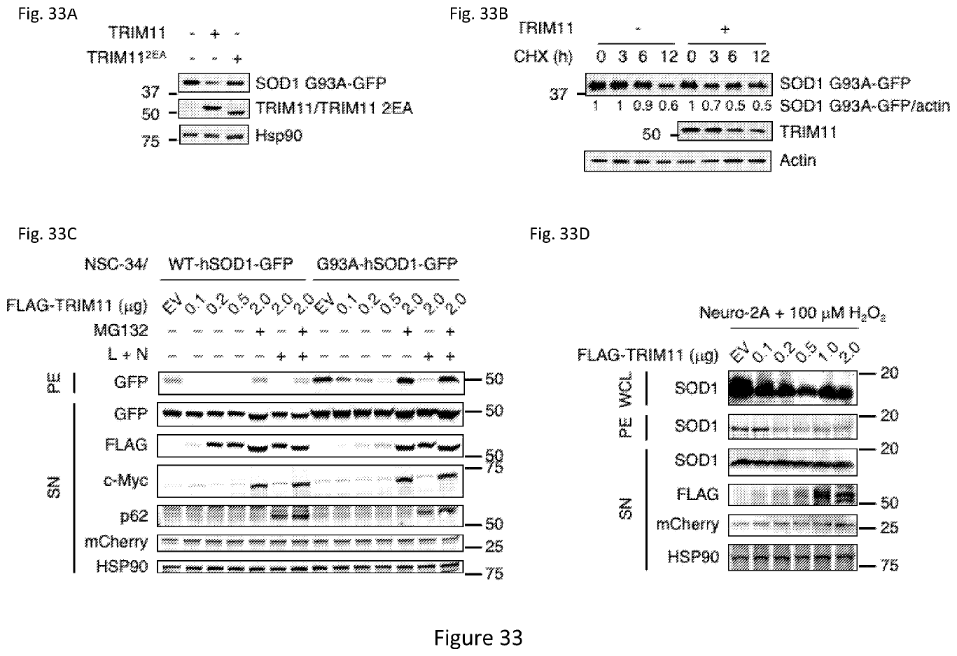

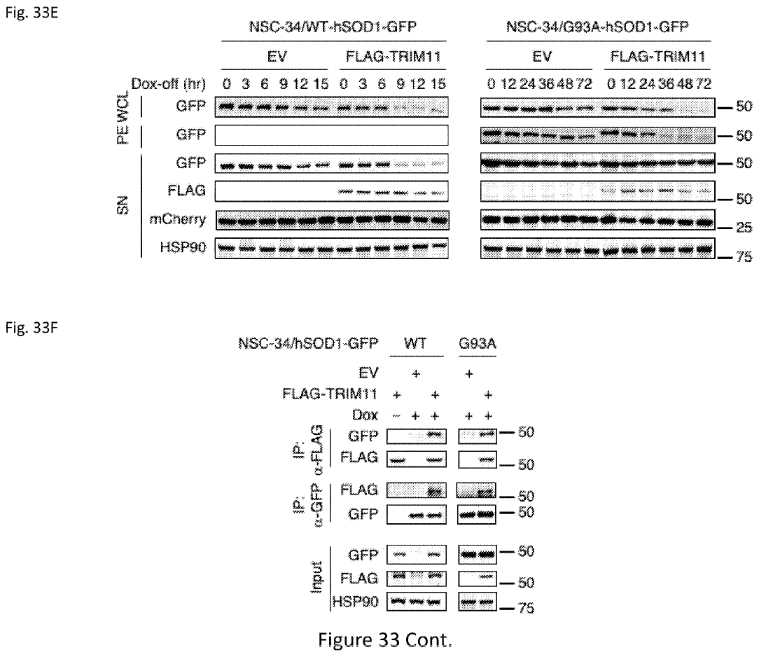

The results described above show that at least several TRIMs, including TRIM11, protect against aggregations of ALS-associated SOD1. To extend this analysis, additional experiments were performed to evaluate the effect of TRIM11 on SOD1. It was previously shown that TRIM proteins possess SUMO E3 ligase activity (L. Guo et al., Mol Cell 55, 15-30, 2014; G. Zhu et al., Cell Rep 33, 108418, 2020; Y. Chu, X. Yang, Oncogene 30, 1108-1116, 2011). Moreover, TRIM11 and TRIM19 (a.k.a. PML) promotes SUMOylation of misfolded proteins, which allows these proteins to be ubiquitinated by SUMO-targeted ubiquitin ligases (STUbLs) and their subsequent degradation in the proteasome (L. Guo et al., Mol Cell 55, 15-30, 2014; G. Zhu et al., Cell Rep 33, 108418, 2020). To evaluate whether TRIM11 reduces levels of SOD1 G93A via its SUMO E3 activity, TRIM11^{2EA} was used, which is defective in SUMO E3 ligase activity due to the replacement of the two conserved Glu residues proximal to the RING domain with Ala) (G. Zhu et al., Cell Rep 33, 108418, 2020). In contrast to TRIM11, TRIM11^{2EA} exhibited minimal activity in reducing GFP-SOD1 G93A in HEK293T cells (FIG. 33A). To verify that TRIM11 promotes the degradation of SOD1 G93A, a cycloheximide (CHX) chase experiment was performed. When co-expressed in HEK293T cells, TRIM11 accelerated the turnover of SOD1 G93A (FIG. 33B), indicating that TRIM11 targets SOD1 G93A for degradation.

To evaluate the effect of TRIM11 on SOD1 in neural cells, mouse NSC-34 cells were used, which exhibit many motor neuron-like properties. NSC-34 cells that express GFP-SOD1 or GFP-SOD1 G93A were used in a doxycycline (Dox)-inducible manner, which are referred to as NSC-34/WT-hSOD1-GFP and NSC-34/G93A-hSOD1-GFP cells, respectively (Y. Mali, N. Zisapels, Neurobiol Dis 32, 133-141, 2008). Introducing TRIM11 into these cells reduced levels of WT-hSOD1-GFP and G93A-hSOD1-GFP, especially the insoluble species, in a dose-dependent manner (FIG. 33C). In mouse neural crest-derived cell line Neuro-2a (N2a), treatment with H₂O₂ resulted in aggregation of endogenous SOD1. Under this condition, TRIM11 still reduced levels of SOD1 protein, especially the insoluble form (FIG. 33D).

The effect of TRIM11 on SOD1 was blocked by the proteasome inhibitor MG132, but not the lysosomal inhibitors leupeptin plus NH₄Cl (L+N) (FIG. 33C). Moreover, TRIM11 accelerated the degradation of WT-hSOD1-GFP and G93A-hSOD1-GFP in NSC-34 cells (FIG. 33E). Reciprocal co-immunoprecipitation assays showed that TRIM11 interacted with both SOD1 and SOD1 G93A in NSC-34 cells (FIG. 33F). Together, these results indicate that TRIM11 interacts with SOD1 and promotes proteasomal degradation of mutant, as well as excess normal, SOD1 protein.

### TRIM11 Ameliorates Protein Aggregation and Neuroinflammation in SOD1-G93A Mice

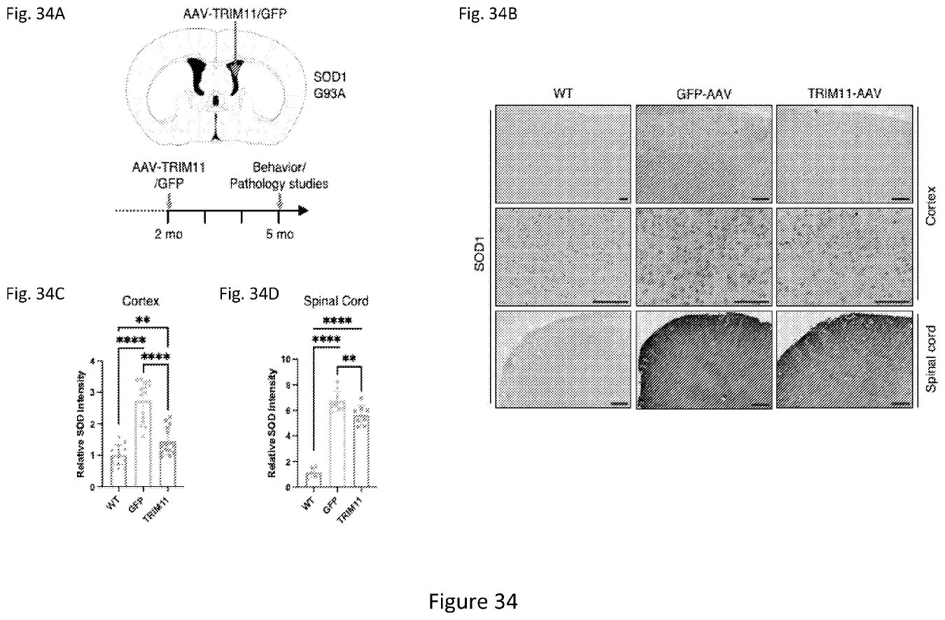

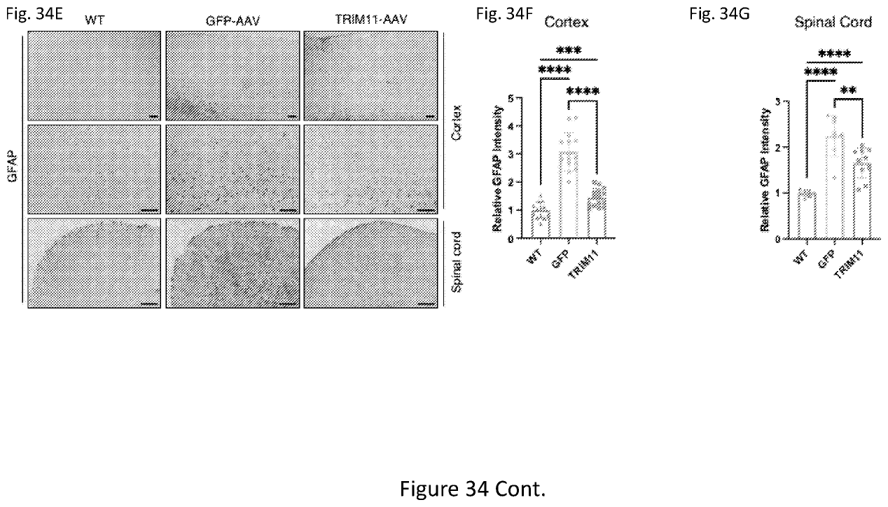

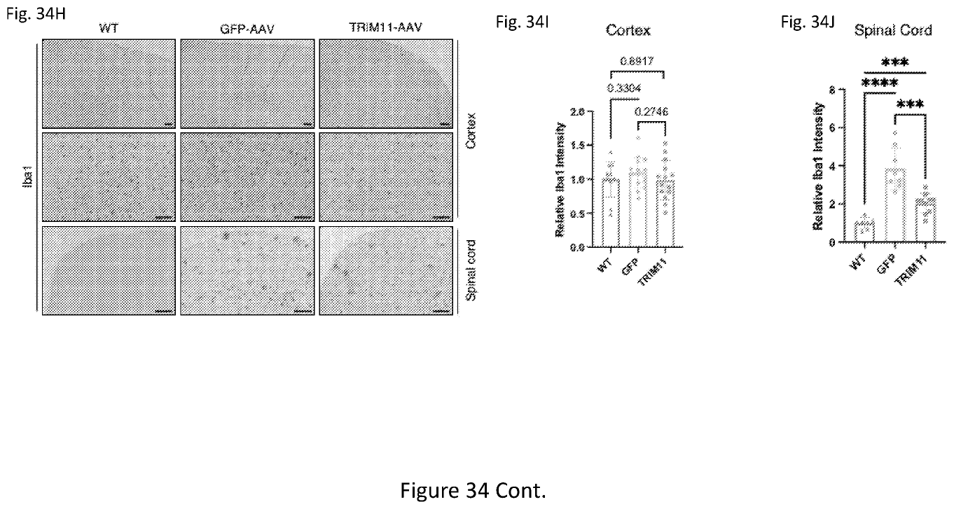

Next, the protective effect of TRIM11 was evaluated in a well-established mouse model of familial ALS that expresses a G93A mutant form of human SOD1. This model reproduces most of the clinical and neuropathological features of ALS (M. E. Gurney et al., Science 264, 1772-1775, 1994). To introduce TRIM11 in this mouse model, human TRIM11 and, as a control, GFP were cloned into adeno-associated virus serotype 9 (AAV9), which has high specificity for neurons and is used in the FDA-approved treatment for spinal muscular atrophy (K. D. Foust et al., Nat Biotechnol 27, 59-65, 2009; K. D. Foust et al., Nat Biotechnol 28, 271-274, 2010). AAV9-TRIM11 or AAV9-GFP vectors were delivered to the SOD1 G93A transgenic mice at 2 months of age via intracerebroventricular (ICV) injection, to achieve a broad distribution in the brain, and brain pathology and animal behaviors analyzed at ~5 months of age (FIG. 34A).

As expected, brains of AAV9-GFP-injected mice accumulated high levels of SOD1 G93A in various regions, including the cortex and the spinal cord (FIG. 34B to FIG. 34D). In comparison, brains of AAV9-TRIM11-injected mice contained substantially less SOD1 G93A, with ~55% reduction in the cortex (to a level close to those in the wild-type mice) (FIG. 34B and FIG. 34C) and ~16% reduction in the spinal cord (FIG. 34B and FIG. 34D).

SOD1-G93A transgenic mice manifest astrogliosis and microgliosis (M. E. Gurney et al., Science 264, 1772-1775, 1994). As shown by immunohistochemistry (IHC), AAV9-GFP-injected mice showed strong immunoreactivity of glial fibrillary acidic protein (GFAP) in the cortex and spinal cord, indicative of astrocyte activation (FIG. 30E through FIG. 6G). In contrast, AAV9-TRIM11-injected brains displayed less GFAP immunoreactivity in the cortex (reduced by ~50%, to a level observed in brains of age-matched wild-type littermates) and the spinal cord (reduced by ~25%) (FIG. 30E to FIG. 30G). AAV9-GFP-injected brains also showed activation of microglia in the spinal cord, but not the cortex, as indicated by increased immunoreactivity to ionized calcium-binding adaptor molecule 1 (Iba1) (FIG. 34H through FIG. 34J). In comparison, AAV9-TRIM11-injected brains showed a ~50% reduction of microgliosis in the spinal cord (FIG. 34H through FIG. 34J).

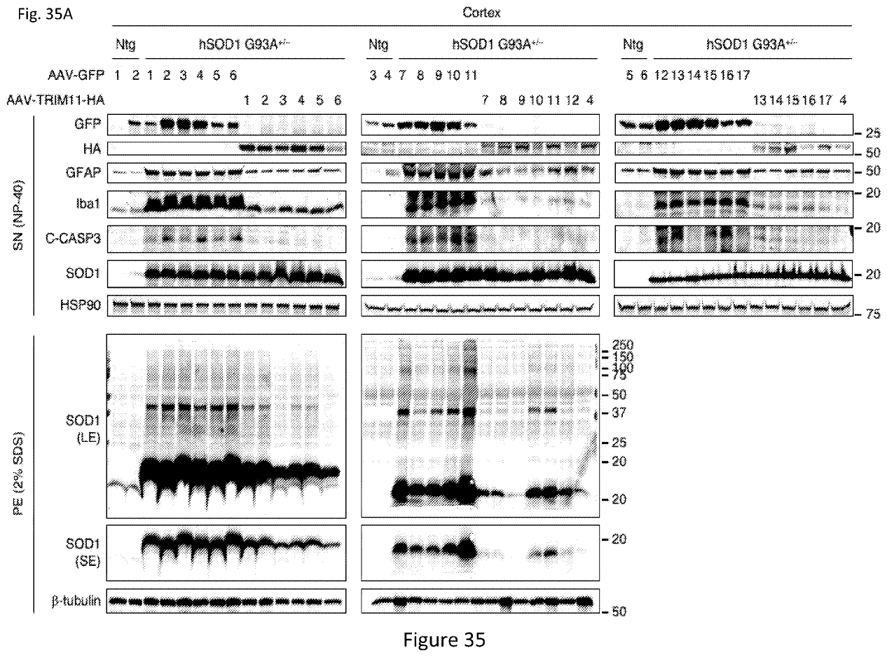

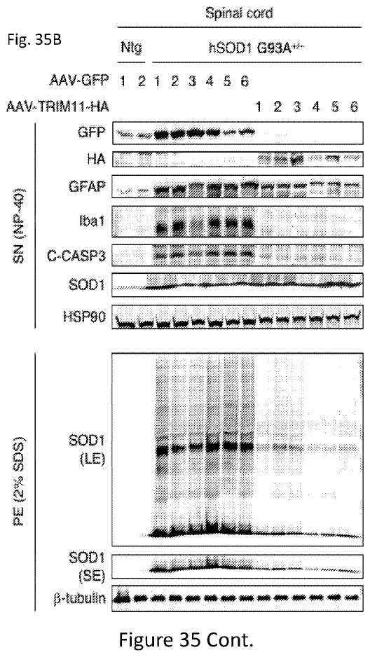

To verify TRIM11-mediated reduction in SOD1-G93A levels and neuroinflammation, immunoblot analysis of brain tissues were performed. An analysis of the cortex of seventeen mice from AAV9-GFP and AAV9-TRIM11 groups each showed that TRIM11 markedly reduced levels of SOD1 G93A protein that was insoluble in NP-40-containing buffer, while having a minimal effect on levels of soluble SOD1 G93A protein (FIG. 35A). TRIM11 also reduced GFAP and Iba1 to levels that were close to those seen in wild-type mice, indicating that TRIM11 strongly prevents neuroinflammation (FIG. 11A). Moreover, activation of caspase-3 was much reduced in AAV-TRIM11-injected mice compared to AAV-GFP-injected mice (FIG. 35A). Analysis of the spinal cord of six mice from AAV9-GFP and AAV9-TRIM11 groups each yielded similar results. Thus, AAV9-TRIM11 markedly reduced levels of SOD1 G93A, GFAP, Iba1, and activated caspase-3 in the SOD1-G93A mice (FIG. 35B). Collectively, these results indicate that TRIM11 reduces aggregated protein, neuroinflammation, and neuronal death associated with SOD1-G93A in mammals.

### TRIM11 Improves Motor Performance of SOD1-G93A Transgenic Mice

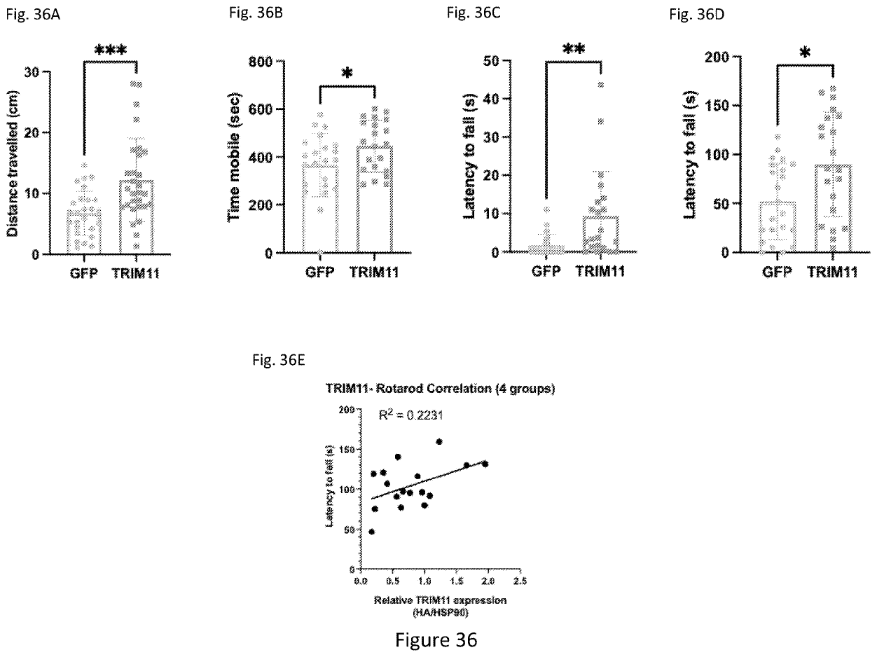

Reflecting the degeneration of motor neurons, ALS is characterized by a progressive muscular paralysis (J. P. Taylor, R. H. Brown, Jr., D. W. Cleveland, Nature 539, 197-206, 2016; R. H. Brown, A. Al-Chalabi, N Engl J Med 377, 162-172, 2017). To evaluate the effect of TRIM11 on motor functions of SOD1-G93A mice, open field, wire hanging, and rotarod tests were performed. In the open field test, the TRIM11 group of mice stayed mobile for more time (~20% increase) and travelled a longer distance (~80% increase) compared to the GFP group of mice (FIG. 36A and FIG. 36B). In a wire hang test, the TRIM11 group of mice performed substantially better than the GFP group of mice, and the latency to fall was ~9 second for the TRIM11 group compared to ~1.7 second for the GFP group (FIG. 36C). In a rotating rod test with increasing speed (from 4-80 rpm over 10 min), the TRIM11 group of mice also showed a prolonged latency to fall (~90 sec) compared to the GFP group of mice (~50 seconds) (FIG. 36D). Moreover, among the seventeen animals in the TRIM11 group, there was a positive correlation between the latency to fall in the rotarod test and the expression of the TRIM11 transgene (FIG. 36E). These results show that intracranial delivery of AAV9-TRIM11 improves motor functions of SOD1-G93A mice.

### Effect of TRIM Proteins on TDP-43 Aggregation

In the vast majority of sporadic ALS cases, TAR DNA-binding protein 43 (TDP-43), a normally nuclear protein, mis-localizes to the cytoplasm and aggregates (M. Neumann et al., Science 314, 130-133, 2006). Aggregation of TDP-43 is also linked to an increasing number of other neurodegenerative diseases collectively referred to as TDP-43 proteinopathies. These diseases include frontotemporal lobar degeneration (FTLD-TDP), multiple system proteinopathy (MSP), Perry disease, facial onset sensory and motor neuronopathy (FOSMN), cerebral age-related TDP-43 with sclerosis (CARTS), limbic-predominant age-related TDP-43 encephalopathy (LATE), sporadic inclusion body myositis (sIBM), and a subset of AD (E. M. J. de Boer et al., J Neurol Neurosurg Psychiatry 92, 86-95, 2020). Moreover, missense mutations in the TARDBP gene, which encodes TDP-43, were identified in a small percentage of familial ALS and FTLD cases.

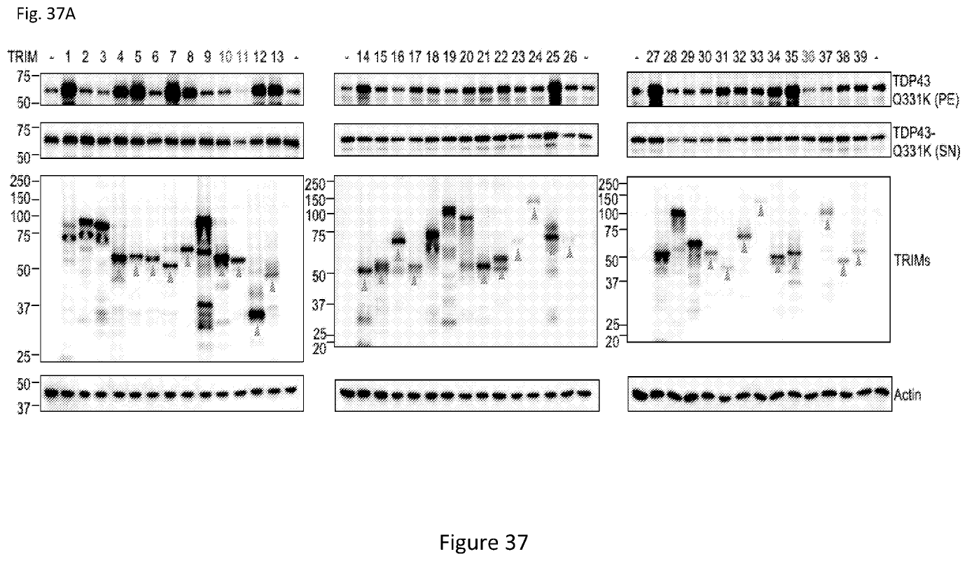

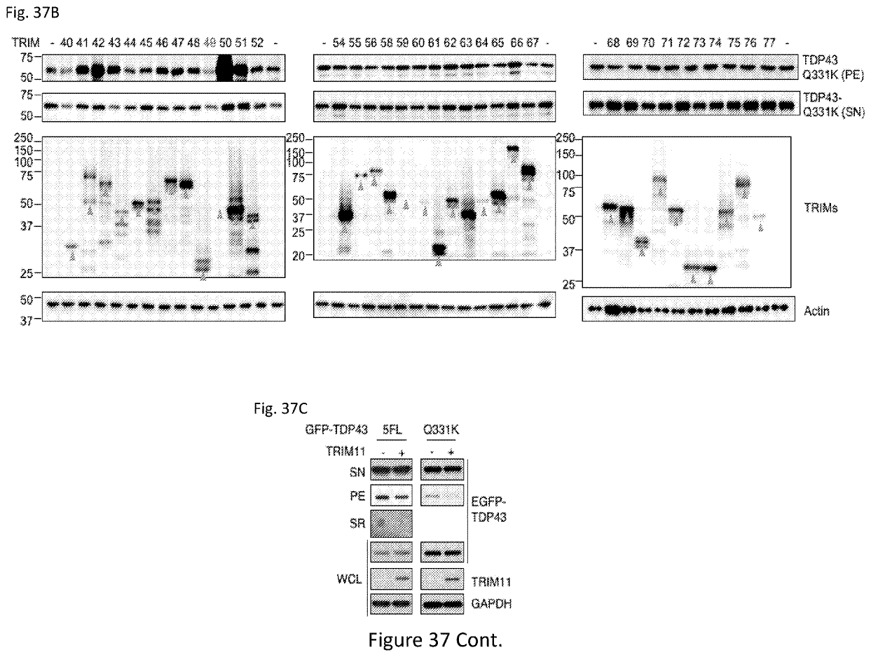

To systematically analyze the effect of TRIMs on TDP-43, a GFP fusion of TDP-43 Q331K, a mutant found in familial TDP-43 proteinopathies, was used. Each TRIM was co-expressed with TDP-43 Q331K-GFP in HEK293T cells. Six TRIMs displayed noticeable activity in removing TDP-43 Q331K-GFP aggregates, including TRIM10, −11, −36, −37, −40, and −49 (FIG. 37A and FIG. 37B).

### TRIM11 Protects Against Aggregation of TDP-43 in Cultured Cells

To investigate the effect of TRIM11 on TDP-43, cell lines stably expressing GFP fusion of TDP-43 5FL or TDP-43 Q331K were established, both of which are found in ALS patients and are defective in RNA binding (J. P. Taylor, R. H. Brown, Jr., D. W. Cleveland, Nature 539, 197-206, 2016; R. H. Brown, A. Al-Chalabi, N Engl J Med 377, 162-172, 2017). These mutants generate NP-40 insoluble aggregates that soluble in (PE) or resistant to (SR) SDS. Forced expression of TRIM11 resulted in a reduction in PE and SR aggregates of TDP-43 mutants, but not the NP-40 soluble (NS) TDP-43 (FIG. 37C). Therefore, TRIM11 reduces the levels of ALS-linked TDP-43 mutants.

### TRIM11 Ameliorates Protein Aggregation and Neuroinflammation in TDP-43 Transgenic Mice

To evaluate the protective effect of TRIM11 on TDP-43 aggregation in mammalian brains, the TAR4 transgenic mouse line was used, in which the human TDP-43-encoding gene TARDBP is preferentially expressed in neurons from the murine Thy-1 promoter (H. Wils et al., Proc Natl Acad Sci USA 107, 3858-3863, 2010). Mice in the TAR4 line develop phenotypes similar to ALS, including severe motor impairment, loss of upper and lower motor neurons, and premature death, in a dose-dependent manner. Homozygous mice (referred to as TAR4/4) manifest ALS-like symptoms very early and do not survive beyond one month of age, while hemizygous mice (referred to as TAR4) exhibit ALS-like symptoms much later and milder (H. Wils et al., Proc Natl Acad Sci USA 107, 3858-3863, 2010)

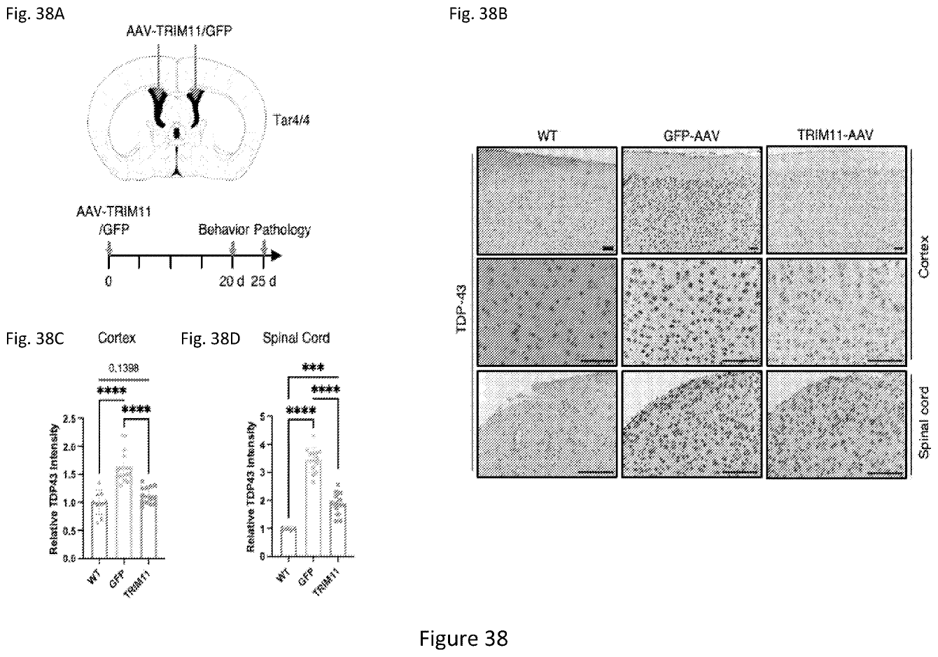

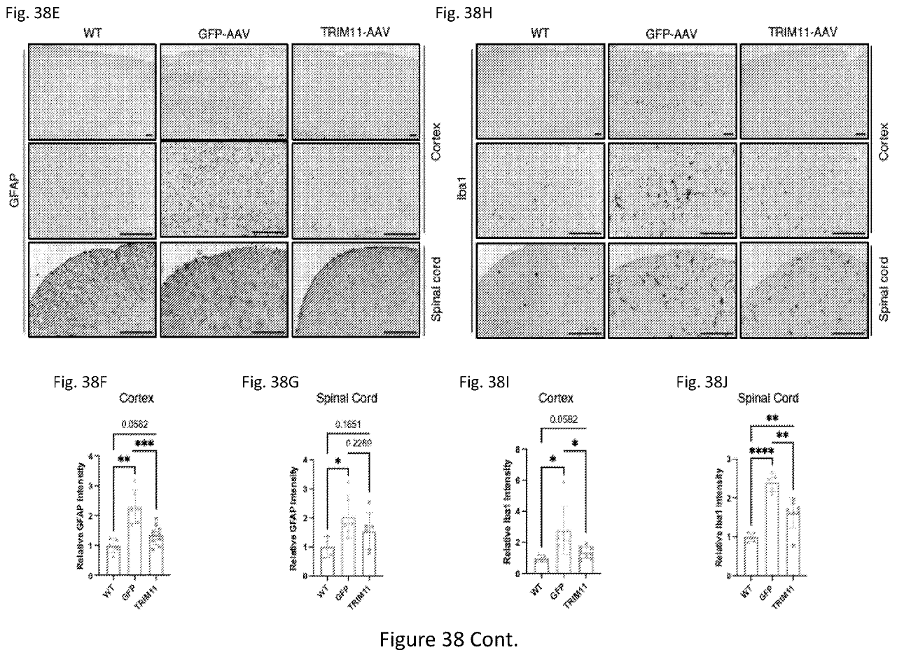

AAV9-GFP and AAV9-TRIM11 were delivered into newborn TAR4/4, and analyzed behavior and pathology at 20- and 25-days post-injection, respectively (FIG. 38A). As shown by IHC, brains of AAV9-TRIM11-injected animal contained much less TDP-43 immunoreactivity in the cortex (~30% reduction, which was similar to that observed in the wild-type littermates) and the spinal cord (~45% reduction), compared to those of AAV9-GFP-injected animals (FIG. 38B through FIG. 38D).

TAR4/4 mice develop astrogliosis and microgliosis in brain and spinal cord areas affected by TDP-43 pathology. Compared to that of the AAV9-GFP group, GFAP immunoreactivity was reduced in the AAV9-TRIM11 group by ~40% in the cortex and ~25% in the spinal cord (FIG. 38E through FIG. 38G). Moreover, Iba1 immunoreactivity was reduced by ~50% in the cortex and ~30% in the spinal cord in the AAV9-TRIM11 group compared to the AAV9-GFP group (FIG. 38H through FIG. 38J).

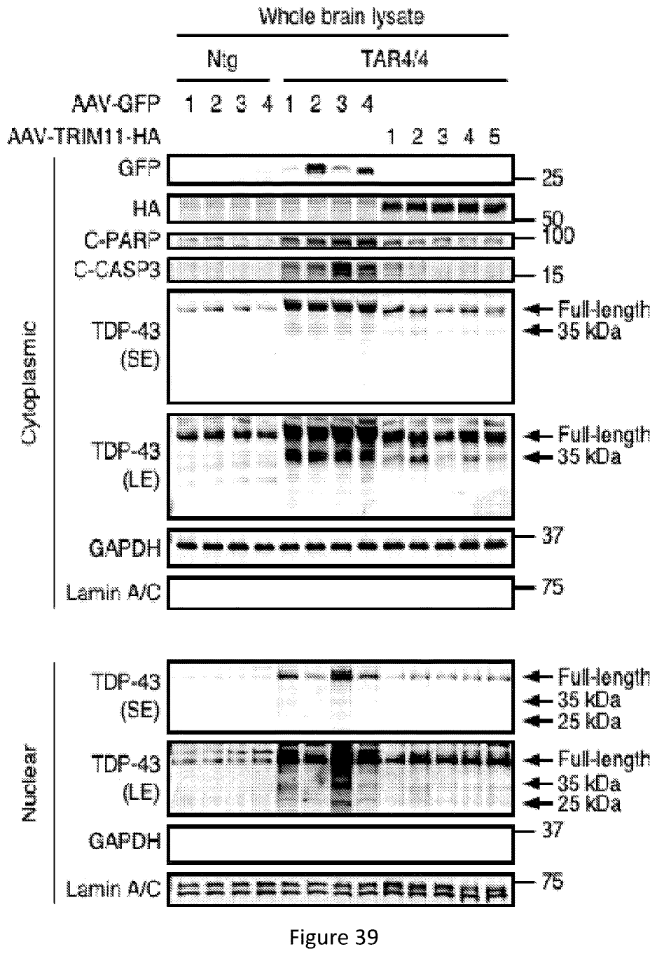

TRIM11-mediated reduction in TDP-43 was verified by western blot, which showed that levels of TDP-43 were strongly reduced in both the nuclear and the cytoplasmic fractions from AAV9-TRIM11-injected mice compared to those from AAV9-TRIM11-injected mice (FIG. 39). Immunoblot analysis also showed that caspase-3 activation was markedly reduced in AAV9-TRIM11-injected mice (FIG. 39). Together, these results indicate that TRIM11 reduces TDP-43 levels, neuroinflammation, and neurodegeneration in TAR4/4 mice.

### TRIM10 Reduces Levels of Various Misfolding-Prone Proteins

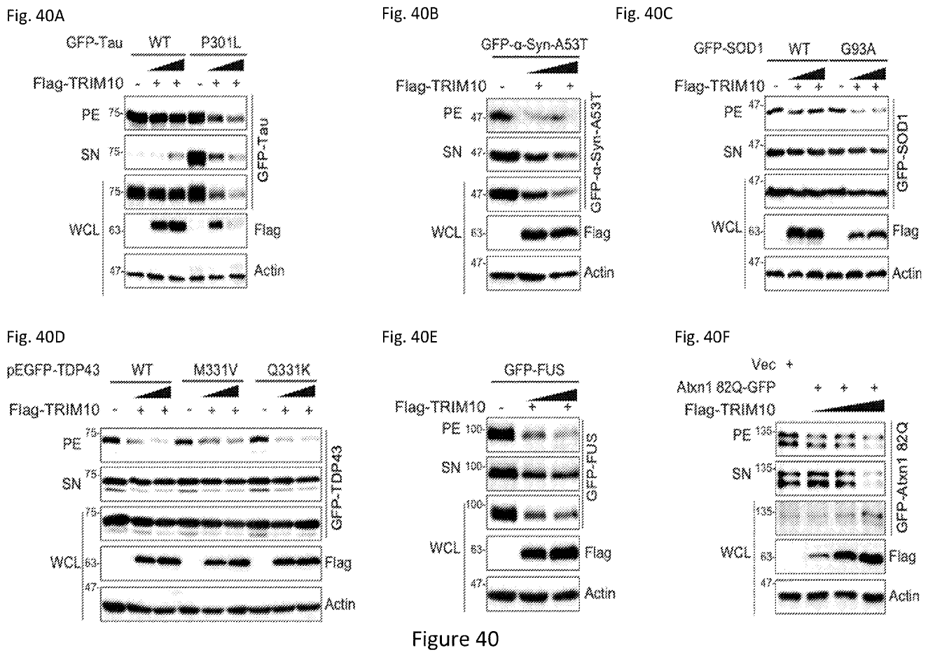

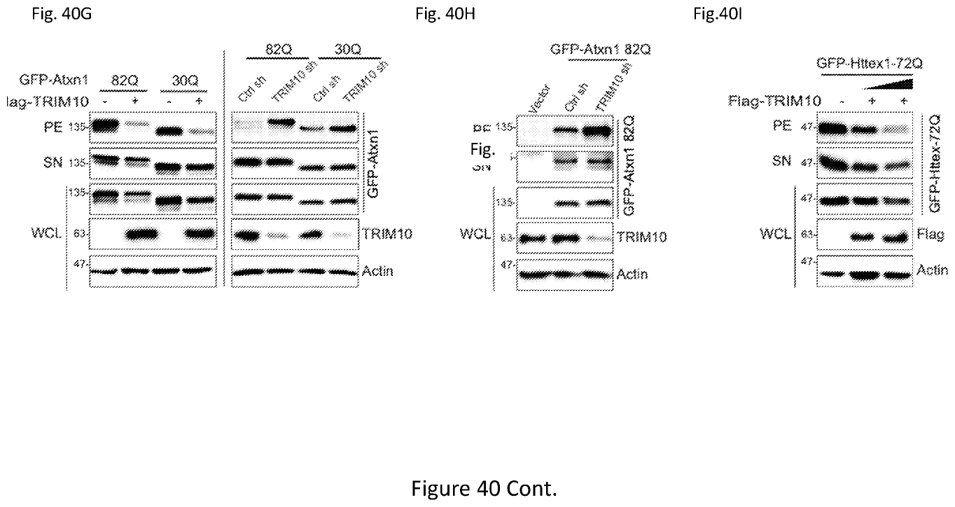

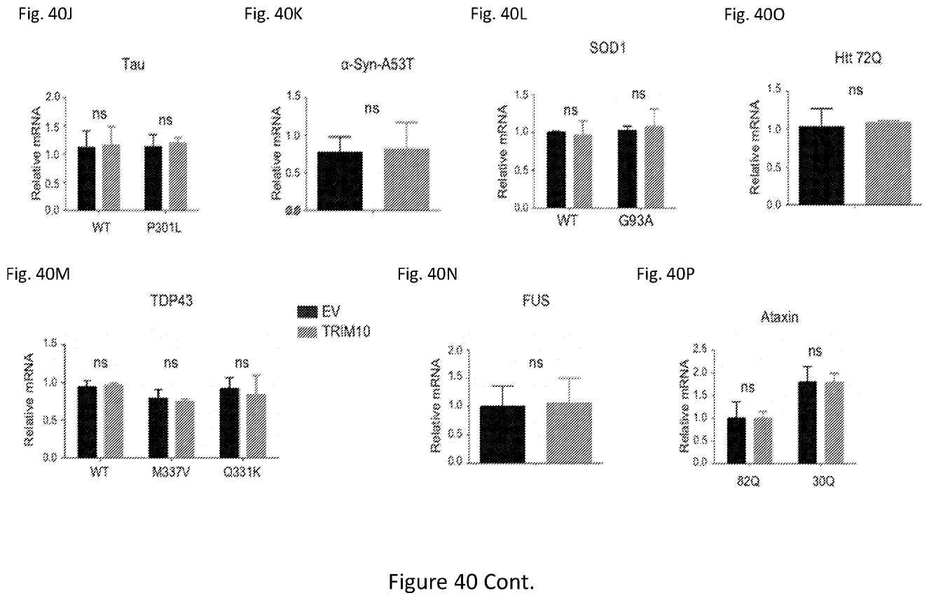

The screening of TRIMs described above suggested that TRIM10, like TRIM11, is a potent enabler for the folding of various disease-associated proteins, including tau, α-Syn, SOD1, and TDP-43. To further characterize the PQC activities of TRIM10, the effect of TRIM10 was examined on wild-type and mutant tau. When co-expressed in HEK293T cells, TRIM10 reduced levels of tau P301L in a dose-dependent manner, while having a moderate effect on wild-type tau (FIG. 40A). Therefore, TRIM10 preferentially clears mutant tau protein. TRIM10 also reduced levels of GFP-α-Syn A53T and GFP-SOD1 G93A, especially the insoluble species, in a dose-dependent manner (FIG. 40B and FIG. 40C). Moreover, TRIM10 reduced the levels of GFP fusions of TDP43 and two ALS-associated TDP-43 mutants, M337V and Q331K (FIG. 40D). Additionally, TRIM10 decreased levels of the RNA-binding protein FUS/TLS (FUsed in Sarcoma/Translocated in LipoSarcoma) (FIG. 40E), whose aggregation is associated with ALS (J. P. Taylor, R. H. Brown, Jr., D. W. Cleveland, Nature 539, 197-206 (2016; R. H. Brown, A. Al-Chalabi, N Engl J Med 377, 162-172, 2017).

The effect of TRIM10 on proteins with polyglutamine expansions was also examined. Forced expression of TRIM10 reduced levels of ataxin 1 with an expanded polyglutamine tract (Atxn1 82Q), which is associated with spinocerebellar ataxia type 1 (SCA1; H. T. Orr, H. Y. Zoghbi, Annu Rev Neurosci 30, 575-621, 2007). TRIM10 reduced levels of this mutant protein in both the soluble and insoluble fractions (FIG. 40F). TRIM10 also reduced levels of a non-pathogenic ataxin-1 protein, Atxn1 30Q; however, this effect of TRIM11 was more pronounced for insoluble species than for the soluble species (FIG. 40G). Conversely, knockdown of TRIM10 by means of small interfering RNA (shRNA) elevated levels of insoluble Atxn1 82Q and Atxn1 30Q (FIG. 40G and FIG. 40H). Moreover, TRIM10 reduced levels of a pathogenic fragment of huntingtin (Htt) encoded by the first exon of the HD gene, Httex1p 97QP (J. S. Steffan et al., Science 304, 100-104, 2004). Thus, TRIM10 also removes pathogenic polyglutamine protein, as well as superfluous non-pathogenic polyglutamine protein that exceeds the solubility limit. Despite its strong inhibitory effect on protein levels, TRIM10 did not affect mRNA levels of tau, α-Syn, TDP-43, SOD1, FUS, Atax1, or Htt72Q (FIG. 40J through FIG. 40P). Together, these results show that TRIM10 effectively removes a wide range of misfolding-prone proteins associated with neurodegenerative diseases, especially insoluble species and mutant variants.

### TRIM10 Promotes Degradation of Misfolding-Prone Proteins in the Proteasome

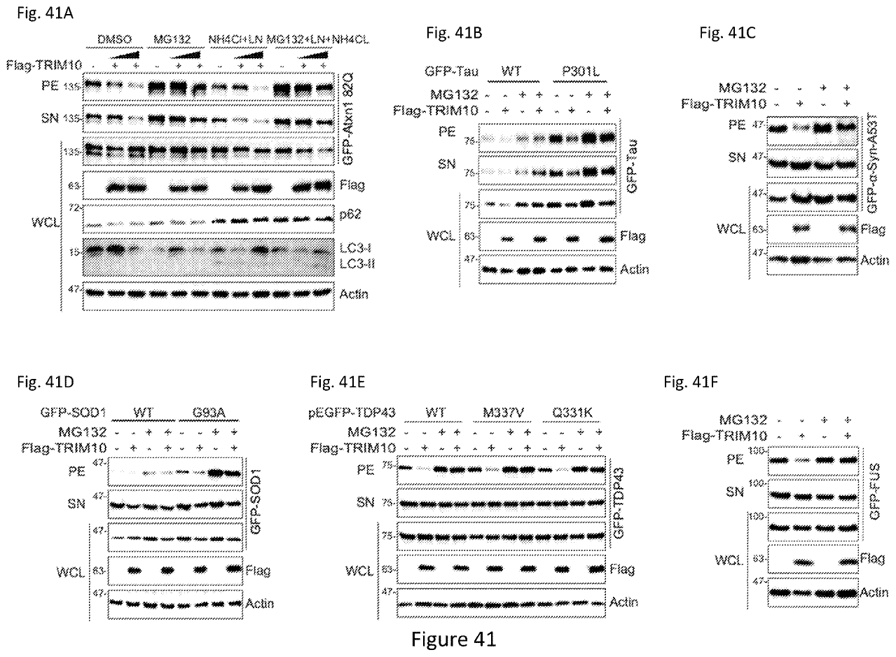

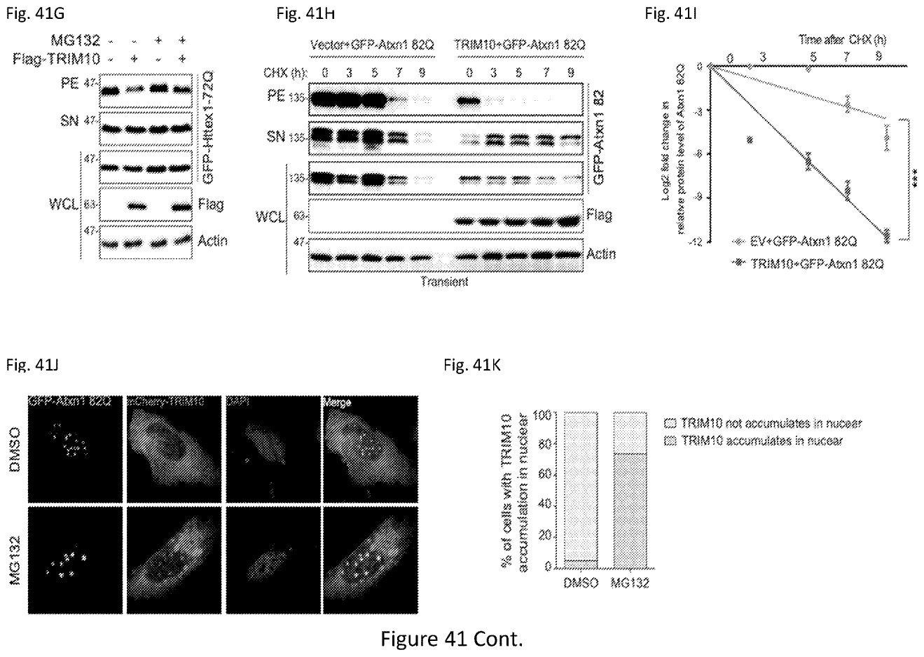

To examine whether TRIM10 promotes proteasomal degradation of the disease-associated proteins, the proteasome inhibitor MG132 and the lysosome inhibitors leupeptin and ammonium chloride (NH₄Cl) in combination (LN) were used. The ability of TRIM10 to remove Atxn1 82Q was blocked by MG132, but not LN (FIG. 41A). Likewise, the ability of TRIM10 to clear tau, α-Syn, TDP-43, SOD1, FUS, and Httex1 72Q was diminished when cells were treated MG132 (FIG. 41B through FIG. 41G). To evaluate whether TRIM10 affects the turnover misfolding-prone proteins, a cycloheximide (CHX) chase assay was performed. TRIM11 accelerated the turnover of Atxn1 82Q and shortened its half-life, especially the insoluble species (FIG. 41H and FIG. 41I).

Atxn1 82Q is mainly a nuclear protein and forms inclusion in this cellular compartment (L. Guo et al., Mol Cell 55, 15-30, 2014; L. Chen et al., Cell Rep 18, 3143-3154, 2017), while TRIM10 is mainly a cytoplasmic protein. Interestingly, however, in cells treated with MG132, TRIM10 accumulated in the nucleus and co-localized with Atxn1 82Q inclusions (FIG. 41J and FIG. 41K). This result suggests that TRIM10 is able to shuttle between the nucleus and the cytoplasm. Hence, TRIM10 is able to recognize Atxn1 82Q in the nucleus and targeting it for proteasomal degradation. The enhanced colocalization of TRIM10 with Atxn1 82Q in MG132-treated cells probably reflected the stalled attempt of TRIM10 to degrade this pathogenic protein upon the blockage of the proteasome. Collectively, these results show that TRIM10 targets various disease-associated proteins for proteasomal degradation.

### TRIM10 is a Molecular Chaperone for Various Client Proteins

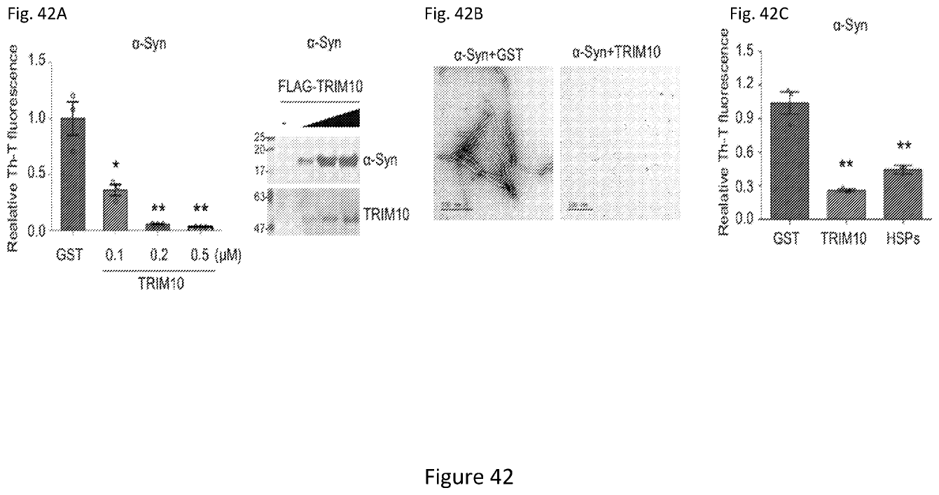

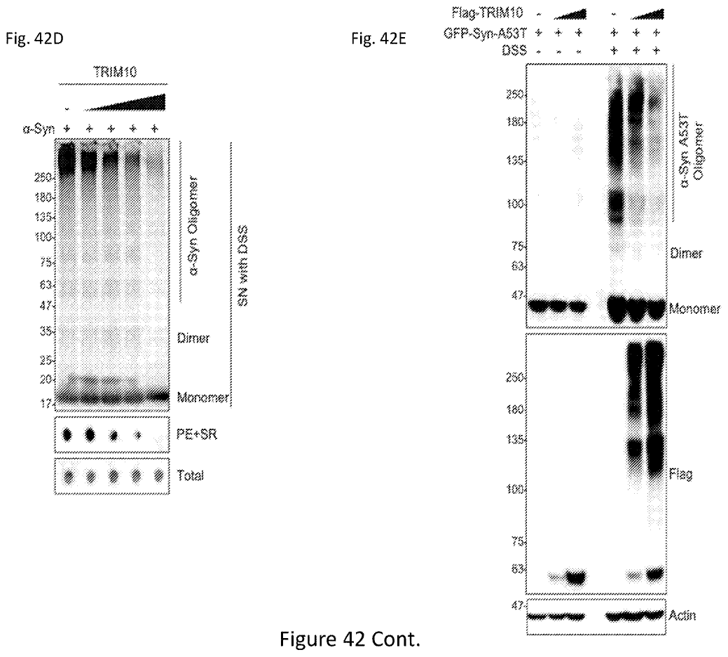

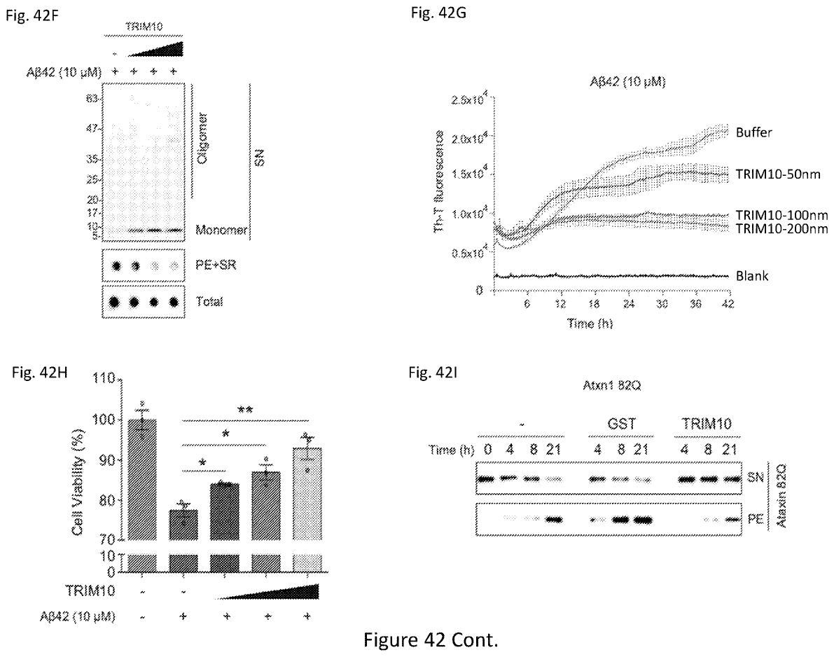

In addition to degradative systems, molecular chaperones and disaggregases prevent and reverse protein aggregation, respectively, maintaining their client proteins in a soluble form. The ability of TRIM10 to function as a molecular chaperone was examined. The PD-associated protein α-Syn forms fibrillar aggregates when incubated along in vitro. This was shown by analyses with the amyloid-specific dye ThT (FIG. 42A) and electron microscopy (EM) (FIG. 42B). TRIM10 reduced α-Syn fibrillization in a dose-dependent manner, reducing its binding to ThT (FIG. 42A) and keeping it soluble as shown by a sedimentation assay (FIG. 42A). TRIM10-mediated blockage of α-Syn fibrillization was verified by the EM (FIG. 42B). At a high dose (~0.5 μM), TRIM10 almost completely blocked α-Syn fibrillization (FIG. 42A and FIG. 42B). Even at this high dose, TRIM10 was still far less than the concentration of α-Syn monomers (~70 μM), with only a ~1:140 molar ratio to α-Syn. Moreover, the effect of TRIM10 was comparable or stronger than that of Hsp70/Hsp40-Hsp104 system, a potent chaperone system found in prokaryote, plant, and yeast (FIG. 42C). Therefore, the chaperone activity of TRIM10 is remarkably potent.

Prior to the formation of insoluble fibrillar aggregates, α-Syn, like other proteins associated with neurodegenerative diseases, generate soluble oligomers that are neurotoxic (R. Kayed et al., Science 300, 486-489, 2003). TRIM10 blocked the formation of α-Syn oligomers of various sizes in vitro (FIG. 42D). At the same time, TRIM10 reduced the levels of pelletable α-Syn aggregates that were soluble (PE) or resistant (SR) in SDS-containing buffer, as detected by dot blot (FIG. 42D). Moreover, TRIM10 abrogated the formation of soluble oligomers formed by α-Syn A53T in cells (FIG. 42C).

To evaluate whether TRIM10 has chaperone activity towards other client proteins, a substrate with a stronger propensity to aggregate was used, the AD-associated amyloid-beta peptide Aβ42. TRIM10 prevented the formation of PE and SR aggregates by Aβ42, keeping virtually all Aβ42 molecules in the soluble form (FIG. 42F). TRIM10 also blocked the formation of fibrils by Aβ42, as shown by a ThT binding assay (FIG. 42G). Aβ42 peptides that were pre-incubated alone were toxic to human neuroblastoma SH-SY5Y cells. However, upon pre-incubation with TRIM10, Aβ42 peptides showed much reduced cytotoxicity (FIG. 42H). Therefore, TRIM10 prevents the formation of toxic prefibrillar Aß42 oligomers.

Atxn1 82Q was used as a substrate. When incubated alone in vitro, Atxn1 82Q formed pelletable aggregates that were soluble in SDS-containing buffer (PE). TRIM10 strongly prevented the aggregation of Atxn1 82Q, keeping virtually all Atxn1 82Q molecules in the supernatant (SN) (FIG. 42I).

Together, these results indicate that TRIM10 is a potent a molecular chaperone that prevents aggregation of various disease-associated protein. Unlike conical molecular chaperones such as Hsp70 and Hsp90, which are multicomponent machineries driven by energy derived from ATP hydrolysis, TRIM10's chaperone activity is independent of other protein co-factors or ATP (FIG. 42).

TRIM10 is a Disaggregase that Dissolves Pre-Existing Protein Aggregates

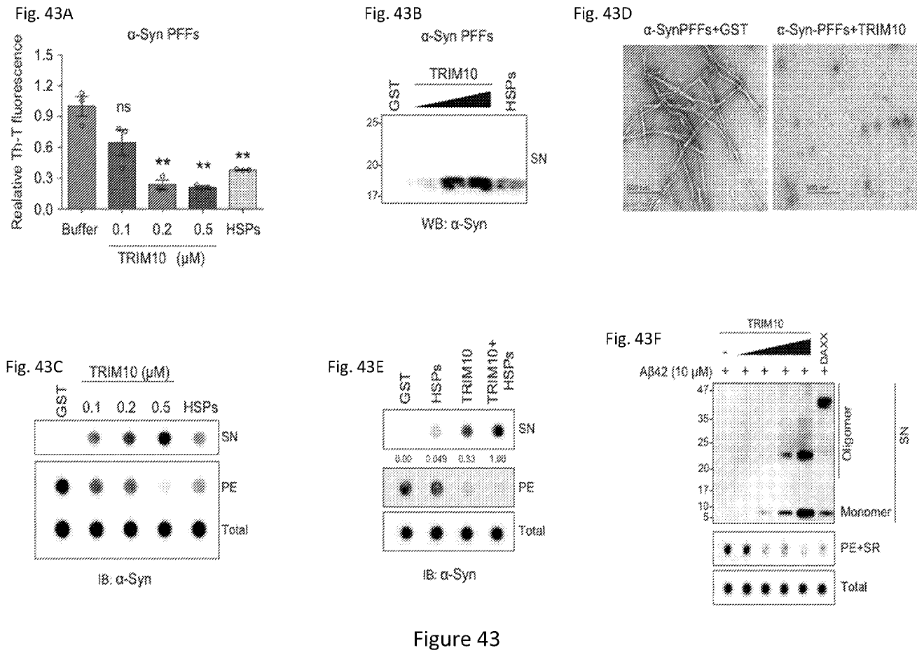

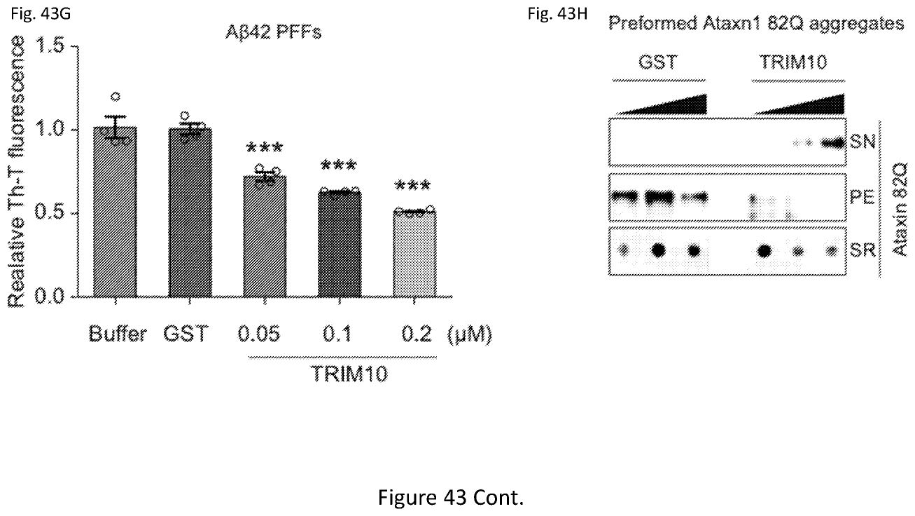

To evaluate whether TRIM10 is a protein disaggregase, pre-formed α-Syn fibrils (PFFs) were incubated with purified recombinant TRIM10 protein. TRIM10 dissolved α-Syn PFFs in a dose-dependent manner, reducing their binding to ThT (FIG. 43A) and converting them to a soluble state (FIG. 43B and FIG. 43C). An EM analysis confirmed that TRIM10 effectively dissolved mature α-Syn fibrils (FIG. 43D). This activity of TRIM10 was also stronger than that of the Hsp70/Hsp40-Hsp104 system (FIG. 43E), a potent disaggregase (J. R. Glover, S. Lindquist, Cell 94, 73-82, 1998). Moreover, TRIM10 and the HSP system may not synergize or cooperate in disaggregation, as their combination did not result in the solubilization of more α-Syn PFFs than TRIM10 did by itself (FIG. 43E). TRIM10 also disassembled Aβ42 fibrils and converted the PE and SR aggregates into soluble monomers (FIG. 43G). Moreover, TRIM10 effectively dissolved amorphous Atxn1 82Q aggregates (FIG. 43H). These results indicate that TRIM10 is also a potent disaggregase, converting various aggregates, including the highly organized amyloid fibrils, into a soluble state. Unlike canonical disaggregases, the activity of TRIM10 is independent of other cofactors or ATP (FIG. 43).

The multiple activities of individual TRIM proteins-degradative, molecular chaperone, and disaggregase activities—in combination makes them highly effective in suppressing aggregates in cells and in animals. AAV-based intracranial delivery of TRIM11 effectively protects against SOD1 and TDP-43 pathology and neuroinflammation in mouse models. TRIM11 also prevents the decline in cognitive and motor functions of these animals. The potency of TRIM11 in rescuing these disease models not only provides compelling evidence for the role of TRIM11 and other TRIMs in protecting against the initiation and progression of various neurodegenerative diseases, but also suggest their clinical efficacy in treating the diseases.

### Example 3: TRIMs Prevent and Reverse Aggregation of Prion-Like RNA-Binding Proteins

RNA-binding proteins (RBPs) with prion-like domains (PrLDs), which are a distinctive class of intrinsically disordered protein (IDP) regions resembling the yeast prion domains in its amino acid composition, constitute a large protein family in eukaryotes (Z. M. March et al. Brain Res 1647, 9-18, 2016). These proteins regulate various aspects of RNA metabolism and drive the assembly of a range of membrane-less organelles that create controlled environments to organize biochemical and biological processes in time and space (A. Patel et al. Cell 162, 1066-1077, 2015; T. Murakami et al. Neuron 88, 678-690, 2015; A. Molliex et al. Cell 163, 123-133, 2015; Y. Lin, D. S et al. Mol Cell 60, 208-219, 2015; I. R. Mackenzie et al. Neuron 95, 808-816 e809, 2017; K. A. Burke et al. Mol Cell 60, 231-241, 2015; J. Wang et al. Cell 174, 688-699 e616, 2018). However, because of the presence of the PrLDs, these prion-like RBPs are intrinsically prone to misfolding and aggregation, causing various degenerative diseases. These diseases include amyotrophic lateral sclerosis (ALS), a fatal movement disorder caused by the loss of motor neurons; frontotemporal dementia (FTD), the second most common dementia after Alzheimer's disease (AD); and multisystem proteinopathy (MSP), a pleiotropic degenerative disorder that affects muscle, bone, and brain (J. P. Taylor et al. Nature 539, 197-206, 2016; R. H. Brown et al. N Engl J Med 377, 162-172, 2017; T. Van Langenhove et al. Ann Med 44, 817-828, 2012; J. Bang et al. Lancet 386, 1672-1682, 2015; J. P. Taylor et al. Neurology 85, 658-660, 2015).

Notable examples of prion-like RBPs that are involved in human diseases include TAR DNA-binding protein 43 (TDP-43), fused in sarcoma (FUS, also known as translated in liposarcoma or TLS), and heterogeneous nuclear ribonucleoprotein A1 (hnRNPA1). Approximately 97% of cases of ALS and nearly 50% of FTD cases (the latter is referred to as Frontotemporal Lobar Degeneration due to TDP-43 proteinopathy, or FTLD-TDP) display aberrant TDP-43 aggregation (M. Neumann et al. Science 314, 130-133, 2006; T. Arai et al. Biochem Biophys Res Commun 351, 602-611, 2006). Mutations in the TDP-43-encoding gene TARDBP are linked to both familial (~5%) and sporadic ALS cases, as well as in rare cases of FTLD-TDP (J. Sreedharan et al. Science 319, 1668-1672, 2008; E. Kabashi et al. Nat Genet 40, 572-574, 2008; V. M. Van Deerlin et al. Lancet Neurol 7, 409-416, 2008; B. Borroni et al. Hum Mutat 30, E974-983, 2009; G. G. Kovacs et al. Mov Disord 24, 1843-1847, 2009). Deposition of TDP-43 is also linked to an increasing number of other neurodegenerative diseases including limbic predominant age-related TDP-43 encephalopathy (LATE) and other Alzheimer's Disease-Related Dementia (ADRD) (E. M. J. de Boer et al. J Neurol Neurosurg Psychiatry 92, 86-95, 2020). It is also a common comorbid pathology in Parkinson's disease (PD), Huntington's disease (HD), and especially AD (>50% of cases) (C. Amador-Ortiz et al. Ann Neurol 61, 435-445, 2007; K. Uryu et al. J Neuropathol Exp Neurol 67, 555-564, 2008). Mutations in the FUS gene causes a subset of familial ALS (fALS), in which the FUS variants form aggregates (T. J. Kwiatkowski et al. Science 323, 1205-1208, 2009; C. Vance et al. Science 323, 1208-1211, 2009). FUS immunoreactive inclusions are also found in the majority of sporadic FTD cases that are negative for tau or TDP-43 pathology (referred to as FTLD-FUS), as well as in other neurodegenerative diseases (M. Neumann et al. Brain 132, 2922-2931, 2009; M. Neumann et al. Acta Neuropathol 118, 605-616, 2009; J. Woulfe et al. Brain Pathol 20, 589-597, 2010; H. Doi et al. Neurosci Res 66, 131-133, 2010). Moreover, mutations in hnRNPA1 cause ALS as well as MSP (H. J. Kim et al. Nature 495, 467-473, 2013). Other prion-like RBPs that are linked to proteinopathies include EWS, TAF15 (both of which are closely related to FUS), and hnRNPA2 (H. J. Kim et al. Nature 495, 467-473, 2013).

In addition to the PrLD, TDP-43, FUS, and hnRNPA1 contain one or two RNA-recognition motifs (RRMs) and a nuclear localization signal (NLS). In heathy cells, TDP-43, FUS, and hnRNP1 are present mainly in the nucleus, although they can shuttle between the nucleus and cytoplasm (J. P. Taylor et al. Nature 539, 197-206, 2016; A. F. Harrison et al. Biochem J 474, 1417-1438, 2017; O. D. King et al. Brain Res 1462, 61-80, 2012; E. B. Lee et al. Nat Rev Neurosci 13, 38-50, 2011 H. Deng et al. Nat Rev Neurol 10, 337-348, 2014). TDP-43 participates in RNA transcription, splicing, and trafficking; mRNA turnover; and microRNA biogenesis. Of note, TDP-43 binds to cryptic exon sequences in pre-mRNA and prevents their mis-incorporation into mature mRNA, hence ensuring accurate production of mRNAs and proteins (J. P. Ling et al. Science 349, 650-655, 2015). Similarly, FUS and hnRNPA1 are involved in various aspects of RNA metabolism. Moreover, FUS can bind to single strain DNA and regulate transcription by both RNA polymerases II and III, as well as DNA damage response to preserve genomic integrity (A. Y. Tan et al. Mol Cell Biol 30, 186-196, 2010; A. Y. Tan et al. Proc Natl Acad Sci USA 109, 6030-6035, 2012; W. Y. Wang et al. Nat Neurosci 16, 1383-1391, 2013). In affected neurons and glial cells of related proteinopathies, TDP-43, FUS, and hnRNPA1 form aggregates in the cytoplasm, accompanied by their depletion in the nucleus. The mis-localization and aggregation cause both toxic gain of function of misfolded species in the cytoplasm and loss of normal functions of the soluble proteins in the nucleus. What regulates the solubility of prion-like RBPs is not well understood.

Tripartite motif (TRIM) proteins are defined by an RBCC domain comprising of a RING domain, a B-box domain(s), and a coiled coil, followed by a more divergent C-terminal region (K. Ozato et al. Nat Rev Immunol 8, 849-860, 2008; S. Hatakeyama et al. Nat Rev Cancer 11, 792-804, 2011). This family of proteins are present only in metazoans, and their number has been expanded substantially during evolution, from ~20 in the nematode Caenorhabditis elegans to over 70 in mice and humans. Emerging evidence indicate that some members of this family can participate in protein quality control (L. Guo et al. Mol Cell 55, 15-30, 2014; L. Chen et al. Cell Rep 18, 3143-3154, 2017; L. Chen et al. Nat Commun 9, 1223, 2018; G. Zhu et al. Cell Rep 33, 108418, 2020; Z. Y. Zhang e et al. Science 381, eadd6696, 2023). Here, the effect of the TRIM system on the cellular localization and solubility of prion-like RBPs including TDP-43, FUS, and hnRNPA1 are analyzed herein.

The materials and methods used in the experiments are now described.

### Antibodies

Antibodies against the following proteins or epitopes were purchased from the indicated sources: TRIM10 (pAb, Abcam, Cat #ab151306), hnRNPA1 (pAb, Abcam, Cat #ab4791), HA (mAb, C29F4, Cell Signaling, Cat #3724), TIA1 (mAb, Santa Cruz, Cat #sc-1751), GAPDH (mAb, Cell Signaling, Cat #2118S), G3BP1 (pAb, Cell Signaling, Cat #17798S), G3BP1 (mAb, Proteintech, Cat #66486-1-Ig), FUS (pAb, Proteintech, Cat #11570-1-AP), GFP (mAb, Santa Cruz, Cat #sc-9996), Lamin A/C (E-1) (mAb, Santa Cruz, Cat #sc-376248), mCherry (pAb, Proteintech, Cat #26765-1-AP), and HSP90 (mAb, Cell Signaling, Cat #4874).

Flag M2 Affinity Gel was purchased from Sigma (Cat #A2220). ChromoTek GFP-Trap® magnetic agarose was purchased from Proteintech. HRP-conjugated anti-rabbit IgG (Cat #7074S) and anti-mouse IgG (Cat #7076S) were purchased from Cell Signaling Technology. Goat Anti-Chicken IgY H&L (Alexa Fluor® 405) (Abcam, Cat #ab175674) was purchased from Abcam. Goat anti-Chicken IgY (H+L) Secondary Antibody, Alexa Fluor™ 647 (cat #A-21449); goat anti-rabbit IgG (H+L) cross-adsorbed secondary antibody, Alexa Fluor™ 488 (cat #A11008); goat anti-rabbit IgG (H+L) cross-adsorbed secondary antibody, Alexa Fluor™ 568 (cat #A11011); goat anti-rabbit IgG (H+L) cross-adsorbed secondary antibody, Alexa Fluor™ 647 (cat #A21214); and donkey anti-Mouse IgG (H+L) highly cross-adsorbed secondary antibody, Alexa Fluor™ 647 (cat #A31571) were purchased from Invitrogen.

### Reagents

The following reagents were purchased from the indicated sources: Ni-NTA agarose (Cat #30230, QIAGEN); amylose resin (Cat #E8021S, New England Biolabs); leupeptin (Cat #108975, Millipore); poly-ethylenimine (PEI), Linear, MW 25000 (Cat #23966-1, Polysciences); BLUEstain™ 2 Protein ladder, 5-245 kDa (Cat #P008-1000, Goldbio); PSC protease (Cat #27-0843-01) and PD-10 desalting columns packed with Sephadex G-25 resin (Cat #17-0851-01) (Cytiva); Alexa Fluor™ 568 C5-maleimide (Cat #A20341) or Alexa Fluor™ 488 carboxylic acid, succinimidyl ester (Cat #A20000) (Invitrogen); ABT888 (sc-202901) (Santa Cruz Biotechnology); Pierce™ Glutathione Superflow agarose (Cat #25236) and Hoechst 33342 (Cat #H3570) (ThermoFisher Scientific); WGA (Cat #L9640), Digitonin (Cat #D141), 3×FLAG® Peptide (Cat #F4799), Benzonase (Cat #E1014), Isopropyl-1-thio-D-galactopyranoside (IPTG) (Cat #16758), complete protease inhibitor cocktail (Cat #11697498001), L-glutathione reduced (Cat #G4251), PEG8000 (Cat #89510), imidazole (Cat #15513), D-(+)-Maltose monohydrate (Cat #M9171), Fluoromount™ Aqueous Mounting Medium (Cat #F4680-25ML) and sodium arsenite (Cat #S7400) (Sigma-Aldrich).

### Single-Guide RNAs, Antisense Oligos, and RNA Ligand

Single-guide (sgRNA) targeting human TRIM10 were synthesized by Integrated DNA Technologies, Inc. The targeting sequence is: 5′-GGCAGTTGACTTCATCTGCC-3′ (SEQ ID NO:27). Antisense oligos (ASOs) targeting mice TRIM10 were designed and synthesized by AUM LifeTech (Philadelphia, PA, USA). ASO sequences are: TRIM10 #1, 5′-TGAGATCACCTGTTGCCTCTT-3′ (SEQ ID NO: 28); TRIM10 #3, 5′-TAGTCAAGAGATACCTGCACC-3′ (SEQ ID NO:29). SCR CTRL-Far Red was tagged with a far-red fluorescent dye with Excitation Max at 646 nm and Emission Max at 669 nm (+/−5) as previously described (Z. Y. Zhang e et al. Science 381, eadd6696, 2023). RNA ligands used in this study are: FUS RNA ligand (J. C. Schwartz et al. Genes & development 26, 2690-2695, 2012): AUUGAGGAGCAGCAGAGAAGUUGGAGUGAAGGCAGAGAGGGGUUAAGG (SEQ ID NO:30); hnRNPA1 RNA ligand (Y. Lin, D. S et al. Mol Cell 60, 208-219, 2015): UAGGGACUUAGGGUUCUCUCUAGGGACUUAGGGUUCUCUCUAGGGA (SEQ ID NO:31).

### DNA Plasmids

For expression in mammalian cells, cDNAs for 75 TRIMs, including 73 human TRIMs and mouse TRIM12 and TRIM30 (Table 3), were synthesized and cloned into pCDH-EF1-FHC (Addgene, #64874), in which each TRIM was fused with a FLAG tag and an HA tag at the C-terminus (Gene Universal Inc., Newark, DE) (G. Zhu et al. Cell Rep 33, 108418, 2020). Human Kapβ2 was also cloned into pCDH-EF1-FHC by Gene Universal Inc. pGFP-N1-FUS/TLS-FLAGC, in which FUS/TLS was fused to both GFP and FLAG at the C-terminus (referred to as GFP-FUS in the current study), was a gift from Dr. P. Calsou (Addgene, Cat #60362) (S. Britton et al. Nucleic Acids Res 42, 9047-9062, 2014). M22: pHR-SFFVp-NLS-iLID::EGFP::FTH1 (Addgene, Cat #122147) and M23: pHR-SFFVp-FUSN: mCherry::SspB (Addgene, Cat #122148) were gifts from Dr. C. Brangwynne (D. Bracha et al. Cell 175, 1467-1480 e1413, 2018). pGFP-N1-FUS/TLS (R521H)-FLAGC and pGFP-N1-FUS/TLS (G156E)-FLAGC, referred to as GFP-FUS (R521H) and GFP-FUS (R156E), respectively, were generated by using QuickChange XL Site-Directed Mutagenesis Kit (Stratagene, La Jolla, CA) and overlap cloning techniques. mCherry-TRIM10 was constructed into mCherry-N1 using EcoR1 and BamH1 sites.

For expression in bacteria, MBP-Psc-FUS WT/(G156E)-GFP-His6 and MBP-Tev-FUS were cloned in pET-28 (+). pMal-Tev-FUS-GFP-Tev-His6, pMal-Tev-FUS (P525L)-GFP-Tev-His6, GCR2-tagRFP2-FUS, and GCR2-tagRFP2-FUS (P525L) were gifts from Dr. D. Dormann (M. Hofweber et al. Cell 173, 706-+, 2018). His-SUMO-hnRNPA1 plasmid, which was constructed into pET SUMO vector, was a gift from Dr. J. P. Taylor (A. Molliex et al. Cell 163, 123-133, 2015). GST-Tev-Kapβ2, GST-Tev-FUS, and GST-Tev-hnRNPA1 were previously described (L. Guo et al. Cell 173, 677-692 e620, 2018).

TABLE 3
TRIM proteins used in this study and their intracellular distribution pattern

| | Sub- | Accession | | Amino | Distribution pattern | | | | |
|---|---|---|---|---|---|---|---|---|---|
| Name | group | number | Isoform | Acid | C | N | P | F | HS |
| TRIM1 | I | NM_012216.4 | 1 | 735 | +++ | −/+ | + | − | − |
| TRIM2 | VII | NM_015271.5 | 1 | 771 | +++ | + | + | + | − |
| TRIM3 | VII | NM_033278.4 | 2 | 744 | +++ | − | −/+ | + | − |
| TRIM4 | IV | NM_033017.4 | α | 500 | +++ | − | + | − | − |
| TRIM5 | IV | NM_033034.3 | α | 493 | +++ | − | + | − | − |
| TRIM6 | IV | NM_001003818.3 | 1 | 516 | +++ | − | + | − | + |
| TRIM7 | IV | NM_203293.3 | 1 | 511 | +++ | −/+ | + | − | + |
| TRIM8 | V | NM_030912.3 | 1 | 551 | + | +++ | + | − | − |
| TRIM9 | I | NM_015163.6 | 1 | 710 | +++ | − | + | +++ | − |
| TRIM10 | IV | NM_006778.4 | 1 | 481 | +++ | + | + | +++ | − |
| TRIM11 | IV | NM_145214.3 | 1 | 468 | +++ | −/+ | − | − | − |
| TRIM12 | V | AF220126.1 | | 284 | +++ | + | + | − | + |
| TRIM13 | XI | NM_001007278.3 | 4 | 410 | +++ | − | −/+ | +++ | −/+ |
| TRIM14 | IV | NM_033219.3 | 2 | 442 | +++ | −/+ | + | − | +++ |
| TRIM15 | IV | NM_033229.3 | | 465 | +++ | − | + | − | + |
| TRIM16 | IV | NM_006470.4 | 3 | 564 | +++ | − | − | − | − |
| TRIM17 | IV | NM_001024940.3 | 2 | 477 | +++ | − | − | − | − |
| TRIM18 | I | NM_001193277.1 | 2 | 667 | +++ | − | − | − | − |
| TRIM19 | V | NM_033238.3 | 1 | 882 | − | +++ | − | − | + |
| TRIM20 | IV | NM_000243.3 | 1 | 781 | +++ | − | + | − | + |
| TRIM21 | IV | NM_003141.4 | | 475 | +++ | −/+ | − | +++ | − |
| TRIM22 | IV | NM_006074.5 | 1 | 498 | + | +++ | + | − | + |
| TRIM23 | IX | NM_001656.4 | α | 574 | +++ | + | + | − | − |
| TRIM24 | VI | NM_015905.3 | 1 | 1050 | − | +++ | − | − | − |
| TRIM25 | IV | NM_005082.5 | | 630 | +++ | − | + | − | − |
| TRIM26 | IV | NM_003449.5 | 1 | 539 | +++ | −/+ | + | − | − |
| TRIM27 | IV | NM_006510.5 | | 513 | +++ | −/+ | + | − | + |
| TRIM28 | VI | NM_005762.3 | | 835 | − | +++ | − | − | − |
| TRIM29 | V | NM_012101.4 | 1 | 588 | +++ | − | − | +++ | − |
| TRIM30 | IV | NM_009099.2 | 1 | 496 | +++ | + | + | + | + |
| TRIM31 | V | NM_007028.5 | 1 | 425 | +++ | −/+ | − | − | − |
| TRIM32 | VII | NM_012210.4 | 1 | 653 | +++ | − | + | − | − |
| TRIM33 | VI | NM_015906.4 | α | 1127 | − | +++ | + | − | + |
| TRIM34 | IV | NM_021616.6 | 1 | 488 | +++ | − | + | − | − |
| TRIM35 | IV | NM_171982.5 | 1 | 493 | + | +++ | + | − | + |
| TRIM36 | I | NM_018700.4 | 1 | 728 | +++ | − | − | +++ | − |
| TRIM37 | VIII | NM_001005207.5 | | 964 | ++ | ++ | − | − | − |
| TRIM38 | IV | NM_006355.5 | | 465 | +++ | + | + | − | − |
| TRIM39 | IV | NM_021253.4 | 1 | 518 | + | +++ | − | − | − |
| TRIM40 | V | BC060785.1 | 2 | 258 | ++ | ++ | − | − | − |
| TRIM41 | IV | NM_033549.5 | 1 | 630 | +++ | −/+ | + | − | + |
| TRIM42 | III | NM_152616.5 | | 724 | +++ | − | + | − | − |
| TRIM43 | IV | NM_138800.3 | | 446 | +++ | − | + | − | + |
| TRIM44 | V | NM_017583.6 | | 344 | +++ | + | − | − | − |
| TRIM45 | X | NM_025188.4 | 1 | 580 | +++ | + | + | − | + |
| TRIM46 | I | NM_025058.5 | 1 | 759 | +++ | − | − | +++ | − |
| TRIM47 | IV | NM_033452.3 | | 638 | +++ | − | − | − | + |
| TRIM48 | IV | NM_024114.5 | | 224 | +++ | + | + | − | + |
| TRIM49 | IV | NM_020358.2 | | 452 | +++ | − | − | − | − |
| TRIM50 | IV | NM_178125.3 | 1 | 487 | +++ | + | + | − | + |
| TRIM51 | IV | NM_032681.4 | | 452 | +++ | + | + | − | − |
| TRIM52 | V | NM_032765.4 | 1 | 297 | +++ | − | − | − | − |
| TRIM54 | II | NM_187841.3 | 2 | 358 | +++ | − | − | + | + |
| TRIM55 | II | NM_184085.2 | 1 | 548 | +++ | − | + | − | + |
| TRIM56 | V | NM_030961.3 | | 755 | +++ | − | + | − | − |
| TRIM58 | IV | NM_015431.4 | | 486 | +++ | −/+ | − | − | − |
| TRIM59 | XI | NM_173084.3 | | 403 | +++ | −/+ | + | − | + |
| TRIM60 | IV | NM_152620.3 | 2 | 471 | + | +++ | + | − | − |
| TRIM61 | V | NM_001012414.3 | | 209 | + | +++ | − | − | − |
| TRIM62 | IV | NM_018207.3 | 1 | 475 | +++ | − | + | − | + |
| TRIM63 | II | NM_032588.4 | | 353 | +++ | − | + | + | + |
| TRIM64 | IV | NM_001164397.3 | | 449 | + | +++ | − | − | − |
| TRIM65 | IV | NM_173547.4 | 1 | 517 | +++ | −/+ | −/+ | − | − |
| TRIM66 | VI | NM_014818.2 | 1 | 1216 | + | +++ | + | − | + |
| TRIM67 | I | NM_001004342.5 | 1 | 783 | +++ | − | + | − | + |
| TRIM68 | IV | NM_018073.8 | 1 | 485 | +++ | + | −/+ | − | − |
| TRIM69 | IV | NM_182985.5 | α | 500 | +++ | − | + | −/+ | + |
| TRIM70 | IV | NM_001037330.3 | | 348 | ++ | ++ | − | − | − |
| TRIM71 | VII | NM_001039111.3 | | 984 | +++ | − | + | − | + |
| TRIM72 | IV | NM_001008274.4 | | 477 | +++ | + | − | + | − |
| TRIM73 | V | NM_198924.4 | | 250 | ++ | ++ | + | − | − |
| TRIM74 | V | NM_198853.3 | 1 | 250 | +++ | + | + | − | + |
| TRIM75 | IV | NM_001396070.1 | | 468 | +++ | + | + | − | − |
| TRIM76 | I | NM_153610.5 | | 751 | +++ | − | − | − | − |
| TRIM77 | IV | NM_001146162.1 | 1 | 450 | ++ | ++ | − | − | − |

All TRIMs are of human origin except TRIM12 and TRIM30, which are of mouse origin with no known human orthologues. TRIM53 (a pseudogene) and TRIM57 (the same as TRIM59) are not listed. For each TRIM, the subfamily (I to XI), accession number, isoform, and number of amino acids are indicated. The isoform for each TRIM is usually the longest one and, when not indicated, the only isoform that has been identified. The intracellular distributions upon transfection in HeLa cells are also indicated. C: Cytoplasm; N: Nuclear; P: Puncta; F: Filament; HS: Hollow Spherical. “+++” indicated the distribution of ~90% of cells, “++” indicated the distribution of ~50% of cells, “+” indicated the distribution of 25% of cells, “/” indicated “−/+FUS & Ars”.

### Cell Culture

HEK293T and HeLa cells were purchased from ATCC and cultured in DMEM medium containing penicillin/streptomycin (1%), L-glutamine, and FBS (10%). Cells were cultured at 37 °C in a humidified incubator with 5% CO₂ unless otherwise indicated. HeLa cell line stably expressing C-terminally GFP-tagged human FUS were a gift from Dr. H. K. Lee and kept in DMEM medium under selection using Geneticin (Gibco, 400 μg/ml) (A. Patel et al. Cell 162, 1066-1077, 2015).

To generate TRIM10-knockout HeLa cell lines, HEK293T cells were co-transfected with control plentiCRISPRv2 vector (Addgene #98290) or plentiCRISPRv2 encoding TRIM10 sgRNA, together with the packaging plasmids pMD2.G (Addgene #12259) and psPAX2 (Addgene #12260) in a 4:1:3 ratio using PEI. The medium was replaced 12 h later. Viral particles were collected 60 h post-transfection and centrifuged at 1,200 rpm for 5 min, filtered with 0.45 μm sterile filter (Millipore), and concentrated by Lenti-X™ concentrator (Takara Bio, Cat #631312) at a ratio of 3:1 overnight at 4 °C, followed by centrifugation at 4 °C at 4500×g for 1 h. HeLa cells were then transduced with the concentrated viral particles in the presence of 8 μg/ml polybrene (Sigma-Aldrich). Lentiviral-transduced cells were selected in 2 μg/mL puromycin for 7 days with the medium changed every two days.

Neuronal cells were prepared from spinal cord and cerebral cortices of 3 days-old wild-type as described previously and plated on poly-Lysine-coated coverslip (D. L. Cao et al. Bio-protocol 7, e2098, 2017). These neurons were then grown and maintained in neurobasal medium containing Neuro basal supplemented with 2% of fetal bovine serum, 2% of heat-inactivated horse serum, 2% of B27 (Thermo Fisher Scientific, Gibco™, catalog number: 17504044), 2 mM GlutaMax, and 1% of penicillin-streptomycin. Neurons were transiently transfected with indicated plasmids by using Lipofectamine 2000 (Invitrogen) at 4 days. Transfected neurons were further incubated for 24 h at 37 °C with 5% CO₂ in neuronal maintenance medium and then treated with or without 0.5 mM sodium arsenite for 60 min. To knock down TRIM10 in neurons, control ASO and TRIM10 ASO #1 or the indicated TRIM10 ASOs were added into medium at a final concentration of 20 μM, and knockdown efficiency were analyzed by western blot 3 days later. Fluorescence-labeled ASOs were used to monitor cellular uptake.

### Immunofluorescence

Cells were plated on coverslips in 12-well plates and transfected with the indicated plasmids. Cells were fixed with 4% paraformaldehyde in PBS for 15 min at room temperature and permeabilized with PBS containing 0.2% Triton X-100 for 10 min. Cells were washed with PBS for five times and blocked with 10% goat serum in PBS for 1 h at room temperature and were incubated with indicated primary antibodies at 4 °C overnight, followed by fluorescence-labelled secondary antibodies at room temperature for 1 h. The coverslips were mounted to glass slides in mounting medium after Hoechst 33342 staining for 5 min. Fluorescence images were acquired by confocal microscope (Carl Zeiss LSM 880 Confocal Microscope and ZEN 2.3 LITE software) and analyzed by Fiji (Image J 1.52p).

To examine the effect of TRIMs on FUS/hnRNPA1 aggregation in stress granules (SGs), HeLa cells were transfected with the indicated TRIM plasmids. Twenty-four hours after transfection, cells were treated with 0.5 mM sodium arsenite for 90 min unless otherwise indicated, fixed, and stained with anti-HA and anti-TIA1 (or anti-G3BP1) antibodies and Hoechst 33342. Cells with at least four FUS/hnRNPA1-positive SGs (i.e., GFP- and G3BP1- or TIA1-positive puncta) are counted as ‘cells with FUS/hnRNPA1-positive SGs’. Every transfected cell in randomly selected fields was counted, and in each experiment, 20-50 cells per slide were counted. The percentage of cells with FUS/hnRNPA1-positive SGs among transfected cells was calculated, and the results of three biological replicates were used to calculate the means and SD.

### Real-Time Quantitative PCR (RT-qPCR)

Total RNA was extracted using TRIzol (Invitrogen, 15596018) according to the manufacturer's instructions. 1 μg of RNA was reversely transcribed using High-Capacity cDNA Reverse Transcription Kit (Themo Fisher Scientific, 4368814). Gene expression was determined by SYBR Green-based RT-PCR using an ABI ViiA 7 PCR system (Applied Biosystems, Foster City, CA, USA). Each gene expression was normalized to GAPDH. The following primers were used (5′ to 3′): FUS-F: GGAACTCAGTCAACTCCCCA (SEQ ID NO:32), FUS-R: TACCGTAACTTCCCGAGGTG (SEQ ID NO:33), TRIM10-F: CTGCCCCATCTGTCAGGGTA (SEQ ID NO:34), TRIM10-R, GGTATCTCACAGTAGCGGGTAA (SEQ ID NO:35), GAPDH-F: GCTAAGGCTGTGGGCAAGG (SEQ ID NO:36), GAPDH-R: GGAGGAGTGGGTGTCGCTG (SEQ ID NO:37).

### Nuclear and Cytoplasmic Fractionation.

HeLa cells plated in 6 well plates were transfected with indicated plasmids. Cells were harvested in ice-cold PBS and collected by centrifugation at 1,000 rpm for 5 min. Cell pellets were resuspended in 250 μl hypotonic lysis buffer (10 mM HEPES-NaOH, pH 7.9, 10 mM KCl, 1.5 mM MgCl₂, 0.5 mM b-mercaptoethanol) supplemented with protease inhibitors and phosphatase inhibitors, vortexed, and incubated on ice for 15 min, followed by the addition of 4 μl 10% NP-40 for another 5 min. Samples were centrifuged at 13,000×g for 10 min, the supernatants were collected as cytoplasmic proteins. The pellets were used for nuclear proteins extraction. Pellets were washed with ice cold PBS four times, and added with 60 μl nuclear lysis buffer (10 mM Tris-HCl, PH 7.6, 420 mM NaCl, 0.5% Nonidet P-40, 2 mM MgCl₂, 1 mM DTT, 1 mM PMSF and protease phosphatase inhibitors), and then dispersed and incubated on ice for 20 min. The extractions were centrifuged at 13,000×g for 10 min for nuclear proteins. Finally, the nuclear proteins were added with low salt buffer (10 mM Tris-HCl, pH 7.6, 2 mM MgCl2, 1 mM DTT, 1 mM PMSF plus protease phosphatase inhibitors) to adjust the concentration of NaCl to 150 mM. All the protein samples were boiled and stored at −80 °C.

### Recombinant Proteins

TEV-His6, GST-Tev-KapB2, GST-Tev-FUS, and GST-Tev-hnRNPA1 were expressed in E. coli BL21 cells and affinity purified using Pierce™ Glutathione Superflow Agarose (L. Guo et al. Cell 173, 677-692 e620, 2018). After washing with resuspension buffer, proteins were eluted in resuspension buffer containing 20 mM reduced L-glutathione.

To purify proteins from HEK293T cells, TRIM10-Flag-HA and TRIM11-Flag-HA were transfected into HEK293T cells. Cells were lysed in IP-lysis buffer (25 mM Tris-HCl, pH 7.4, 300 mM NaCl, 1% Triton X-100, and 1 mM EGTA) with sonication. After centrifugation, supernatants were incubated with anti-Flag M2 Affinity Gel at 4 °C overnight. The gel was washed sequentially with lysis buffers containing 0, 0.25, 0.5, 1, 0.5, 0.25, and 0 M KCl, and then with Tris buffer or sodium phosphate buffer. Recombinant proteins were eluted with 3×Flag peptide by vortex on ice for 3 h (L. Huang et al. Nature 597, 132-137, 2021).

Sedimentation Assay for FUS and hnRNPA1 In Vitro

For aggregation of GST-Tev-FUS and GST-Tev-hnRNPA1 without dwelling in a liquid phase state, samples were cleaved using TEV in 20 μl buffer (40 mM HEPES, pH 7.5, 150 mM NaCl, 1 μM DTT) at 25 °C for 24 h, followed by centrifugation at room temperature at 13,000 rpm for 15 min. For dissolvement of preformed FUS and hnRNPA1 fibrils, samples in the absence or presence of TRIM10 or Kapβ2 were centrifuged at room temperature at 13,000 rpm for 15 min.

Equal volumes of supernatant and pellet fraction were analyzed by western blot with anti-GFP, anti-HA, anti-FUS, and/or anti-hnRNPA1 antibody. After blocking with 5% non-fat milk (Labscientific, M0841) in tris-buffered saline with Tween (TBST), the membrane was incubated with appropriate primary antibodies, followed by HRP-conjugated secondary antibodies. Signals were detected by using a Western Blot Substrates and Substrate Kits (Thermo Fisher Scientific) in an ECL detection system (Bio-Rad Chemidoc Touch Imaging System Chemiluminescence/Fluorescence Detection). The sample loading and exposure time of the immunoblots were controlled so that all bands of western blots were detected within the linear range.

### Prevention of Protein Misfolding and Aggregation

Aggregation of FUS and hnRNPA1 was assayed by both sedimentation assay and electron microscopy assays at the Electron Microscopy Resource Laboratory at the University of Pennsylvania. Samples were stained via negative staining and scanned by FEI Tecnai-12 electron microscope.

### Fibril Formation and Dissolution

Spontaneous FUS fibrillization was initiated by adding PSC protease to MBP-FUS-GFP-His6 (5 μM) or TEV to GST-Tev-FUS (5 μM) in FUS assembly buffer (40 mM HEPES, 150 mM NaCl and 1 mM DTT). Spontaneous FUS fibrillization reactions were incubated at 25 °C with agitation at 700 rpm in an Eppendorf Thermomixer for 24 h, at which time fibrillization was complete with nearly 100% of FUS being in the aggregated state. For hnRNPA1 fibrillization, GST-Tev-hnRNPA1 (5 μM) were incubated with TEV protease in A1 assembly buffer (40 mM HEPES-NaOH, pH 7.4, 150 mM NaCl, 5% glycerol, 1 mM DTT, and 20 mM glutathione). hnRNPA1 fibrillization reaction was incubated at 25 °C with agitation at 1,200 rpm in an Eppendorf Thermomixer for 16 h, at which time fibrillization was complete with nearly 100% of the hnRNPA1 being in the aggregated state. Spontaneous fibrillization reactions were performed in the presence or absence of the indicated concentration of TRIM10 or Kapβ2. Preformed FUS and hnRNPA1 (5 μM monomer) fibrils generated as described above were incubated at 25 °C with indicated concentration of TRIM10 or Kapβ2 for 24 h and were assessed by both sedimentation assay and EM.

### Hydrogel Formation and Disassembly

Formation of FUS and hnRNPA1 hydrogels were performed as previously described (L. Guo et al. Cell 173, 677-692 e620, 2018). Briefly, GST-Tev-FUS (40 mg/ml or ~480 μM monomer concentration) in 40 μl or 60 μl buffer containing 50 mM Tris-HCl, pH 8, 200 mM trehalose, and 20 mM glutathione, and GST-Tev-hnRNPA1 (200 mg/ml or ~3.1 mM monomer concentration) in 40 μl buffer containing 40 mM HEPES-NaOH, pH 7.4, 150 mM NaCl, 5% glycerol, and 20 mM glutathione, were incubated in a PCR tube (Eppendorf 022431064) on ice for at least one month to form hydrogels. For the prevention of FUS hydrogel formation, 20 μl buffer, Kapβ2 (36 μM monomer, final concentration) or TRIM10 (13.3 μM monomer, final concentration) to give a molar ratio of Kapβ2: FUS=1:8.8, TRIM10: FUS=1:24; For the prevention of hnRNPA1 hydrogel formation, 30 μl buffer, Kapβ2 (180 μM monomer, final concentration) or TRIM10 (13.3 μM monomer, final concentration) to give a Kapβ2/hnRNPA1 molar ratio of 1:12 and a TRIM10/hnRNPA1 molar ratio of 1:165. For the dissolvement of FUS hydrogel, 50 μl buffer, Kapβ2 (54 μM monomer, final concentration) or TRIM10 (20 μM monomer) to give a Kapβ2/FUS molar ratio of 1:4.5 and a TRIM10/FUS molar ratio of 1:12; For the dissolvement of hnRNPA1 hydrogel, 40 μl buffer, Kapβ2 (270 μM monomer, final concentration) or TRIM10 (20 μM monomer, final concentration) to give a Kapβ2/hnRNPA1 molar ratio of 1:6 and a TRIM10/hnRNPA1 molar ratio of 1:83. Samples were processed for photography and EM. For EM, the hydrogel samples were prepared following the same protocol as above but in the cold room.

### Software

GraphPad Prism 7. Image J 1.52p. Carl Zeiss LSM 880 Confocal Microscope and ZEN 2.3 LITE software.

### Statistical Analysis

Data are presented as mean±standard deviation (SD). A two-tailed Student's t-test was used to evaluate the statistical significance in the mean value between two populations (*P<0.05; **P<0.01; ***P<0.001). Prism GraphPad 7 was used to create the figures and to complete the statistical analysis.

The results of the experiments are now described.

### Systematic Analysis of TRIMs on TDP-43 Localization

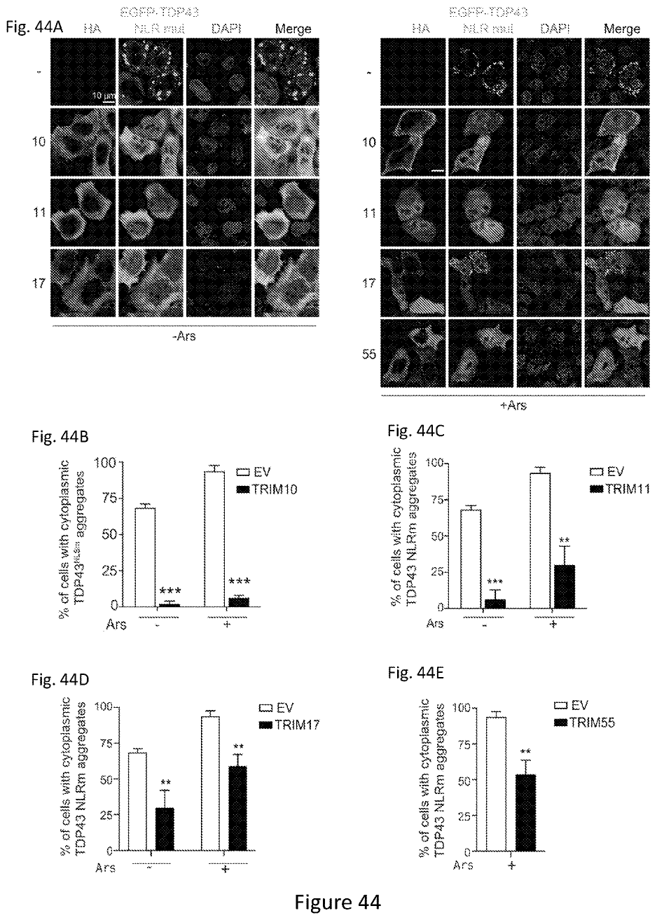

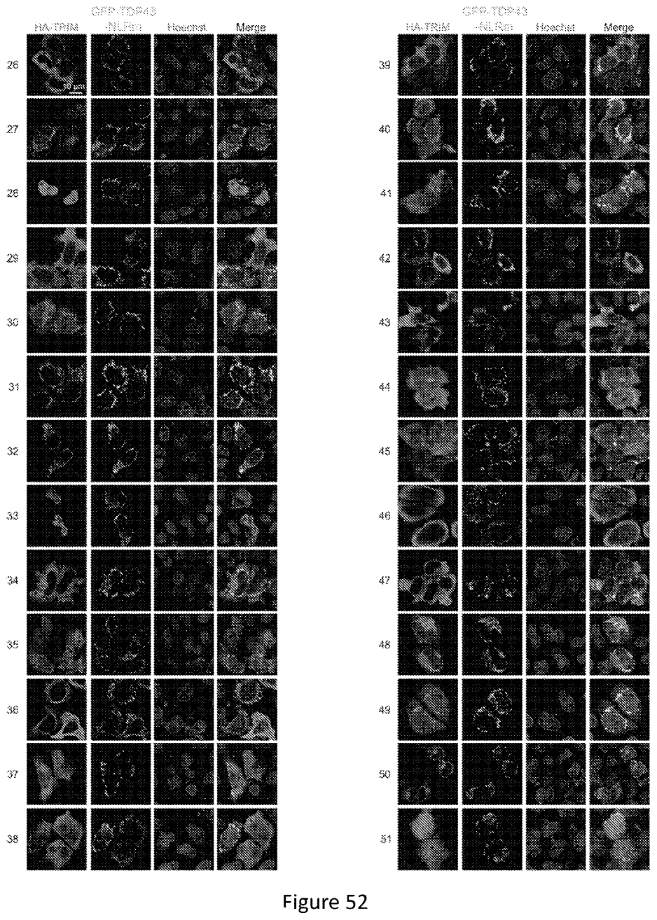

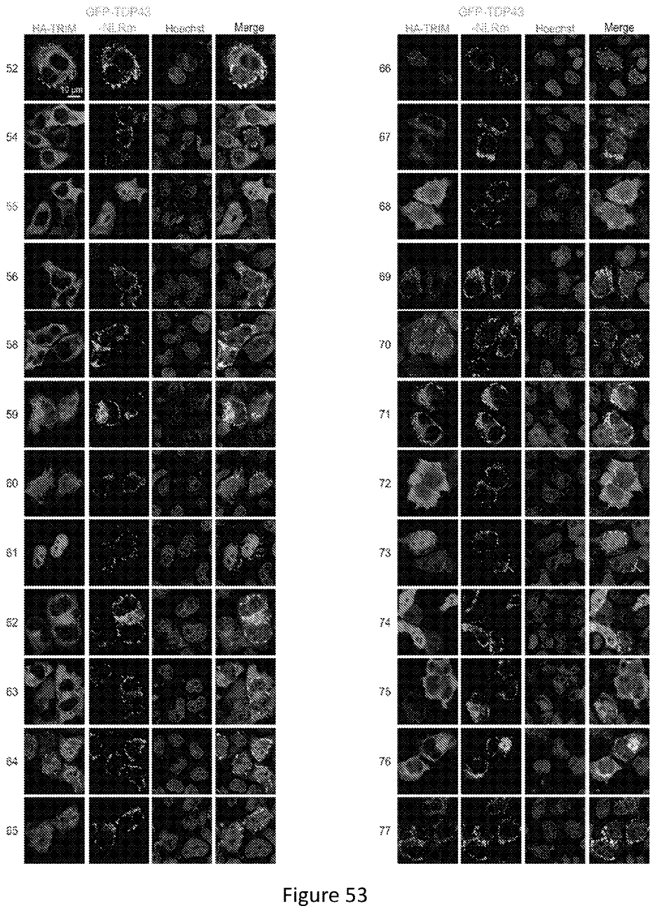

TDP-43 proteinopathies are defined by cytoplasmic aggregation and nuclear depletion of TDP-43. To determine the effect of TRIMs on TDP-43 localization, the 75 TRIMs were systematically examined on a GFP fusion of TDP-43^{NLSm}, which harbors mutations in one site within the bipartite NLS (S. Porta et al. Nat Commun 9, 4220, 2018). When introduced into HeLa cells alone, GFP-TDP-43^{NLSm} formed puncta in the cytoplasm of ~70% of the cells. The cytoplasmic localization was further increased to ~90% of the cell upon treatment with sodium arsenite, which elicits oxidative stress (FIG. 44). When co-expression with GFP-TDP-43^{NLSm}, many TRIMs did not appear to affect the localization of the TDP-43 mutant (FIG. 51 through FIG. 53). However, several TRIMs, including TRIM10, 11, 17, and TRIM55, effectively block cytoplasmic mis-localization of GFP-TDP-43^{NLSm} and restored its nuclear localization (FIG. 44). At doses that did not alter the protein or RNA levels of GFP-TDP-43^{NLSm}, these four TRIMs strongly reduced cytoplasmic GFP-TDP-43^{NLSm} puncta in both untreated and arsenite-treated cells (FIG. 44 and FIG. 51 through FIG. 53). The ability of these four TRIMs to restore TDP-43^{NLSm} nuclear localization is remarkable considering that this TDP-43 mutant is partially impaired for nuclear localization (S. Porta et al. Nat Commun 9, 4220, 2018).

### Downregulation of TRIM11 and TRIM10 Expression in FTLD-TDP Brains

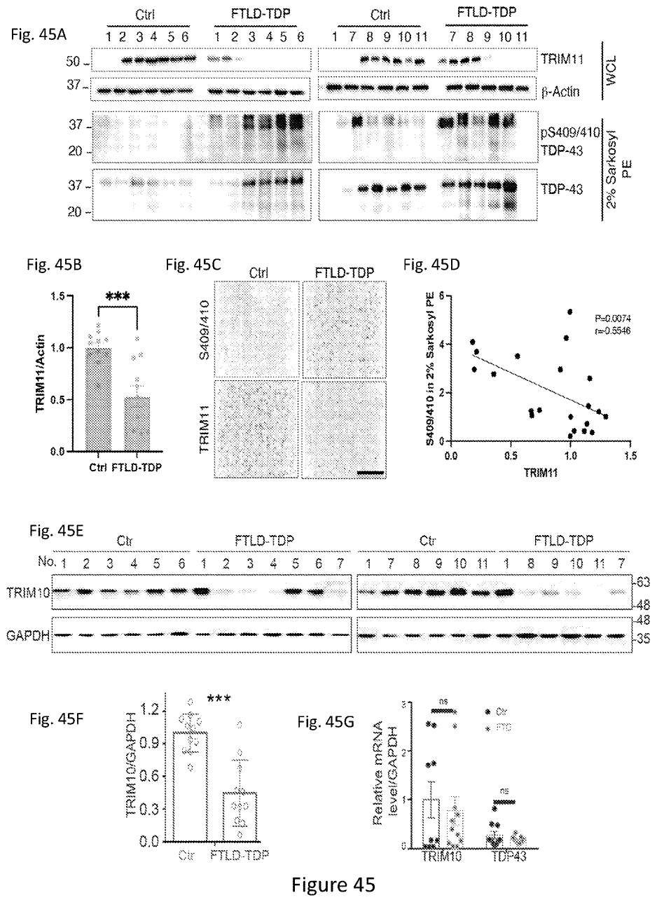

To evaluate the relevance of TRIM11 in the pathogenesis of TDP-43 proteinopathies, its expression was analyzed in postmortem brain tissues from 11 FTLD-TDP individuals and 11 age- and sex-matched control individuals with no known neurodegenerative diseases. As expected, FTLD-TDP, but not control, brain tissues showed aggregated TDP-43 species that were insoluble in the zwitterionic detergent sarkosyl and enriched for S409/410 phosphorylation (FIG. 45A), indicative of pathologic TDP-43 inclusions. Of note, levels of TRIM11 protein, but not mRNA, were markedly decreased in FTLD-TDP brains compared to control brains, with an average reduction of ~50% (FIG. 45A and FIG. 45B). The reduction of TRIM11 in FTLD-TDP brains was corroborated by immunohistochemistry (IHC) (FIG. 45C). Across control and FTLD-TDP brain tissues, there was a strong inverse correlation between TRIM11 expression and levels of pS409/410 species (FIG. 45D).

Expression of TRIM10 was also analyzed in these postmortem brain tissues. Similarly, levels of TRIM10 protein, but not mRNA, were markedly decreased in FTLD-TDP brains compared to control brains, with an average reduction of ~55% (FIG. 45E through FIG. 45G). Moreover, expression of TRIM10 was barely detected in several FTLD-TDP samples (FIG. 45E through FIG. 45G). Together, these results suggest that downregulation of TRIM11 and/or TRIM10 may contribute to the pathogenesis of FTLD-TDP.

### Role of Endogenous TRIM11 and TRIM10 in Regulating TDP-43

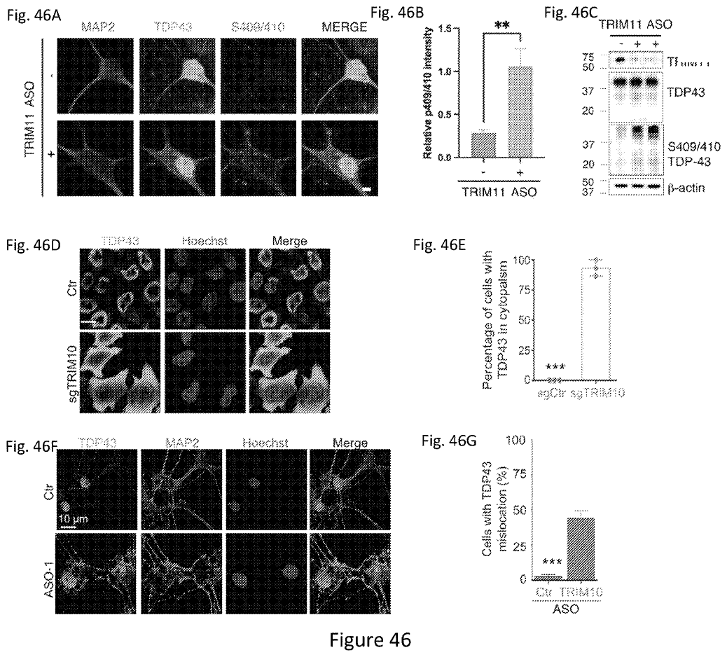

To address the role of endogenous TRIM11 in mammalian brain, the expression of Trim1l was reduced in cortical and spinal cord neurons derived from wild-type mice by antisense oligos (ASOs) (Z. Y. Zhang e et al. Science 381, eadd6696, 2023). Silencing Trim11 increased cytoplasmic mis-localization and aberrant aggregation of TDP-43 (FIG. 46A through FIG. 46C).

To evaluate the role of endogenous TRIM10, TRIM10 was knocked out in HeLa cells using CRISPR-mediated genome editing. Knockout of TRIM10 resulted in cytoplasmic mis-localization of endogenous TDP-43 in over 90% of the cells (FIG. 46D and FIG. 46E). Moreover, silencing TRIM10 expression in mouse primary cortical or spinal cord motor neurons via antisense oligonucleotides (ASOs) led to cytoplasmic mis-localization of endogenous TDP-43 in ~44% of the cells (FIG. 46F and FIG. 46G) (Z. Y. Zhang e et al. Science 381, eadd6696, 2023). These results indicate that both endogenous TRIM11 and TRIM10 may be critical for maintaining nuclear localization and solubility of TDP-43.

### TRIM10 Prevents and Reverses TDP-43 Aggregation

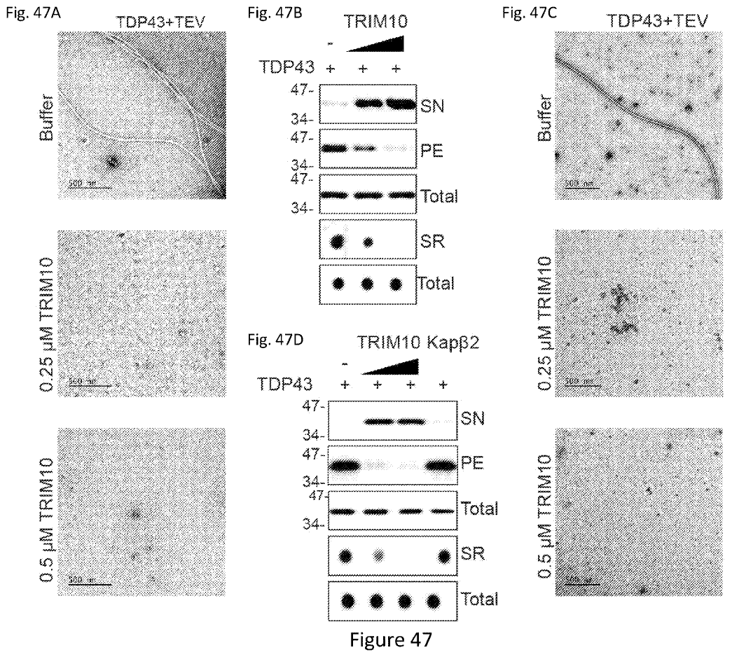

To maintain protein solubility, cells employ molecular chaperone that prevent protein misfolding and aggregation and disaggregases that dissolve pre-existing protein deposits. It was previously shown that TRIM11 functions as both a molecular chaperons and a disaggregase (Z. Y. Zhang e et al. Science 381, eadd6696, 2023; G. Zhu et al. Cell Rep 33, 108418, 2020). To assess the effect of TRIM10 on TDP-43 fibrillization, recombinant MBP-Tev-TDP-43, in which TDP-43 is fused to maltose binding protein (MBP) to enhance solubility, was purified with a TEV protease site protease site inserted between MBP and TDP-43 (L. Guo et al. Cell 173, 677-692 e620, 2018). Upon the cleavage of MBP, TDP-43 monomers were quickly formed mature amyloid fibrils containing cross-b structures, which was detected by electron microscopy (EM) (FIG. 47A), as well as pelletable aggregates that were SDS-soluble (PE; detected by Western blot) or SDS-resistant (SR; detected by dot blot) (FIG. 47B) (M. Kato et al. Cell 149, 753-767, 2012). Of note, TRIM10 showed a strong molecular chaperon activity towards TDP-43, nearly completely preventing TDP-43 fibrillization (FIG. 47A and FIG. 47B). Unlike canonical molecular chaperones such as HSP60 and HSP70, which are multi-component systems driven by energy derived from ATP hydrolysis, TRIM10 prevents TDP-43 aggregation by itself, in the absence of any other protein cofactors or ATP (FIG. 47A and FIG. 47B) (S. Wolff et al. Cell 157, 52-64, 2014; D. Balchin et al. Science 353, aac4354, 2016).

Compared to prevention of protein aggregation, dissolution of pre-existing protein deposits is a much more challenging process, often requiring participation of additional enzymes as well as ATP (H. Saibil et al. Nat Rev Mol Cell Biol 14, 630-642, 2013; S. M. Doyle et al. Nat Rev Mol Cell Biol 14, 617-629, 2013). To examine whether TRIM10 possesses disaggregase activity towards TDP-43, pre-existing TDP-43 aggregates, including amyloid fibrils and PE and SR aggregates, were treated with recombinant TRIM10 protein. Remarkably, TRIM10 nearly completely dissolve preformed TDP-43 aggregates (FIG. 47C and FIG. 47D). As for aggregation prevention, TRIM10 dissolved TDP-43 aggregates by itself independently of ATP.

Recent studies showed that nuclear import receptors (NIRs; e.g., Impa/Kapβ1 and Kapβ2) prevent aggregation of prion-like RBPs including TDP-43 and FUS (L. Guo et al. Cell 173, 677-692 e620, 2018; T. Yoshizawa et al. Cell 173, 693-705 e622, 2018; M. Hofweber et al. Cell 173, 706-719 e713, 2018). To be effective, NIRs need to be present at an equal molar ratio to their client proteins, as they stably interact with the NLS on RBPs (L. Guo et al. Cell 173, 677-692 e620, 2018; T. Yoshizawa et al. Cell 173, 693-705 e622, 2018; M. Hofweber et al. Cell 173, 706-719 e713, 2018). By contrast, TRIM10 prevents and reverses TDP-43 fibrillization even at a low sub-stoichiometric ratio to TDP-43 (1:10 or 1:20) (FIG. 47). These results indicate that TRIM10 is a potent molecular chaperone and disaggregase for TDP-43, keeping TDP-43 in a functional soluble form.

### Identification of TRIM10 as a Potent Regulator of FUS

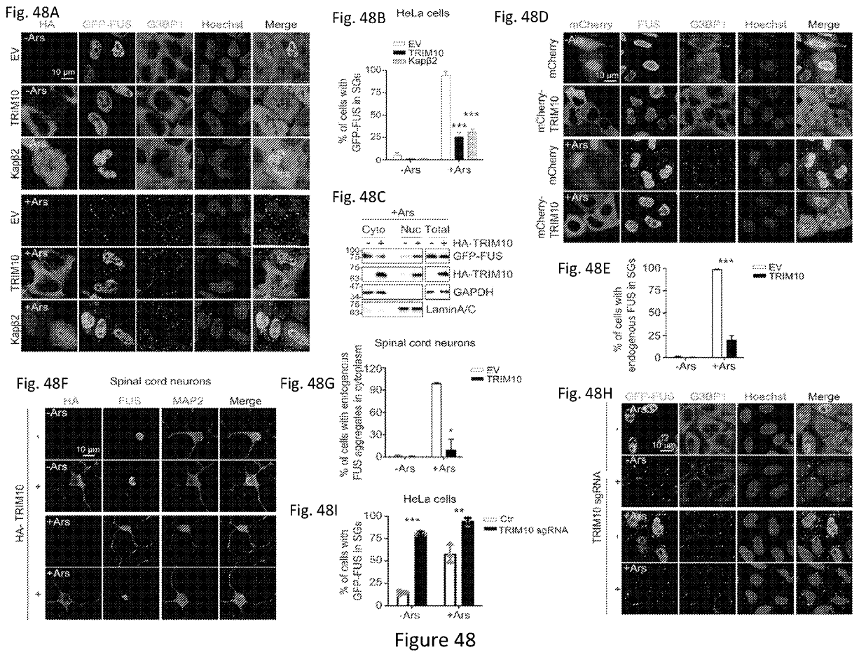

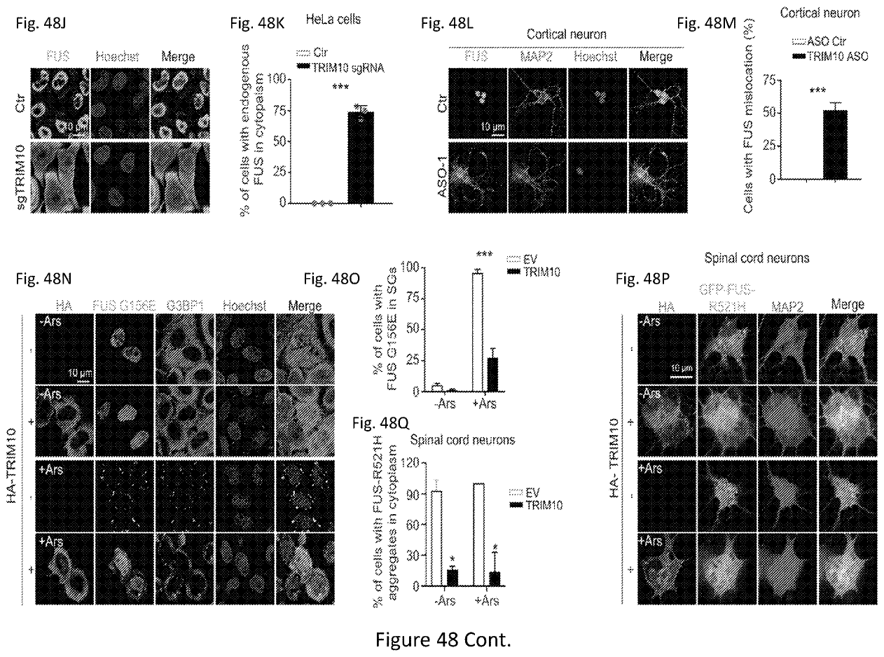

Like TDP-43, aggregation of FUS is associated with ALS and FTD. To identify TRIMs that may regulate FUS, TRIM proteins on the cellular localization of FUS were surveyed under stress conditions. In cultured cells experiencing stress, as in degenerating neurons in the affected ALS/FTD patients, FUS often mis-localizes to the cytoplasm and accumulates in SGs (D. Dormann et al. EMBO J 29, 2841-2857, 2010). This SG localization increases the chance of aberrant phase transitions and pathological fibrillization of FUS due to its elevated local concentrations. Thus, preventing cytoplasmic mis-localization and SG association of FUS could be neuroprotective (Y. R. Li et al. J Cell Biol 201, 361-372, 2013). The effect of 75 TRIMs were analyzed, including virtually all known human TRIMs (Table 3), on a green fluorescent protein fusion of FUS (GFP-FUS). Like endogenous FUS, GFP-FUS is predominantly in the nucleus under unstressed conditions. However, upon treatment with sodium arsenite, which elicits oxidative stress, FUS translocated to the cytoplasm and partitioned to G3BP1-positive SGs in nearly 95% of the treated cells (FIG. 48A and FIG. 48B).

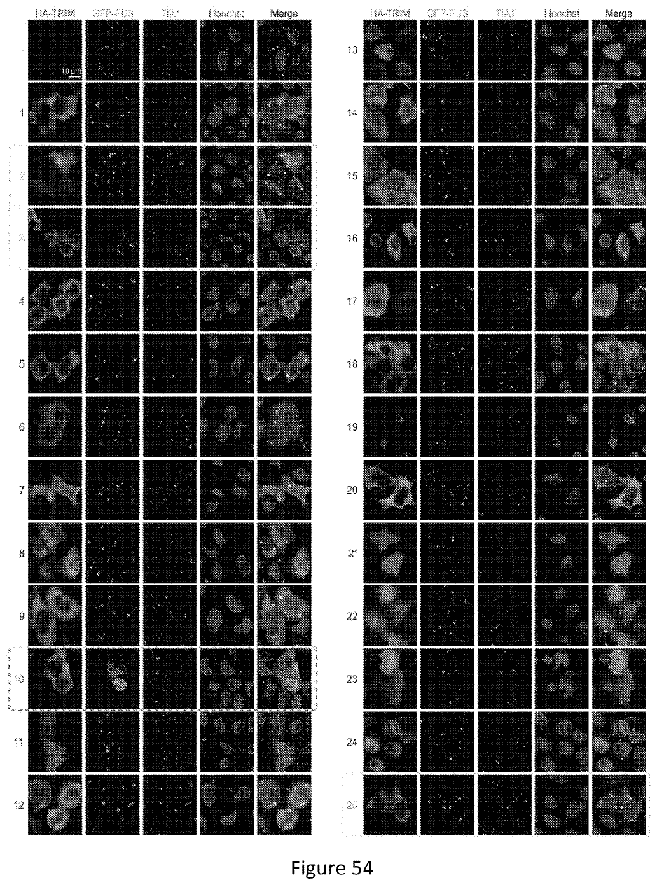

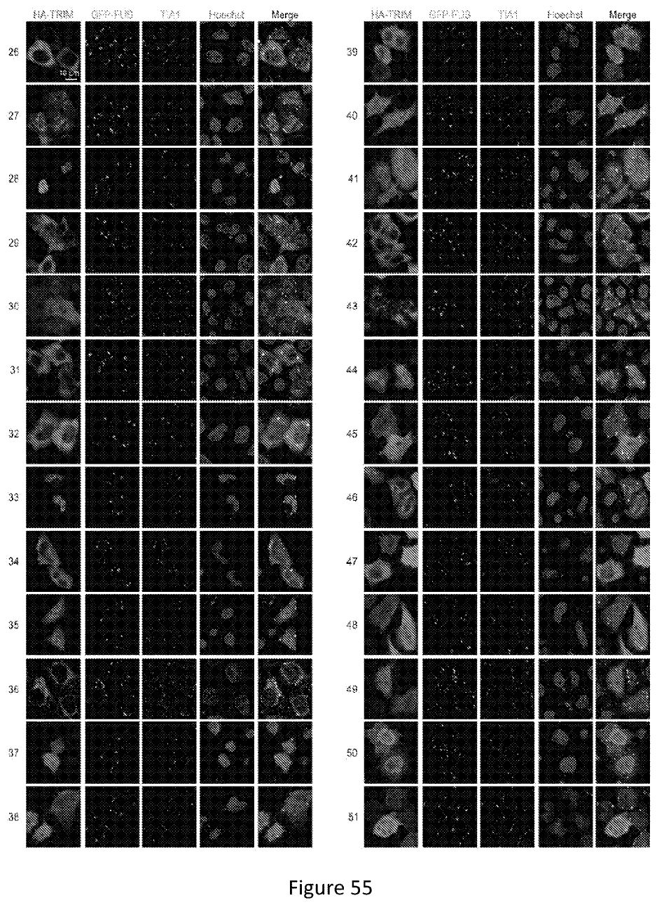

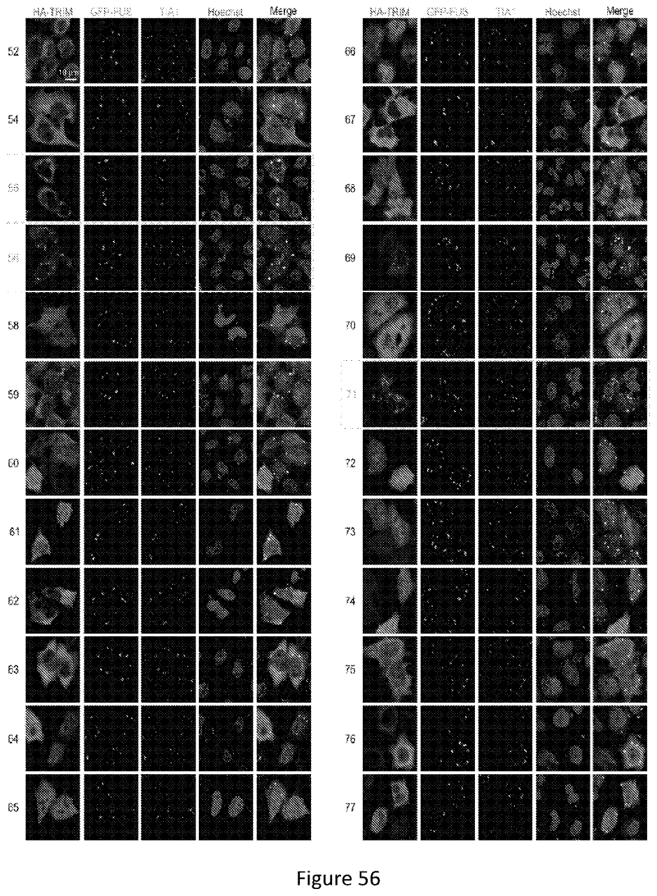

Upon co-expression with GFP-FUS in arsenite-treated HeLa cells, many TRIMs did not change their own localization. However, several TRIMs (i.e., TRIM2, 3, 25, 55, 56, and 71) became (partially) co-localized with GFP-FUS in G3BP1-positive SGs (FIG. 54 through FIG. 56).

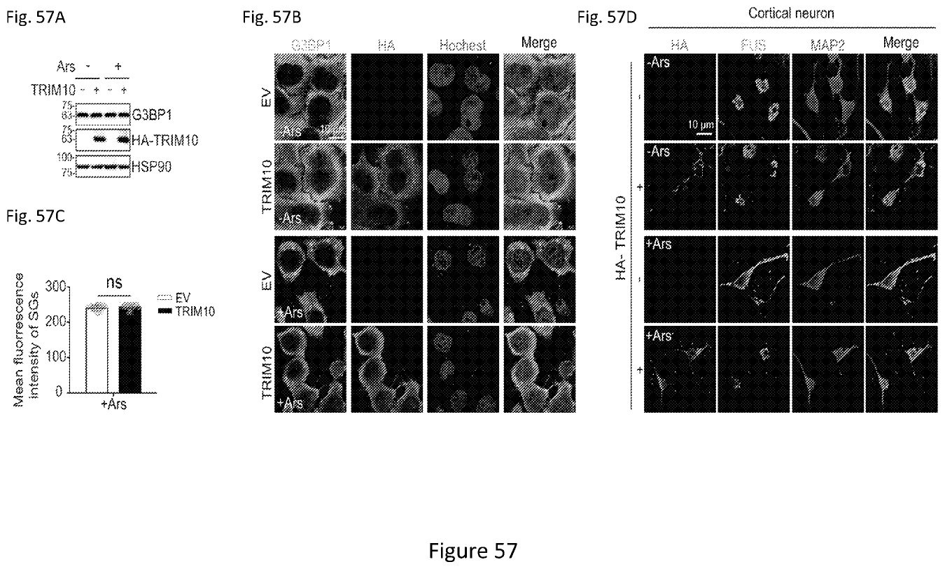

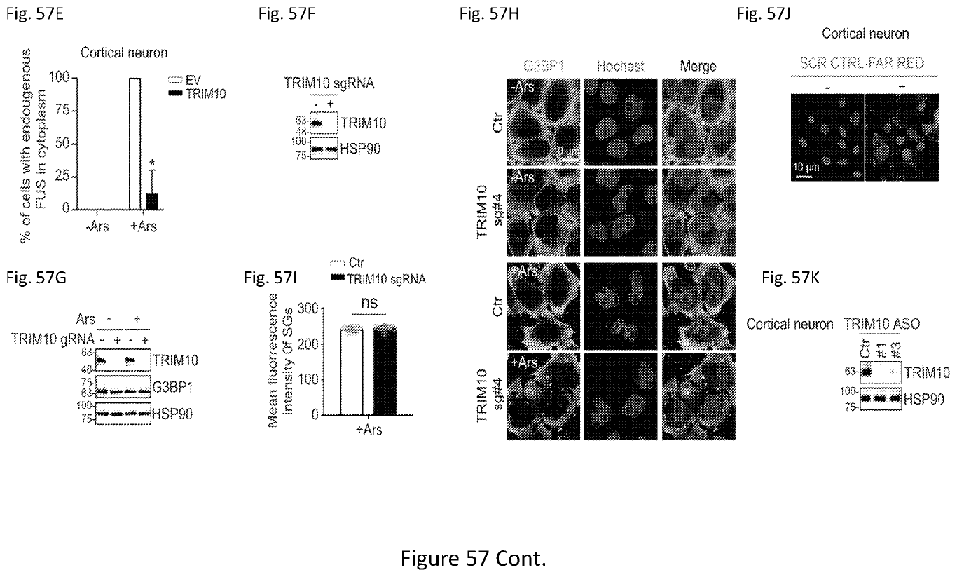

Virtually all the 75 TRIMs, including those that were accumulated SGs, were unable to prevent the cytoplasmic mis-localization of GFP-FUS in arsenite-treated cells (FIG. 54 through FIG. 56). However, TRIM10 was a notable exception. It reduced SG association and maintained nuclear localization of GFP-FUS (FIG. 48A through FIG. 48C). TRIM10 conferred this protective effect on GFP-FUS without altering its total levels (FIG. 48C), levels of the key SG component G3BP1 (FIG. 57A), or the biogenesis of SGs (FIG. 57B and FIG. 57C). In addition, TRIM10 nearly completely prevented endogenous FUS accumulating in SGs and maintained its nuclear localization (FIG. 48D and FIG. 48E). The potency of TRIM10 was comparable to that of karyopherin-β2 (Kapβ2, also called Transportin-1 or Importin-b2) (FIG. 48A and FIG. 48B), an NIR that effectively suppresses cytoplasmic mis-localization of FUS (L. Guo et al. Cell 173, 677-692 e620, 2018; T. Yoshizawa et al. Cell 173, 693-705 e622, 2018; M. Hofweber et al. Cell 173, 706-+, 2018; S. Qamar et al. Cell 173, 720-734 e715, 2018).

To extend these analyses, primary spinal cord and cortical neurons derived from wild-type mice were used. In these neurons, endogenous FUS exited the nucleus upon arsenite treatment, forming inclusions in the cell body and dendrites (FIG. 48F, FIG. 48G, FIG. 57D, and FIG. 57E). Elevating TRIM10 expression also prevented cytoplasmic aggregation of endogenous FUS and restricted it to the nucleus (FIG. 48F, FIG. 48G, FIG. 57D, and FIG. 57E).

To evaluate the effect of endogenous TRIM10, TRIM10 was knocked out in Hela cells by means of CRISPR-mediated genome editing (FIG. 57F). Knocking out TRIM10 noticeably increased the fraction of HeLa cells with GFP-FUS in SGs in the presence of arsenite (FIG. 48H and FIG. 48I), without affecting G3BP1 levels or SG biogenesis (FIG. 57G through FIG. 57I). Notably, knocking out TRIM10 also strongly promoted cytoplasmic mis-localization of both GFP-FUS and endogenous FUS even in the absence of any stress (FIG. 48H through FIG. 48K).

To downregulate endogenous TRIM10 in primary mouse neurons, antisense oligonucleotides (ASOs) composed of 2′-deoxy-2′fluoro-D-arabinonucleic acid (FANA) were generated (A. Kalota et al. Nucleic Acids Res 34, 451-461, 2006). These FANA-ASOs effectively entered cultured neurons and silenced Trim10 expression (FIG. 57J and FIG. 57K). This silencing led to cytoplasmic mis-localization of FUS in over 50% of cultured neurons (FIG. 48L and FIG. 48M). These results indicate that TRIM10 is critical for maintaining nuclear localization of FUS in transformed cell lines and primary neurons under both unstressed and stress conditions.

### TRIM10 Inhibits Cytoplasmic Mis-Localization of FUS Mutants

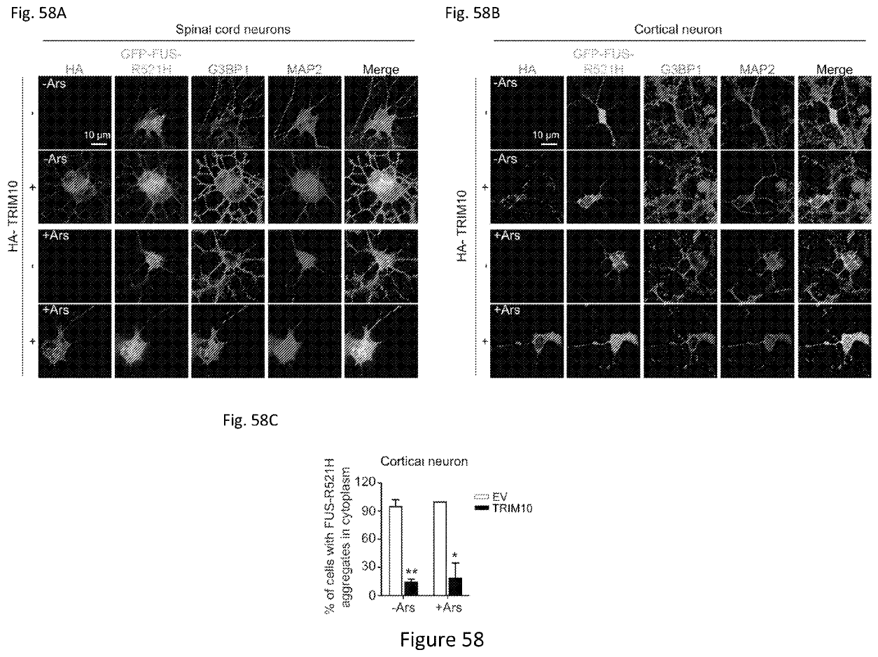

ALS/FTD-linked FUS variants map mainly in the PrLD and the non-classic proline-tyrosine NLS (PY-NLS) (A. F. Harrison et al. Biochem J 474, 1417-1438, 2017). The former tend to increase the intrinsic propensity of FUS to aggregate, while the latter lead to FUS accumulation in the cytoplasm, rendering it prone to aggregation as well. To evaluate the effect of TRIM10 on FUS mutants, G156E, a PrLD mutation that increases the tendency of FUS to aggregate, and R521H, a mutation in the PY-NLS were used (T. Nomura et al. J Biol Chem 289, 1192-1202, 2014; T. J. Kwiatkowski et al. Science 323, 1205-1208, 2009; C. Vance et al. Science 323, 1208-1211, 2009). GFP fusion of FUS (G156E) formed nuclear inclusion in unstressed HeLa cells and translocated to the cytoplasm and associated with SGs upon arsenite treatment (FIG. 48N and FIG. 48O) (T. Nomura et al. J Biol Chem 289, 1192-1202, 2014). TRIM10 noticeably reduced nuclear inclusions of GFP-FUS (G156E) in unstressed cells, and it also prevented cytoplasmic mis-localization of GFP-FUS (G156E) in arsenite-treated cells (FIG. 48N and FIG. 48O). In mouse primary spinal cord and cortical neurons, GFP-FUS (R521H) formed inclusions in the cell body and dendrite, in the presence or absence of arsenite. TRIM10 markedly reduced the formation of GFP-FUS (R521H) foci under both conditions (FIG. 48P, FIG. 48Q, and FIG. 58). Thus, TRIM10 also inhibits aggregation and SG association of disease-linked FUS variants.

TRIM10 Prevents SG Association of hnRNPA1

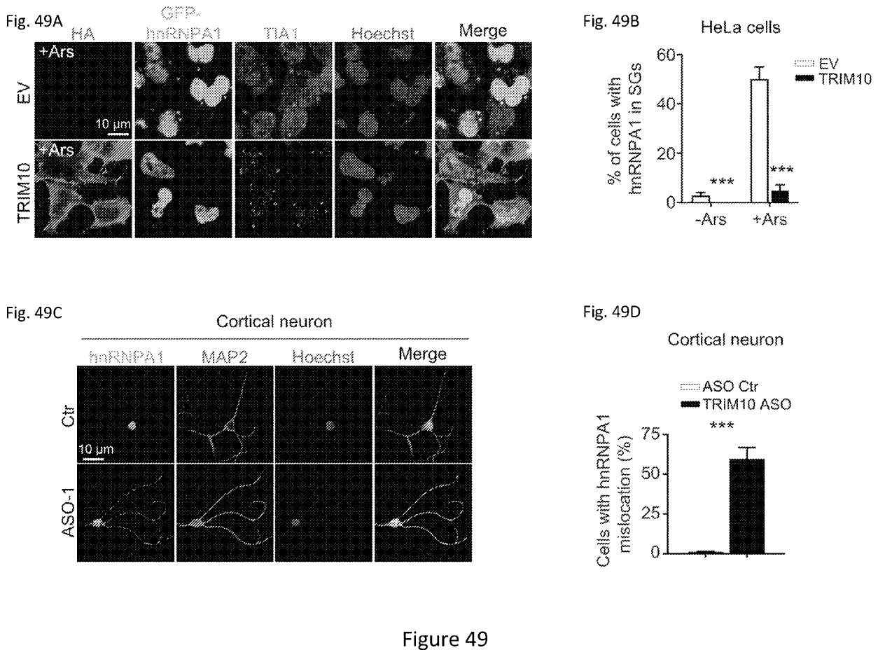

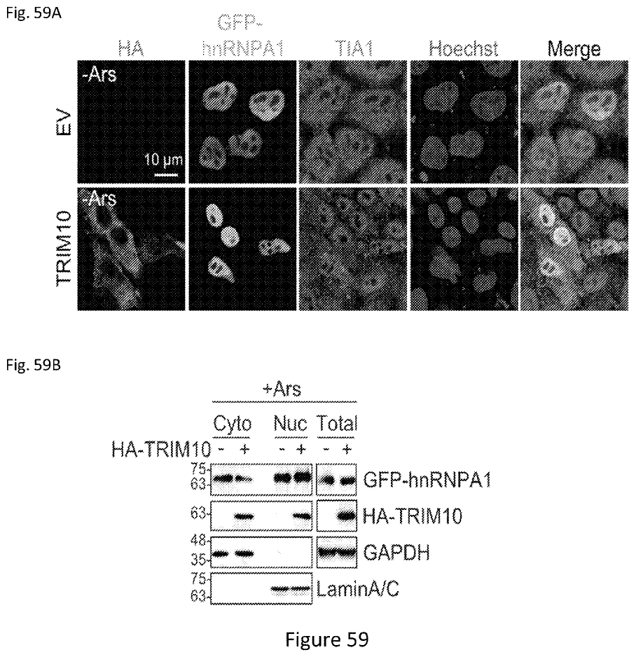

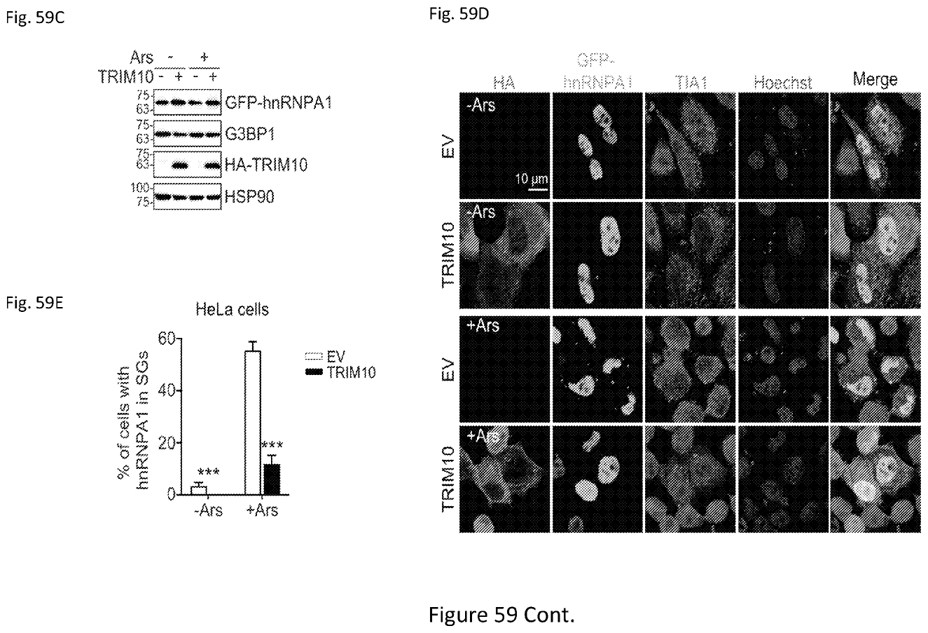

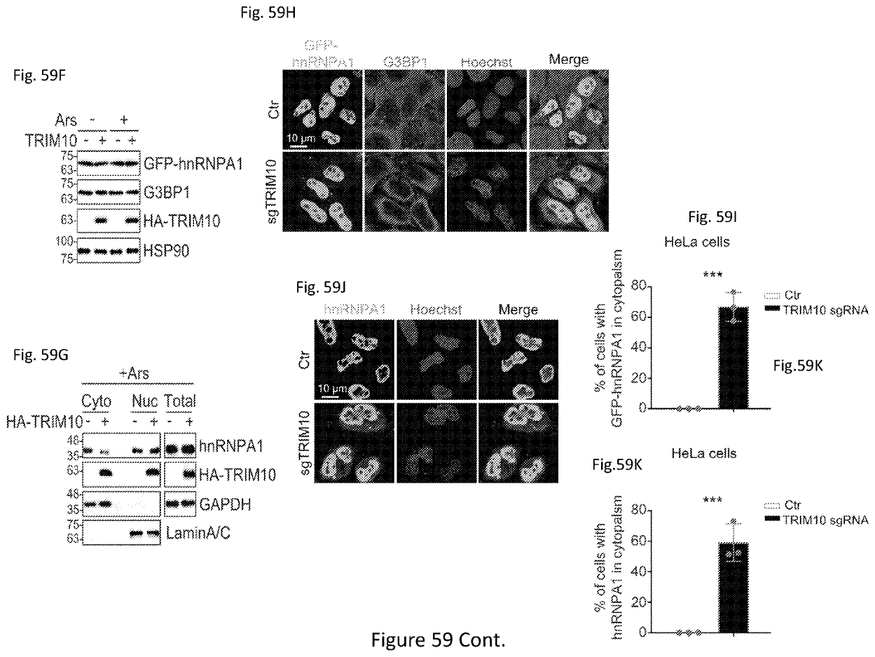

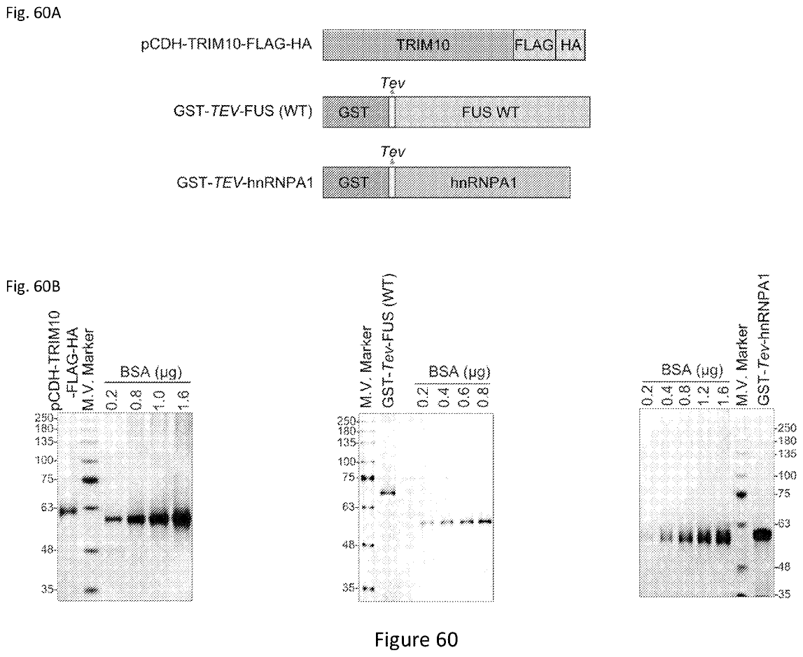

To evaluate whether TRIM10 can broadly regulate prion-like RBPs, the analyses was extended to hnRNPA1. Mutations in hnRNPA1 cause ALS as well as MSP, a pleiotropic degenerative disorder that affects muscle, bone, and brain (J. P. Taylor et al. Neurology 85, 658-660, 2015; H. J. Kim et al. Nature 495, 467-473, 2013). Like FUS, hnRNPA1 can be recruited to SGs under stress conditions (H. J. Kim et al. Nature 495, 467-473, 2013; S. Guil et al. Mol Cell Biol 26, 5744-5758, 2006). GFP-hnRNPA1, which was normally present in the nucleus, partially translocated to the cytosol upon arsenite treatment and coalesced into SGs (FIG. 49A, FIG. 49B, and FIG. 59A) (A. Molliex et al. Cell 163, 123-133, 2015; S. Guil et al. Mol Cell Biol 26, 5744-5758, 2006). Without altering the expression of GFP-hnRNPA1 or G3BP1 (FIG. 59B and FIG. 59C), TRIM10 potently prevented the coalescence of GFP-hnRNPA1 with SGs and restored its nuclear localization (FIG. 49A, FIG. 49B, FIG. 59A, and FIG. 59B). This protective effect of TRIM10 was evident even when it was introduced into cells after GFP-hnRNPA1 (FIG. 59D through FIG. 59F). Moreover, TRIM10 maintained nuclear localization of endogenous hnRNPA1 in arsenite-treated cells (FIG. 59G).

Conversely, knockout of TRIM10 increased cytoplasmic mis-localization of both GFP-hnRNPA1 (FIG. 59H and FIG. 59I) and endogenous hnRNPA1 (FIG. 59J and FIG. 59K). Silencing Trim10 expression in cortical neurons by ASO also led to cytoplasmic mis-localization and aggregation of endogenous hnRNPA1 (FIG. 49C and FIG. 49D). Thus, TRIM10 maintains solubility and nuclear localization of hnRNPA1.

### TRIM10 is a Molecular Chaperone for Prion-Like RBPs

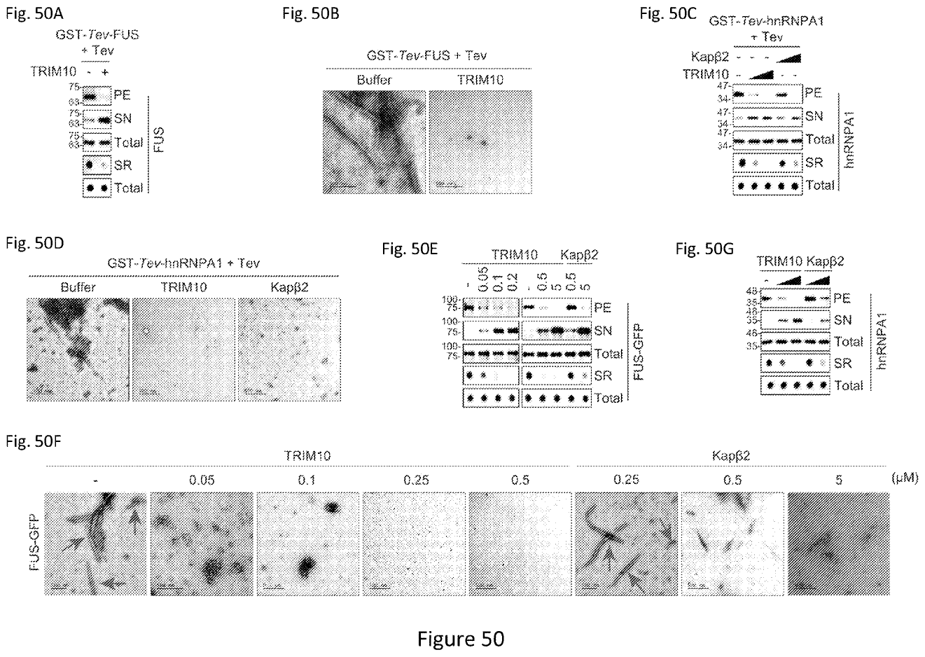

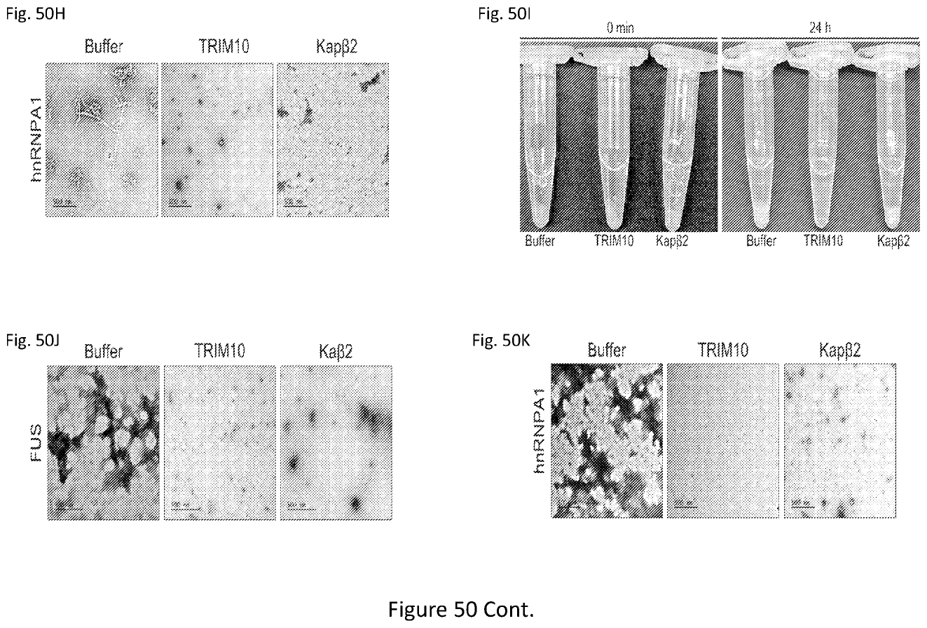

The ability of TRIM10 to protect against cytoplasmic mis-localization and aggregation of FUS and hnRNPA1 prompted us to examine whether it functions as a molecular chaperone for these prion-like RBPs. For this, the effect of TRIM10 on FUS and hnRNPA1 under conditions where aggregation, rather than phase separation, is the dominant pathway for these RBPs, were examined. GST-Tev-FUS and GST-Tev-hnRNPA1 (FIG. 60A and FIG. 60B) were used (L. Guo et al. Cell 173, 677-692 e620, 2018). When the GST tag was selectively removed by TEV protease, FUS rapidly assembled into aggregates without dwelling in the state of microscopically visible liquid phases (L. Guo et al. Cell 173, 677-692 e620, 2018). These aggregates included PE and SR aggregates (FIG. 50A) and mature fibrils (FIG. 50B). TRIM10 effectively prevented the formation of various FUS aggregates at a sub-stoichiometric ratio to FUS (~1:10), keeping virtually all FUS molecules in a soluble state (FIG. 50A and FIG. 50B).

Similarly, upon separation from the GST tag, hnRNPA1 produced PE and SR aggregates and amyloid fibrils (FIG. 50C and FIG. 50D) (L. Guo et al. Cell 173, 677-692 e620, 2018). At a ~1:10 molar ratio to hnRNPA1, TRIM10 nearly completely prevented the formation of various hnRNPA1 aggregates (FIG. 50C and FIG. 50D). In comparison, the chaperone activity of Kapβ2 was evident only when it was present at an approximately equal molar ratio to hnRNPA1 (FIG. 50C and FIG. 50D). Unlike canonical molecular chaperones such as HSP60 and HSP70, which are multi-component systems driven by energy derived from ATP hydrolysis, TRIM10 averts FUS and hnRNPA1 aggregation by itself, in the absence of any other protein cofactors or ATP (FIG. 50A through FIG. 50D) (S. Wolff et al. Cell 157, 52-64, 2014; D. Balchin et al. Science 353, aac4354, 2016). These results indicate that TRIM10 is a potent molecular chaperone for FUS and hnRNPA1, and that it operates in an ATP-independent manner.

### TRIM10 is a Disaggregase for Prion-Like RBPs

Can TRIM10 itself dissolve pre-existing prion-like RBP aggregates at a low sub-stoichiometric ratio, hence acting as a single-component catalyst for protein disaggregation? To answer this question, pre-existing FUS and hnRNPA1 aggregates were treated with TRIM10 at various molar ratios (based on monomer concentrations). TRIM10 could disassemble pre-existing SR and PE aggregates (FIG. 50E) and fibrils (FIG. 50F) of FUS. Of note, TRIM10 dissolved a substantial fraction of these aggregates at merely 1:100 molar ratio to FUS and dissolved virtually all aggregates at a 1:10 molar ratio (FIG. 50E and FIG. 50F). Likewise, TRIM10 effectively dissolved various forms of pre-existing hnRNPA1 aggregates and returned them to a soluble state at a 1:10 molar ratio (FIG. 50G and FIG. 50H). In contrast, the dissolving activity of Kapβ2 was detectable only when it was present at an approximately same concentration as its client protein (FIG. 50G and FIG. 50H), as expected (L. Guo et al. Cell 173, 677-692 e620, 2018; T. Yoshizawa et al. Cell 173, 693-705 e622, 2018; M. Hofweber et al. Cell 173, 706-+, 2018; S. Qamar et al. Cell 173, 720-734 e715, 2018).

Next, the effect of TRIM10 on preformed FUS and hnRNPA1 hydrogels was tested. At a 1:12 molar ratio to FUS, TRIM10 nearly completely dissolved the stable FUS hydrogels by dismantling the underlying fibrillar matrix (FIG. 50I and FIG. 50J). Similarly, TRIM10 nearly completely dissolve preformed hnRNPA1 hydrogels and disrupted the underlying fibril network. This effect was especially potent, as TRIM10 was present at merely a ~1:83 molar ratio to hnRNPA1 (FIG. 50K). In comparison, it was previously shown that Kapβ2 needs to be present at a much higher molar ratio (~1:2 to FUS and ~1:7.7 to hnRNPA1) to completely dissolve these hydrogels (L. Guo et al. Cell 173, 677-692 e620, 2018). Consistently, it was found that Kapβ2 showed only moderate activity at a ~1:4.5 molar ratio to FUS (FIG. 50I and FIG. 50J) and at a ~1:6 molar ratio to hnRNPA1 (FIG. 50K) (L. Guo et al. Cell 173, 677-692 e620, 2018). As for aggregation prevention, TRIM10 dissolved FUS and hnRNPA1 aggregates by itself independently of ATP. These results indicate that TRIM10 is a catalytic disaggregase for FUS and hnRNPA1.

### Outlook

Prion-like RBPs organize diverse membrane-less organelles and play a versatile role in DNA and RNA metabolism; however, they frequently mis-localize and aggregate in patients suffering from degenerative diseases. Here, it was shown that TRIMs provide an effective quality control mechanism for prion-like RBPs including TDP-43, FUS, and hnRNPA1. TRIMs prevent cytoplasmic mis-localization and SG association of these prion-like RBPs and preserve their nuclear localization, solubility, and function, both in unstressed cells and in cells experiencing strong oxidative stress. TRIMs also prevent the assembly of these RBPs into solids in monodisperse solution, and virtually completely dissolve pre-existing RBP aggregates and hydrogels. When tested, TRIMs exert these potent protective effects at a very low sub-stoichiometric molar ratio to prion-like RBPs. Thus, TRIMs likely represent a new type of powerful modulators for prion-like RBPs. The current work, along with previous studies demonstrating the protective effect of TRIMs on other misfolding-prone proteins such as tau, a-synuclein, and polyglutamine proteins, emphasizes the importance of the TRIM system in contending with proteins aggregation and preserving their function in human cells. Without being bound by theory, the results herein demonstrate that the TRIM system should be exploited for the development of therapies to address the root cause of these incurable and fatal protein misfolding diseases.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition for treating or preventing a disease or disorder associated with aggregation of one or more selected from the group consisting of tau, α-Synuclein (α-Syn), superoxide dismutase 1 (SOD1), TAR DNA binding protein 43 (TDP-43), FUsed in Sarcoma/Translocated in LipoSarcoma (FUS/TLS), ataxin 1, huntingtin (Htt), Aβ42, and heterogeneous ribonucleoprotein A1 (hnRNPA1), the composition comprising an activator of the level or activity of one or more tripartite motif (TRIM) proteins
wherein the one or more TRIM proteins are one or more selected from the group consisting of human TRIM10, TRIM2, TRIM3, TRIM4, TRIM5, TRIM9, TRIM11, TRIM17, TRIM18, TRIM19, TRIM21, TRIM24, TRIM26, TRIM29, TRIM30, TRIM31, TRIM34, TRIM36, TRIM37, TRIM39, TRIM40, TRIM41, TRIM42, TRIM43, TRIM46, TRIM47, TRIM48, TRIM49, TRIM52, TRIM54, TRIM55, TRIM56, TRIM58, TRIM63, TRIM64, TRIM65, TRIM68, TRIM69, TRIM70, TRIM71, TRIM73, and TRIM77.

2. The composition of claim 1, wherein the activator is one or more selected from the group consisting of a chemical compound, a protein, a peptide, a peptidomimetic, an antibody, a ribozyme, a small molecule chemical compound, a nucleic acid, a vector, an antisense nucleic acid, siRNA, shRNA, and a guide RNA.

3. The composition of claim 1, wherein the disease or disorder is associated with aggregation of tau; and
wherein the one or more TRIM proteins are selected from the group consisting of TRIM10, TRIM2, TRIM3, TRIM4, TRIM5, TRIM9, TRIM11, TRIM12, TRIM17, TRIM18, TRIM19, TRIM21, TRIM26, TRIM29, TRIM30, TRIM31, TRIM34, TRIM36, TRIM39, TRIM40, TRIM42, TRIM43, TRIM46, TRIM47, TRIM48, TRIM49, TRIM52, TRIM54, TRIM55, TRIM58, TRIM63, TRIM64, TRIM65, TRIM68, TRIM69 and TRIM70.

4. The composition of claim 1, wherein the disease or disorder is associated with aggregation of α-Syn; and
wherein the one or more TRIM proteins are selected from the group consisting of TRIM10, TRIM2, TRIM3, TRIM17, TRIM18, TRIM19, TRIM26, TRIM29, TRIM30, TRIM31, TRIM36, TRIM41, TRIM42, TRIM43, TRIM46, TRIM49, TRIM55, TRIM56, TRIM63, TRIM64, TRIM68, TRIM69, TRIM70, TRIM71, and TRIM73.

5. The composition of claim 1, wherein the disease or disorder is associated with aggregation of SOD1; and
wherein the one or more TRIM proteins are selected from the group consisting of TRIM10, TRIM11, TRIM24, TRIM36, and TRIM58.

6. The composition of claim 1, wherein the disease or disorder is associated with aggregation of TDP-43; and
wherein the one or more TRIM proteins are selected from the group consisting of TRIM10, TRIM11, TRIM17, TRIM36, TRIM 37, TRIM 40, TRIM49, and TRIM55.

7. The composition of claim 1, wherein the disease or disorder is associated with aggregation of FUS/TLS, ataxin 1, Htt, Aβ42, and hnRNPA1; and
wherein the one or more TRIM protein is TRIM10.

8. The composition of claim 1, wherein the activator of the TRIM protein is:
a peptide comprising the amino acid sequence of the TRIM protein or a functional variant thereof;
a nucleic acid encoding the TRIM protein or a functional variant thereof; or
a vector comprising a nucleic acid encoding the TRIM protein or a functional variant thereof.

9-10. (canceled)

11. The composition of claim 8, wherein the vector is a virus.

12. The composition of claim 11, wherein the virus is an adeno-associated virus.

13. A method of treating or preventing a neurodegenerative disease or disorder associated with aggregation of one or more proteins selected from the group consisting of tau, α-Syn, SOD1, TDP-43, FUS/TLS, ataxin 1, Htt, Aβ42, and hnRNPA1 comprising administering to the subject a composition according to claim 1.

14. The method of claim 13, wherein the neurodegenerative disease or disorder associated with tau is selected from the group consisting of Alzheimer's disease, frontotemporal lobar degeneration (FTLD-tau), progressive supranuclear palsy (PSP), corticobasal degeneration (CBD), argyrophilic grain disease (AGD), frontotemporal dementia and parkinsonism linked to chromosome 17 (FTDP-17), vacuolar tauopathy, Lytico-bodig disease, globular glial tauopathy (GGT), ageing-related tau astrogliopathy (ARTAG), Pick's disease, and amyotrophic lateral sclerosis (ALS), primary age-related tauopathy (PART), tangle only dementia (TOD), chronic traumatic encephalopathy (CTE), anti-IgLON5-related tauopathy, Guadeloupean parkinsonism, multisystem proteinopathy (MSP) Nodding Syndrome (NS), ganglioglioma, gangliocytoma, meningioangiomatosis, postencephalitic parkinsonism, subacute sclerosing panencephalitis (SSPE), lead encephalopathy, tuberous sclerosis, pantothenate kinase-associated neurodegeneration, and lipofuscinosis; and
wherein the activator of the one or more TRIM proteins is an activator of one or more selected from the group consisting of TRIM10, TRIM11, and TRIM55.

15. The method of claim 14, wherein administering the composition is effective in one or more selected from the group consisting of:
a) reducing tau aggregates by at least about 60%;
b) reducing the ratio of insoluble tau to soluble tau by at least about 50%; and
c) reducing tau aggregates by about 90% six to eight days after administration of the composition.

16. The method of claim 13, wherein the neurodegenerative disease or disorder associated with α-Syn is selected from the group consisting of Parkinson's disease (PD), dementia with Lewy bodies (DLB), multiple system atrophy (MSA), Shy-Drager syndrome, striatonigral degeneration, olivopontocerebellar atrophy, Hallervorden-Spatz syndrome, REM sleep behavior disorder (RPD), and Alzheimer's disease with amygdala restricted Lewy bodies (AD/ALB); and
wherein the activator of one or more TRIM proteins is an activator of one or more selected from the group consisting of TRIM10, TRIM36, TRIM55, and TRIM68.

17. The method of claim 13, wherein the neurodegenerative disease or disorder associated with SOD1 is selected from the group consisting of amyotrophic lateral sclerosis (ALS) and Parkinson's disease (PD); and
wherein the activator of one or more TRIM proteins is an activator of one or more selected from the group consisting of TRIM10, TRIM11, TRIM24, TRIM36, and TRIM58.

18. The method of claim 13, wherein the neurodegenerative disease or disorder associated with TDP-43 is selected from the group consisting of amyotrophic lateral sclerosis (ALS), frontotemporal dementia (FTD), frontotemporal lobar degeneration (FTLD-TDP), multiple system proteinopathy (MSP), Perry disease, facial onset sensory and motor neuronopathy (FOSMN), Alzheimer's disease (AD), cerebral age-related TDP-43 with sclerosis (CARTS), limbic-predominant age-related TDP-43 encephalopathy (LATE), sporadic inclusion body myositis (sIBM), chronic traumatic encephalopathy (CTE), primary lateral sclerosis (PLS), progressive muscular atrophy (PMA), Guam Parkinson-dementia complex (G-PDC), Guam amyotrophic lateral sclerosis (G-ALS); Parkinson's disease (PD), and Huntington's disease (HD); and
wherein the activator of one or more TRIM proteins is an activator of one or more selected from the group consisting of TRIM10, TRIM11, TRIM17, TRIM36, TRIM37, TRIM40, TRIM49, and TRIM55.

19. The method of claim 13, wherein the composition is administered to the subject in their cerebrospinal fluid (CSF).

20. The method of claim 19, wherein the composition is administered by intracerebroventricular (ICV) injection.

21. The method of claim 13, wherein the composition is administered to the subject before onset of symptoms of the disease or disorder or after onset of symptoms of the disease or disorder.

22. (canceled)

23. The method of claim 13, wherein the method further comprises administering one or more additional therapeutic agents.


SEQUENCE LISTING (37 sequences)

SEQ ID NO: 1 - DNA, 25 bp, synthetic construct
  source 1..25: mol_type = other DNA
ggttaatcgcgtataatacgcgtat

SEQ ID NO: 2 - DNA, 20 bp, synthetic construct
  source 1..20: mol_type = other DNA
ggcagttgacttcatctgcc

SEQ ID NO: 3 - DNA, 20 bp, synthetic construct
  source 1..20: mol_type = other DNA
gagccagcggcagaacgtgc

SEQ ID NO: 4 - DNA, 20 bp, synthetic construct
  source 1..20: mol_type = other DNA
gccgctcaatgttctccacc

SEQ ID NO: 5 - DNA, 20 bp, synthetic construct
  source 1..20: mol_type = other DNA
taccattaagaatatcgaaa

SEQ ID NO: 6 - DNA, 20 bp, synthetic construct
  source 1..20: mol_type = other DNA
aacccgtatttgcccacaag

SEQ ID NO: 7 - DNA, 21 bp, synthetic construct
  source 1..21: mol_type = other DNA
ataaacagcagcgacccatcc

SEQ ID NO: 8 - DNA, 21 bp, synthetic construct
  source 1..21: mol_type = other DNA
acttagtgctttggtgagagc

SEQ ID NO: 9 - DNA, 21 bp, synthetic construct
  source 1..21: mol_type = other DNA
actgtagaatgagagatggcc

SEQ ID NO: 10 - DNA, 21 bp, synthetic construct
  source 1..21: mol_type = other DNA
tagaatgagagatggccagct

SEQ ID NO: 11 - DNA, 21 bp, synthetic construct
  source 1..21: mol_type = other DNA
atttgtttccgtaggtgctcc

SEQ ID NO: 12 - DNA, 20 bp, synthetic construct
  source 1..20: mol_type = other DNA
ccttccctgaaggttcctcc

SEQ ID NO: 13 - DNA, 28 bp, synthetic construct
  source 1..28: mol_type = other DNA
gaggcgggaaggtgcagataattaataa

SEQ ID NO: 14 - DNA, 22 bp, synthetic construct
  source 1..22: mol_type = other DNA
ctggtttatgatggatgttgcc

SEQ ID NO: 15 - DNA, 20 bp, synthetic construct
  source 1..20: mol_type = other DNA
ctgccccatctgtcagggta

SEQ ID NO: 16 - DNA, 22 bp, synthetic construct
  source 1..22: mol_type = other DNA
ggtatctcacagtagcgggtaa

SEQ ID NO: 17 - DNA, 21 bp, synthetic construct
  source 1..21: mol_type = other DNA
tactgggaggtggaggttggg

SEQ ID NO: 18 - DNA, 24 bp, synthetic construct
  source 1..24: mol_type = other DNA
ggatctcgggaaagatgaatagca

SEQ ID NO: 19 - DNA, 21 bp, synthetic construct
  source 1..21: mol_type = other DNA
tgcactactactgtgaggacg

SEQ ID NO: 20 - DNA, 21 bp, synthetic construct
  source 1..21: mol_type = other DNA
tccttagggtactcaggtggt

SEQ ID NO: 21 - DNA, 21 bp, synthetic construct
  source 1..21: mol_type = other DNA
gagctgtttacccacccattg

SEQ ID NO: 22 - DNA, 22 bp, synthetic construct
  source 1..22: mol_type = other DNA
ctgatcccacatcgttgaatga

SEQ ID NO: 23 - DNA, 21 bp, synthetic construct
  source 1..21: mol_type = other DNA
ttgtcagcacaacctgtgtag

SEQ ID NO: 24 - DNA, 23 bp, synthetic construct
  source 1..23: mol_type = other DNA
cccatgtctatccaaaaccactt

SEQ ID NO: 25 - DNA, 19 bp, synthetic construct
  source 1..19: mol_type = other DNA
gctaaggctgtgggcaagg

SEQ ID NO: 26 - DNA, 19 bp, synthetic construct
  source 1..19: mol_type = other DNA
ggaggagtgggtgtcgctg

SEQ ID NO: 27 - DNA, 20 bp, synthetic construct
  source 1..20: mol_type = other DNA
ggcagttgacttcatctgcc

SEQ ID NO: 28 - DNA, 21 bp, synthetic construct
  source 1..21: mol_type = other DNA
tgagatcacctgttgcctctt

SEQ ID NO: 29 - DNA, 21 bp, synthetic construct
  source 1..21: mol_type = other DNA
tagtcaagagatacctgcacc

SEQ ID NO: 30 - DNA, 48 bp, synthetic construct
  source 1..48: mol_type = other DNA
attgaggagcagcagagaagttggagtgaaggcagagaggggttaagg

SEQ ID NO: 31 - DNA, 46 bp, synthetic construct
  source 1..46: mol_type = other DNA
tagggacttagggttctctctagggacttagggttctctctaggga

SEQ ID NO: 32 - DNA, 20 bp, synthetic construct
  source 1..20: mol_type = other DNA
ggaactcagtcaactcccca

SEQ ID NO: 33 - DNA, 20 bp, synthetic construct
  source 1..20: mol_type = other DNA
taccgtaacttcccgaggtg

SEQ ID NO: 34 - DNA, 20 bp, synthetic construct
  source 1..20: mol_type = other DNA
ctgccccatctgtcagggta

SEQ ID NO: 35 - DNA, 22 bp, synthetic construct
  source 1..22: mol_type = other DNA
ggtatctcacagtagcgggtaa

SEQ ID NO: 36 - DNA, 19 bp, synthetic construct
  source 1..19: mol_type = other DNA
gctaaggctgtgggcaagg

SEQ ID NO: 37 - DNA, 19 bp, synthetic construct
  source 1..19: mol_type = other DNA
ggaggagtgggtgtcgctg